(19) United States
(12) Patent Application Publication
(10) Pub. No.: US 2024/0024509 A1
(43) Pub. Date: Jan. 25, 2024

(54) COMPOSITIONS AND METHODS FOR TREATING CEREBROSPINAL FLUID DISORDERS

(71) Applicant: The Children's Medical Center Corporation, Boston, MA (US)

(72) Inventors: Maria K. LEHTINEN, Boston, MA (US); Ryann M. FAME, Boston, MA (US); Cameron SADEGH, Boston, MA (US); Huixin XU, Boston, MA (US)

(73) Assignee: The Children's Medical Center Corporation, Boston, MA (US)

(21) Appl. No.: 18/360,505

(22) Filed: Jul. 27, 2023

Related U.S. Application Data
(63) Continuation of application No. PCT/US2021/015863, filed on Jan. 29, 2021.

(51) Int. Cl. A61K 48/00; C07K 14/705; C12N 15/86

(52) U.S. Cl. CPC A61K 48/005; C07K 14/705; C12N 15/86; A61K 48/0075; C12N 2750/14143

(57) ABSTRACT

Provided are methods and compositions for treating cerebrospinal fluid disorders. In embodiments, the methods comprise administering an agent to a subject altering activity, expression, or level of NKCC1 in a cell.

### CROSS-REFERENCE TO RELATED APPLICATION

This application is a continuation under 35 U.S.C. § 111(a) of PCT International Patent Application No. PCT/US2021/015863, filed Jan. 29, 2021, designating the United States and published in English, the entire contents of which are incorporated by reference herein.

### STATEMENT OF RIGHTS TO INVENTIONS MADE UNDER FEDERALLY SPONSORED RESEARCH

This invention was made with government support under Grant No. HL 110852, NS088566, and RF1DA048790 awarded by the National Institutes of Health. The government has certain rights in the invention.

### SEQUENCE LISTING

The present application contains a Sequence Listing which has been submitted electronically in XML format following conversion from the originally filed TXT format.

The content of the electronic XML Sequence Listing, (Date of creation: Jul. 26, 2023; Size: 10,646 bytes; Name: 167705-026001US-Sequence_Listing.xml), and the original TXT format, is herein incorporated by reference in its entirety.

### BACKGROUND OF THE INVENTION

A balance between cerebrospinal fluid (CSF) production and clearance (influx/efflux) is essential for normal brain function and development. Disrupted cerebrospinal fluid (CSF) volume homeostasis with excessive cerebrospinal fluid (CSF) accumulation is implicated in pediatric and adult brain disorders. In congenital hydrocephalus, pediatric patients suffer from a potentially life-threatening accumulation of cerebrospinal fluid (CSF). Children with excessive cerebrospinal fluid frequently develop neurological deficits that last through childhood and into adult life.

Intraventricular hemorrhage (IVH) resulting from hemorrhagic stroke is a common cause of post-hemorrhagic hydrocephalus (PHH), an excessive accumulation of cerebrospinal fluid (CSF) accompanied by neurologic decline. Accounting for nearly a quarter of cases, intraventricular hemorrhage (IVH) is the leading etiology of pediatric hydrocephalus in North America and represents one of the most devastating complications of preterm birth. Approximately 20% of infants with intraventricular hemorrhage (IVH) develop permanent disruptions of cerebrospinal fluid (CSF) homeostasis and require repeated neurosurgical interventions throughout life, which encumbers their families and strains the healthcare system. Over 80% of severe cases also develop cerebral palsy, epilepsy, or other neurologic impairments. Adults are similarly afflicted by post-hemorrhagic hydrocephalus (PHH) following hemorrhagic stroke complicated by intraventricular hemorrhage (IVH), with modern rates of adult post-hemorrhagic hydrocephalus (PHH) also approximating 20%.

Treatments for cerebrospinal fluid imbalances include surgical insertion of a shunt in the brain. Such treatments lead to complications including recurrent infections. Such treatments also often lead to the need for multiple surgeries during the span of a patient's life.

Thus, there is a need for improved compositions and methods for treating cerebrospinal fluid disorders. involving cerebrospinal fluid (CSF) dysregulation in pediatric and adult patients.

### SUMMARY OF THE INVENTION

As described below, the present invention features methods and compositions for treating cerebrospinal fluid disorders. In embodiments, the methods comprise administering an agent to a subject altering activity, expression, or level of NKCC1 in a cell.

In one aspect, the invention features a method for restoring cerebrospinal fluid ionic homeostasis in a subject. The method involves administering to a subject in need thereof an agent that increases NKCC1 activity, expression, or level in a cell, thereby restoring cerebrospinal fluid ionic homeostasis in the subject.

In one aspect, the invention features a method for mitigating an intracranial fluid imbalance associated with a hemorrhage in a subject. The method involves administering to a subject in need thereof an agent that increases NKCC1 activity, expression, or level in a cell, thereby mitigating the intracranial fluid imbalance.

In one aspect, the invention features a method for treating a cerebrospinal fluid disorder in a subject. The method involves administering to a subject in need thereof an agent that increases NKCC1 activity, expression, or level in a cell, thereby treating the cerebrospinal fluid disorder.

In one aspect, the invention features a method for treating a subject having or having a propensity to develop an intraventricular hemorrhage. The method involves administering to the subject an adeno-associated virus (AAV) vector containing a polynucleotide encoding an NKCC1 polypeptide, thereby increasing NKCC1 polypeptide expression levels in choroid plexus epithelial cells in the subject and reducing an intracranial fluid imbalance associated with the intraventricular hemorrhage. In embodiments, the AAV vector is an AAV 2/5 vector.

In one aspect, the invention features a kit for use in the method of any one of the above aspects, where the kit contains the agent that increases NKCC1 activity, expression, or level in a cell.

In any of the above aspects, the cell is a choroid plexus cell. In any of the above aspects, the cell is an epithelial cell.

In any of the above aspects, the agent contains a polypeptide, polynucleotide, or small compound. In any of the above aspects, the agent comprises an adeno-associated virus (AAV) vector. In embodiments, AAV vector is an AAV2/5 vector contains an NKCC1 polynucleotide.

In any of the above aspects, the agent contains a polynucleotide encoding NKCC1. In embodiments, the polynucleotide contains mRNA.

In any of the above aspects, the agent contains a small-molecule that increases expression, activity, or level of NKCC1. In embodiments, the small molecule is associate with an increase in NKCC1 phosphorylation.

In any of the above aspects, the cerebrospinal fluid disorder is associated with a congenital disorder, a trauma, or an ischemic event. In any of the above aspects, the cerebrospinal fluid disorder is hydrocephalus, hyperkalirrhachia, or ventriculomegaly. In any of the above aspects, the subject has or has propensity to develop an intraventricular hemorrhage.

In embodiments, the agent is first administered to the patient within hour or days of the intraventricular hemorrhage. In embodiments, the intraventricular hemorrhage is grade 2, 3, or 4. In any of the above aspects, the subject has a loss-of-function CHD4 mutation. In any of the above aspects, the subject has or has a propensity to develop hyperkalirrhachia. In any of the above aspects, the subject has or has a propensity to develop hydrocephalus. In any of the above aspects, the subject has or has a propensity to develop a brain aneurysm or a stroke. In any of the above aspects, the subject is a mammal. In any of the above aspects, the subject is a human. In any of the above aspects, the subject is a pediatric subject. In any of the above aspects, the subject is an adult. In any of the above aspects, the subject is prenatal. In any of the above aspects, the subject is neonatal. In any of the above aspects, the subject is treated during the first postnatal month.

In any of the above aspects, the agent is administered by intracerebroventricular injection.

In any of the above aspects, the method further involves installing a shunt, optionally a ventriculoperitoneal shunt, in the subject, performing a ventriculostomy, optionally an endoscopic third ventriculostomy, on the subject, and/or puncturing the lumbar.

In any of the above aspects, administration of the agent is associated with a decrease in cerebrospinal fluid levels or an increase in cerebrospinal fluid clearance. In any of the above aspects, administration of the agent is associated with a decrease in intracranial pressure. In any of the above aspects, administration of the agent is associated with a decrease in ventricle size.

In any of the above aspects, administration of the agent is associated with an increase in cerebrospinal fluid clearance capacity. In any of the above aspects, administration of the agent is associated with a reduction in need for permanent shunting.

In any of the above aspects, the increase in NKCC1 expression, activity, or level is associated with a reduction in incidence or severity of hydrocephalus and/or ventriculomegaly.

In any of the above aspects, the increase in NKCC1 expression, activity, or level is associated with an increase in cerebrospinal fluid K⁺ clearance, increased cerebrospinal compliance, and reduced circulating cerebrospinal fluid in the brain. In any of the above aspects, intracranial pressure is reduced or remains unchanged following the increase in NKCC1 expression, activity, or level.

In one aspect, the invention features a cell containing the vector of any one of the above aspects or the polynucleotide of any one of the above aspects. In embodiments, the cell is a choroid plexus epithelial cell.

In one aspect, the invention features a vector containing a polynucleotide encoding NKCC1. In embodiments, the vector is an adeno-associated virus vector. In embodiments, the AAV vector is an AAV2/5 vector.

The invention provides methods and compositions for treating cerebrospinal fluid disorders. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “hydrocephalus” is meant a condition associated with an increase in fluid in the brain. In some embodiments, the excess fluid increases the size of ventricles (cavities) and puts pressure on the brain. It is within the skill of a trained medical professional to diagnose hydrocephalus. Methods for diagnosing hydrocephalus include, but are not limited to, carrying out neurological exams to characterize alterations in muscle strength, reflexes, coordination, balance, vision, eye movement, hearing, cognition, or mood. Other methods for diagnosing hydrocephalus include imaging studies (e.g., ultrasound, magnetic resonance imaging, and computed tomography. Other tests that might assist in diagnosing or characterizing hydrocephalus include, but are not limited to, spinal tap (lumbar puncture), intracranial pressure monitoring, and fundoscopic examination.

By “ventriculomegaly” is meant a condition in which ventricles are larger than normal. In various embodiments, the ventricles are larger than normal on an ultrasound. In various embodiments, the ultrasound is a prenatal ultrasound or a neonatal ultrasound. It is within the skill of a trained medical professional to diagnose ventriculomegaly.

By “sodium-potassium-chloride transporter 1 (NKCC1)” is meant a polypeptide or fragment thereof having at least about 85% amino acid identity to NCBI Accession No. NP_001243390.1 that transports water and/or ions across a cell membrane. In some embodiments, NKCC1 provides for the passage of water and/or ions across membrane. NKCC1, is a water and ion cotransporter that follows the combined gradient of Na⁺, K⁺, and Cl⁻ between cerebrospinal fluid (CSF) and choroid plexus (ChP) epithelial cytosol. In embodiments, NKCC1 facilitates ion/water clearance out of the ventricle through Na—K—Cl and water cotransport. Among these ions, cerebrospinal fluid (CSF) K⁺ plays a role in determining NKCC1 transport directionality, as the concentration in the cerebrospinal fluid (CSF) is naturally at very low (˜3.5 mM in adult brains) and therefore sensitive to fluctuations. The sequence at NCBI Accession No. NP_001243390.1 is shown below:

(SEQ ID NO: 1)
MEPRPTAPSSGAPGLAGVGETPSAAALAAARVELPGTAVPSVPEDAAPA
SRDGGGVRDEGPAAAGDGLGRPLGPTPSQSRFQVDLVSENAGRAAAAAA
AAAAAAAAAGAGAGAKQTPADGEASGESEPAKGSEEAKGRFRVNFVDPA
ASSSAEDSLSDAAGVGVDGPNVSFQNGGDTVLSEGSSLHSGGGGGSGHH
QHYYYDTHTNTYYLRTFGHNTMDAVPRIDHYRHTAAQLGEKLLRPSLAE
LHDELEKEPFEDGFANGEESTPTRDAVVTYTAESKGVVKFGWIKGVLVR
CMLNIWGVMLFIRLSWIVGQAGIGLSVLVIMMATVVTTITGLSTSAIAT
NGFVRGGGAYYLISRSLGPEFGGAIGLIFAFANAVAVAMYVVGFAETVV
ELLKEHSILMIDEINDIRIIGAITVVILLGISVAGMEWEAKAQIVLLVI
LLLAIGDFVIGTFIPLESKKPKGFFGYKSEIFNENFGPDFREEETFFSV
FAIFFPAATGILAGANISGDLADPQSAIPKGTLLAILITTLVYVGIAVS
VGSCVVRDATGNVNDTIVTELTNCTSAACKLNFDFSSCESSPCSYGLMN
NFQVMSMVSGFTPLISAGIFSATLSSALASLVSAPKIFQALCKDNIYPA
FQMFAKGYGKNNEPLRGYILTFLIALGFILIAELNVIAPIISNFFLASY
ALINFSVFHASLAKSPGWRPAFKYYNMWISLLGAILCCIVMFVINWWAA
LLTYVIVLGLYIYVTYKKPDVNWGSSTQALTYLNALQHSIRLSGVEDHV
KNFRPQCLVMTGAPNSRPALLHLVHDFTKNVGLMICGHVHMGPRRQAMK
EMSIDQAKYQRWLIKNKMKAFYAPVHADDLREGAQYLMQAAGLGRMKPN
TLVLGFKKDWLQADMRDVDMYINLFHDAFDIQYGVVVIRLKEGLDISHL
QGQEELLSSQEKSPGTKDVVVSVEYSKKSDLDTSKPLSEKPITHKESKG
PIVPLNVADQKLLEASTQFQKKQGKNTIDVWWLFDDGGLTLLIPYLLTT
KKKWKDCKIRVFIGGKINRIDHDRRAMATLLSKFRIDFSDIMVLGDINT
KPKKENIIAFEEIIEPYRLHEDDKEQDIADKMKEDEPWRITDNELELYK
TKTYRQIRLNELLKEHSSTANIIVMSLPVARKGAVSSALYMAWLEALSK
DLPPILLVRGNHQSVLTFYS.

By “sodium-potassium-chloride transporter 1 (NKCC1) gene” “or “sodium-potassium-chloride transporter 1 (NKCC1) polynucleotide” is meant a polynucleotide encoding an NKCC1 polypeptide. An exemplary NKCC1 polynucleotide sequence is provided at positions 190 to 3780 (boldface text) of NCBI Accession No. NM_001256461.2, which is reproduced below:

(SEQ ID NO: 2)
ACACTCGCGCGCTCGCTCGGCTGCCGGTGGCCTCTGTGGCCGTCCAGGCTAGCGGCG
GCCCGCAGGCGGCGGGGAGAAAGACTCTCTCACCTGGTCTTGCGGCTGTGGCCACC
GCCGGCCAGGGGTGTGGAGGGCGTGCTGCCGGAGACGTCCGCCGGGCTCTGCAGTT
CCGCCGGGGGTCGGGCAGCTATGGAGCCGCGGCCCACGGCGCCCTCCTCCGGCG
CCCCGGGACTGGCCGGGGTCGGGGAGACGCCGTCAGCCGCTGCGCTGGCCGC
AGCCAGGGTGGAACTGCCCGGCACGGCTGTGCCCTCGGTGCCGGAGGATGCT
GCGCCCGCGAGCCGGGACGGCGGCGGGGTCCGCGATGAGGGCCCCGCGGCGG
CCGGGGACGGGCTGGGCAGACCCTTGGGGCCCACCCCGAGCCAGAGCCGTTT
CCAGGTGGACCTGGTTTCCGAGAACGCCGGGCGGGCCGCTGCTGCGGCGGCG
GCGGCGGCGGCGGCAGCGGCGGCGGCTGGTGCTGGGGCGGGGGCCAAGCAG
ACCCCCGCGGACGGGGAAGCCAGCGGCGAGAGCGAGCCGGCTAAAGGCAGCG
AGGAAGCCAAGGGCCGCTTCCGCGTGAACTTCGTGGACCCAGCTGCCTCCTCG
TCGGCTGAAGACAGCCTGTCAGATGCTGCCGGGGTCGGAGTCGACGGGCCCAA
CGTGAGCTTCCAGAACGGCGGGGACACGGTGCTGAGCGAGGGCAGCAGCCTG
CACTCCGGCGGCGGCGGCGGCAGTGGGCACCACCAGCACTACTATTATGATAC
CCACACCAACACCTACTACCTGCGCACCTTCGGCCACAACACCATGGACGCTG
TGCCCAGGATCGATCACTACCGGCACACAGCCGCGCAGCTGGGCGAGAAGCTG
CTCCGGCCTAGCCTGGCGGAGCTCCACGACGAGCTGGAAAAGGAACCTTTTGA
GGATGGCTTTGCAAATGGGGAAGAAAGTACTCCAACCAGAGATGCTGTGGTCA
CGTATACTGCAGAAAGTAAAGGAGTCGTGAAGTTTGGCTGGATCAAGGGTGTA
TTAGTACGTTGTATGTTAAACATTTGGGGTGTGATGCTTTTCATTAGATTGTCA
TGGATTGTGGGTCAAGCTGGAATAGGTCTATCAGTCCTTGTAATAATGATGGCC
ACTGTTGTGACAACTATCACAGGATTGTCTACTTCAGCAATAGCAACTAATGGA
TTTGTAAGAGGAGGAGGAGCATATTATTTAATATCTAGAAGTCTAGGGCCAGA
ATTTGGTGGTGCAATTGGTCTAATCTTCGCCTTTGCCAACGCTGTTGCAGTTGC
TATGTATGTGGTTGGATTTGCAGAAACCGTGGTGGAGTTGCTTAAGGAACATTC
CATACTTATGATAGATGAAATCAATGATATCCGAATTATTGGAGCCATTACAGT
CGTGATTCTTTTAGGTATCTCAGTAGCTGGAATGGAGTGGGAAGCAAAAGCTC
AGATTGTTCTTTTGGTGATCCTACTTCTTGCTATTGGTGATTTCGTCATAGGAA
CATTTATCCCACTGGAGAGCAAGAAGCCAAAAGGGTTTTTTGGTTATAAATCTG
AAATATTTAATGAGAACTTTGGGCCCGATTTTCGAGAGGAAGAGACTTTCTTTT
CTGTATTTGCCATCTTTTTTCCTGCTGCAACTGGTATTCTGGCTGGAGCAAATA
TCTCAGGTGATCTTGCAGATCCTCAGTCAGCCATACCCAAAGGAACACTCCTAG
CCATTTTAATTACTACATTGGTTTACGTAGGAATTGCAGTATCTGTAGGTTCTT
GTGTTGTTCGAGATGCCACTGGAAACGTTAATGACACTATCGTAACAGAGCTAA
CAAACTGTACTTCTGCAGCCTGCAAATTAAACTTTGATTTTTCATCTTGTGAAA
GCAGTCCTTGTTCCTATGGCCTAATGAACAACTTCCAGGTAATGAGTATGGTGT
CAGGATTTACACCACTAATTTCTGCAGGTATATTTTCAGCCACTCTTTCTTCAG
CATTAGCATCCCTAGTGAGTGCTCCCAAAATATTTCAGGCTCTATGTAAGGACA
ACATCTACCCAGCTTTCCAGATGTTTGCTAAAGGTTATGGGAAAAATAATGAAC
CTCTTCGTGGCTACATCTTAACATTCTTAATTGCACTTGGATTCATCTTAATTGC
TGAACTGAATGTTATTGCACCAATTATCTCAAACTTCTTCCTTGCATCATATGCA
TTGATCAATTTTTCAGTATTCCATGCATCACTTGCAAAATCTCCAGGATGGCGT
CCTGCATTCAAATACTACAACATGTGGATATCACTTCTTGGAGCAATTCTTTGT
TGCATAGTAATGTTCGTCATTAACTGGTGGGCTGCATTGCTAACATATGTGATA
GTCCTTGGGCTGTATATTTATGTTACCTACAAAAAACCAGATGTGAATTGGGGA
TCCTCTACACAAGCCCTGACTTACCTGAATGCACTGCAGCATTCAATTCGTCTT
TCTGGAGTGGAAGACCACGTGAAAAACTTTAGGCCACAGTGTCTTGTTATGAC
AGGTGCTCCAAACTCACGTCCAGCTTTACTTCATCTTGTTCATGATTTCACAAA
AAATGTTGGTTTGATGATCTGTGGCCATGTACATATGGGTCCTCGAAGACAAGC
CATGAAAGAGATGTCCATCGATCAAGCCAAATATCAGCGATGGCTTATTAAGAA
CAAAATGAAGGCATTTTATGCTCCAGTACATGCAGATGACTTGAGAGAAGGTG
CACAGTATTTGATGCAGGCTGCTGGTCTTGGTCGTATGAAGCCAAACACACTTG
TCCTTGGATTTAAGAAAGATTGGTTGCAAGCAGATATGAGGGATGTGGATATG
TATATAAACTTATTTCATGATGCTTTTGACATACAATATGGAGTAGTGGTTATTC
GCCTAAAAGAAGGTCTGGATATATCTCATCTTCAAGGACAAGAAGAATTATTGT
CATCACAAGAGAAATCTCCTGGCACCAAGGATGTGGTAGTAAGTGTGGAATAT
AGTAAAAAGTCCGATTTAGATACTTCCAAACCACTCAGTGAAAAACCAATTACA
CACAAAGAATCCAAAGGCCCTATTGTGCCTTTAAATGTAGCTGACCAAAAGCTT
CTTGAAGCTAGTACACAGTTTCAGAAAAAACAAGGAAAGAATACTATTGATGTC
TGGTGGCTTTTTGATGATGGAGGTTTGACCTTATTGATACCTTACCTTCTGACG
ACCAAGAAAAAATGGAAAGACTGTAAGATCAGAGTATTCATTGGTGGAAAGAT
AAACAGAATAGACCATGACCGGAGAGCGATGGCTACTTTGCTTAGCAAGTTCC
GGATAGACTTTTCTGATATCATGGTTCTAGGAGATATCAATACCAAACCAAAGA
AAGAAAATATTATAGCTTTTGAGGAAATCATTGAGCCATACAGACTTCATGAAG
ATGATAAAGAGCAAGATATTGCAGATAAAATGAAAGAAGATGAACCATGGCGA
ATAACAGATAATGAGCTTGAACTTTATAAGACCAAGACATACCGGCAGATCAG
GTTAAATGAGTTATTAAAGGAACATTCAAGCACAGCTAATATTATTGTCATGAG
TCTCCCAGTTGCACGAAAAGGTGCTGTGTCTAGTGCTCTCTACATGGCATGGTT
AGAAGCTCTATCTAAGGACCTACCACCAATCCTCCTAGTTCGTGGGAATCATCA
GAGTGTCCTTACCTTCTATTCATAAATGTTCTATACAGTGGACAGCCCTCCAGAAT
GGTACTTCAGTGCCTAGTGTAGTAACTGAAATCTTCAATGACACATTAACATCACAA
TGGCGAATGGTGACTTTTCTTTCACGATTTCATTAATTTGAAAGCACACAGGAAAGT
TGCTCCATTGATAACGTGTATGGAGACTTCGGTTTTAGTCAATTCCATATCTCAATCT
TAATGGTGATTCTTCTCTGTTGAACTGAAGTTTGTGAGAGTAGTTTTCCTTTGCTACT
TGAATAGCAATAAAAGCGTGTTAACTTTTTGATTGATGAAAGAAGTACAAAAAGCC
TTTAGCCTTGAGGTGCCTTCTGAAATTAACCAAATTTCATCCATATATCCTCTTTTAT
AAACTTATAGAATGTCAAACTTTGCCTTCAACTGTTTTTATTTCTAGTCTCTTCCACT
TTAAAACAAAATGAACACTGCTTGTCTTCTTCCATTGACCATTTAGTGTTGAGTACTG
TATGTGTTTTGTTAATTCTATAAAGGTATCTGTTAGATATTAAAGGTGAGAATTAGG
GCAGGTTAATCAAAAATGGGGAAGGGGAAATGGTAACCAAAAAGTAACCCCATGG
TAAGGTTTATATGAGTATATGTGAATATAGAGCTAGGAAAAAAAGCCCCCCCAAAT
ACCTTTTTAACCCCTCTGATTGGCTATTATTACTATATTTATTATTATTTATTGAAACC
TTAGGGAAGATTGAAGATTCATCCCATACTTCTATATACCATGCTTAAAAATCACGT
CATTCTTTAAACAAAAATACTCAAGATCATTTATATTTATTTGGAGAGAAAACTGTC
CTAATTTAGAATTTCCCTCAAATCTGAGGGACTTTTAAGAAATGCTAACAGATTTTT
CTGGAGGAAATTTAGACAAAACAATGTCATTTAGTAGAATATTTCAGTATTTAAGTG
GAATTTCAGTATACTGTACTATCCTTTATAAGTCATTAAAATAATGTTTCATCAAATG
GTTAAATGGACCACTGGTTTCTTAGAGAAATGTTTTTAGGCTTAATTCATTCAATTGT
CAAGTACACTTAGTCTTAATACACTCAGGTTTGAACAGATTATTCTGAATATTAAAA
TTTAATCCATTCTTAATATTTTAAAACTTTTGTTAAGAAAAACTGCCAGTTTGTGCTT
TTGAAATGTCTGTTTTGACATCATAGTCTAGTAAAATTTTGACAGTGCATATGTACTG
TTACTAAAAGCTTTATATGAAATTATTAATGTGAAGTTTTTCATTTATAATTCAAGGA
AGGATTTCCTGAAAACATTTCAAGGGATTTATGTCTACATATTTGTGTGTGTGTGTGT
ATATATATGTAATATGCATACACAGATGCATATGTGTATATATAATGAAATTTATGT
TGCTGGTATTTTGCATTTTAAAGTGATCAAGATTCATTAGGCAAACTTTGGTTTAAGT
AAACATATGTTCAAAATCAGATTAACAGATACAGGTTTCATAGAGAACAAAGGTGA
TCATTTGAAGGGCATGCTGTAATTTCACACAATTTTCCAGTTCAAAAATGGAGAATA
CTTCGCCTAAAATACTGTTAAGTGGGTTAATTGATACAAGTTTCTGTGGTGGAAAAT
TTATGCAGGTTTTCACGAATCCTTTTTTTTTTTTTTTTTTTTTTTTGAGACGGAGTCTT
GCTCTGTTGCCACGCTGGAATGCAGTAACGTGATCTTGGCTCACTGCGACCTCCACC
TCCCCAGTTCAAGCGATTCTCCTGCCTCAGCCTCCCTAGTAGCTGGGACTACGGGTG
CACGCCACCATGCCCAGCTAATTTTTGTATTTTGAGTAGAGACAGGGTTTCACCGTG
TTGGCTAGGATGGTGTCTATCTCTTGACCTTGTGATCCACCCGCCTCAGCCTCCCAGA
GTGCTGGGATTACAGGTGCGAGCCACTGCGCCTGGCTGGTTTTCATGAATCTTGATA
GACATCTATAACGTTATTATTTTCAGTGGTGTGCAGCATTTTTGCTTCATGAGTATGA
CCTAGGTATAGAGATCTGATAACTTGAATTCAGAATATTAAGAAAATGAAGTAACT
GATTTTCTAAAAAAAAAAAAAAAAAAAATTTCTACATTATAACTCACAGCATTGTTC
CATTGCAGGTTTTGCAATGTTTGGGGGTAAAGACAGTAGAAATATTATTCAGTAAAC
AATAATGTGTGAACTTTTAAGATGGATAATAGGGCATGGACTGAGTGCTGCTATCTT
GAAATGTGCACAGGTACACTTACCTTTTTTTTTTTTTTTTTTAAGTTTTTCCCATTCAG
GAAAACAACATTGTGATCTGTACTACAGGAACCAAATGTCATGCGTCATACATGTG
GGTATAAAGTACATAAAATATATCTAACTATTCATAATGTGGGGTGGGTAATACTGT
CTGTGAAATAATGTAAGAAGCTTTTCACTTAAAAAAAATGCATTACTTTCACTTAAC
ACTAGACACCAGGTCGAAAATTTTCAAGGTTATAGTACTTATTTCAACAATTCTTAG
AGATGCTAGCTAGTGTTGAAGCTAAAAATAGCTTTATTTATGCTGAATTGTGATTTTT
TTATGCCAAATTTTTTTTAGTTCTAATCATTGATGATAGCTTGGAAATAAATAATTAT
GCCATGGCATTTGACAGTTCATTATTCCTATAAGAATTAAATTGAGTTTAGAGAGAA
TGGTGGTGTTGAGCTGATTATTAACAGTTACTGAAATCAAATATTTATTTGTTACATT
ATTCCATTTGTATTTTAGGTTTCCTTTTACATTCTTTTTATATGCATTCTGACATTACA
TATTTTTTAAGACTATGGAAATAATTTAAAGATTTAAGCTCTGGTGGATGATTATCT
GCTAAGTAAGTCTGAAAATGTAATATTTTGATAATACTGTAATATACCTGTCACACA
AATGCTTTTCTAATGTTTTAACCTTGAGTATTGCAGTTGCTGCTTTGTACAGAGGTTA
CTGCAATAAAGGAAGTGGATTCATTAAACCTA.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. “

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Non-limiting examples of diseases include cerebrospinal fluid disorders, hydrocephalus, intraventricular hemorrhage, hyperkalirrhachia, ventriculomegaly. In embodiments, the hydrocephalus is acute post-hemorrhagic hydrocephalus, or congenital hydrocephalus. In embodiments, the intraventricular hemorrhage is grade 2, 3, or 4 according to the original Papile classification of preterm germinal matrix hemorrhage and intraventricular hemorrhage.

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In some embodiments, an effective amount of an agent described herein increases the activity, expression, or level of NKCC1 by at least about 10, 20, 30, 40, 50, 60, 70, 80, 90, or 100%. In some embodiments, an effective amount of an agent reduces and/or stabilizes the level, production, or pressure of cerebrospinal fluid in the brain, and or increases the clearance of cerebrospinal fluid in the brain.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

A “host cell” or “cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increases” is meant a positive alteration of at least 10%, 25%, 50%, 75%, or 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “operably linked” refers to a functional linkage between a regulatory sequence and a coding sequence, where a first polynucleotide is positioned adjacent to a second polynucleotide that directs transcription of the first polynucleotide when appropriate molecules (e.g., transcriptional activator proteins) are bound to the second polynucleotide. The described components are therefore in a relationship permitting them to function in their intended manner. For example, placing a coding sequence under regulatory control of a promoter means positioning the coding sequence such that the expression of the coding sequence is controlled by the promoter.

By “positioned for expression” is meant that the polynucleotide of the invention positioned adjacent to a DNA sequence that directs transcription and translation of the sequence. In embodiments, the polynucleotide is a DNA molecule or an RNA molecule.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

The term “promoter” as used herein refers to a polynucleotide sequence that directs the expression of a gene. In embodiments, expression involves transcription. A promoter may direct the transcription of a prokaryotic or eukaryotic gene. A promoter may be “inducible”, initiating transcription in response to an inducing agent or, in contrast, a promoter may be “constitutive”, whereby an inducing agent does not regulate the rate of transcription. A promoter may be regulated in a tissue-specific or tissue-preferred manner, such that it is only active in transcribing the operable linked coding region in a specific tissue type or types.

The term “recombinant” as used herein in the context of proteins or nucleic acids refers to proteins or nucleic acids that do not occur in nature or in a naturally occurring protein or nucleic acid sequence, but are the product of human engineering. Human engineering often or typically utilizes molecular biological or molecular genetic tools and techniques practiced by the skilled practitioner in the art. For example, in some embodiments, a recombinant protein or nucleic acid molecule comprises an amino acid or nucleotide sequence that comprises at least one, at least two, at least three, at least four, at least five, at least six, at least seven, or at least eight mutations as compared to any naturally occurring sequence.

By “reduces” is meant a negative alteration of at least 10%, 25%, 50%, 75%, or 100%.

By “reference” is meant a standard or control condition. In some embodiments, a reference is the condition of a control subject (e.g., a subject with a CSF disorder) that has not been treated with an agent of the invention. In other embodiments, a control subject is a healthy control.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.10% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal. In some embodiments, the mammal is a human or non-human mammal, such as a bovine, equine, canine, ovine, or feline.

“Transduction” refers to a process by which a polynucleotide contained in a virus or virus vector is introduced or transferred into a cell by the virus or virus vector, wherein polynucleotide is expressed. In embodiments, the polynucleotide contains one or more transgenes. In an embodiment, the DNA or polynucleotide transduced into a cell by a virus vector, such as an rAAV (recombinant adeno-associated virus) vector as described herein, is stably expressed in the cell. In some cases, a virus or virus vector is said to infect a cell. In embodiments the rAAV vector is an AAV2/5 vector. In embodiments, the rAAV vector has a tropism for choroid plexus. Non-limiting examples of AAV vectors suitable for use in embodiments of the invention are described in Chen, B., et al., Cell 174:521-535, doi: doi:10.1016/j.cell.2018.06.005; in Deverman, B., et al, Nat. Biotechnol, 34:204-209, doi: 10.1038/nbt.3440; in Tervo, D. et al., Neuron, 92:372-382, doi: 10.1016/j.neuron.2016.09.021; and in Lin, K., et al., doi: 10.21203/rs.3.rs-52147/v1.

The term “transfection” or “transfecting” is defined as a process of introducing nucleic acid molecules into a cell. The introduction may be accomplished by non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art.

By “transformed cell” is meant a cell into which, or into an ancestor of which, has been introduced, by means of recombinant DNA techniques, a polynucleotide molecule encoding a protein of the invention.

By “vector” is meant a nucleic acid molecule or an agent containing a nucleic acid molecule that is capable of replication in a host cell. In embodiments, the agent or nucleic acid molecule is a plasmid, cosmid, virus, or bacteriophage. In one embodiment, a vector is an expression vector that is a nucleic acid construct, generated recombinantly or synthetically, bearing a series of specified nucleic acid elements that enable transcription of a nucleic acid molecule in a host cell. Typically, expression is placed under the control of certain regulatory elements, including constitutive or inducible promoters, tissue-preferred regulatory elements, and enhancers.

By “viral vectors” or “viral-based vector” is meant a viral delivery means. Viral vectors include, but are not limited to, adenovirus, adeno-associated virus (AAV), retroviral, lentiviral systems, hepatitis B virus, herpes simplex virus, and baculovirus.

By “virus-like particles (VLPs)” is meant virus particles made up of one or more viral structural proteins, but lacking the viral genome. Because VLPs lack a viral genome, they are non-infectious and yield safer and potentially more-economical products. In addition, VLPs can be produced by heterologous expression and can be purified. Many VLPs comprise at least a viral core protein that drives budding and release of particles from a host cell.

As used herein, the term “vehicle” refers to a solvent, diluent, or carrier component of a pharmaceutical composition.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1L are plots, cell images, bar graphs, and schematics demonstrating postnatal cerebrospinal fluid (CSF) [K+] decrease coincides with increased choroid plexus metabolism. FIG. 1A intracranial pressure (ICP)-OES quantification of cerebrospinal fluid (CSF) [K+]. ***p=0.0009, ****p<0.0001; Sidak's test. FIG. 1B Developmental cerebrospinal fluid (CSF)/Serum [K⁺] ratios. Ratio=cerebrospinal fluid (CSF) [K⁺]/average serum [K+]; Sidak's test. * p=0.0202. FIG. 1C Transmission micrographs of Lateral Ventricle choroid plexus (LVChP) mitochondria. d-f Quantification of mitochondrial number (FIG. 1D), area (FIG. 1E), and % area occupancy (FIG. 1F) in choroid plexus (ChP) epithelial cells. d: *p=0.0269; e: E16 vs. P7 ** p=0.0025; E16 vs. adult * p=0.0115; P0 vs. P7 **p=0.0062; P0 vs. adult * p=0.0344; f: E16 vs. P7 * p=0.0112; E16 vs. adult ***p=0.0008; P0 vs. P7 ** p=0.0010; P0 vs. adult ****p<0.001; P7 vs. adult ** p=0.0010; Welch's two-tailed unpaired t-test. FIG. 1G Schematic of explant-based Agilent Seahorse XFe96 test. FIGS. 1H and 1I Oxidative respiration metrics over development; OCR, oxygen consumption rate. h: E16 vs. P0 ****p<0.0001; E16 vs. P7 ***p=0.0025; E16 vs. Adult ****p=0.0037 0.0001; i: E16 vs. P0 ****p<0.0001; E16 vs. P7 ** p=0.0022; E16 vs. Adult ** p=0.0037; P0 vs. Adult ** p=0.0012; P7 vs. Adult * p=0.0480.; Welch's ANOVA with Dunnett's T3 multiple comparison test. FIGS. 1J and 1K Mitochondrial distribution apical: basal proximity ratio: 1=apical surface. 0=basal surface. Solid line: median; dashed line: upper/lower quartiles. ****p<0.0001; Kolmogorov-Smirnov test. FIG. 1L Cumulative distribution of mitochondrial localization. Solid lines: mean; shaded area: range. Scale bar=(FIG. 1C) 250 nm, (FIG. 1J) 2 μm. All quantitative data presented as mean±SEM. Lateral Ventricle (LV) choroid plexus (ChP), lateral ventricle choroid plexus; E, embryonic day; P, postnatal day.

FIGS. 2A-2J are images, a heat map, a bar graph, a schematic, a box-and-wisker plot, immunoblot images, and plots demonstrating choroid plexus epithelial cells display age-dependent translation of ion and water transporters, in particular NKCC1. FIG. 2A Rpl10a-conjugated EGFP expression in choroid plexus (ChP) epithelial cells after Foxj1-Cre recombination in TRAP-BAC mice. Scale bars=500 μm. Representative of 2 experiments, each with 2 biologically independent replicates. FIG. 2B Heatmap and hierarchical clustering of differentially expressed genes (adjusted p<0.05). Right on the colorscale: enriched adult expression. Left on the colorscale: enriched E16.5 expression. FIG. 2C Top 4 gene functional clusters shown by DAVID to be enriched in Adult choroid plexus (ChP) epithelial cells over E16.5 choroid plexus (ChP) epithelial cells. FIG. 2D Top 10 significantly enriched gene ontology (GO) terms for “Biological processes”. Plotted with horizontal lines for medians, bounds of boxes for quartiles, and whiskers for maximum and minimum values. The log 10 fold change (Log FC) is plotted for each expressed gene for the network. Positive values (right):Adult enrichment; negative values (left): E16.5 enrichment. Multiple measures were corrected using Bonferroni correction. ** p≤0.01, ***p≤0.001, ****p≤0.0001. FIG. 2E Schematics depicting the interaction of NKCC1, Na⁺/K⁺-ATPase, and Klotho on the apical membrane of a choroid plexus (ChP) epithelial cell. FIGS. 2F and 2G RT-qPCR and immunoblotting of Lateral Ventricle choroid plexus (LVChP) during postnatal development. f: N=4 biologically independent animals from two experiments at each timepoint. Colors are matched to the gene's protein name in FIG. 2E. FIG. 2G: representative of 3 independent experiments, each with tissues from 1-2 animals (2 animals for ages under P14, 1 animal for ages of P14 and older) pooled for each timepoint. FIG. 2H Fluorescence images of Calcein-AM labeled epithelial cells from Lateral Ventricle choroid plexus (LVChP) explants under high extracellular K⁺ challenge. Scale bar=50 μm, representative of 4 biological replicates collected from 3 independent experiments. Biological replicates with poor calcein labeling or visible damage were excluded prior to K+ challenge. FIGS. 2I and 2J Quantification of choroid plexus (ChP) epithelia cellular volume by IMARIS 3D analysis. Percent volume increase=dV/V0 for each timepoint (t). V0=initial volume of the cell; t 1070=subsequent timepoint after challenge; dV=Vt−V0×100%. At least 5 cells were analyzed for each explant from one animal; N=4 animals. All quantitative data are presented as mean±SD.

FIGS. 3A-3I. are a heat map, plots, and immunoblot images demonstrating NKCC1 temporal expression requires CHD4/NuRD complex. FIG. 3A RNAseq data showing expression of CHD and other NuRD units by the choroid plexus (ChP). Yellow: low expression; Dark brown: high expression. FIG. 3B Immunofluorescence images of CHD4 in the choroid plexus (ChP) epithelia at E16.5, P0, and adult; Scale bar=30 μm. A total of 4 animals at each age were imaged in two independent experiments. FIG. 3C Immunoblots of Co-IP by CHD4 antibody. Representative of two independent experiments, each contain more than 10 animals for each age. FIG. 3D RT-qPCR of CHD4 transcripts in choroid plexus (ChP) with AAV2/5-Cre transduction. Grey: AAV-GFP; orange: AAV-cre (same color scheme is used for the rest of the figure). ****p<0.0001, N=7, Welch's two-tailed unpaired t-test. FIG. 3E Immunoblot of CHD4 in AAV-cre mice choroid plexus (ChP) lysate, representative of 3 independent experiment, each contain 2 or 3 biologically independent animals. FIG. 3F RT-qPCR of NKCC1 expression in AAV-cre vs. AAV-GFP mice choroid plexus (ChP). All values were normalized to P7 AAV-GFP control mice. ** p=0.0015, ***p=0.0009, N=7, Welch's two-tailed unpaired t1086 test. FIG. 3G Immunoblot of NKCC1 in Lateral Ventricle choroid plexus (LVChP) lysates from AAV-cre vs. AAV-GFP mice, representative of 3 independent experiments, each containing 2 or 3 biologically independent animals (same samples as those collected for FIG. 2E). FIGS. 3H and 3I CHD4 and NKCC1 RT-qPCR in 4VChP. ** p=0.0083, ***p=0.0005, **** p<0.0001, N=7, Welch's two-tailed unpaired t-test. All quantitative data are presented as mean±SD. When immunoblots were quantified, all samples for quantitative comparison were on the same blot.

FIGS. 4A-4J are bar graphs, immunoblot images, and immunofluorescence images demonstrating choroid plexus (ChP) NKCC1 actively mediates cerebrospinal fluid (CSF) K+ clearance in the first postnatal week. FIG. 4A RT1095 qPCR of NKCC1 mRNA levels in P0 mice. Grey: AAV-GFP; purple: AAV-NKCC1 (same color scheme is used for the rest of this figure). ** p=0.009, N=3; Welch's two-tailed unpaired t-test. FIG. 4B Immunoblots from AAV-NKCC1 vs. AAV-GFP P0 mice choroid plexus (ChP) lysates, representative of 3 independent experiments, each contained multiple biological replicates. FIGS. 4C-4D Quantification of all immunoblots of NKCC1 (FIG. 4C) ***p=0.0009, N=7; Welch's two tailed unpaired t-test; and pNKCC1 (FIG. 4D) * p=0.0355, N=5; Welch's two-tailed unpaired t-test. All samples for direct quantitative comparison were on the same blot. FIG. 4E Immunofluorescence images showing co-localization of 3×HA tag and NKCC1 in P0 choroid plexus (ChP). Scale bar=50 μm, representative of 3 independent experiments, each with 2 biological replicates. FIGS. 4F-4I Immunofluorescence images of HA in AAV2/5-NKCC1 transduced brain at P1: the Lateral Ventricle choroid plexus (LVChP) (f), 3rd ventricle choroid plexus (ChP) (3VChP; (FIG. 4G), 4VChP (FIG. 4H), and the spinal cord (FIG. 4I; sc=spinal cord). Traces of HA is shown in the meninges near the injection site (grey arrow). Scale bar=500 μm. FIGS. 4F-4I are representative of 2 independent experiments, each with 3 biological replicates. FIG. 4J intracranial pressure (ICP)-OES measurements of cerebrospinal fluid (CSF) [K+] from AAV-NKCC1 vs. AAV-GFP P1 mice (N=8 in AAV-GFP cohort; N=7 in AAV-NKCC1 cohort). **p=0.0033; Welch's two-tailed unpaired t-test. All quantitative data are presented as mean±SD.

FIGS. 5A-5I are plots, schematics, and live MRI images demonstrating choroid plexus (ChP) NKCC1 overexpression reduces brain ventricular volume and increases intracranial compliance. FIG. 5A T2-weighted live MRI images. Only slices with visible lateral and 3rd ventricles are shown (for NKCC1 overexpression (OE) mice, slices matching with control mice are shown regardless of ventricles). FIG. 5B Lateral ventricle volumes. Uninjected N=4; AAV-GFP and AAV-NKCC1 N=6, from two independent experiments; Black with no fill: uninjected; grey: AAV-GFP; purple: AAV-NKCC1 (the same color scheme is used for the rest of this figure). ****p<0.0001; Welch's two-tailed unpaired t-test. FIG. 5C Brain sizes, which are presented as the average coronal section area from all images with visible lateral and 3rd ventricles (NKCC1 overexpression (OE) data were calculated using the matching images to the controls, regardless of ventricles visibility). Uninjected N=4; AAV-GFP and AAV-NKCC1 N=6 (same as mice included in panel b); Welch's two-tailed unpaired t-test. FIG. 5D Schematic of in vivo constant rate cerebrospinal fluid (CSF) infusion test. FIG. 5E Example of intracranial pressure (ICP) curve during the infusion test (infusion begins at 0 min) in an AAV-GFP mouse, fitted to Marmarou's model. Values extracted include: baseline intracranial pressure (ICP) (ICPb), a pressure-independent compliance coefficient (Ci) and the resistance to cerebrospinal fluid (CSF) outflow (R_{CSF}). FIG. 5F Example intracranial pressure (ICP) recordings from AAV-NKCC1 mice and controls. For clarity, data have been low pass filtered to remove the waveform components. FIG. 5G Compliance coefficients. Uninjected N=4; AAV-GFP N=8; AAV-NKCC1 N=9; 3 total independent experiments. *p=0.0384; Welch's two-tailed unpaired t-test. FIGS. 5H and 5I Plots of baseline intracranial pressure (ICP) and resistance to cerebrospinal fluid (CSF) outflow (R_{CSF}) at 5-7 weeks. Uninjected N=4, AAV-GFP N=8, AAV1133 NKCC1 N=9; 3 total independent experiments (same experiments as those included in g). Welch's two tailed unpaired t-test. All quantitative data are presented as mean SD.

FIGS. 6A-6D are schematics, brain images, a reconstructed 3D image, and a plot demonstrating choroid plexus (ChP) NKCC1 overexpression mitigates ventriculomegaly in an obstructive hydrocephalus model. FIG. 6A Schematics showing the workflow: E14.5 in utero intracerebroventricular (ICV) injection of AAV2/5-NKCC1 or AAV2/5-GFP, followed by intracerebroventricular (ICV) injection of kaolin at P4, and MRI at P14. FIG. 6B Representative sequential brain images (rostral to caudal) by T2-weighted live MRI images. Lighter shaded arrows: Lateral Ventricle (LV). Darker shaded arrows: kaolin. FIG. 6C 3D reconstruction of the Lateral Ventricle (LV) and kaolin deposition. Lateral Ventricle (LV): lighter shading. Kaolin: darker shading. FIG. 6D Lateral ventricle volumes. N=3 from two biologically independent litters under each condition; Light grey: AAV-GFP mice with kaolin; darker grey: AAV-NKCC1 mice with kaolin. *p=0.0.0235; Welch's two-tailed unpaired t-test. All quantitative data are presented as 1144 mean±SD.

FIG. 7 is a schematic presenting a working model of choroid plexus (ChP) NKCC1 mediating K+-driven cerebrospinal fluid (CSF) outflow. The schematics depict choroid plexus (ChP) NKCC1 mediated K⁺ and water clearance from cerebrospinal fluid (CSF) in neonatal mice, in comparison to the adult scenario. For simplicity and clarity, only K⁺ is depicted among all ions and only NKCC1 and Na⁺/K⁺-ATPase are included. Neonatal (P0-7, above) choroid plexus (ChP) has high pNKCC1 than adult, albeit lower total NKCC1. Neonate cerebrospinal fluid (CSF) [K⁺] is 2-3 fold higher than adult. With similar [Na⁺] and [Cl⁻], this [K⁺] difference is sufficient to alter the total Nernst potential of epithelial cells and bias NKCC1 transport of K⁺, together with water, out of cerebrospinal fluid (CSF) into the choroid plexus (ChP) in neonates.

FIGS. 8A and 8B are a plot and transmission electron micrographs showing glycogen load in choroid plexus (ChP) epithelial cells. FIG. 8A Representative transmission electron micrographs of E16.5, P0, P7, and adult Lateral Ventricle (LV) choroid plexus (ChP). Glycogen granules are highlighted in light grey shading. Scale bar=2 μm. N=Nucleus. FIG. 8B Proportion of TEM fields of view that are filled with glycogen granules, N=3 animals from 2 independent experiments (same animals as those used in FIGS. 1D-1F), 10-15 fields of view (FOV) per animal, distinct cells were captured in each FOV. E16 vs. P7: ** p=0.0030; E16 vs. adult: ** p=0.0029; P0 vs. P7:* p=0.0124; P0 vs. adult * p=0.0117; P7 vs. adult * p=0.0136; Welch's two-tailed unpaired t-test. CEPC, choroid plexus epithelial cell.

FIGS. 9A and 9B are a schematic and a plot showing seahorse XF Cell mito stress test profile and representative curves. FIG. 9A Schematic of the Agilent Cell Mito Stress Test showing the experimental design to quantify mitochondria basal respiration and ATP production. FIG. 9B Representative experiment of choroid plexus (ChP) in Cell Mito Stress Test; N=12 E16.5 animals and N=4 adult animals. Lighter grey (top line): adult; darker grey (bottom line): E16.5.

FIGS. 10A and 10B are images and distribution plots relating to supportive analysis of TRAP sequencing providing representative images and quantification of choroid plexus (ChP) epithelial mitochondria distribution analysis. FIG. 10A Representative transmission electron micrographs of E16.5, P0, P7, and adult Lateral Ventricle (LV) choroid plexus (ChP). Mitochondria (light circles), apical membrane (upper line), and basal membrane (lower line) are labeled. Scale bar=2 μm. Images are representative of 2 independent experiments with a total of 3 biologically independent animals at each age. FIG. 10B Mitochondrial distribution plots from each animal (same as described in panel a). Apical: basal ratio: 1 is touching the apical surface and 0 is touching the basal surface. Solid thick line indicates median and thinner line indicates upper/lower quartiles.

FIGS. 11A-11H are images, plots, box-and-wisker plots, a schematic, and charts relating to supporting analysis of TRAP sequencing. FIGS. 11A and 11B Rpl10a-conjugated EGFP expression in choroid plexus (ChP) epithelial cells after Foxj1-Cre recombination in TRAP-BAC mice. Aqp1 marked choroid plexus (ChP) epithelial apical membrane. Scale bars=500 μm (a) and 100 μm (b). Representative of 2 experiments, each with 2 biologically independent replicates. FIG. 11C Perturbation vs. overrepresentation analysis via iPathway (Advaita) reveals enriched pathways at E16.5 (lightest grey shading) and Adult (medium-shade grey). * indicates pathways that are only overrepresented, but not predicted to be additionally perturbed at the network level. FIG. 11D Top 10 significantly enriched GO terms for “molecular function”. Plotted with center bars as median, bounds of boxes for quartiles, and whiskers for maximum and minimum values. The log 10 fold change (Log FC) is plotted for each expressed gene for the network. Positive values (right) indicate Adult enrichment and negative values (left) indicate E16.5 enrichment. p values are corrected for multiple measures using Bonferroni correction. **p 0.01, ***p 0.001, ****p 0.0001. FIGS. 11E and 11F Proportion of enriched genes in E16.5 and Adult choroid plexus (ChP) with predicted transmembrane domains using TMHMM. FIG. 11G Schematic of choroid plexus (ChP) localization within brain ventricles, relative position to blood and cerebrospinal fluid (CSF), and transporters. Lighter grey/bold text: significantly enriched in Adult vs. E16.5 TRAP (adjusted p<0.05). FIG. 11H FPKM values from TRAP of transcripts associated with choroid plexus (ChP) transport.

FIGS. 12A and 12B are a plot and an immunoblot image relating to TRAP candidate validation in 4V choroid plexus (ChP). FIGS. 12A and 12B RT-qPCR (N=4 biologically independent animals from two experiments for each timepoint, colors were chosen to match with FIG. 2F) and immunoblotting of 4V choroid plexus (ChP) during postnatal development, representative of 3 independent experiments, each with tissues from 1-2 animals pooled (2 animals for ages under P14, 1 animal for ages of P14 and older) for each timepoint FIG. 13 presents a workflow of IMARIS demonstrating the cell volume quantification process. At each time point, the reconstructed 3D cell mask is highlighted and views from x-y plane and y-z plane are displayed. Raw images from a single plane at the last time point are shown on the right end. Scale bar=10 μm (P4) and 50 μm (Adult). A total of 3 independent experiments were conducted, each contained 2-3 biological replicates, only tissues with good quality (i.e. tissues without tears or appearing stretched/crumpled due to mounting, and with good calcein signal indicating viability) were included in quantification, resulting in N=4 for each age.

FIG. 14 presents images showing localization of NKCC1(light shading) at the apical membrane of choroid plexus (ChP) epithelial cells across development (P0, P7, P14, and Adult). AQP1 (medium-grey) marks the apical membrane; the white dashed line marks the basal membrane. Scale bar=20 m. N=4 biological replicates were included for each age, from 2 independent experiments.

FIG. 15 provides Co-IP immunoblots using adult mouse cerebellum to validate the Co-IP protocol, representative of 3 independent experiments, each contained tissues collected from 1-2 mice. An antibody targeting CHD4 co-immunoprecipitated several other complex members including HDAC1, HDAC2, and MBD3, from mouse cerebellar lysate, while a negative control performed with a control antibody of the same host species (in this case anti-NKCC1 antibody) failed to pull down NuRD-CHD4 complex members.

FIGS. 16A-16C are immunoblots and a plot relating to validation of AAV2/5-NKCC1 transduction efficiency and specificity. FIG. 16A Immunoblots of AAV2/5-NKCC1 transduced choroid plexus (ChP) showing successful but variable transduction rate within one litter. N=5 biologically independent animals collected from two experiments. FIG. 16B Immunoblot of AAV transduced choroid plexus (ChP) and meninges showing non-detectable meningeal transduction by AAV2/5-NKCC1. Representative of 2 experiments, each with 2 biologically independent replicates. FIG. 16C RT-qPCR analysis of all other K⁺ transporters and channels in the choroid plexus (ChP) after in utero viral transduction, showing no significant changes; Light grey: AAV-GFP; darker grey: AAV-NKCC1. (>0.05, multiple t-Test corrected for multiple comparisons using the Holm-Sidak method. N=3 biologically independent animals collected from 2 experiments.

FIGS. 17A-17C are a schematic and plots relating to mechanisms of constant cerebrospinal fluid (CSF) infusion test by Marmarou's model. FIG. 17A Marmarou's model of cerebrospinal fluid (CSF) dynamics. In this model, the physiologic processes of the cycle of cerebrospinal fluid (CSF) turnover are represented by analogous electric circuit elements, with intracranial pressure (ICP) expressed as a solution to the circuit model in terms of lumped parameters describing the net effect of the processes on the level of intracranial pressure (ICP) without attributing them to specific microscopic pathways. At the most basic level, the model is a statement of conservation of mass, with the rate of cerebrospinal fluid (CSF) production balanced by the rate of cerebrospinal fluid (CSF) storage in intracranial and spinal compartments plus the rate of cerebrospinal fluid (CSF) reabsorption. FIGS. 17B and 17C Higher magnification of the intracranial pressure (ICP) data of infusion test showing the normal arterial and respiratory components of the intracranial pressure (ICP) waveform at the beginning of the test (FIG. 17B). The increase in waveform amplitude with intracranial pressure (ICP) is expected with increasing volume load (FIG. 17C).

FIGS. 18A-18H are schematics, plots, and images demonstrating that intraventricular blood leads to ventriculomegaly in mouse models of pediatric post-hemorrhagic hydrocephalus (PHH). (FIG. 18A) Schematics of prenatal intraventricular hemorrhage (IVH) model at E14.5 (modeling very preterm human infants of 12-20 gestational weeks (GW)) and at P4 (modeling preterm human infants of 24-32 GW). Age-matched blood from donors (litter a) was obtained and immediately delivered into the lateral ventricles of recipient embryos (litter b). Sterile PBS was used as control (litter c). For P4 pups, age-matched blood from a donor pup was drawn and immediately delivered to the lateral ventricle of recipient pups. PBS used as control. Donor, recipients, and PBS controls were all littermates. Schematic: grown mice with pediatric intraventricular hemorrhage (IVH) have no overt changes in head shape, suggesting compensated ventriculomegaly. (FIG. 18B) Workflow for intraventricular hemorrhage (IVH) induction and hydrocephalus evaluation at E14.5 and P4. (FIG. 18C) Representative T2-weighted MRI images (slice #16 of 20-slice series) showing ventriculomegaly in E14.5 and P4 intraventricular hemorrhage (IVH) model mice (red arrow) vs PBS control (white arrow), imaged at P14 and P21, respectively. Scale bar=5 mm. (FIG. 18D) Quantification of total lateral ventricle volumes in E14.5 and P4 intraventricular hemorrhage (IVH) models at P14 and P21, respectively. E14.5: PBS N=9, intraventricular hemorrhage (IVH) N=11, ***p=0.0008; P4: PBS N=8, intraventricular hemorrhage (IVH) N=9, ****p<0.0001. Welch's two-tail unpaired t-test. Data presented as mean±SD. (FIG. 18E) Schematic depicting the infusion test and data from an adult control mouse for analysis using the Marmarou model of cerebrospinal fluid (CSF) dynamics. (FIGS. 18F-18H) Quantitative measurements from infusion test, including compliance, baseline intracranial pressure (ICP), and capacity for cerebrospinal fluid (CSF) efflux. E14.5: PBS N=11, intraventricular hemorrhage (IVH) N=9; P4: PBS N=4, intraventricular hemorrhage (IVH) N=4. (FIG. 18F) * p=0.023, ** p=0.0020; (FIG. 18G) not significant; (FIG. 18H) * p=0.0144, ** p=0.001. All analysis in FIGS. 18F-18H were done by Welch's two-tail unpaired t-test. Data presented as mean±SD. Each data point represents one mouse.

FIGS. 19A-19K are schematics, images, plots, bar graphs, and immunoblot images demonstrating the choroid plexus (ChP) responds rapidly to ventricular blood exposure. (FIG. 19A) Schematic depicting choroid plexus (ChP) explant imaging in response to blood exposure. (FIG. 19B) Two-photon calcium imaging of a Lateral Ventricle (LV) choroid plexus (ChP) explant (FoxJ1-Cre:: GCaMP6f mice) shows increased response following application of plasma. Scale bar=100 m. Top: 10 seconds prior to blood, vs. bottom: 0.25 sec after plasma exposure. (FIG. 19C) Two-photon calcium activity of epithelial cells shown in (FIG. 19B), sorted by peak activity within 25 seconds following application of plasma. Black arrow indicates onset of plasma. Dashed line indicates cutoff of non-responder cells (cutoff of responder cells was defined as 6 standard deviations or more above the pre-stimulation mean within 25 seconds of stimulus exposure), indicating that 91% (987 out of 1080) of choroid plexus (ChP) epithelial cells are sensitive to plasma. (FIG. 19D) Epifluorescence calcium activity of entire Lateral Ventricle (LV) choroid plexus (ChP) explant following addition of plasma and aCSF. (FIGS. 19E and 19F) Quantitative analysis of Lateral Ventricle (LV) choroid plexus (ChP) calcium activity showing (FIG. 19E) peak intensity, (FIG. 19F) rise time, and half-life of plasma-induced epifluorescence. Shaded color zones (FIG. 19D) indicate standard deviation (n=7 mice for plasma, 3 for aCSF). (FIGS. 19G-19I) intraventricular hemorrhage (IVH) increased total choroid plexus (ChP) NKCC1 and pNKCC1 after 48 hr. (H) E14.5: PBS N=5, intraventricular hemorrhage (IVH) N=10, ** p=0.0012; P4: PBS N=6, intraventricular hemorrhage (IVH) N=5, * p=0.02852. (I) E14.5: PBS N=5, intraventricular hemorrhage (IVH) N=10, * p=0.0104; P4: PBS N=6, intraventricular hemorrhage (IVH) N=5, * p=0.0272. Welch's two-tail unpaired t-test. Data presented as mean±SD. (FIG. 19J) cerebrospinal fluid (CSF) [K⁺] increased 2 days after intraventricular hemorrhage (IVH) and returned to baseline 4 days after intraventricular hemorrhage (IVH), * p=0.0448. N=5 for each condition, Welch's two-tail unpaired t-test. Data presented as mean SD. (FIG. 19K) cerebrospinal fluid (CSF) osmolarity remain unchanged by intraventricular hemorrhage (IVH). N=5 for each condition except for uninjected Day 21 where N=3. Each data point represents one mouse, except for cerebrospinal fluid (CSF) data where each data point represents one biological replicate, and 2-3 mice were pooled in each biological replicate to reach sufficient cerebrospinal fluid (CSF) volume for performing assay.

FIGS. 20A-20L are schematics, plots, bar graphs, immunoblot images, and a pie chart demonstrating disrupted choroid plexus (ChP) epithelial physiology leads to decompensated pediatric post-hemorrhagic hydrocephalus (PHH). (FIG. 20A) Model of NKCC1-mediated cerebrospinal fluid (CSF) homeostasis by choroid plexus (ChP) epithelial cells following intraventricular hemorrhage (IVH) and hyperkalirrhachia. (FIGS. 20B and 20C) Epifluorescence calcium responses to plasma in epithelial cells from a Lateral Ventricle (LV) choroid plexus (ChP) explant (FoxJ1-Cre:: GCaMP6f mice) were disrupted by LiCl, 2-APB, and chelation (EDTA, EGTA, BAPTA). No treatment N=7, others N=4 each. (FIG. 20D) Schematic depicting LiCl pretreatment in adult intraventricular hemorrhage (IVH) model. (FIGS. 20E and 20F) LiCl pretreatment blocked pNKCC1 increase in intraventricular hemorrhage (IVH) mice. PBS N=12, intraventricular hemorrhage (IVH) N=9, ***p=0.0007. Welch's two-tail unpaired t-test. Data presented as mean±SD. (FIGS. 20G and 20H) Representative T2-MRI images and ventricular volume quantification showing a range of moderate to severe ventriculomegaly by LiCl pretreatment in pediatric intraventricular hemorrhage (IVH) model. LiCl+PBS N=7, LiCl+IVH N=15 * p=0.0175. Welch's two-tail unpaired t-test. Data presented as mean±SD. (FIG. 201) Distribution of ventriculomegaly severity. (FIGS. 20J-20L) baseline intracranial pressure (ICP) (FIG. 20J) and capacity for cerebrospinal fluid (CSF) efflux (FIG. 20K). Low capacity for cerebrospinal fluid (CSF) efflux correlated with greater ventriculomegaly (FIG. 20L). LiCl+PBS N=5, LiCl+IVH N=8. ***p=0.0008. Welch's two-tail unpaired t-test. Data presented as mean SD. Each data point represents one mouse.

FIGS. 21A-21Q are schematics, images, and plots demonstrating choroid plexus (ChP) NKCC1 overexpression reverses intraventricular hemorrhage (IVH)-induced ventriculomegaly in mouse models of pediatric post-hemorrhagic hydrocephalus (PHH). (FIG. 21A) Timeline of AAV-NKCC1 pretreatment in the pediatric intraventricular hemorrhage (IVH) model. (FIG. 21B) Representative images showing AAV-NKCC1 (with HA tag) transduction of choroid plexus (ChP) epithelial cells with apical localization. Scale bar=100 μm. (FIGS. 21C-21F) Representative T2-MRI images showing ventricle sizes in mice pretreated with AAV-NKCC1 (C-D, light purple arrows: ventricles from NKCC1+PBS mice; dark purple arrows: ventricles from NKCC1+IVH mice) and mice without pretreatment (E-F, white arrows: ventricles from PBS mice; grey arrows: ventricles from intraventricular hemorrhage (IVH) mice). Scale=2 mm. (FIG. 21G) Ventricle volumes in intraventricular hemorrhage (IVH) vs. PBS control mice with AAV-NKCC1 pre-treatment. NKCC1+PBS N=5, NKCC1+IVH N=6, not significant. Welch's two-tail unpaired t-test. Data presented as mean±SD. For comparison with values from unpretreated mice, see FIG. 18G. (FIGS. 21H-21J) Compliance, baseline intracranial pressure (ICP), and capacity for cerebrospinal fluid (CSF) efflux in intraventricular hemorrhage (IVH) models +/−AAV-NKCC1 pretreatment. NKCC1+PBS N=3, NKCC1+IVH N=4, * p=0.0235. Welch's two-tail unpaired t-test. Data presented as mean±SD. (FIG. 21K) Timeline of AAV-NKCC1 treatment following intraventricular hemorrhage (IVH). AAV-GFP was used as control. (FIGS. 21L-21N) Representative T2-MRI images and quantification of ventricular volume in intraventricular hemorrhage (IVH) mice treated with AAV-GFP (L, grey arrows showing ventricles) vs AAV-NKCC1 (M, light purple arrows showing ventricles). Scale=2 mm. intraventricular hemorrhage (IVH)+GFP N=13, intraventricular hemorrhage (IVH)+NKCC1 N=11, * p=0.0104. Welch's two-tail unpaired t-test. Data presented as mean SD. For comparison with values from unpretreated mice, see FIG. 18G. (FIGS. 21O and 21P) baseline intracranial pressure (ICP) and capacity for cerebrospinal fluid (CSF) efflux. intraventricular hemorrhage (IVH)+GFP N=4, intraventricular hemorrhage (IVH)+NKCC1 N=4, * p=0.015. Welch's two-tail unpaired t-test. Data presented as mean±SD. (FIG. 21Q) cerebrospinal fluid (CSF) [K⁺] was reduced by AAV-NKCC1 treatment 3 days following intraventricular hemorrhage (IVH). intraventricular hemorrhage (IVH)+GFP N=3, intraventricular hemorrhage (IVH)+NKCC1 N=6, ** p=0.0035. Welch's two-tail unpaired t-test. Data presented as mean±SD. Each data point represents one mouse, except for cerebrospinal fluid (CSF) data in FIG. 21Q where each data point represents one biological replicate, and 2-3 mice were pooled in each biological replicate to reach sufficient cerebrospinal fluid (CSF) volume for performing assay.

FIGS. 22A-22J are schematics, images, and plots demonstrating the adult mouse model of post-hemorrhagic hydrocephalus (PHH) mirrors key aspects of pediatric post-hemorrhagic hydrocephalus (PHH) and responds to choroid plexus (ChP)-NKCC1 therapy. (FIG. 22A) Schematic depicting the devices and surgical implantation for in vivo choroid plexus (ChP) live-imaging with two-photon microscopy. (FIGS. 22B and 22C) Calcium responses to serum in choroid plexus (ChP) epithelial cells (FoxJ1-Cre::GCaMP6f mice) in vivo. Scale bar=100 μm. (FIGS. 22D and 22E) Representative fluorescence intensity tracing with serum infusion and quantification of peak intensity. aCSF N=4, serum N=3. * p=0.0318. Welch's two-tail unpaired t-test. Data presented as mean±SD. (FIG. 22F) Schematic depicting adult intraventricular hemorrhage (IVH) serial infusion test post-IVH. (FIG. 22G) Adult cerebrospinal fluid (CSF) efflux capacity post-IVH measured by infusion test. Naïve: N=3, 1-3 days: N=7, 21 days: N=5, 46 days: N=6. ** p=0.0016, ***p=0.0009. Welch's two-tail unpaired t-test. Data presented as mean SD. (FIG. 22H) Schematic depicting the therapeutic approach in adult post-hemorrhagic hydrocephalus (PHH) model and the time-course of serial MRI. (FIG. 22I) Representative T2-MRI images showing rapid reversal of ventriculomegaly in adult post-hemorrhagic hydrocephalus (PHH) model by choroid plexus (ChP)-NKCC1 overexpression. AAV-GFP was used as control. Scale=5 mm. (FIG. 22J) Relative ventricular volume in adult post-hemorrhagic hydrocephalus (PHH) mice with AAV-NKCC1 treatment or control. For each mouse, the ventricular volumes on different days were normalized to the value of Day 2 post-IVH, immediately prior to AAV transduction. N=4, * p<0.05 (Day 3: p=0.0138; Day 6: p=0.0386; Day 13: p=0.0152). Data analyzed by multiple t-test and corrected for multiple comparisons using Holm-Sidak method. Data presented as mean±SD. Each data point represents one mouse.

FIGS. 23A-23F are a schematic, images, and plots demonstrating adult human cerebrospinal fluid (CSF) shows positive correlation between post-SAH/intraventricular hemorrhage (IVH) ionic disequilibrium and shunted outcome. (FIG. 23A) Schematic depicting study design. (FIG. 23B) Representative CT images from patients with resolved (#1) vs. shunted (#2) hydrocephalus. (FIGS. 23C and 23D) cerebrospinal fluid (CSF) osmolarity decreased in some cerebrospinal fluid (CSF) samples regardless of disease course. Shaded area shows 95% normal values (65). Resolved, N=18 (7 patients, 2-3 timepoints per patients).Shunted, N=21 (8 patients, 2-3 timepoints per patient). Data presented as mean±SD. (FIGS. 23E and 23F) Absolute cerebrospinal fluid (CSF) [K⁺] was not altered but relative cerebrospinal fluid (CSF) [K⁺] to total osmolarity was higher in individuals requiring permanent shunting vs. individuals who recovered and had initial ventricular catheter removed. Resolved, N=18 (7 patients, 2-3 timepoints per patients). Shunted, N=21 (8 patients, 2-3 timepoints per patient). * p=0.016. Welch's two-tailed unpaired t-test. Data presented as mean±SD. Each data point in FIGS. 6A-6F represents one cerebrospinal fluid (CSF) sample collected from one patient at a single timepoint.

FIG. 24 is a schematic presenting a non-limiting summary of a model of choroid plexus (ChP) compensatory actions in pediatric and adult post-hemorrhagic hydrocephalus (PHH). The schematics depict three scenarios of choroid plexus (ChP) activities post-IVH in both pediatric and adult cases. Top: Without any intervention, the choroid plexus (ChP) responds with acute calcium activation following blood exposure in the ventricles. After 48 hrs., the cerebrospinal fluid (CSF)-[K⁺] increases, likely due to blood cells lysis. In parallel, the choroid plexus (ChP) activated SPAK increases NKCC1 activation, via phosphorylation. pNKCC1 transports Na⁺, K⁺, Cl⁻, and water following combined electrochemical gradients of the three ions. With elevated cerebrospinal fluid (CSF)-[K⁺], NKCC1 co-transports ions and water from cerebrospinal fluid (CSF) into the choroid plexus (ChP), resulting in compensated post-hemorrhagic hydrocephalus (PHH) where only mild ventriculomegaly is observed with enhanced cerebrospinal fluid (CSF) efflux capacity. Middle: With augmentation of choroid plexus (ChP) function by AAV-mediated NKCC1 overexpression, cerebrospinal fluid (CSF)-K⁺ and water clearance is enhanced, preventing ventriculomegaly. Bottom: When choroid plexus (ChP) epithelial physiology is disrupted (e.g., by LiCl), NKCC1 phosphorylation is reduced and the capacity for cerebrospinal fluid (CSF) efflux is limited, thereby resulting in decompensated post-hemorrhagic hydrocephalus (PHH) with severe ventriculomegaly.

FIGS. 25A-25H are a flow chart, volcano plots, bar graphs, and an image of a clustering analysis relating to a plasma proteomic study showing age-dependent differences in blood proteome relevant to clotting capacity. (FIG. 25A) Flowchart showing proteomic study design. (FIGS. 25B and 25C) PCA and unsupervised hierarchical clustering analysis showing sample clustering by age. (FIGS. 25D-25F) Volcano plots showing age-dependent difference in blood composition. (FIG. 25G) Plasma plasminogen (PLG), alpha-2-macroglobulin (A2 mg), and kininogen-1 (KNG1) levels showing each biological replicate (Embryo: N=9, adult N=10; 50% male, 50% female). PLG: ***p=0.0002, A2 mg and KNG1: **** p<0.0001. Data analyzed by multiple t-test and corrected for multiple comparisons using Holm-Sidak method. Data presented as mean±SD. (FIG. 25H) cerebrospinal fluid (CSF) PLG, A2 mg, and KNG1 levels from a separate dataset (N=2).

FIGS. 26A and 26B are images showing blood clotting outcomes differed in E14.5 vs. P4 post-hemorrhagic hydrocephalus (PHH) models. (FIG. 26A) Representative images of blot clots isolated from P4 post-IVH brains. Scale bar=5 mm. (FIG. 26B) Representative images of E14.5 vs P4 post-IVH brains showing lack of blood clotting in E14.5 brains.

FIG. 27 provides a schematic, an image, and a bar graph relating to immediate early gene activation by intraventricular hemorrhage (IVH). Representative RNAScope image and quantification showing increased c-fos mRNA expression 30 min. following blood exposure. Scale=200 μm. N=6; ****p<0.0001, Welch's two-tail unpaired t-test. Data presented as mean±SD.

FIGS. 28A-28C are plots demonstrating LiCl pretreatment alone does not change compliance (FIG. 28A), baseline intracranial pressure (ICP) (FIG. 28B), or cerebrospinal fluid (CSF) clearance capacity (conductance) (FIG. 28C). Data presented as mean±SD. FIGS. 29A-29D are a schematic, images, and plots relating to a model of decompensated pediatric post-hemorrhagic hydrocephalus (PHH) with EDTA. (FIG. 29A) Workflow schematic. (FIG. 29B) Representative T2-MRI images showing severe ventriculomegaly by EDTA in intraventricular hemorrhage (IVH) mice. (FIGS. 29C and 29D) Normalized ventricle volume quantification of mice receiving PBS alone (N=9) vs. PBS+EDTA (N=9), and EDTA+PBS (N=9) vs. EDTA+blood (N=17). Data presented as mean±SD (C) * p=0.0307; (D) * p=0.039, Welch's two-tail unpaired t-test. Data presented as mean±SD. Each data point represents one individual mouse.

FIGS. 30A and 30B are an image and a plot relating to quality control of adult cranial window and cannula placement. (FIG. 30A) CT images of cranial window and cannula following placement. Sagittal views of the cranial window (a) and cannula (b) confirm their correct localization to lateral ventricles. (FIG. 30B) Cranial window and cannula placement did not change baseline intracranial pressure (cannula alone N=5, cranial window and cannula N=4).

FIGS. 31A-31F are a schematic, images, a plot, and heat maps demonstrating adult choroid plexus (ChP) explants respond robustly to plasma stimulation but less so to osmolarity challenge. (FIG. 31A) Schematic of adult choroid plexus (ChP) explant imaging with application of plasma from age-matched blood. (FIG. 31B) Epifluorescence calcium activity of epithelial cells from a Lateral Ventricle (LV) choroid plexus (ChP) explant (FoxJ1-Cre:: GCaMP6f mice) increased in response to plasma (left: t=−5 s before plasma; middle: t=0 s when plasma was applied; right: t=5 s after plasma). (FIG. 31C) Lighter grey: representative fluorescence trace of full image field shown in (FIG. 31B) across time. Black: aCSF control trace. (FIG. 31D) Representative epi-fluorescence intensity cellular tracing with plasma stimulation. (FIG. 31E) Representative fluorescence intensity cellular tracing with extreme low osmolarity aCSF-based solution (91.7mEq/L) stimulation. (FIG. 31F) Representative fluorescence intensity tracing with aCSF-based solutions of various osmolarity values. (FIGS. 31D-31F) Line indicates cutoff for non-responders, based on peak activity within 25 seconds of solution application.

FIGS. 32A-32D are plots relating to supportive clinical information for study design. Patient information showing equal distribution of demographics (FIG. 32A), illness severity (FIG. 32B), and time (year (FIG. 32C) and hour of day (FIG. 32D)) of cerebrospinal fluid (CSF) collection.

FIGS. 33A-33D are plots showing patients requiring shunting presented with larger range of cerebrospinal fluid (CSF) [K⁺] fluctuation. (FIG. 33A) relative cerebrospinal fluid (CSF) [K⁺] in both groups of patients (shunted and resolved) over time. (FIG. 33B) Percent fluctuation of cerebrospinal fluid (CSF) [K⁺] averaged across time; Resolved, N=7. Shunted, N=8. * p=0.011, Welch's two-tail unpaired t-test. Data presented as mean±SD. (FIG. 33C) Maximum percent fluctuation of cerebrospinal fluid (CSF) [K⁺] from each patient; Resolved, N=7. Shunted, N=8. * p=0.044, Welch's two-tail unpaired t-test. Data presented as mean±SD. (FIG. 33D) Average change of osmolarity. Data presented as mean±SD.

FIGS. 34A-34F are plots demonstrating common clinical parameters did not correlate with hydrocephalus outcome. (FIGS. 34A-34C) Imaging parameters evaluating intraventricular hemorrhage (IVH) volumes and ventricle volumes. (FIGS. 34D-34F) cerebrospinal fluid (CSF) hydrodynamic parameters evaluating baseline intracranial pressure (ICP) and cerebrospinal fluid (CSF) drainage.

### DETAILED DESCRIPTION OF THE INVENTION

The invention features compositions and methods that are useful for treating cerebrospinal fluid disorders and various associated conditions.

The invention is based, at least in part, on the discovery that overexpression of NKCC1 in the choroid plexus (ChP) resulted in increased cerebrospinal fluid (CSF) K⁺ clearance, increased cerebral compliance, and reduced circulating cerebrospinal fluid (CSF) in the brain without changes in intracranial pressure. Accordingly, the invention provides methods for regulating CSF accumulation in the brain involving increasing the expression, activity, or level of NKCC1 in the choroid plexus, for example.

### Cerebrospinal Fluid (CSF) Regulation

Cerebrospinal fluid (CSF) provides vital support for the brain. Abnormal cerebrospinal fluid (CSF) accumulation, such as hydrocephalus, can negatively affect perinatal neurodevelopment. The mechanisms regulating cerebrospinal fluid (CSF) clearance during the postnatal critical period are unclear. A subset of infants and adults with hemorrhagic stroke and intraventricular hemorrhage (IVH) ultimately develop a life-threatening accumulation of cerebrospinal fluid (CSF), termed post-hemorrhagic hydrocephalus (PHH). An incomplete understanding of this variably progressing condition has hampered the development of new therapies outside of the current framework of serial neurosurgical interventions.

In the Examples provided herein, it is shown that cerebrospinal fluid (CSF) K⁺, accompanied by water, is cleared through the choroid plexus (ChP) during mouse early postnatal development. At this developmental stage, the choroid plexus (ChP) showed increased ATP production and increased expression of ATP-dependent K⁺ transporters, particularly the Na⁺, K⁺, Cl⁻, and water cotransporter NKCC1. Overexpression of NKCC1 in the choroid plexus (ChP) resulted in increased cerebrospinal fluid (CSF) K⁺ clearance, increased cerebral compliance, and reduced circulating cerebrospinal fluid (CSF) in the brain without changes in intracranial pressure in experiments carried out in vivo. Moreover, choroid plexus (ChP)-specific NKCC1 overexpression in an obstructive hydrocephalus mouse model resulted in reduced ventriculomegaly. Collectively, the results described herein below indicate that NKCC1 regulates cerebrospinal fluid (CSF) K⁺ clearance through the choroid plexus (ChP) during postnatal neurodevelopment in mice.

The invention is also based in part upon the discovery of a targetable choroid plexus (ChP) mechanism of cerebrospinal fluid (CSF) surveillance and potassium homeostasis that decreased ventricle size in embryonic, neonatal, and adult mouse models of intraventricular hemorrhage (IVH). Intraventricular blood triggered a rapid rise in cytosolic calcium in most choroid plexus (ChP) epithelial cells in vivo and ex vivo, followed by activation of a trans-choroidal route of cerebrospinal fluid (CSF) ion/water clearance out of the ventricle through a Na—K—Cl and water cotransporter, NKCC1. This choroid plexus (ChP)-driven cerebrospinal fluid (CSF) homeostasis was sensitive to perturbations in calcium signaling; interruption of choroid plexus (ChP) intracellular calcium signaling with LiCl worsened ventriculomegaly. AAV-targeted augmentation of NKCC1 expression rapidly reversed blood-induced ventriculomegaly and led to persistently increased cerebrospinal fluid (CSF) clearance capacity, validating the model and treating the most salient clinical features of post-hemorrhagic hydrocephalus (PHH). Finally, it was found that the degree of cerebrospinal fluid (CSF) ionic disequilibrium correlated with clinical outcome in humans following hemorrhagic stroke, suggesting that the findings regarding choroid plexus (ChP) function are applicable to patients. Collectively, the examples provided herein highlight a novel role of the choroid plexus (ChP) to rapidly compensate for alterations in cerebrospinal fluid (CSF) homeostasis following intraventricular hemorrhage (IVH), provide a new framework for the pathogenesis of post-hemorrhagic hydrocephalus (PHH), and demonstrate the utility of targeted gene therapy to mitigate intracranial fluid imbalance following hemorrhages. In embodiments, the methods of the invention involve augmenting NKCC1 activity in a cell through supplemental expression via AAV, forced activation via altered forms of NKCC1, pharmacological manipulation, and/or regulation of upstream components of factors regulating NKCC1 expression, activity, or levels.

### Cerebrospinal Fluid (CSF) and Brain Function and Development

A balance between cerebrospinal fluid (CSF) production and clearance (influx/efflux) is essential for normal brain function and development. Disrupted cerebrospinal fluid (CSF) volume homeostasis with excessive cerebrospinal fluid (CSF) accumulation is implicated in pediatric brain disorders, in particular congenital hydrocephalus, where patients suffer from a potentially life-threatening accumulation of cerebrospinal fluid (CSF) and frequently develop neurological deficits that last through childhood and into adult life. A better understanding of developing cerebrospinal fluid (CSF) dynamics may help explain why early phases of brain development (e.g. from third trimester to 6 months after birth in human) represent a period of high vulnerability to certain congenital disorders.

Critically, how cerebrospinal fluid (CSF) is cleared during this perinatal period remains a mystery. Progress in cerebrospinal fluid (CSF) dynamics research has identified several putative cerebrospinal fluid (CSF) clearance routes including arachnoid villi and granulations in human (and only arachnoid villi in non-human mammalian species), perineural and paravascular pathways, and meningeal lymphatics. While many mechanistic questions and validation work remain regarding the identified routes, their developmental time-courses are even less understood. In human, arachnoid granulations are not fully formed until 2 years of age. In other mammalian species (rats, pigs, and sheep), lymphatic vessels contribute to cerebrospinal fluid (CSF) clearance by connecting with cerebrospinal fluid (CSF) spaces around the cribriform plate and olfactory nerve roots during perinatal brain development. Similarly, lymphatic structures begin to sprout at the base of the skull around birth in mice, but mature throughout the first postnatal month. Thus, available data suggest that the abovementioned cerebrospinal fluid (CSF) clearance routes are unlikely to fully account for cerebrospinal fluid (CSF) outflow during the critical and sensitive early phase of brain development. Identifying and manipulating the early endogenous cerebrospinal fluid (CSF) clearance mechanisms could provide one powerful approach for tackling neurodevelopmental disorders involving cerebrospinal fluid (CSF) dysregulation, and may also be applied to fluid disorders affecting adults.

Specific viral-based therapeutic products, compositions, methods and approaches for treating neurological, neurodevelopmental, or neurogenetic diseases, disorders, and pathologies are described herein. In embodiments, virus vectors and vehicles for gene delivery are designed and produced to contain a polynucleotide sequence of a gene of interest, such as NKCC1, which is specifically and functionally expressed in specific cell populations (e.g., choroid plexus epithelial cells) following transduction of the cells by the virus vector or vehicle. In an embodiment, an enhancer harbored by the virus is capable of restricting the expression of the transgene to certain cells. In embodiments, expression of the transgene is restricted to expression in a population of cells. In an embodiment, the expression of the transgene is specifically modulated in a cell.

### Choroid Plexus (ChP)

The choroid plexus (ChP) is an intraventricular epithelial structure that forms the majority of the blood-CSF barrier and develops prenatally. It contains diverse ion and fluid transporters along its vast surface area. Although the prevailing model posits that the choroid plexus (ChP) provides net luminal secretion of ions and water to form cerebrospinal fluid (CSF) in adult brains, historical clinical observations suggest some absorptive functions of the choroid plexus (ChP), which is supported by animal studies. Moreover, extensive studies have documented ion gradients driving bi-directional trafficking at the choroid plexus (ChP)-CSF interface through various choroid plexus (ChP) transporters. Furthermore, broad transcriptional changes of the machinery regulating fluid/ion transport support the concept of temporally dynamic and possibly context-dependent choroid plexus (ChP) functions in determining net directionality of cerebrospinal fluid (CSF) transport.

As the principal brain structure that regulates cerebrospinal fluid (CSF) properties and influences cerebrospinal fluid (CSF) hydrodynamics, the choroid plexus (ChP) represents a promising target for the treatment of post-hemorrhagic hydrocephalus (PHH). The choroid plexus (ChP) forms the dominant physiologic interface between the bloodstream and cerebrospinal fluid (CSF). Located within each brain ventricle, the choroid plexus (ChP) and its microvilli have a total surface area ranging from 5 to 50% that of the human blood-brain barrier. This relatively large choroid plexus (ChP) epithelial surface area enables unique roles for the surveillance and regulation of cerebrospinal fluid (CSF) volume and composition, using mechanisms that include regulated water and ion exchange. In addition to its dynamic and multivariate roles in cerebrospinal fluid (CSF) regulation, the choroid plexus (ChP) is accessible to adeno-associated viral (AAV)-based gene therapy with high tissue specificity. Transduction of choroid plexus (ChP) epithelial cells or ventricular ependymal cells has also been shown to improve neurologic symptoms in rodent models of Huntington's disease, lysosomal storage disorders, and Alzheimer's disease, suggesting that the choroid plexus (ChP) is a viable target for sustained CNS therapeutics. Therefore, genetic manipulation of the choroid plexus (ChP) represents a favored methodology for new therapies that aim to correct cerebrospinal fluid (CSF) properties including ionic equilibrium and hydrodynamics.

Multiple lines of evidence suggest that the choroid plexus (ChP) is centrally involved during the pathogenesis of post-hemorrhagic hydrocephalus (PHH), although its precise role in cerebrospinal fluid (CSF) regulation has been understudied due to limits in methodology, including targeted genetic manipulation, live imaging modalities, and reliable techniques to evaluate cerebrospinal fluid (CSF) hydrodynamics. As a consequence, it is still unclear whether post-hemorrhagic cerebrospinal fluid (CSF) accumulation is a consequence of inadequate clearance/reabsorption, excess production of cerebrospinal fluid (CSF) (from the choroid plexus (ChP) or extra-choroidal sources), or a combination of these effects.

To further explore potentially absorptive properties of the early choroid plexus (ChP), the expression of transporters, the energy systems and ionic gradients that govern their activity, and their physiological effects across the timespan of early postnatal development in mice were studied in the Examples provided herein. Taken together the data of the Examples support a previously undescribed developmental mechanism for net cerebrospinal fluid (CSF) clearance by the Na⁺—K⁺—Cl⁻ and water co-transporter, NKCC1, on the apical membrane of the choroid plexus (ChP) during a specific stage. While NKCC1 retains bi-directional transporter potential throughout life, its role in mediating net cerebrospinal fluid (CSF)-to-ChP transport during the early postnatal period of brain development critically influenced the establishment of cerebrospinal fluid (CSF) ion and fluid homeostasis. These results have implications for the pathophysiology of congenital disorders accompanied by dysregulated cerebrospinal fluid (CSF) and could inform strategies for treatment of neonatal hydrocephalus and perhaps other disorders.

### Intraventricular Hemorrhage

Intraventricular hemorrhage (IVH) resulting from hemorrhagic stroke is a common cause of post-hemorrhagic hydrocephalus (PHH), an excessive accumulation of cerebrospinal fluid (CSF) accompanied by neurologic decline. Accounting for nearly a quarter of cases, intraventricular hemorrhage (IVH) is the leading etiology of pediatric hydrocephalus in North America and represents one of the most devastating complications of preterm birth. Approximately 20% of infants with intraventricular hemorrhage (IVH) develop permanent disruptions of cerebrospinal fluid (CSF) homeostasis and require repeated neurosurgical interventions throughout life, which encumbers their families and strains the healthcare system. Over 80% of severe cases also develop cerebral palsy, epilepsy, or other neurologic impairments. Adults are similarly afflicted by post-hemorrhagic hydrocephalus (PHH) following hemorrhagic stroke complicated by intraventricular hemorrhage (IVH), with modern rates of adult post-hemorrhagic hydrocephalus (PHH) also approximating 20%.

In the Examples provided herein, the role of NKCC1 in pediatric and adult mouse models of intraventricular hemorrhage (IVH) with acquired ventriculomegaly was studied and it was found that augmenting NKCC1 activity specifically in the choroid plexus (ChP) accelerated cerebrospinal fluid (CSF) clearance following intracerebroventricular (ICV) blood injections, restored cerebrospinal fluid (CSF) ionic homeostasis, and mitigated ventriculomegaly. Conversely, disruption of early choroid plexus (ChP) responses exacerbated the hydrocephalic outcomes. Not wishing to be bound by theory, the data of the Examples support the interpretation that inadequate cerebrospinal fluid (CSF) clearance is the primary driver of post-hemorrhagic cerebrospinal fluid (CSF) accumulation and post-hemorrhagic hydrocephalus (PHH). The findings also support the concept of the choroid plexus (ChP) as a first line of defense to maintain cerebrospinal fluid (CSF) homeostasis and that increasing the expression, activity, or levels of NKCC1 is useful for the treatment of conditions such as post-hemorrhagic hydrocephalus (PHH) following pediatric and adult hemorrhagic strokes.

### Nucleic Acid Therapy

Methods of introducing exogenous nucleic acid molecules into a cell (e.g., choroid plexus cell) are known in the art. For example, eukaryotic cells can take up nucleic acid molecules from the environment via transfection (e.g., calcium phosphate-mediated transfection). Transfection does not employ a virus or viral vector for introducing the exogenous nucleic acid into the recipient cell. Stable transfection of a eukaryotic cell comprises integration into the recipient cell's genome of the transfected nucleic acid, which can then be inherited by the recipient cell's progeny.

Eukaryotic cells (e.g., a choroid plexus cell) can be modified via transduction, in which a virus or viral vector stably introduces an exogenous nucleic acid molecule to the recipient cell. Eukaryotic transduction delivery systems are known in the art. Transduction of most cell types can be accomplished with retroviral, lentiviral, adenoviral, adeno-associated, and avian virus systems, and such systems are well-known in the art. While retroviruses systems are generally not compatible with neuronal cell transduction, lentiviruses are a genus of retroviruses well-suited for transducing stem cells as well as neuronal cells. Thus, in some embodiments of the present disclosure, the viral vector system is a lentiviral system. In some embodiments, the viral vector system is an avian virus system, for example, the avian viral vector system described in U.S. Pat. No. 8,642,570, DE102009021592, PCT/EP2010/056757, and EP2430167, the contents of which are incorporated herein by reference in their entirety. In some embodiments, the viral vectors are assembled or packaged in a packaging cell prior to contacting the intended recipient cell. In some embodiments, the vector system is a self-inactivating system, wherein the viral vector is assembled in a packaging cell, but after contacting the recipient cell, the viral vector is not able to be produced in the recipient cell.

The components of a viral vector are encoded on plasmids, and because efficiencies of transduction decrease with large plasmid size, multiple plasmids that have different viral sequences necessary for packaging may be necessary. For example, in a lentiviral vector system, a first plasmid may comprise a nucleotide sequence encoding a Group antigens (gag) and/or a reverse transcriptase (pol) gene, while a second plasmid encodes regulator of expression of virion proteins (rev) and/or envelope (env) genes. The exogenous nucleic acid molecule comprising a transgene can be packaged into the vector and delivered into a recipient cells where the transgene is integrated into the recipient cell's genome. Additionally, the transgene may be packaged using a split-packaging system as described in U.S. Pat. No. 8,642,570, DE102009021592, PCT/EP2010/056757, and EP2430167.

Expression control sequences include transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and sequences that enhance secretion of the encoded product. A great number of expression control sequences, including promoters which are native, constitutive, inducible and/or tissue-specific, are known in the art and are suitable for use in embodiments of the present invention.

In some embodiments of the present invention a polyadenylation sequence can be inserted following a heterologous gene sequence. In various embodiments, the polyadenylation sequence is inserted before a 3′ adeno-associated virus (AAV) inverted terminal repeat (ITR) sequence. A rAAV vector useful in the present invention may also comprise an intron sequence. A non-limiting example of an intron sequence is an intron derived from SV-40, and is referred to as the SV-40 T intron sequence. Vectors of the present invention in various embodiments comprise an internal ribosome entry site (IRES). An IRES sequence is used to produce more than one polypeptide from a single gene transcript. An IRES sequence may be used to produce a protein that includes more than one polypeptide chain. AAV vectors are described in greater detail below.

The precise nature of sequences needed for gene expression in host cells may vary between species, tissues or cell types. In some embodiments, vectors of the present invention comprise 5′ non-transcribed and 5′ non-translated sequences involved with the initiation of transcription and translation respectively of a heterologous gene, such as, to provide non-limiting examples, a TATA box, a capping sequence, a CAAT sequence, an enhancer elements, and the like. In various embodiments, a 5′ non-transcribed sequences can include a promoter region that includes a promoter sequence for transcriptional control of an operably joined gene. In some embodiments, vectors of the present invention include enhancer sequences or upstream activator sequences as desired. The vectors of the invention may optionally include 5′ leader or signal sequences. The choice and design of an appropriate vector is within the ability and discretion of one of ordinary skill in the art.

Examples of suitable promoters include, but are not limited to the retroviral Rous sarcoma virus (RSV) LTR promoter (optionally with the RSV enhancer), the cytomegalovirus (CMV) promoter (optionally with the CMV enhancer) (see, e.g., Boshart et al (1985) Cell, 41:521-530), the SV40 promoter, the dihydrofolate reductase promoter, the β-actin promoter (e.g., chicken β-actin promoter), the phosphoglycerol kinase (PGK) promoter, the EF1α promoter, the CBA promoter, UBC promoter, GUSB promoter, NSE promoter, Synapsin promoter, MeCP2 (methyl-CPG binding protein 2) promoter, GFAP; CBh promoter and the like. Exemplary promoters include, but are not limited to, the MoMLV LTR, a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the cytomegalovirus enhancer/chicken beta-actin/Rabbit β-globin promoter (CAG promoter; Niwa et al., Gene, 1991, 108(2):193-9) and the elongation factor 1-alpha promoter (EF1-alpha) promoter (Kim et al., Gene, 1990, 91(2):217-23 and Guo et al., Gene Ther., 1996, 3(9):802-10). The promoter can be a constitutive, inducible, or repressible promoter.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Non-limiting examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (see, e.g., WO 98/10088); the ecdysone insect promoter (see, e.g., No et al, Proc. Natl. Acad. Sci. USA, 93:3346-3351 (1996)), the tetracycline-repressible system (see, e.g., Gossen et al, Proc. Natl. Acad. Sci. USA, 89:5547-5551 (1992)), the tetracycline-inducible system (see, e.g., Gossen et al, Science, 268:1766-1769 (1995), and Harvey et al, Curr. Opin. Chem. Biol., 2:512-518 (1998)), the RU486-inducible system (see, e.g., Wang et al, Nat. Biotech., 15:239-243 (1997) and Wang et al, Gene Ther., 4:432-441 (1997)) and the rapamycin-inducible system (see, e.g., Magari et al, J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for a heterologous gene comprised by the vector will be used. The native promoter may be preferred when it is desired that expression of the heterologous gene should mimic the native expression. The native promoter may be used when expression of the heterologous gene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the promoter expresses the heterologous gene in a brain cell (e.g., a choroid plexus cell). In some embodiments, the promoter expresses the heterologous gene in a choroid plexus cell. Choroid plexus-specific promoters are known in the art and described, for example, by Regev et al., PNAS Mar. 2, 2010 107 (9) 4424-4429, which is incorporated herein by reference in its entirety. In one embodiment, the promoter comprises 2.5 kb of the 5′-flanking region of the CRFR2β gene. In some embodiments, the heterologous gene is exclusively expressed in choroid plexus epithelial cells.

In some embodiments, vectors of the present invention comprise expression control sequences imparting tissue-specific gene expression capabilities. In some cases, the tissue-specific expression control sequences bind tissue-specific transcription factors that induce transcription in a tissue specific manner. In some embodiments, the expression control sequence allows for specific expression in a choroid plexus cell.

In some embodiments, one or more inhibitory nucleic acids are incorporated in a gene of a vector to inhibit the expression of a heterologous gene in one or more tissues of a subject harboring the heterologous gene.

In some embodiments, a vector of the present invention may comprise a replication open reading frame (Rep) from an adeno-associated virus (AAV) serotype that differs from that serotype which corresponds to a capsid open reading frame (Cap) comprised by the vector. In one embodiment, the Rep and Cap are expressed from separate sources (e.g., separate vectors, or a cell and a vector). In embodiments, the AAV vector is an AAV2/5 vector.

After the introduction of one or more vector(s), host cells are cultured prior to administration to a subject. In some embodiments, the expression of recombinant proteins can be detected by immunoassays including Western blot analysis, immunoblot, and immunofluorescence. Purification of recombinant proteins can be carried out by any of the methods known in the art or described herein, for example, any conventional procedures involving extraction, precipitation, chromatography, and electrophoresis. A further purification procedure that may be used for purifying proteins is affinity chromatography using monoclonal antibodies which bind a target protein. Generally, crude preparations containing a recombinant protein are passed through a column on which a suitable monoclonal antibody is immobilized. The protein usually binds to the column via the specific antibody while the impurities pass through. After washing the column, the protein is eluted from the gel by changing pH or ionic strength, for example.

Recombinant Adeno-Associated Viruses (rAAV)

In some embodiments, a nucleic acid molecule encoding NKCC1 is delivered to a cell (e.g., cell of the choroid plexus) of a subject having or at risk of developing an intracranial fluid imbalance using an adeno-associated virus or virus-like particle. Adeno-associated virus is a small (20-26 nm), icosahedral, and nonenveloped virus. AAV particles contain a single-stranded DNA genome comprising approximately 4.7 kb. The genome contains three open reading frames (ORFs) encoding replication proteins (Rep), capsid proteins (Caps), and the assembly activating protein (AAP), and is flanked by two inverted terminal repeats (ITRs). Interestingly, recombinant adeno-associated virus (rAAV) particles have tissue-specific targeting capabilities, such that a heterologous gene of the rAAV will be delivered specifically to one or more predetermined tissue(s) or cell(s). A capsid protein encoded by the rAAV facilitates the tissue-specific targeting. In various embodiments, the recombinant adeno-associated virus (rAAV) particles disclosed herein are encoded by any one of the vectors and/or polynucleotides described herein or produced by any one of the methods known in the art.

More than 30 naturally occurring serotypes of AAV are available and are useful in the particles, vectors, nucleotide molecules, and methods described herein. Many natural variants in the adeno-associated virus (AAV) capsid exist, allowing identification and use of an AAV with properties specifically suited for neural cells as well as other cell types. AAV viruses (i.e., AAV particles) can be engineered by conventional molecular biology techniques, making it possible to optimize these particles for cell specific delivery of the desired nucleic acid sequences, for minimizing immunogenicity, for tuning stability and particle lifetime, for efficient degradation, for accurate delivery to the nucleus, etc.’

The use of adeno-associated viruses (AAVs) is a common mode of exogenous delivery of polynucleotides because AAVs are relatively non-toxic, provide efficient gene transfer, and can be easily optimized for specific purposes. Among the serotypes of AAVs isolated from human or non-human primates (NHP) and well characterized, human serotype 2 is the first AAV that was developed as a gene transfer vector. This serotype has been widely used for efficient gene transfer experiments in different target tissues and animal models. Other AAV serotypes useful in the methods of this disclosure include, but are not limited to, AAV1, AAV3, AAV4, AAV5, AAV6, AAV6.2, AAV7, AAV8, AAV9, AAV10, rh.10, rh.39, rh,43, CSP3, and the like (see, e.g., WO 2005/033321 and U.S. Pat. No. 7,198,951 for a discussion of various AAV serotypes). In certain embodiments the serotype is selected to optimize a desired mode of delivery. In embodiments, the AAV has an AAV5 serotype. In embodiments, the AAV is an AAV2/5 vector.

Adeno-associated virus components suitable for inclusion in particles and vectors of the present invention include the capsid proteins, including the virion particle (VPs) proteins VP1, VP2, VP3, and hypervariable regions, the replication proteins (rep), including rep 78, rep 68, rep 52, and rep 40, and the sequences encoding these proteins. These components may be readily utilized in a variety of vector systems and cells. Such components maybe used alone or in combination with other adeno-associated virus (AAV) serotype sequences or fragments, or in combination with elements from other AAV or non-AAV viral sequences. In some embodiments, the recombinant adeno-associated virus (rAAV) particle comprises or is a vector. In some embodiments, the viral particle is a recombinant AAV particle comprising a nucleic acid comprising a heterologous gene flanked by one or two AAV inverted terminal repeats (ITRs). In various embodiments, the heterologous gene is encapsidated in the AAV particle. The AAV particle comprises capsid proteins. In some embodiments, the vector comprises a heterologous gene operatively linked to control sequences including promoters and transcription initiation and termination sequences, thereby forming an expression cassette. In embodiments, the AAV vector of the invention is an AAV2/5 vector.

### Polypeptide Expression

In order to express the polypeptides (e.g., NKCC1) described herein, DNA or RNA molecules obtained by any of the methods described herein or those that are known in the art, can be inserted into appropriate expression vectors by techniques known in the art. For example, a double stranded DNA can be cloned into a suitable vector by restriction enzyme linking involving the use of synthetic DNA linkers or by blunt-ended ligation. DNA ligases are usually used to ligate the DNA molecules and undesirable joining can be avoided by treatment with alkaline phosphatase.

Therefore, the invention includes vectors that include nucleic acid molecules (e.g., genes or recombinant nucleic acid molecules encoding genes) as described herein. The term “recombinant vector” includes a vector (e.g., plasmid, phage, phasmid, virus, cosmid, fosmid, or other purified nucleic acid vector) that has been altered, modified or engineered such that it contains greater, fewer or different nucleic acid sequences than those included in the native or natural nucleic acid molecule from which the recombinant vector was derived. For example, a recombinant vector may include a gene, or fragment thereof, operatively linked to regulatory sequences, e.g., promoter sequences, terminator sequences, and the like, as defined herein. Recombinant vectors which allow for expression of the genes or nucleic acids included in them are referred to as “expression vectors.”

The methods of the present invention can involve contacting a cell with an mRNA molecule encoding a polypeptide (e.g., NCC1) to cause the cell to express the encoded polypeptide. Non-limiting examples of methods for production and delivery of mRNA molecules for expression of a polypeptide in a cell are described in, e.g., U.S. Pat. No. 9,750,824, the disclosure of which is incorporated herein by reference in its entirety for all purposes.

In some of the molecules of the invention described herein, one or more DNA or RNA molecules having a nucleotide sequence encoding one or more polypeptides of the invention (e.g., NKCC1) are operatively linked to one or more regulatory sequences, which are capable of integrating the desired DNA or RNA molecule into a cell. Cells which have been stably transformed by the introduced DNA or RNA can be selected, for example, by introducing one or more markers which allow for selection of host cells which contain the expression vector. A selectable marker gene can either be linked directly to a nucleic acid sequence to be expressed, or be introduced into the same cell by co-transfection. Additional elements may also be needed for optimal synthesis of proteins described herein. It would be apparent to one of ordinary skill in the art which additional elements to use.

It can be advantageous to codon-optimize a nucleotide sequence encoding one or more polypeptides of the invention for expression in a host organism. For example, a nucleotide sequence encoding a polypeptide of the invention can be codon-optimized for expression in a human cell. Also, polypeptide sequences of the invention can be humanized to facilitate expression in a human cell.

Factors of importance in selecting a particular vector include, but are not limited to, the ease with which recipient cells that contain the vector are recognized and selected from those recipient cells which do not contain the vector; the number of copies of the vector which are desired in a particular host; and whether it is desirable to be able to “shuttle” the vector between host cells of different species.

Once the vector(s) is constructed to include a DNA or RNA sequence for expression, it may be introduced into an appropriate host cell by one or more of a variety of suitable methods that are known in the art, including but not limited to, for example, transformation, transfection, conjugation, protoplast fusion, electroporation, calcium phosphate-precipitation, direct microinjection, etc.

After the introduction of one or more vector(s), host cells can be grown in a selective medium, which selects for the growth of vector-containing cells. Expression of recombinant proteins can be detected by immunoassays including Western blot analysis, immunoblot, and immunofluorescence. Purification of recombinant proteins can be carried out by any of the methods known in the art or described herein, for example, any conventional procedures involving extraction, precipitation, chromatography and electrophoresis. A further purification procedure that may be used for purifying proteins is affinity chromatography using monoclonal antibodies which bind a target protein. Generally, crude preparations containing a recombinant protein are passed through a column on which a suitable monoclonal antibody is immobilized. The protein usually binds to the column via the specific antibody while the impurities pass through. After washing the column, the protein is eluted from the gel by changing pH or ionic strength, for example.

### Treatment Approaches

In general, the delivery of an NKKC1 encoding polynucleotide may be achieved using appropriate and effective vectors, such as viral or virus vectors (e.g., AAV2/5) described herein. The use of a rAAV vector provides efficient delivery of therapeutic genes to a cell where the genes are expressed. Other methods and approaches for delivering genes to cells involve, for example, the use of purified DNA or RNA under hydrodynamic pressure, a shotgun approach using DNA or RNA adhering to gold particles, or lipid-DNA/RNA complexes. Viruses represent natural vectors for the delivery and expression of exogenous genes in host cells in vivo. An advantage of delivering a transgene to a cell using an mRNA molecule is that expression of the molecule in embodiments is transient and there is a reduced risk of manipulation of the host cell genome.

To achieve enhanced therapy or treatment, a dose of a vector that is required for a therapeutic response may be reduced, e.g., by using certain rAAV serotypes. Alternatively, the surface of an rAAV vector capsid may be altered to include specific ligands for attachment to target tissues and cells as described above. Another approach takes into consideration the trafficking of the virus particle from the endocytoplasmic vesicle to the nucleus. (Zhao, W. et al., 2007, Gene Ther., 14:545-550; Daya, S. and Berns, K. I., 2008, Clin. Microbiol. Rev., 21(4):583-593). Typically, the virus particle-to-infectivity ratio of rAAV vector preparations ranges from 10:1 to 100:1.

For direct delivery to the brain, agents of the invention may be administered by open neurosurgical procedure or by focal injection in order to bypass the blood-brain barrier, to temporally and spatially restrict transgene expression, and to target specific areas of the brain. In some embodiments, the composition is delivered directly to the brain via intracerebroventricular or intrathecal administration. In embodiments, agents of the invention are delivered by intraventricular or intrathecal injection (e.g., to modify choroid plexus epithelial cells). Methods of administration useful in the present disclosure are described, for example in International Application No. PCT/US2020/045106, which is incorporated by reference in its entirety.

Systemic delivery (by intravenous injection) provides a non-invasive alternative for broad gene delivery to a subject. Several groups have developed rAAV capsids that enhance gene transfer to the CNS and certain tissues and cell populations after intravenous delivery. Other modes of vector administration may include lipid-mediated vector delivery, hydrodynamic delivery, and a gene gun.

### Pharmaceutical Compositions

Compositions contemplated in the present disclosure include pharmaceutical compositions comprising polynucleotides encoding NKCC1. In some embodiments, the polynucleotide is present in a vector (e.g., a viral vector described herein). Pharmaceutical compositions as described herein can be provided as sterile liquid preparations, e.g., isotonic aqueous solutions, suspensions, emulsions, dispersions, or viscous compositions, which may be buffered to a selected pH. A liquid preparation may be easier to prepare than a gel, another viscous composition, and a solid composition. Additionally, a liquid composition may be more convenient to administer (i.e., by injection). Viscous compositions, on the other hand, can be formulated within the appropriate viscosity range to provide longer contact periods with specific tissues. Liquid or viscous compositions can comprise a carrier, which can be a solvent or dispersing medium comprising, for example, water, saline, phosphate buffered saline, polyol (e.g., glycerol, propylene glycol, liquid polyethylene glycol, and the like), and suitable mixtures thereof.

Sterile injectable solutions can be prepared by incorporating the polynucleotides described herein in a sufficient amount of an appropriate diluent. Such compositions may be in admixture with a suitable carrier or excipient such as sterile water, physiological saline, glucose, dextrose, or another carrier or excipient suitable for delivering polynucleotides to a subject. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “Remington's Pharmaceutical Science”, 17th edition, 1985, incorporated herein by reference, may be consulted to prepare suitable preparations, without undue experimentation. Additives that enhance the stability and sterility of the cellular compositions, including antimicrobial preservatives, antioxidants, chelating agents, and buffers, can be added. Prevention of the action of microorganisms can be ensured by an antibacterial or antifungal agent including, but not limited to, parabens, chlorobutanol, phenol, and sorbic acid. According to the present disclosure, however, any vehicle, diluent, or additive used must be compatible with injection into the brain (e.g., intracerebroventricular injection).

The compositions can be isotonic, i.e., they have the same osmotic pressure as blood and cerebrospinal fluid. The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride may be suitable for buffers containing sodium ions.

Viscosity of the compositions, if desired, can be maintained at a selected level using a pharmaceutically acceptable thickening agent. In some embodiments, the thickening agent is methylcellulose, which is readily and economically available and is easy to work with. Other suitable thickening agents include, but are not limited to, xanthan gum, carboxymethyl cellulose, hydroxypropyl cellulose, and carbomer. The concentration of the thickener will depend upon the agent selected and the amount of the agent used. Suitable carriers and other additives may be chosen depending on the route of administration and the nature of the dosage form (e.g., a liquid dosage form can be formulated into a solution, a suspension, a gel, or another liquid form, such as a time release formulation or liquid-filled form).

An effective amount of polynucleotides or viral particles to be administered can vary for the subject being treated.

In some embodiments, the pharmaceutical compositions contain a diuretic. In some embodiments, the pharmaceutical compositions of the disclosure contain acetazolamine, furosemide, a derivative thereof, or various combinations thereof.

The skilled artisan can readily determine the amounts of polynucleotides or viral particles and optional additives, vehicles, and/or carrier in compositions to be administered. In one embodiment any additive (in addition to the polynucleotides is present in an amount of about 0.001% to about 50% (weight) solution in phosphate buffered saline, and the active ingredient is present in the order of micrograms to milligrams, such as about 0.0001% to about 5 wt %. In another embodiment, the active ingredient is present at about 0.0001% to about 1 wt %. In yet another embodiment, the active ingredient is present at about 0.0001% to about 0.05 wt %. In still other embodiments, the active ingredient is present at about 0.001% to about 20 wt %. In some embodiments, the active ingredient is present at about 0.01% to about 10 wt %. In another embodiment, the active ingredient is present at about 0.05% to about 5 wt %. For any composition to be administered to an animal or human, and for any particular method of administration, toxicity can be determined by measuring the lethal dose (LD) and LD₅₀ in a suitable animal model e.g., a rodent such as mouse. The dosage of the composition(s), concentration of components therein, and timing of administering the composition(s), which elicit a suitable response can also be determined. Such determinations do not require undue experimentation in light of the knowledge of the skilled artisan, this disclosure, and the documents cited herein. The time for sequential administrations can also be ascertained without undue experimentation.

### Method of Treatment

The present invention provides methods of treating disease and/or disorder (e.g., hydrocephalus or intracranial fluid imbalances) or symptoms thereof, where the methods involve administering a therapeutically effective amount of a pharmaceutical composition of the present disclosure to a subject (e.g., a mammal such as a human). The method includes the step of administering to a subject a therapeutic amount of an agent described herein sufficient to treat the disease or disorder or a symptom thereof, under conditions such that the disease or disorder is treated. In some embodiments, the composition is a pharmaceutical composition described herein. In embodiments, an agent of the invention selectively increases activity, expression, and/or levels of NKCC1 in a cell (e.g., a choroid plexus cell). In embodiments, an agent of the invention indirectly selectively increases activity, expression, and/or levels of NKCC1 in a cell (e.g., a choroid plexus cell).

The subject method has wide applicability to the treatment of conditions associated with intracranial fluid imbalances. In some embodiments, the intracranial fluid imbalance is associated with an intraventricular hemorrhage or a congenital condition. In this regard, the subject method is useful for, but not limited to, treatment of hemorrhagic injury to the brain due to traumas, infectious diseases, cancers, autoimmune diseases, intraventricular hemorrhage, stroke, aneurism, head trauma, cerebral hemorrhage, brain tumors, hydroencephalitis, and operative and postoperative brain injury. Non-limiting examples of diseases that may be treated using the compositions and/or methods of the present disclosure include cerebrospinal fluid disorders, hydrocephalus, intraventricular hemorrhage, hyperkalirrhachia, and ventriculomegaly.

The methods herein include administering to a subject (including a subject identified as in need of such treatment) an effective amount of an agent described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention in general comprise administration of a therapeutically effective amount of the compositions described herein, such as a composition comprising a small molecule activator of NKCC1 activity or expression or any of the agents described herein, to a subject (e.g., animal, human) in need thereof, including a mammal, particularly a human. Such treatment in embodiments is suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any injury, disorder, or disease associated with an intracranial fluid imbalance, optionally following a hemorrhage.

In some embodiments, the therapeutic methods of the invention involve administering a diuretic to a subject. In some embodiments, the diuretic contains acetazolamine, furosemide, a derivative thereof, or various combinations thereof.

The pharmaceutical compositions of this invention can be administered by any suitable routes including, by way of illustration, oral, topical, rectal, transdermal, subcutaneous, intravenous, intramuscular, intranasal, intracranial, intracerebral, intraventricular, intrathecal, and the like. In some embodiments, the administration modalities as described in U.S. Pat. Nos. 5,543,158; 5,641,515 and 5,399,363 may be used to deliver compositions of the present invention. In some embodiments, a preferred mode of administration is intraventricular injection.

For therapeutic uses, the compositions and agents disclosed herein may be administered by any convenient method; for example, parenterally, conveniently in a pharmaceutically or physiologically acceptable carrier, e.g., phosphate buffered saline, saline, deionized water, or the like. The compositions may be added to a retained physiological fluid such as blood or synovial fluid. For central nervous system (CNS) administration, a variety of techniques are available for promoting transfer of an agent across the blood brain barrier including disruption by surgery or injection, drugs which transiently open adhesion contact between central nervous system (CNS) vasculature endothelial cells, and compounds which facilitate translocation through such cells. As examples, many of the disclosed compositions are amenable to be directly injected or infused or contained within implants e.g. osmotic pumps, grafts comprising appropriately transformed cells. Compositions of the present invention may also be amenable to direct injection or infusion, topical, intratracheal/nasal administration e.g. through aerosol, intraocularly, or within/on implants e.g. fibers e.g. collagen, osmotic pumps, or grafts comprising appropriately transformed cells. Generally, the amount administered will be empirically determined. Other additives may be included, such as stabilizers, bactericides, etc. In various embodiments, these additives can be present in conventional amounts.

In various embodiments, the agents of the present invention are administered in sufficient amounts to provide sufficient levels of the agent in a target cell without undue adverse effects. Conventional and pharmaceutically acceptable routes of administration include, but are not limited to, direct delivery to a selected organ or tissue (e.g., the choroid plexus), oral, inhalation (including intranasal and intratracheal delivery), intraocular, intravenous, intramuscular, subcutaneous, intradermal, intratumoral, and other parental routes of administration. Routes of administration may be combined, if desired.

The dose of an agent used to achieve a particular “therapeutic effect” will vary based on several factors including, but not limited to: the route of administration, the level of gene or RNA expression used to achieve a therapeutic effect, the specific disease or disorder being treated, and the stability of the gene or RNA product. One of skill in the art can readily determine a dose range to treat a patient having a particular disease, injury, or condition based on the aforementioned factors, as well as other factors that are well known in the art. In some embodiments, the therapeutic effect is restoration of intracranial fluid balance (e.g., by reducing intracranial pressure).

Administration of agents of the present invention to a subject may be by, for example, intraventricular injection or administration into the bloodstream of the subject. Administration into the bloodstream may be by injection into a vein, an artery, or any other vascular conduit. In some embodiments, the agents are administered into the bloodstream by way of isolated limb perfusion, a technique well known in the surgical arts, the method essentially enabling the artisan to isolate a limb from the systemic circulation prior to administration. A variant of the isolated limb perfusion technique, described in U.S. Pat. No. 6,177,403, can also be employed by the skilled artisan to administer the agent into the vasculature of an isolated limb to potentially enhance transduction into muscle cells or tissue. Moreover, in certain instances, it may be desirable to deliver the agent to the central nervous system (CNS) of a subject. In various embodiments, by “CNS” is meant all cells and tissue of the brain and spinal cord of a vertebrate. Thus, the term can include, but is not be limited to, neuronal cells, glial cells, astrocytes, cereobrospinal fluid (CSF), interstitial spaces, bone, cartilage and the like. An agent may be delivered directly to the central nervous system (CNS) or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection.

Agents of the present invention can be inserted into a delivery device which facilitates introduction by injection or implantation into a subject. Such delivery devices include tubes, e.g., catheters, for injecting cells and fluids into the body of a recipient subject. In a preferred embodiment, the tubes additionally have a needle, e.g., a syringe, through which the cells of the invention can be introduced into the subject at a desired location. Agents of the invention can be inserted into such a delivery device, e.g., a syringe, in different forms. For example, an agent can be suspended in a solution or embedded in a support matrix when contained in such a delivery device. As used herein, the term “solution” includes a pharmaceutically acceptable carrier or diluent in which the agent of the invention remain functional and/or viable. Pharmaceutically acceptable carriers and diluents include saline, aqueous buffer solutions, solvents and/or dispersion media. The use of such carriers and diluents is well known in the art. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. In some embodiments, the selection of the carrier is not a limitation of the present invention. The solution is preferably sterile and fluid. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating recombinant adeno-associated virus particles, nucleotide molecules, and/or vectors as described herein in a pharmaceutically acceptable carrier or diluent and, as other ingredients enumerated herein, followed by filtered sterilization. Optionally, an agent may be administered on support matrices. Support matrices in which an agent can be incorporated or embedded include matrices which are recipient-compatible and which degrade into products which are not harmful to the recipient. Natural and/or synthetic biodegradable matrices are examples of such matrices. Natural biodegradable matrices include plasma clots, e.g., derived from a mammal, and collagen matrices. Synthetic biodegradable matrices include synthetic polymers such as polyanhydrides, polyorthoesters, and polylactic acid. Other examples of synthetic polymers and methods of incorporating or embedding cells into these matrices are known in the art. These matrices provide support and protection for the cells in vivo.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a bioactive factor at a particular target site.

One feature of certain embodiments of an implant can be the linear release of an agent of the present invention, which can be achieved through the manipulation of the polymer composition and form. By choice of monomer composition or polymerization technique, the amount of water, porosity and consequent permeability characteristics can be controlled. The selection of the shape, size, polymer, and method for implantation can be determined on an individual basis according to the disorder, injury, or disease to be treated and the individual patient response. The generation of such implants is generally known in the art.

In another embodiment of an implant an agent of the invention is encapsulated in implantable hollow fibers or the like. Such fibers can be pre-spun and subsequently loaded with the agent, or can be co-extruded with a polymer which acts to form a polymeric coat about the agent.

In addition to the methods of delivery described above, the following techniques are also contemplated as alternative methods of delivering an agent to a subject. Ultrasound has been used as a device for enhancing the rate and efficacy of drug permeation into and through a circulatory system. Other drug delivery alternatives contemplated are intraosseous injection (see, e.g., U.S. Pat. No. 5,779,708), microchip devices (see, e.g., U.S. Pat. No. 5,797,898), ophthalmic formulations, transdermal matrices (see, e.g., U.S. Pat. Nos. 5,770,219 and 5,783,208), and feedback-controlled delivery (see, e.g., U.S. Pat. No. 5,697,899).

### Kits

The invention provides kits for the treatment or prevention of a disease or disorder. The agents described herein may, in some embodiments, be assembled into pharmaceutical or diagnostic or research kits to facilitate their use in therapeutic, diagnostic, or research applications. In certain embodiments agents in a kit may be in a pharmaceutical formulation and dosage suitable for a particular application and for a method of administration of the agents. Kits for research purposes may contain the components in appropriate concentrations or quantities for running various experiments.

Kits may include dose-size-specific ampules or aliquots of compositions of the present invention. Kits may also contain devices to be used in administering the compositions. In some embodiments, the kit comprises a sterile container which contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The kit may be designed to facilitate use of the methods described herein. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or another suitable solvent), which may or may not be provided with the kit.

The kit may contain any one or more of the components described herein in one or more containers. As an example, in one embodiment, the kit may include instructions for mixing one or more components of the kit and/or isolating and mixing a sample and administering to a subject. The kit may include a container housing agents described herein. The agents may be in the form of a liquid, gel or solid (powder). The agents may be prepared sterilely, packaged in syringe and shipped refrigerated. A second container may comprise other agents prepared sterilely. Alternatively the kit may include agents premixed and shipped in a syringe, vial, tube, or other container. The kit may have one or more or all of the components useful to administer the agents to a subject, such as a syringe, topical application devices, or intravenous needle tubing and bag.

If desired, an agent of the invention is provided together with instructions for administering the agent to a subject having or at risk of developing a disease or disorder described herein. The instructions will generally include information about the use of the composition for the treatment or prevention of the disease or disorder. In other embodiments, the instructions include at least one of the following: description of the therapeutic agent; dosage schedule and administration for treatment or prevention of a disease or disorder described herein; precautions; warnings; indications; counter-indications; overdosage information; adverse reactions; animal pharmacology; clinical studies; and/or references. The instructions may be printed directly on the container (when present), provided on a transportable storage medium, stored on a remote server, or provided as a label applied to the container, or as a separate sheet, pamphlet, card, or folder supplied in or with the container. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Examples

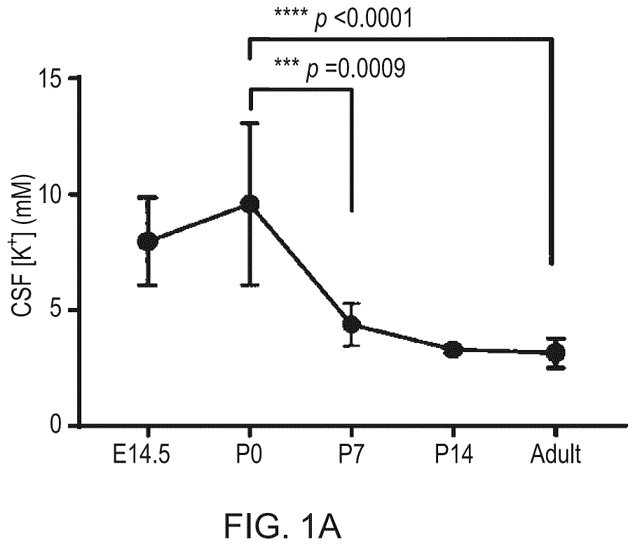

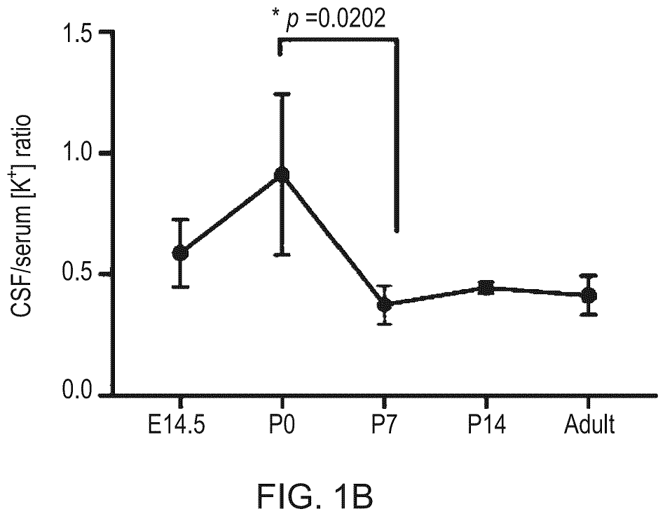

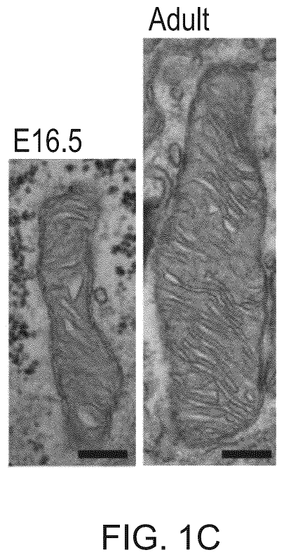

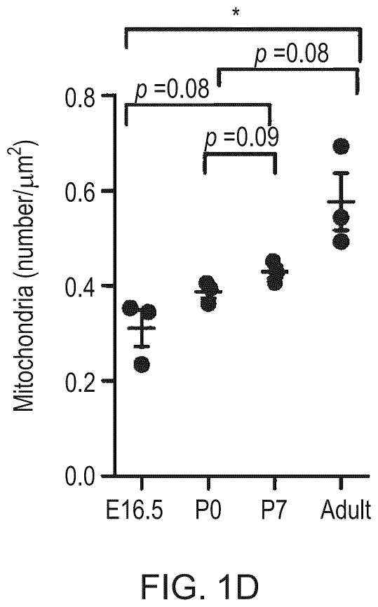

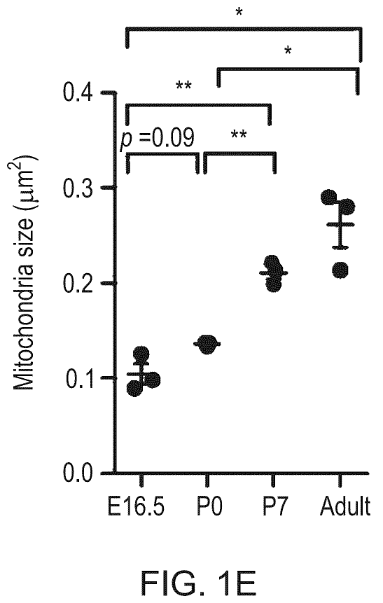

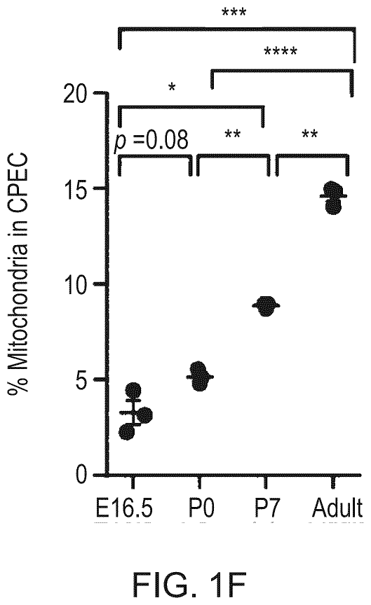

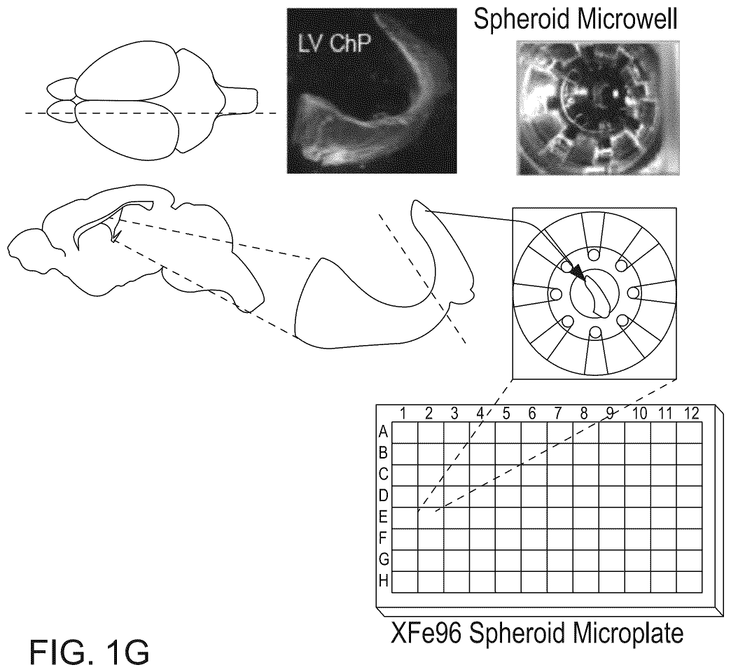

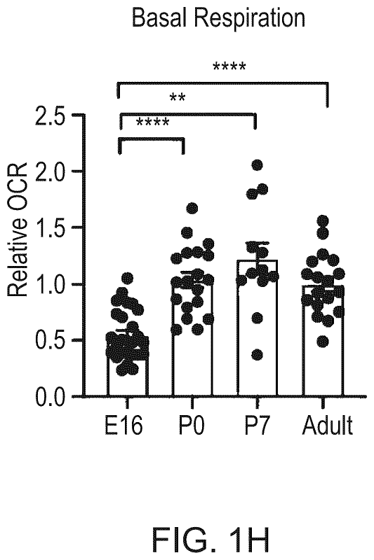

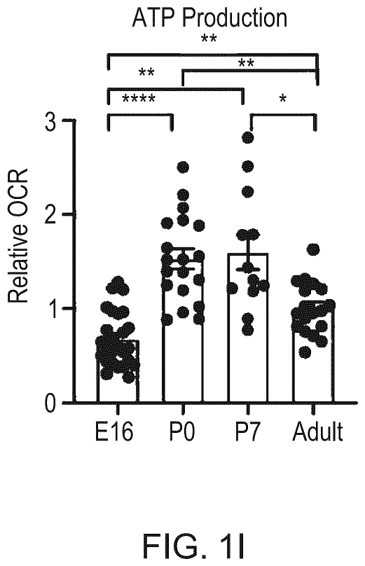

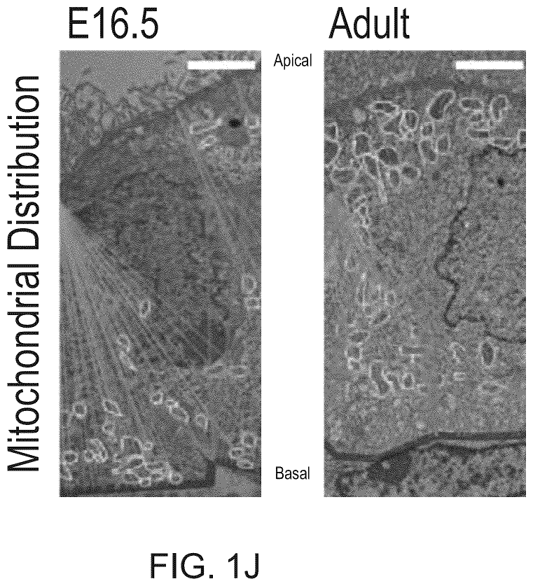

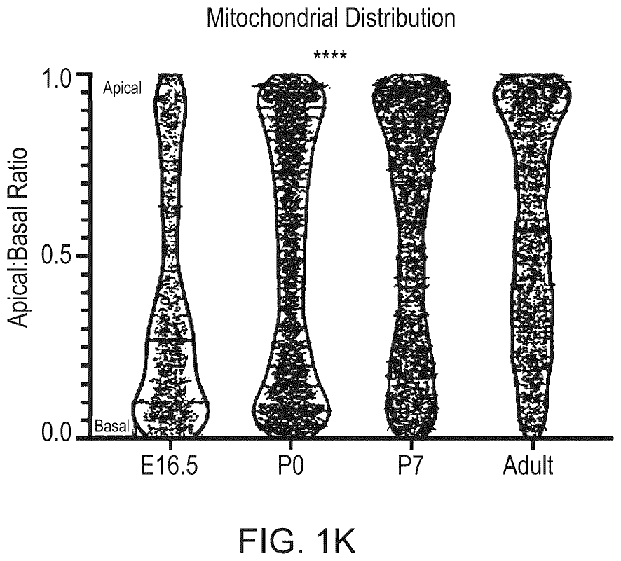

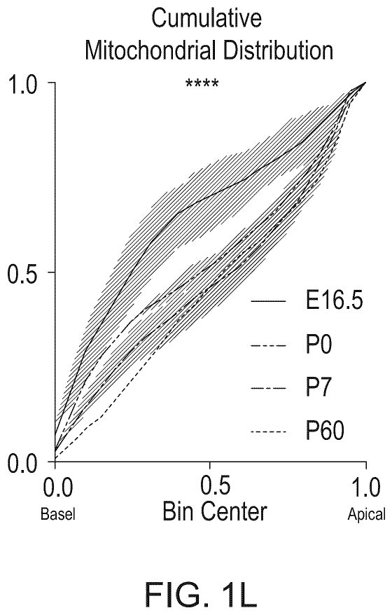

Example 1.: Cerebrospinal fluid (CSF) K⁺ declines precipitously during a specific perinatal period A unique and transient phase of neurodevelopment was discovered when cerebrospinal fluid (CSF) [K⁺] decreased rapidly. Coupled plasma optical emission spectrometry (ICP-OES) and ion chromatography (IC) were used to measure levels of key ions likely to govern cerebrospinal fluid (CSF) flux including Nat, K⁺, and Cl⁻ at several developmental timepoints. cerebrospinal fluid (CSF) [K⁺] was remarkably high at birth (9.6±3.5 mm), decreased rapidly to 4.4±0.9 mM by P7 (FIG. 1A), and later achieved adult levels of 3.1±0.6 mM (FIG. 1A) while [Na⁺] was minimally changed and [Cl⁻] slightly increased (Table 1 shows developmental cerebrospinal fluid (CSF) [K⁺], [Na⁺], [Ca²⁺], [Mg²⁺], [Cl⁻]). Notably, the cerebrospinal fluid (CSF)-to-serum [K⁺] ratio decreased sharply from P0 to P7 (FIG. 1B), suggesting that the onset of cerebrospinal fluid (CSF) clearance occurs during this window²⁵. Similar trends of cerebrospinal fluid (CSF) [K⁺] decrease as development proceeds have been reported in many other species perinatally, although by far none have shown a decrease as drastic as in mice, perhaps reflecting different stages of brain development at the times of sampling^{17,26} Studies in adult animals have shown rapid clearance of K⁺ out of cerebrospinal fluid (CSF) when the concentrations are higher than homeostatic levels²⁷⁻²⁹, supporting the concept that cerebrospinal fluid (CSF) [K⁺]-sensitive mechanisms exist and can mediate K⁺ clearance in a timely manner. Notably, K⁺ transport has been associated with water co-transport by several K⁺ transporters in various tissues and cell types³⁰⁻³², suggesting that cerebrospinal fluid (CSF) [K⁺] changes could drive water movement in the brain as well. Therefore, work was initiated to identify mechanisms underlying this fast clearance of cerebrospinal fluid (CSF) K⁺, which may shed light on cerebrospinal fluid (CSF) outflow during this time.

### Example 2.: Choroid Plexus (ChP) Metabolic Rate Increases During the Early Postnatal Transition Phase

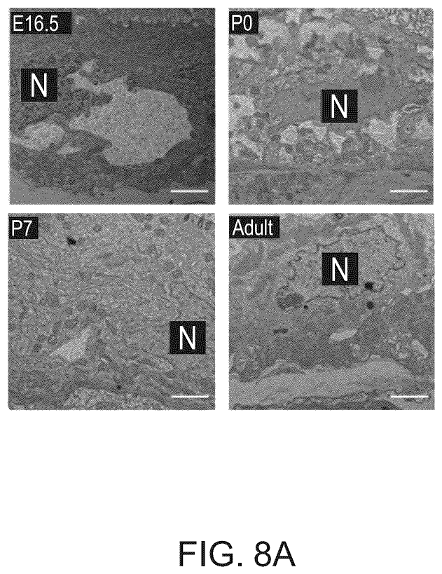

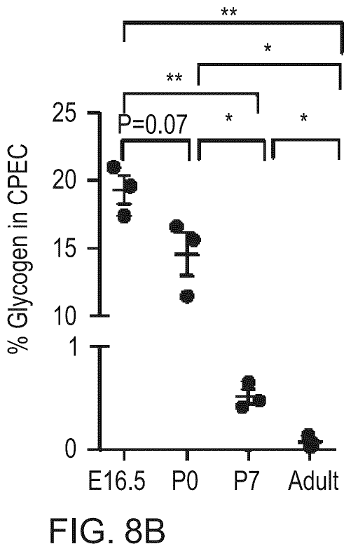

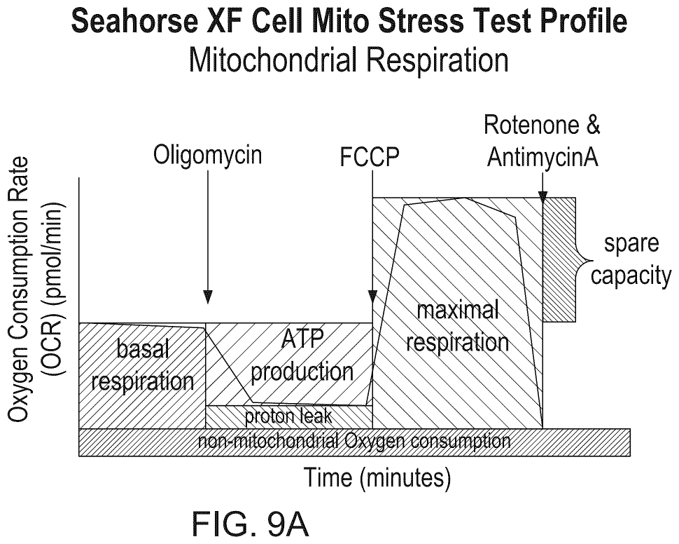

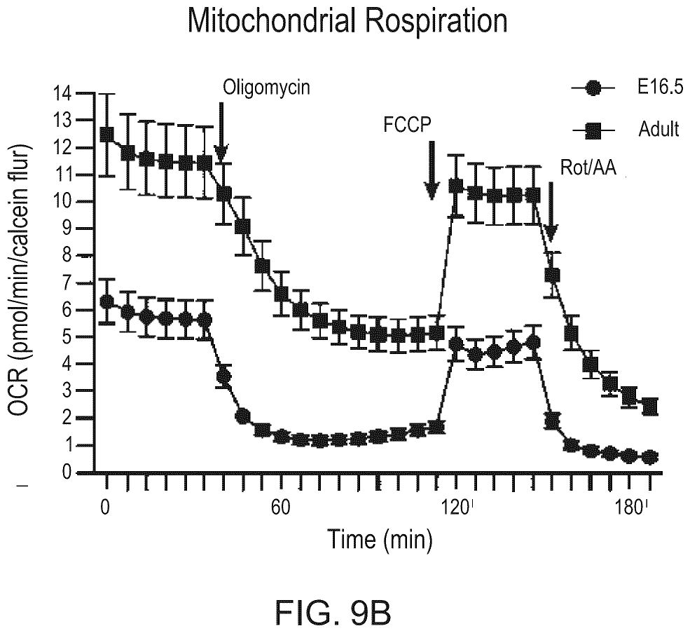

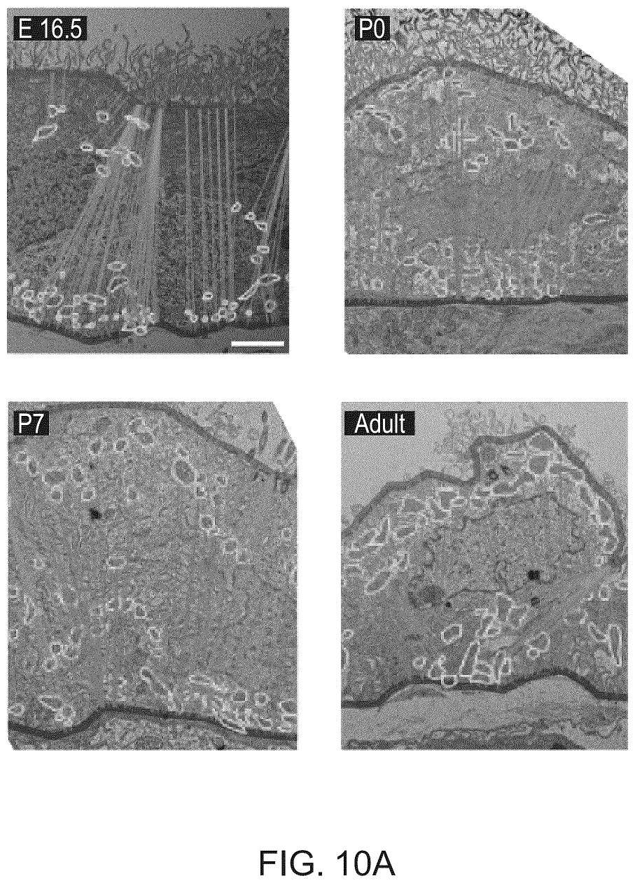

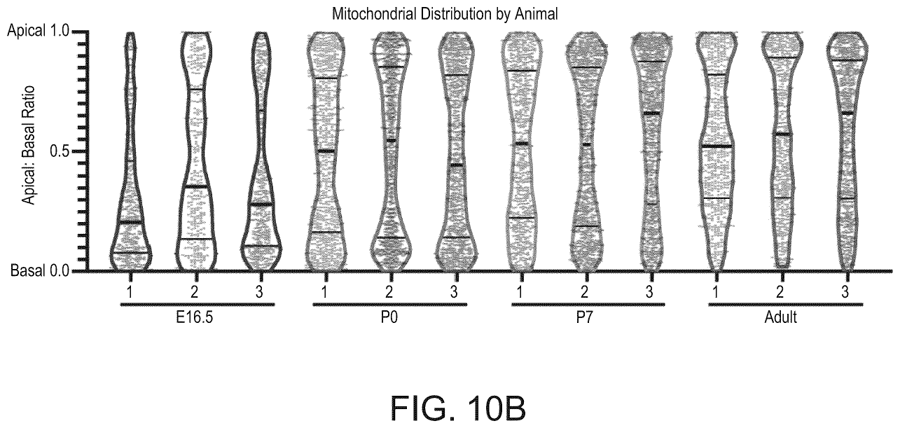

The transitional period of rapid cerebrospinal fluid (CSF) K⁺ clearance coincided with high choroid plexus (ChP) metabolism (FIGS. 1A, 1B, 1H, and 1I). It was reasoned that K⁺ clearance during this period could be choroid plexus (ChP)-mediated because the choroid plexus (ChP) expresses high levels of K⁺ co-transporters on its large cerebrospinal fluid (CSF)-contacting surface area^{15,33} Similar to water and ion transport by other epithelial structures such as kidney proximal and distal tubes 34, K⁺ clearance from cerebrospinal fluid (CSF) by the choroid plexus (ChP) would be energy-dependent and therefore be accompanied by upregulation of ATP production and mitochondrial activity. Therefore, the metabolic status and ATP production capacity of the choroid plexus (ChP) epithelium before, during, and after the time period of cerebrospinal fluid (CSF) [K⁺] reduction was evaluated. It was found that both mitochondria number and size increased from E16.5 to 2 months old (2 mo, referred to as “adult”) (FIGS. 1C-1F), while cellular glycogen load gradually decreased (FIGS. 8A-8B). Both observations are consistent with reports from choroid plexus (ChP) in other mammalian species^{33,35} and suggest functional changes in choroid plexus (ChP) oxidative metabolism. Agilent Seahorse XFe technology was used to monitor oxygen consumption as an index of the metabolic status of the choroid plexus (ChP) in explants from embryonic day 16.5 (E16.5), postnatal day 0 (P0), P7, and adult mice. Basal metabolism and ATP production was then calculated (FIGS. 1G and 9A-9B). E16.5 choroid plexus (ChP) had the lowest basal respiration of all tested ages (FIGS. 1H and 9A-9B). Adult had a higher capacity for overall ATP production than E16.5 choroid plexus (ChP), but surprisingly, P0-P7 choroid plexus (ChP) were the most metabolically active as per ATP production (FIGS. 1H and 1I). In addition, mitochondrial subcellular distribution in choroid plexus (ChP) epithelium was biased toward the apical surface as postnatal development proceeded, with E16.5 mitochondria heavily distributed along the basal side of epithelial cells, P0 mitochondria intermediately localized, and P7 and adult mitochondria having more apical distribution (FIGS. 1I-1L and 10A-10B). Mitochondrial subcellular localization responds to regional energy demand in other cellular processes such as migration of mouse embryonic fibroblasts and during axonal outgrowth^{36,37}. Together with the increase in ATP production postnatally, the shift in choroid plexus (ChP) epithelial mitochondria distribution over postnatal development suggests increasing ATP supply to meet high demand at the apical choroid plexus (ChP) surface during the early postnatal phase, concurrent with the rapid clearance of cerebrospinal fluid (CSF) K⁺. Although many processes at the choroid plexus (ChP) apical surface consume ATP and could drive the mitochondrial distribution shift during this phase, this concurrence prompted the investigation of mechanisms whereby choroid plexus (ChP) epithelial cells might contribute to K⁺ clearance through ATP dependent mechanisms.

### Example 3.: The Choroid Plexus (ChP) Increases Production of Cerebrospinal Fluid (CSF)-Facing Ion and Water Transporters Postnatally

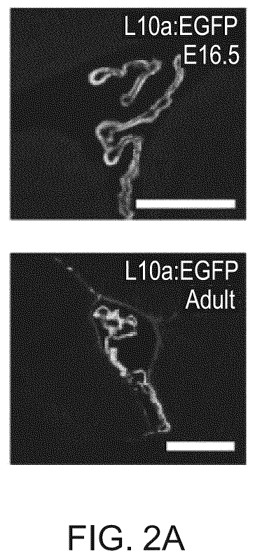

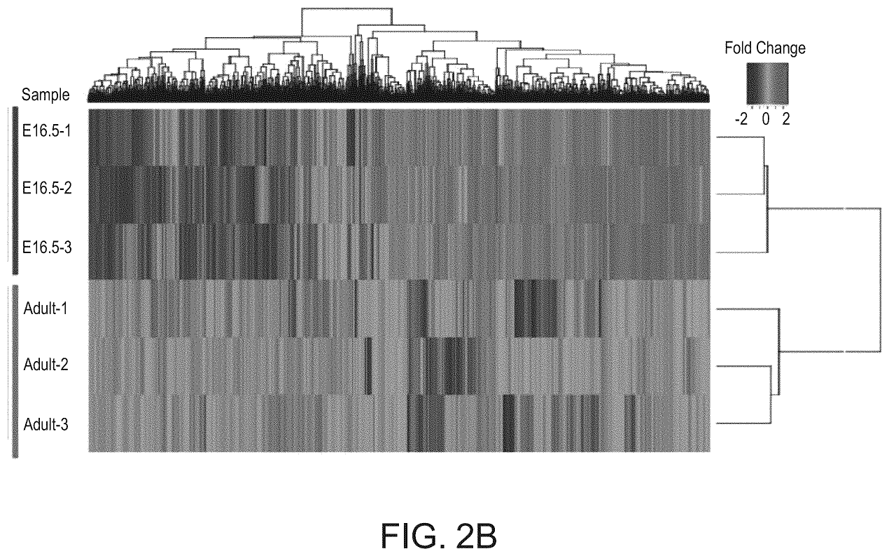

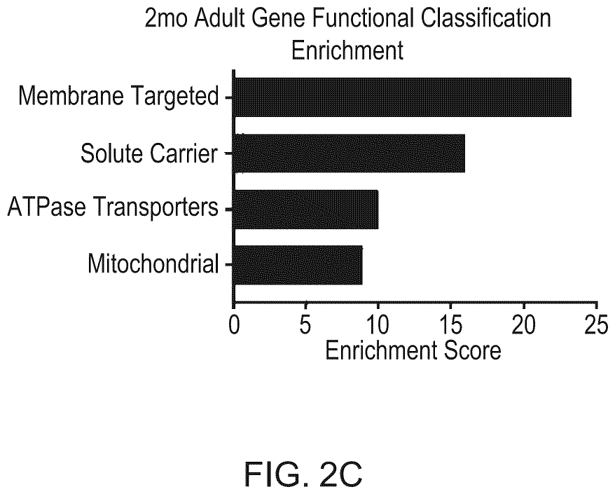

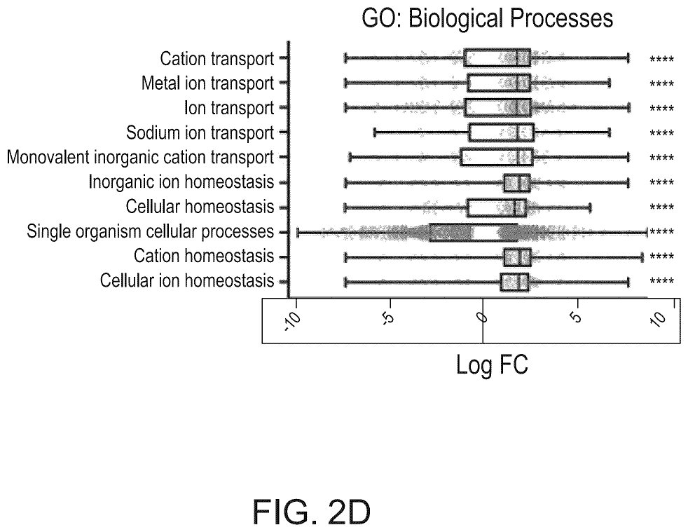

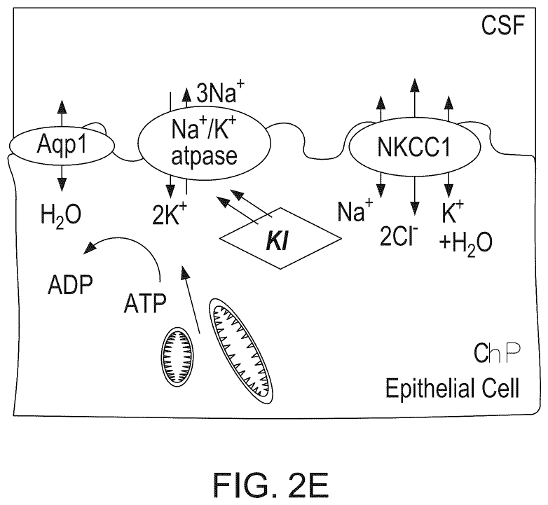

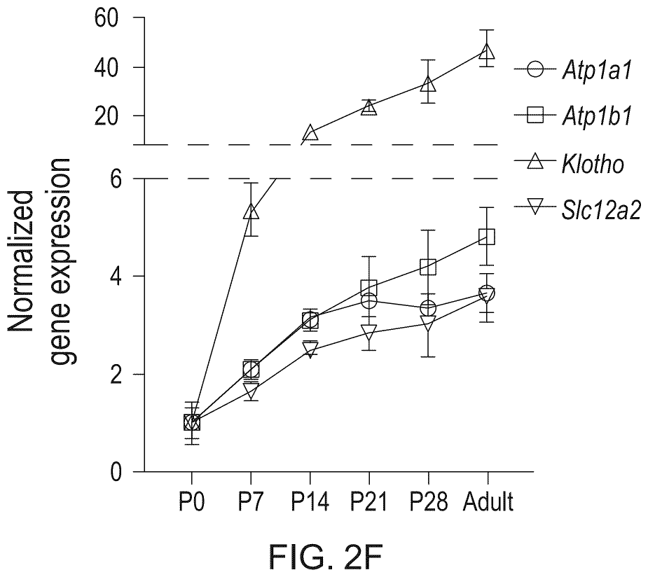

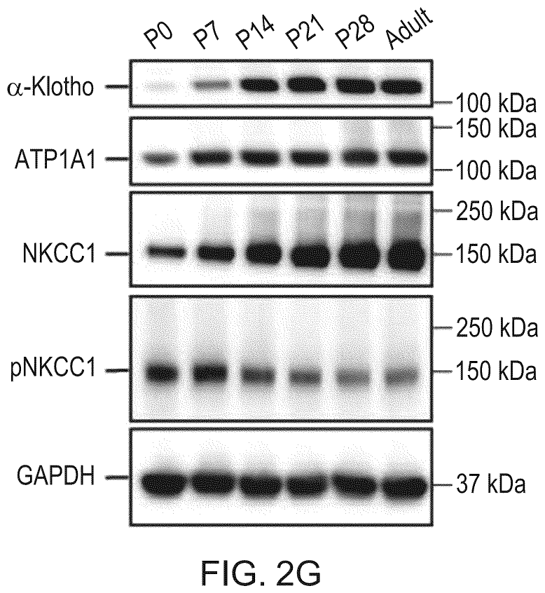

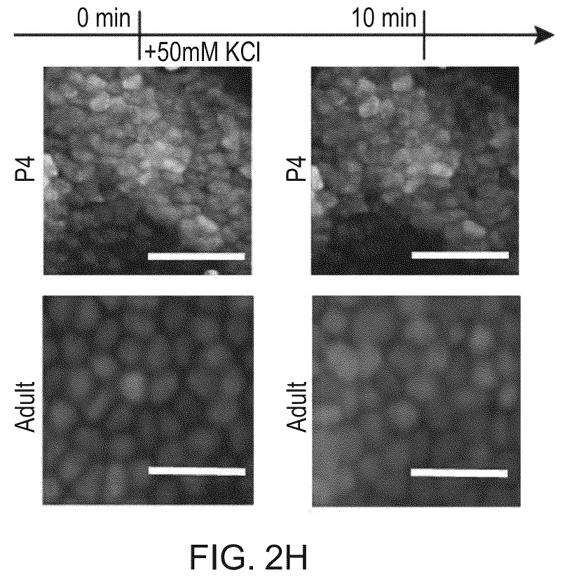

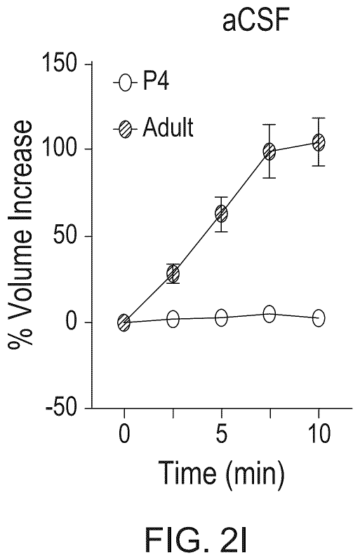

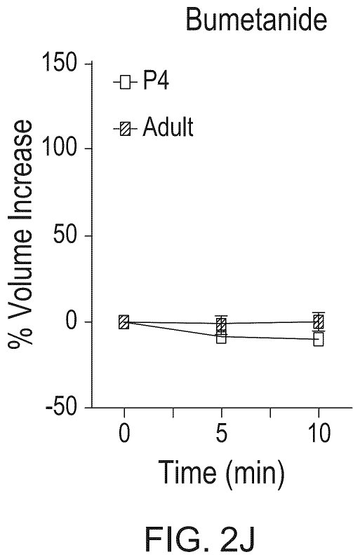

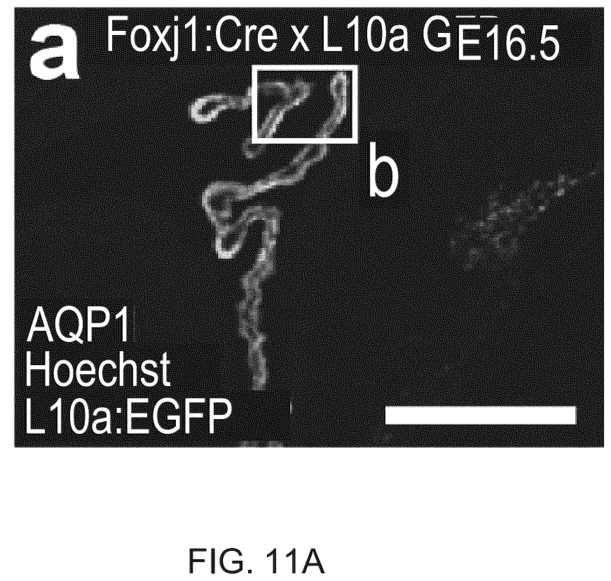

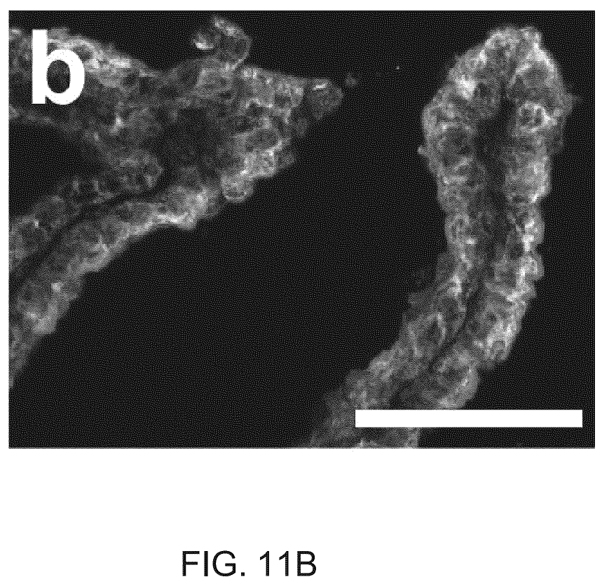

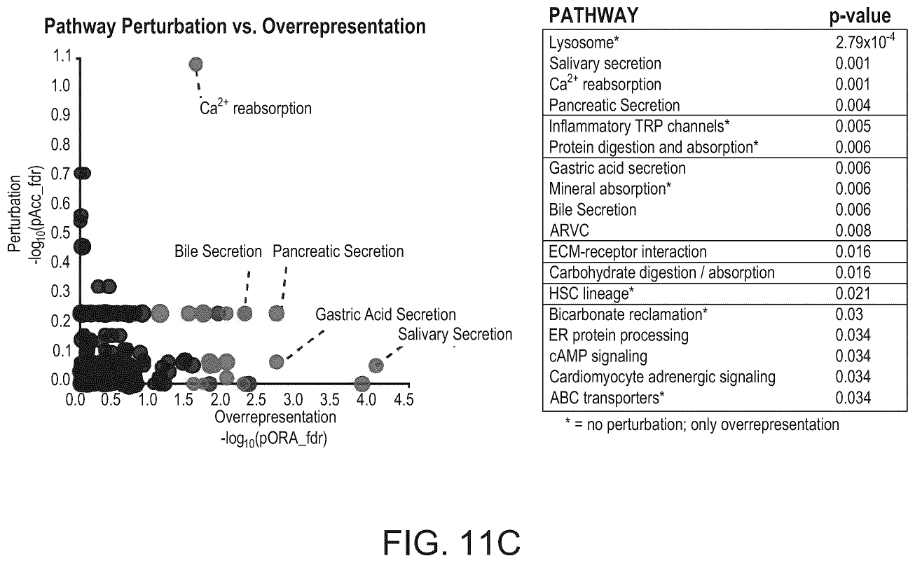

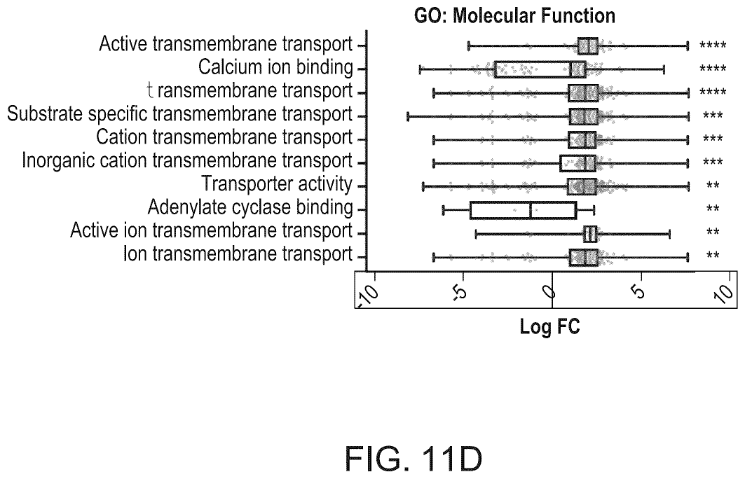

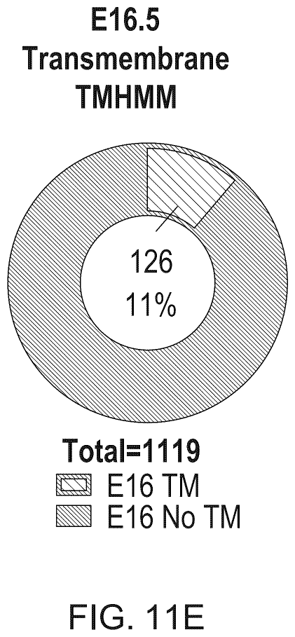

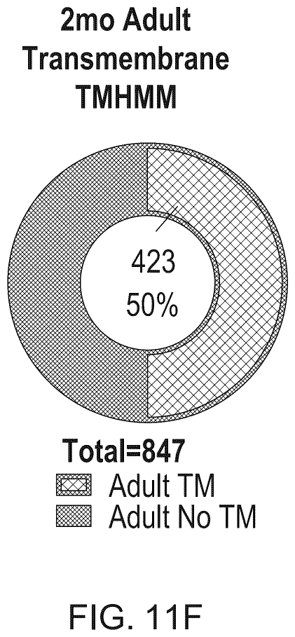

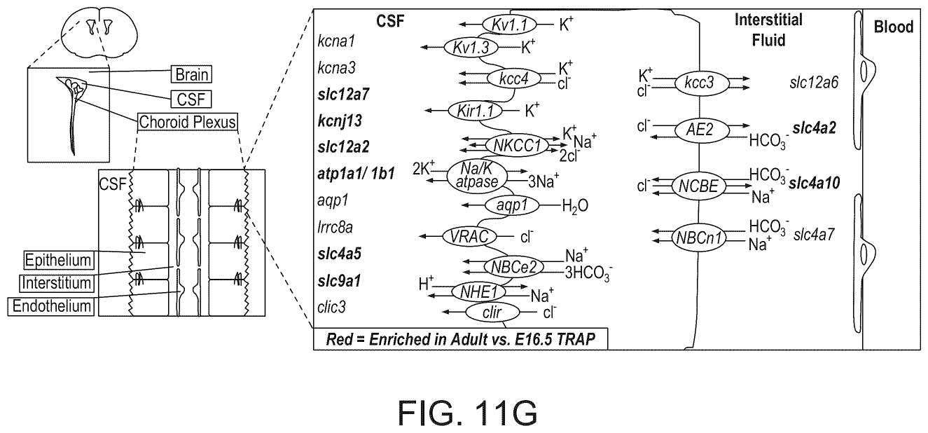

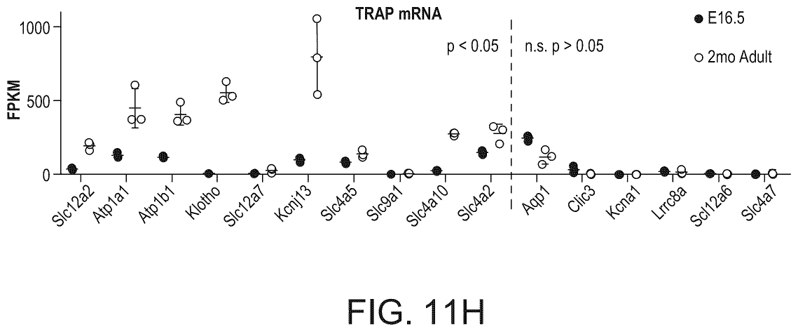

Consistent with rapid cerebrospinal fluid (CSF) K⁺ clearance and high choroid plexus (ChP) metabolism, it was found that expression of the energy-dependent cation transport pathway components was upregulated in choroid plexus (ChP) postnatally. To identify which of the choroid plexus (ChP) transporters are likely candidates controlling postnatal cerebrospinal fluid (CSF) clearance through the choroid plexus (ChP), ribosomal profiling was conducted to investigate transcripts that are prioritized for translation in embryonic (E16.5) and adult choroid plexus (ChP), using Translating Ribosomal Affinity Purification (TRAP;³⁸). choroid plexus (ChP) epithelial cells were targeted by crossing FoxJ1:cre mice³⁹ with TRAP (EGFP:L10α) mice 3¹ (FIGS. 2A and 11A-11B), and mRNA associated with the L10a ribosomal subunit were purified for sequencing.

TRAP analyses revealed 1967 differentially translated transcripts (adjusted p<0.05) between E16.5 and adult choroid plexus (ChP): 1119 enriched at E16.5 and 847 enriched in adults (FIG. 2B). Gene set and pathway analyses revealed developmentally regulated choroid plexus (ChP) programs. Adult choroid plexus (ChP) had enriched functional gene sets associated with active transmembrane membrane transport and mitochondria, which is consistent with the abovementioned findings on metabolism changes (FIG. 2C). Notably, cation transport was enriched, supporting the choroid plexus (ChP) mediating cerebrospinal fluid (CSF) K⁺ transport postnatally (FIGS. 2C and 2D). Enriched pathways in the adult included secretion associated pathways named for other, better studied secretory processes, including salivary and pancreatic secretion, all of which have a special emphasis on water and ion transmembrane transport (FIGS. 11C and 11D). Consistent with a rise in fluid and ion modulating machinery, there was a striking enrichment of more transmembrane and signal peptide-containing transcripts in adult choroid plexus (ChP) (FIGS. 11E and 11F). In contrast, the water channel AQP1, which is not directly responsive toward any given ion gradient, but rather depends on the overall osmolarity, remained unchanged (FIG. 11H). These results indicated that the choroid plexus (ChP) gained fluid and ion modulatory functions postnatally, with an emphasis on ion-related transporters and channels.

### Example 4.: NKCC1 is Poised to Mediate Perinatal Choroid Plexus (ChP) Cerebrospinal Fluid (CSF) K⁺ and Water Clearance

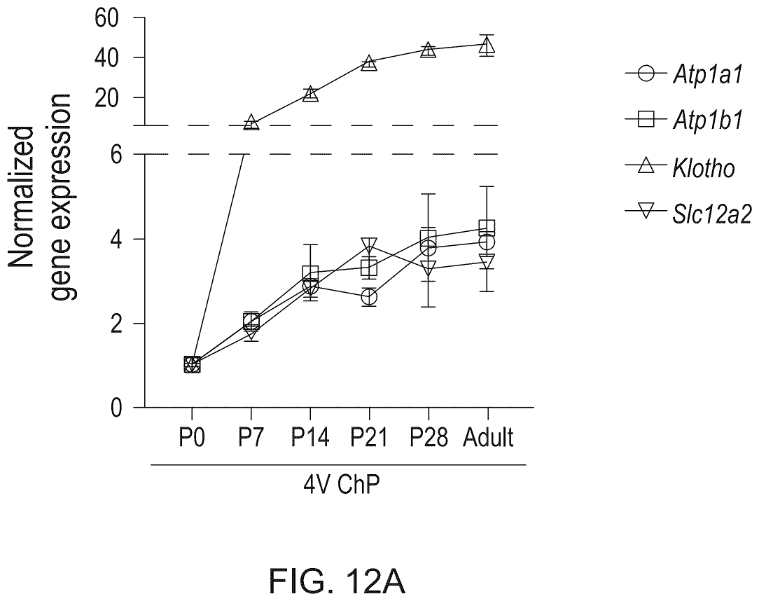

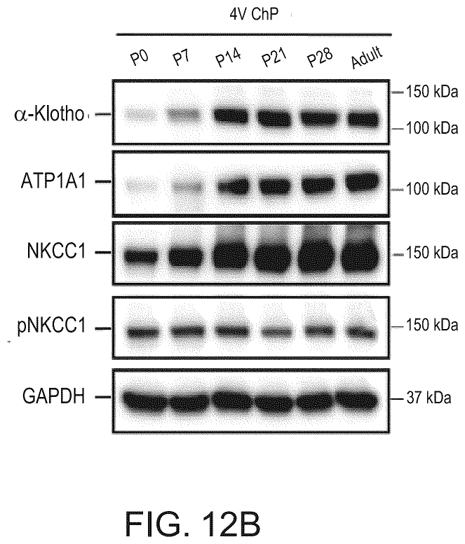

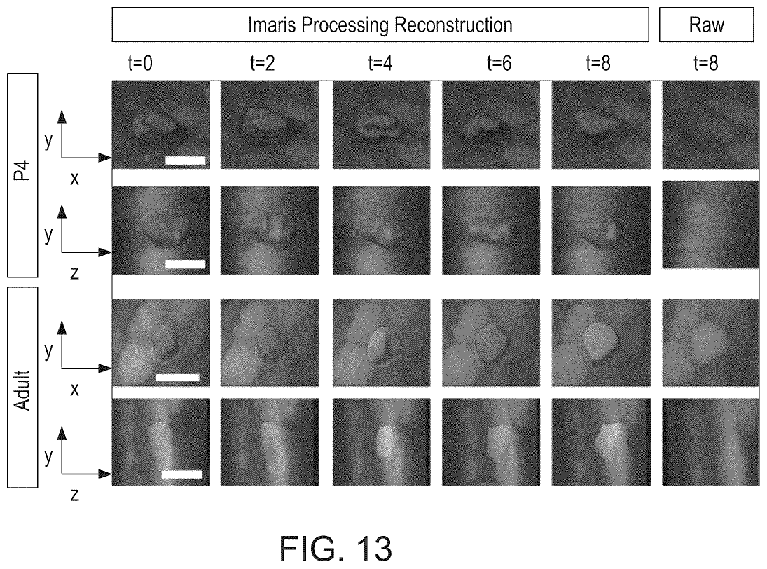

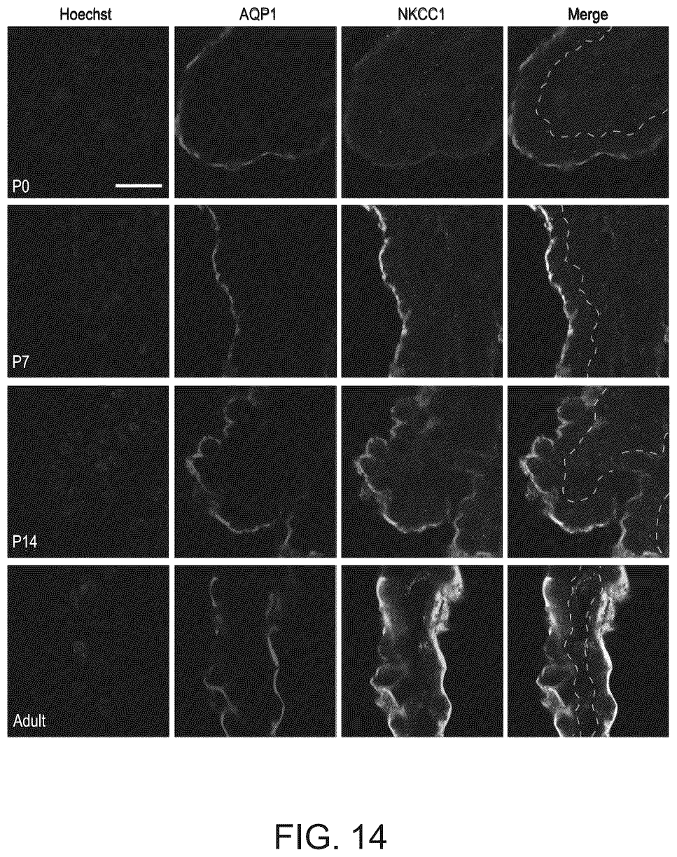

Among all fluid and ion modulating candidates with increasing postnatal expression (FIGS. 11G and 11H), NKCC1 (Slc12α2) was identified as the candidate most likely to mediate cerebrospinal fluid (CSF) clearance. NKCC1 is functionally related to Na⁺/K⁺-ATPase (Atp1a1 and Atp1b1), as the latter actively maintains the Na⁺/K⁺ gradient that powers NKCC1. Both the Na⁺/K⁺-ATPase and NKCC1 are capable of cerebrospinal fluid (CSF) K⁺ clearance, but NKCC1 was of particular interest because (1) it is a co-transporter of K⁺ and water^{30,40}; and (2) the activity of NKCC1 can be further modified by phosphorylation⁴¹, lending additional control to its fluid/ion modulatory capacity. In addition, NKCC1 is particularly enriched in the choroid plexus (ChP) and does not impact broad functionality like the Na⁺/K⁺-ATPase does, both of which are important features for a functional therapeutic intervention target. Temporal expression analyses of NKCC1, ATP1a1, ATP1bT, and Klotho (Kl), which contributes to the membrane localization of the Na⁺/K⁺-ATPase^{42,43} (FIG. 2E), were refined by sampling weekly from P0 to P28 and then from adult, and confirmed increased expression of transcript and protein for each component across developmental time (FIGS. 2F, 2G, and 12A-12B). The observed changes in NKCC1 total protein were corroborated by an independent approach where the rate of choroid plexus (ChP) epithelial cell swelling under high [K⁺] challenge⁴⁰ reflected the abundance of NKCC1 protein (FIGS. 2H-2J and 13). Artificial cerebrospinal fluid (CSF) (aCSF) recipes that reflect the [K⁺] of neonatal vs. adult cerebrospinal fluid (CSF) were used for respective ages (see Table 1 for recipes). Immunolabeling of NKCC1 showed its apical membrane localization at all ages, regardless of abundance (FIG. 14).

TABLE 1
cerebrospinal fluid (CSF) ion concentrations at developmental stages.

| | K⁺ | | | Na⁺ | | | | | Cl⁻ | | |
|---|---|---|---|---|---|---|---|---|---|---|---|
| | mM | p | | N | mM | p | | N | mM | p | |
| E14.5 | 7.969 ± 1.90 | **** | <0.0001 | 8 | 141.1 ± 6.10 | ns | 0.1851 | 5 | 104.9 ± 5.28 | ** | 0.0054 |
| P0 | 9.590 ± 3.50 | ** | 0.0015 | 5 | 126.2 ± 11.84 | ns | 0.03605 | 4 | 104.0 ± 3.27 | ** | 0.0078 |
| P4 | 4.903 ± 0.47 | ** | 0.0013 | 4 | 131.9 ± 4.52 | ns | 0.1 | 4 | 111.0 ± 2.00 | * | 0.035 |
| P7 | 4.363 ± 0.92 | * | 0.0348 | 4 | 144.5 ± 6.65 | ns | 0.8313 | 4 | 125.0 ± 2.00 | ns | 0.7499 |
| P14 | 3.283 ± 0.18 | ns | 0.6672 | 4 | 137.3 ± 3.22 | ns | 0.2686 | 4 | 115.1 ± 6.00 | ns | 0.1124 |
| Adult | 3.142 ± 0.61 | / | / | 6 | 146.2 ± 14.40 | / | / | 5 | 127.1 ± 12.01 | / | / |

| | Cl⁻ | Ca²⁺ | | | | Mg²⁺ | | | |
|---|---|---|---|---|---|---|---|---|---|
| | N | mM | p | | N | mM | p | | N |
| E14.5 | 5 | 3.42 ± 0.47 | * | 0.0205 | 3 | 1.76 ± 0.07 | **** | <0.0001 | 4 |
| P0 | 4 | 3.70 ± 0.84 | * | 0.0456 | 3 | 1.07 ± 0.04 | *** | 0.0002 | 4 |
| P4 | 4 | 2.63 ± 0.21 | ns | 0.1273 | 3 | 1.09 ± 0.01 | **** | <0.0001 | 4 |
| P7 | 4 | 3.13 ± 0.39 | * | 0.0328 | 3 | 2.04 ± 0.19 | ** | 0.001 | 4 |
| P14 | 4 | 2.51 ± 0.36 | ns | 0.3443 | 4 | 0.53 ± 0.05 | **** | <0.0001 | 4 |
| Adult | 5 | 2.24 ± 0.28 | / | / | 3 | 0.89 ± 0.04 | / | / | 5 |

p: statistical comparison to adult values.
* p < 0.05;
** p < 0.01;
*** p < 0.001;
**** p < 0.0001;
ns = not significant.

In addition, particularly high levels of phosphorylated were found, therefore activated⁴¹, NKCC1 (pNKCC1) in the choroid plexus (ChP) of P0-P7 pups, with P7 having peak pNKCC1 levels among all postnatal ages, indicative of increased NKCC1 activity during the first postnatal week (FIG. 2G). Similar to the timeline of choroid plexus (ChP) ATP production (FIG. 1I), the timeframe of high choroid plexus (ChP) pNKCC1 was concurrent with the fast cerebrospinal fluid (CSF) [K⁺] decrease during the first postnatal week (FIGS. 1A and 1B), suggesting a functional correlation and further confirming the significance of the early postnatal transitional period. Taken together, choroid plexus (ChP) NKCC1 was identified as the top candidate for mediating postnatal cerebrospinal fluid (CSF) K⁺ and water clearance.

### Example 5.: NKCC1 Temporal Regulation Requires Epigenetic Control that is Implicated in Congenital Hydrocephalus

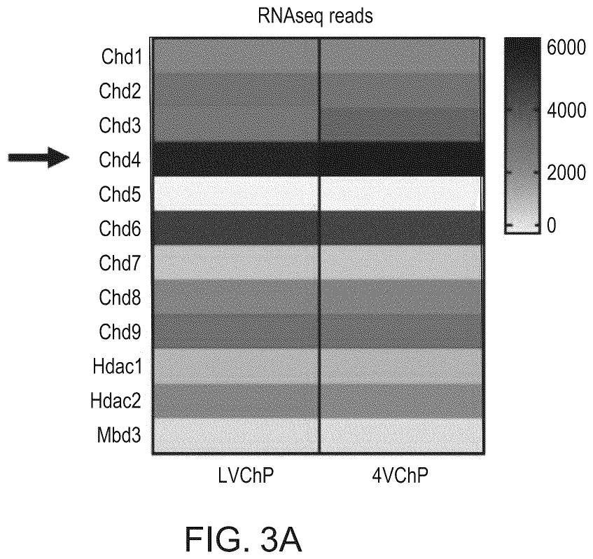

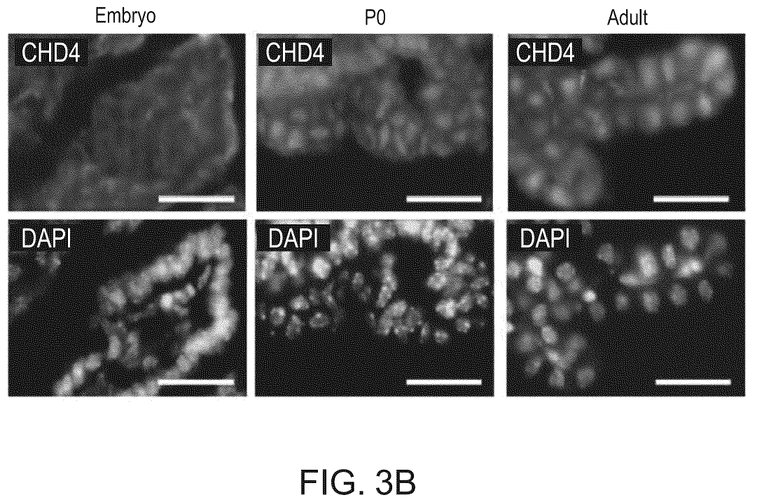

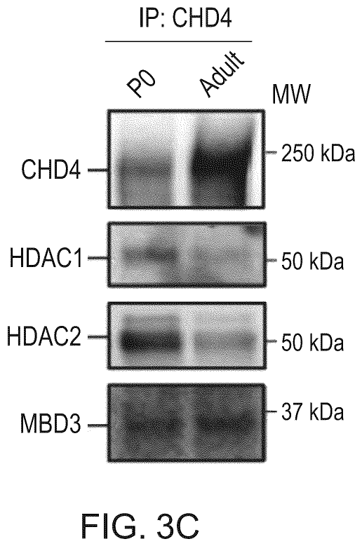

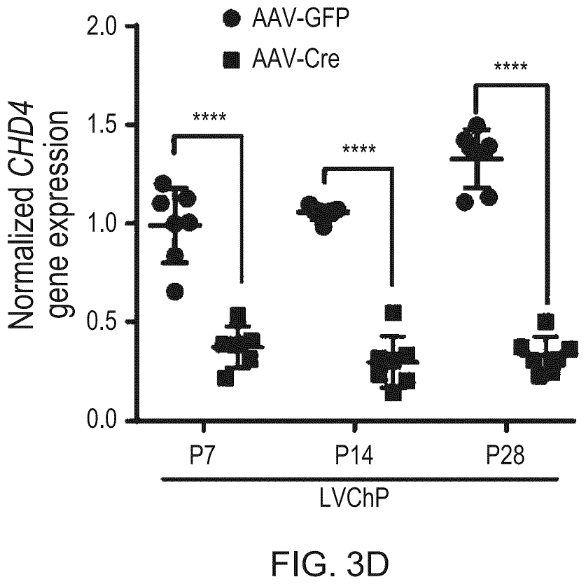

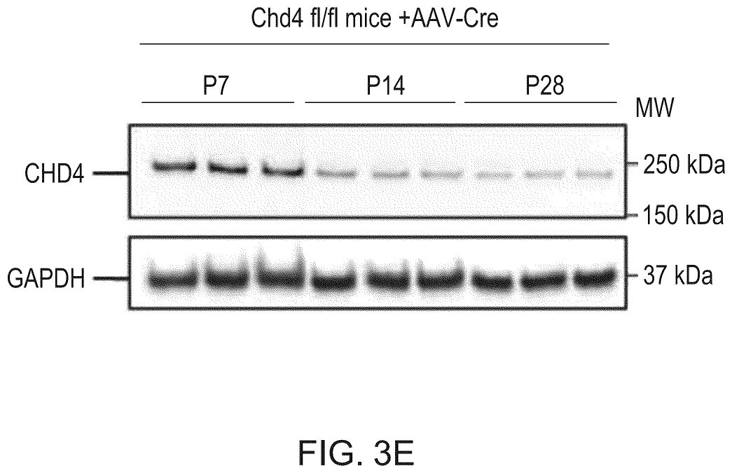

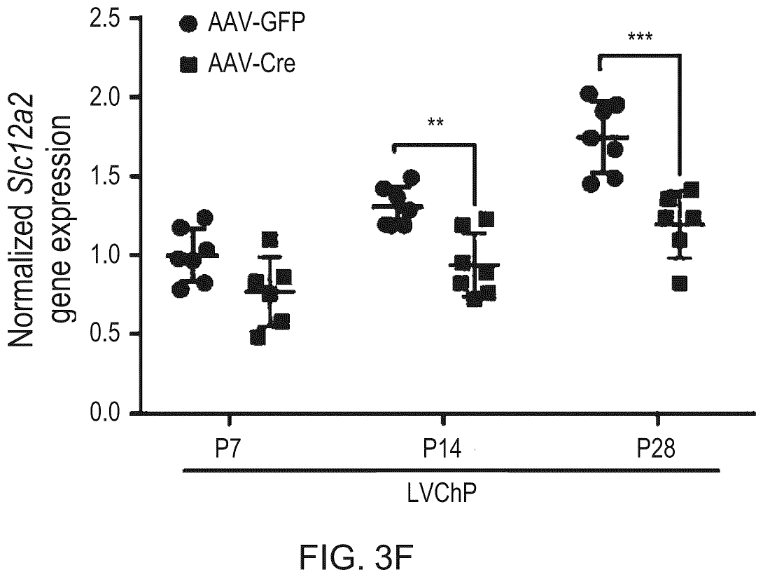

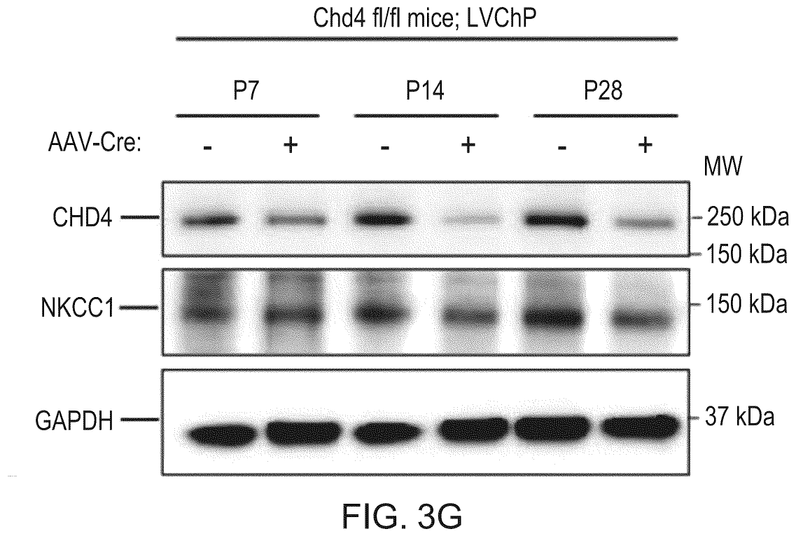

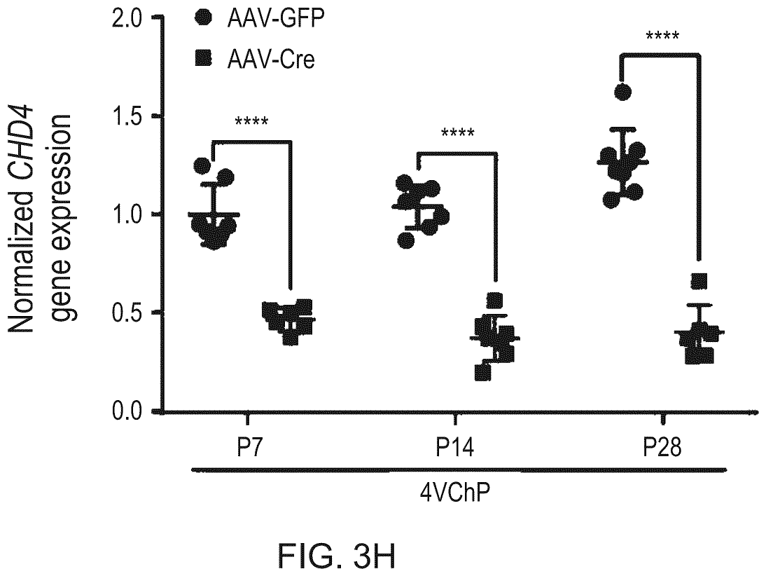

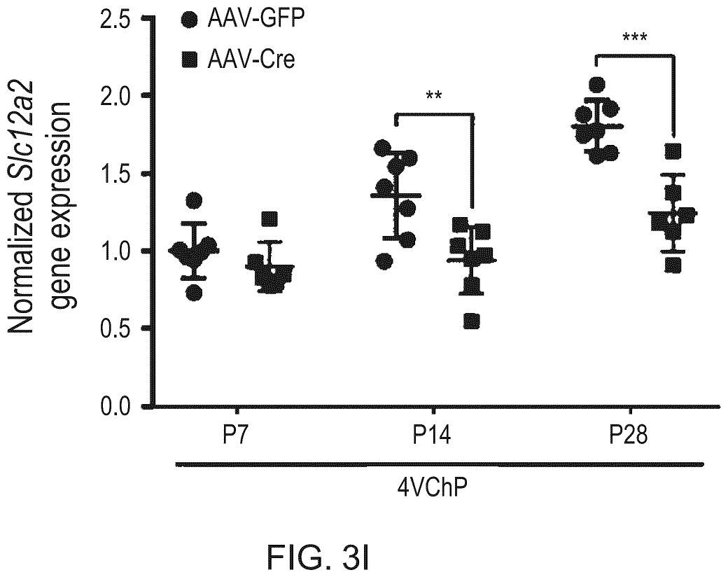

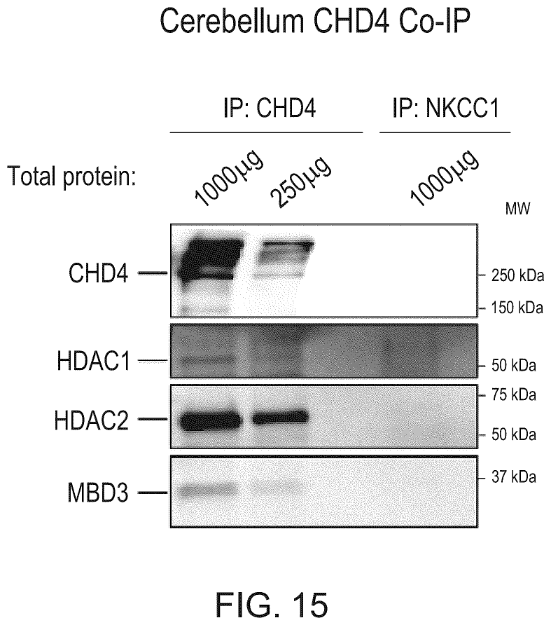

It was found that the temporal profile of NKCC1 expression was tightly regulated at the epigenetic level by modulators implicated in some forms of congenital hydrocephalus. The NuRD complex governs differentiation and maturation of diverse cells and tissues 44. Previously published RNA sequencing studies 45 identified NuRD components, including the ATPase CHD family members (Chd4 being the most highly expressed), the histone deacetylases HDAC1/2, and methyl CpG-binding domain protein MBD3 in the choroid plexus (ChP) (FIG. 3A). De novo loss-of-function CHD4 mutations are implicated in some groups of children with congenital hydrocephalus and ventriculomegaly⁴⁶. It was found that CHD4 localized to nuclei in mouse choroid plexus (ChP) epithelial cells beginning at P0 (FIG. 3B). Immunoprecipitation of CHD4 identified HDAC1, HDAC2, and MBD3 by immunoblotting in mouse choroid plexus (ChP) (FIG. 3C, technical control for Co-IP protocol is shown in FIG. 15), confirming the existence of the CHD4/NuRD complex in developing choroid plexus (ChP). The complex was then disrupted by generating choroid plexus (ChP)-Chd4 deficient mice. Cre was expressed in Chd4 floxed mice (Chd4 fl/fl)⁴⁷ using an adeno-associated viral vector (AAV) with tropism for the choroid plexus (ChP) (AAV2/5)⁴⁸, delivered by in utero intracerebroventricular (ICV) injection at E14.5. Chd4 transcript levels dropped to <50% by P7 (FIG. 3D). While CHD4 protein levels only substantially decreased by P14 (FIGS. 3E and 3G), it was found that the developmental increase of choroid plexus (ChP) NKCC1 expression was disrupted as soon as the CHD4 protein decreased and lasted at least until P28 (FIGS. 3F and 3G). Similar results were also observed in 4VChP (FIGS. 3H and 3I). These data confirm that the NuRD/ChD4 complex is one of the required components tightly regulating choroid plexus (ChP) NKCC1 developmental expression.

### Example 6.: Choroid Plexus (ChP) NKCC1 Actively Mediates Cerebrospinal Fluid (CSF) Clearance During the Early Postnatal Transition Phase

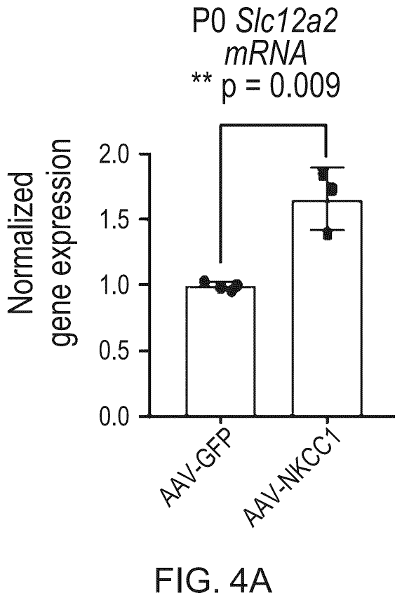

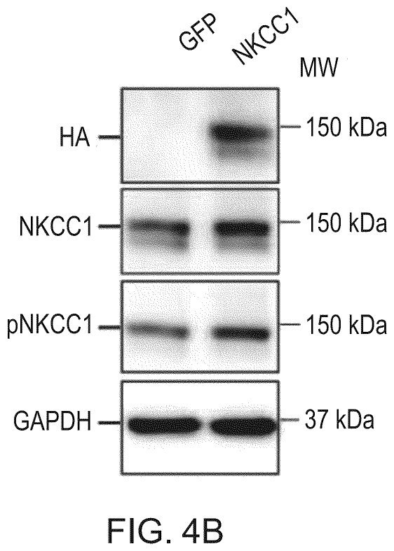

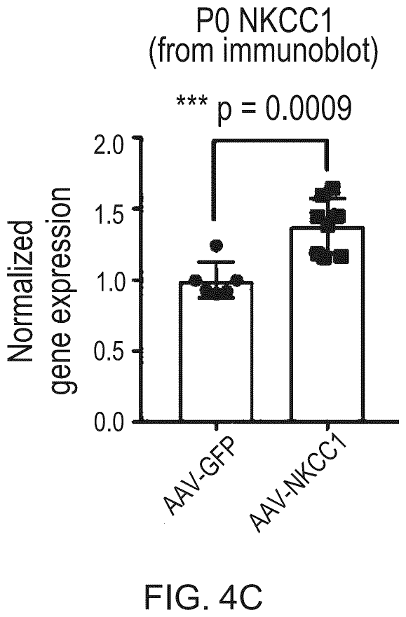

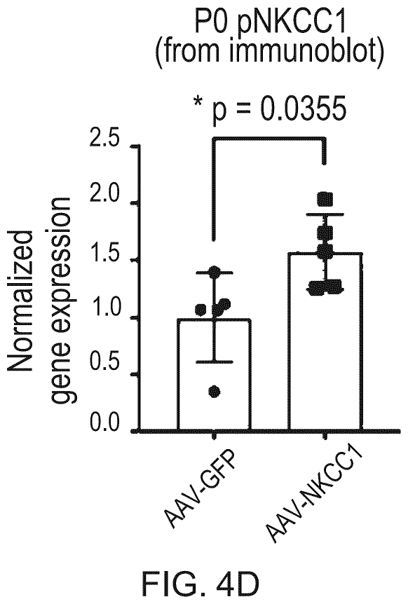

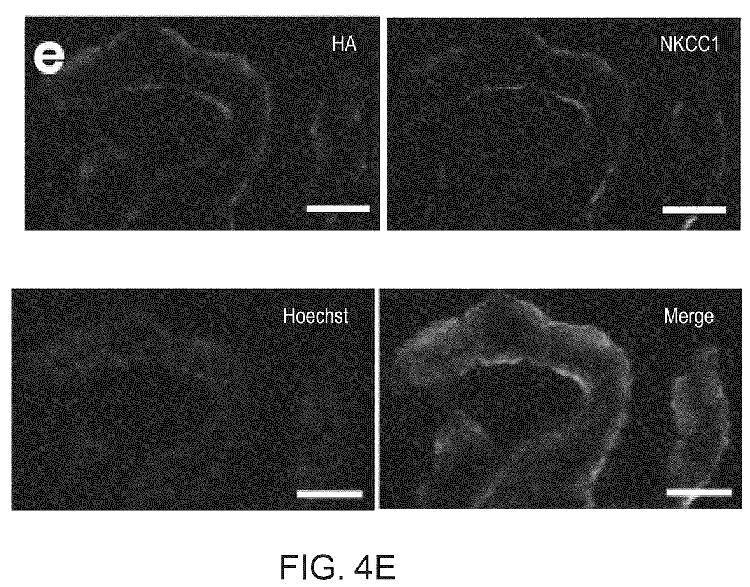

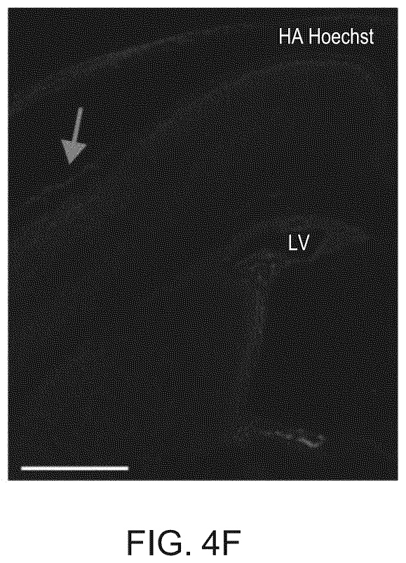

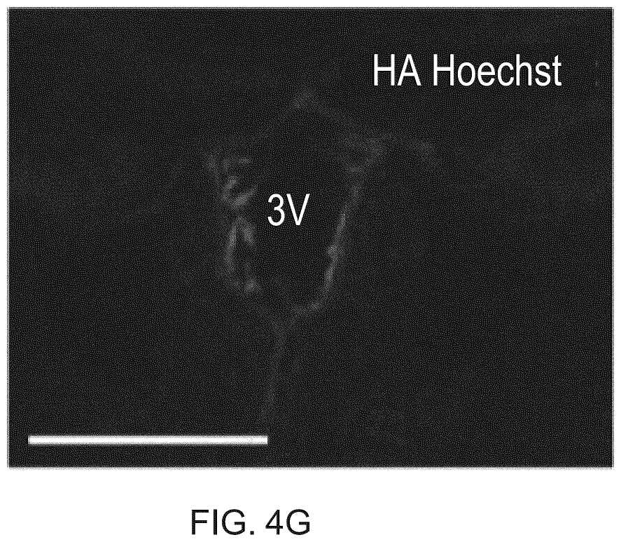

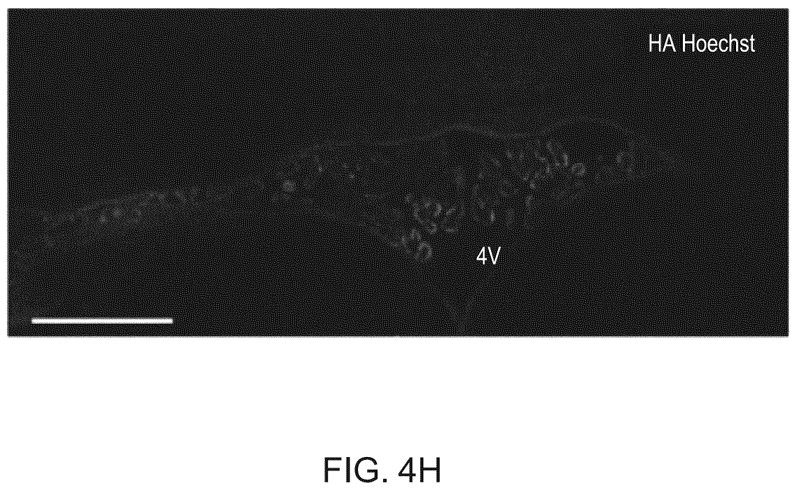

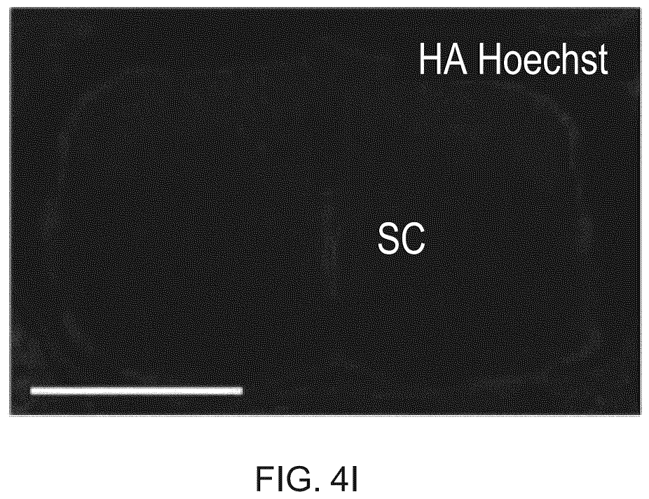

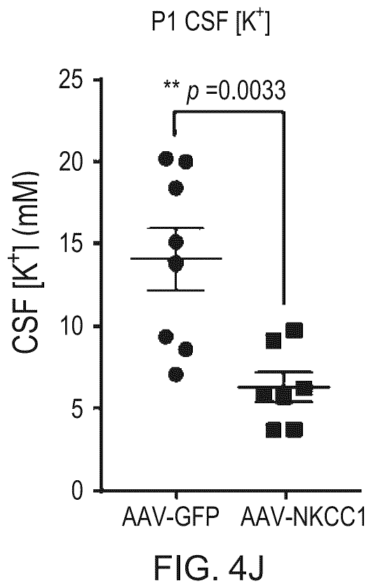

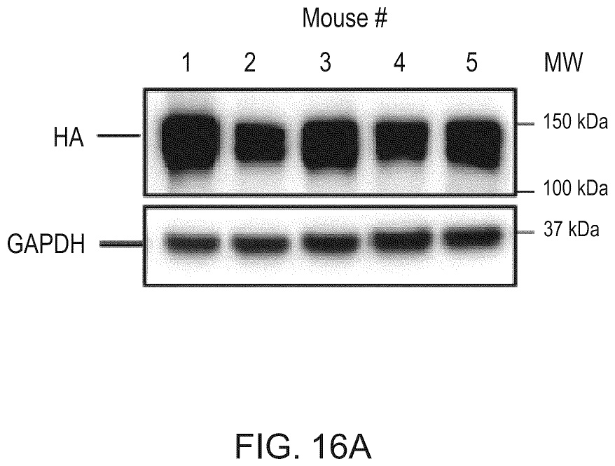

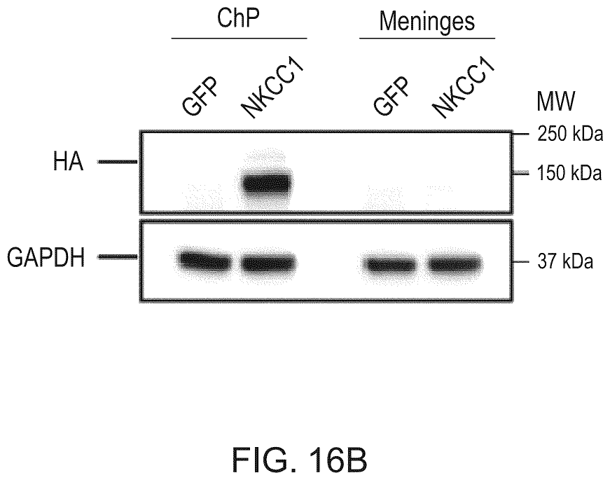

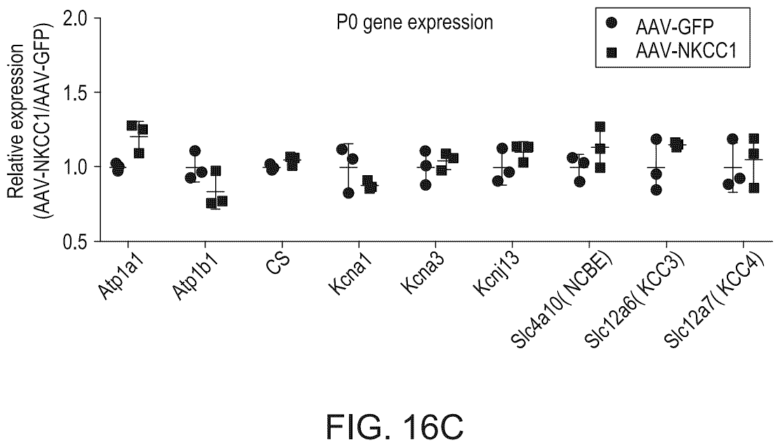

To test whether NKCC1 is indeed capable of transporting from cerebrospinal fluid (CSF) into the choroid plexus (ChP) during the period of rapid cerebrospinal fluid (CSF) [K⁺] decline, NKCC1 overexpression (OE) was induced in developing choroid plexus (ChP) epithelial cells using AAV2/5. NKCC1 transport directionality follows combined Na⁺, K⁺, Cl⁻ gradients, which are close to neutral in adult brains and likely to bias towards the cerebrospinal fluid (CSF)-to-choroid plexus (ChP) direction during the early postnatal phase. NKCC1 protein level would be rate-limiting during the early postnatal time when it is already highly phosphorylated, unlike in older mice where pNKCC1 only represented a small portion of total NKCC1. The goal of this overexpression (OE) approach was to accelerate endogenous choroid plexus (ChP) NKCC1 transport, thereby revealing its directionality based on whether cerebrospinal fluid (CSF) [K⁺] clearance was enhanced or delayed. AAV2/5-NKCC1, which expresses NKCC1 fused to an HA tag 49, or control GFP virus was delivered by in utero intracerebroventricular (ICV) injection at E14.5. Successful NKCC1 overexpression (OE) and increased pNKCC1 was confirmed in choroid plexus (ChP) at P0 (FIGS. 4A-4D). Appropriate localization to apical membranes of epithelial cells, transduction efficiency, and tissue specificity were also validated (FIGS. 4E-4I and 16A-16B). Transcript levels of other K⁺ transporters or channels did not change following AAV2/5-NKCC1 transduction (FIG. 16C). Because cerebrospinal fluid (CSF) [K⁺] sharply decreased from P0 to P7 (FIGS. 1A and 1B), cerebrospinal fluid (CSF) was sampled from choroid plexus (ChP) NKCC1 overexpression (OE) and control mice at P1. It was found that choroid plexus (ChP) NKCC1 overexpression (OE) reduced cerebrospinal fluid (CSF) [K⁺] more than controls, with their P1 cerebrospinal fluid (CSF) [K⁺] values closely approximating those normally observed at P7 (FIG. 4J), indicating accelerated K⁺ clearance from cerebrospinal fluid (CSF) after enhanced choroid plexus (ChP) NKCC1 activity. cerebrospinal fluid (CSF) total protein levels were not affected (AAV2/5-GFP=2.50±0.20 mg/ml vs. AAV2/5-NKCC1=2.71±0.46 mg/ml; N=6 from two litters each; p=0.34, unpaired t-test). Overall, these findings support a model in which, under physiological conditions with high early postnatal cerebrospinal fluid (CSF) [K⁺], choroid plexus (ChP) NKCC1 transports K⁺ out of cerebrospinal fluid (CSF).

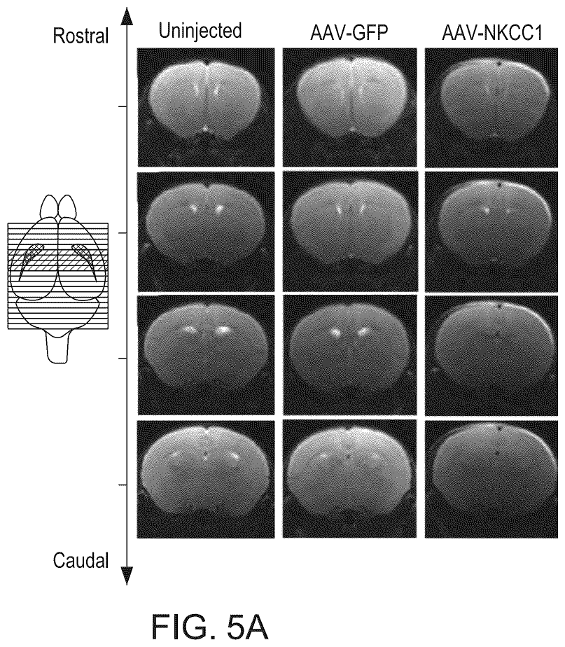

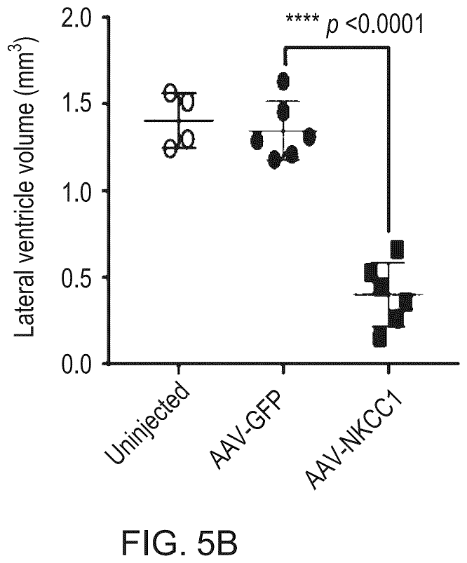

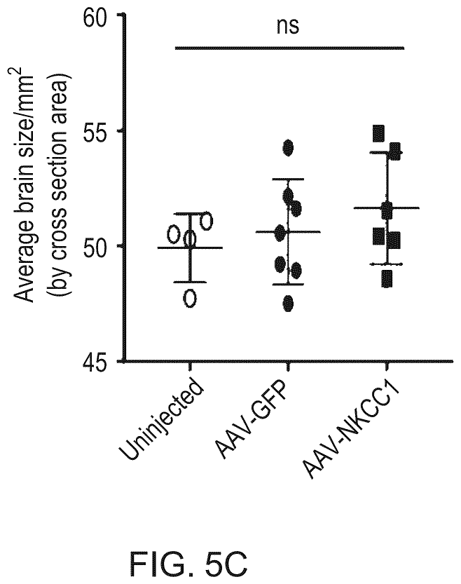

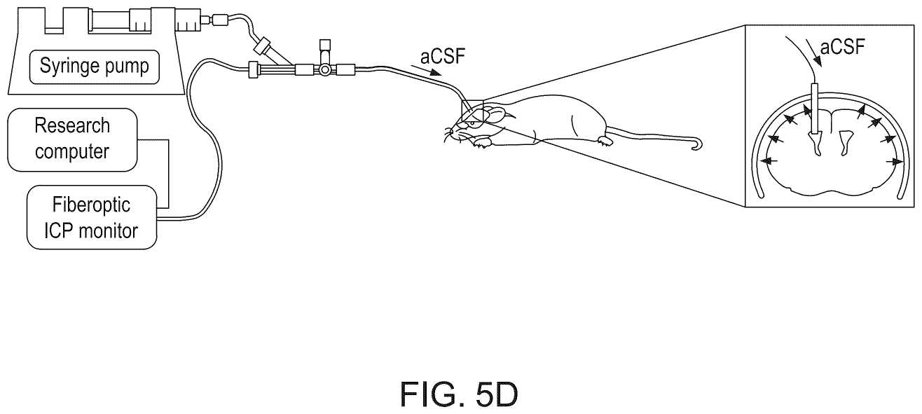

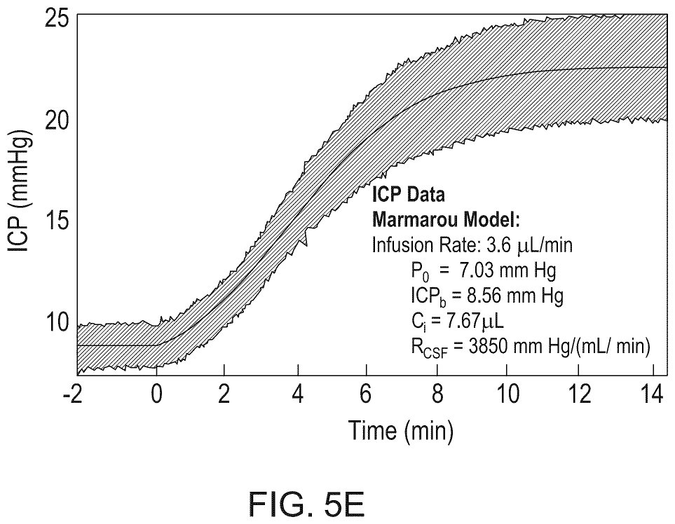

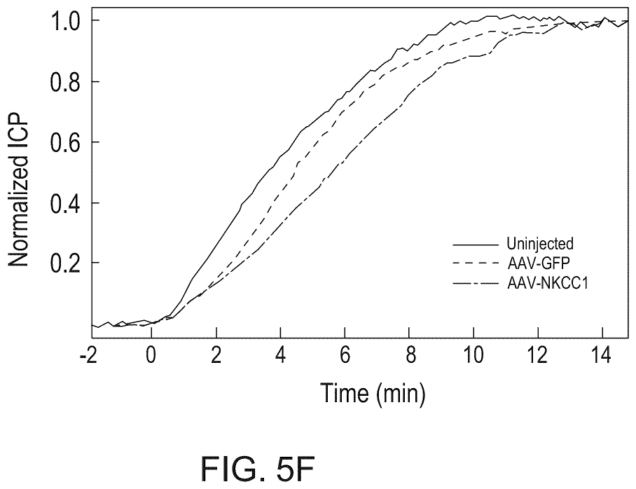

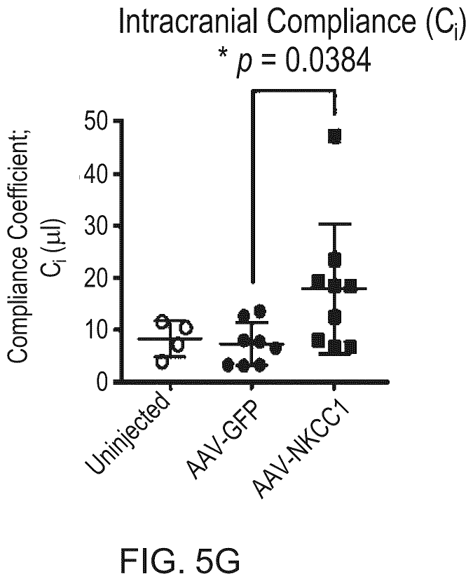

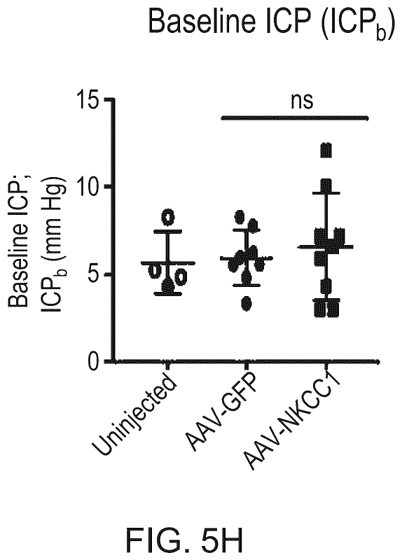

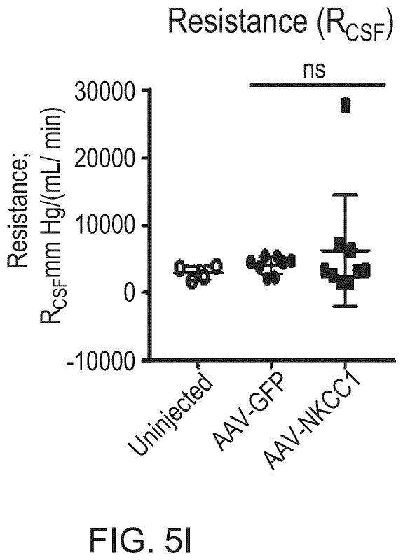

Next, it was found that the circulating cerebrospinal fluid (CSF) volume in choroid plexus (ChP) NKCC1 overexpression (OE) mice was reduced, as reflected by smaller lateral ventricles. To avoid any tissue processing artifacts, live T2-weighted magnetic resonance imaging (MRI) (FIG. 5A) was conducted to quantify lateral ventricle volume. AAV-GFP mice were indistinguishable from naive wild-type mice at P14. In contrast, NKCC1 overexpression (OE) mice had reduced lateral ventricle volumes (FIGS. 5A and 5B), without decrease in overall brain size (FIG. 5C), reflecting less circulating cerebrospinal fluid (CSF). The difference in ventricle sizes from these same mice was sustained when measured again at P50 (AAV-GFP: 3.12±0.59 mm³ vs. AAV-NKCC1: 1.28±0.28 mm³, * p=0.0182), suggesting ventricular deflation established early could dictate long-term changes in ventricle structure. The observed lack of ventricle enlargement at a later stage is also consistent with the finding that only a small portion of NKCC1 was phosphorylated in adult choroid plexus (ChP) (FIG. 2G), and therefore overexpression without increased activation via phosphorylation was unlikely to influence total NKCC1 transport in adulthood. While the exact transport direction of NKCC1 in adult choroid plexus (ChP) is still under debate²⁴, the consistency in ventricular volume from P14 into later life supports the working model that because a relatively small proportion of choroid plexus (ChP) NKCC1 was phosphorylated in mice P14 and older (FIG. 2G), NKCC1 levels are not rate-limiting and thus overexpression (OE) would not as substantially impact choroid plexus (ChP) functions in older animals. Collectively, the findings demonstrate that choroid plexus (ChP) NKCC1 mediated cerebrospinal fluid (CSF) clearance during the first postnatal week. Augmenting this process impacted cerebrospinal fluid (CSF) volume homeostasis in the long term.

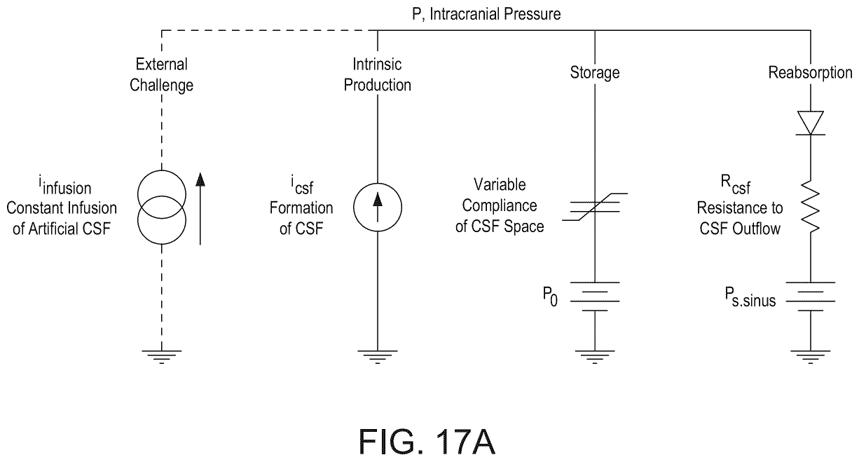

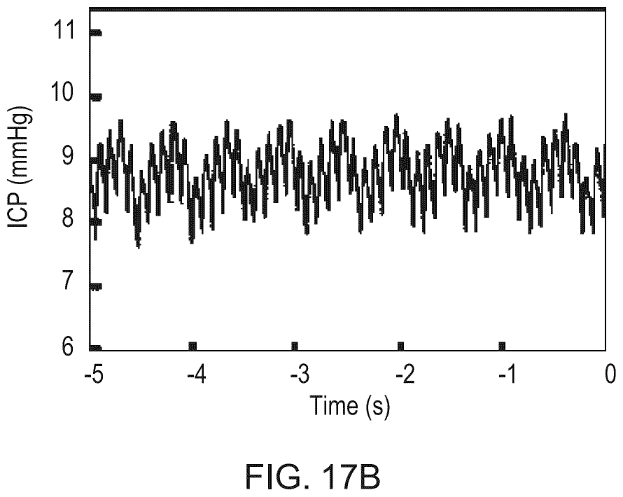

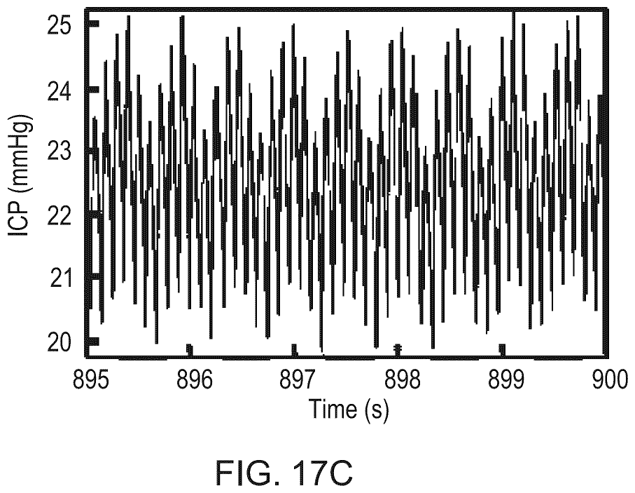

It was found that enhancing cerebrospinal fluid (CSF) clearance through choroid plexus (ChP) NKCC1 overexpression (OE) changed how the brain and cranial space adapted to cerebrospinal fluid (CSF) volume changes. Intracranial compliance (Cᵢ) and cerebrospinal fluid (CSF) resistance (R_{CSF}) describe the ability of the entire intracranial space (including brain, meninges, and outflow routes) to accommodate an increasing cerebrospinal fluid (CSF) volume that would otherwise increase intracranial pressure (ICP). In humans, these parameters are measured by a cerebrospinal fluid (CSF) constant rate infusion test⁵⁰⁻⁵² and can aid in diagnosis and evaluation of conditions like hydrocephalus, which has decreased Cᵢ². intracranial pressure (ICP) and resistance to cerebrospinal fluid (CSF) outflow (R_{CSF}) have been previously measured in rats⁵³ using a similar test, quantifying intracranial pressure (ICP) based on mmH₂O in a glass capillary. It was noted that resistance to cerebrospinal fluid (CSF) outflow (R_{CSF}) changes with infusion rate in a non-linear way, and resistance to cerebrospinal fluid (CSF) outflow (R_{CSF}) was estimated based on linear parts of the curve each representing high or low infusion rates. A miniaturized version of the human test was developed with a pressure sensor coupled with infusion tubing implanted inside the lateral ventricles to determine the Cᵢ and resistance to cerebrospinal fluid (CSF) outflow (R_{CSF}) in mice. The constant rate infusion test artificially increases cerebrospinal fluid (CSF) volume by intracerebroventricular infusion of aCSF, causing intracranial pressure (ICP) to rise and plateau at a new level (FIGS. 5D and 5E). The Cᵢ and resistance to cerebrospinal fluid (CSF) outflow (R_{CSF}) are estimated from the intracranial pressure (ICP) vs. time curve using Marmarou's model of cerebrospinal fluid (CSF) dynamics⁵⁴ (FIG. 17A). Simply put, the Cᵢ is inversely proportional to the rate of intracranial pressure (ICP) increase, and the resistance to cerebrospinal fluid (CSF) outflow (R_{CSF}) is related to the level of the post-infusion intracranial pressure (ICP) plateau (FIG. 5E). As a quality control for the correct placement of infusion and measurement catheter, arterial and respiratory pulsations were clearly visible in the intracranial pressure (ICP) waveform and their amplitude increased with volume load as expected (FIGS. 17B and 17C). Using this approach, it was found that choroid plexus (ChP) NKCC1 overexpression (OE) significantly increased Cᵢ at an age of 5-7 weeks (FIGS. 5F and 5G), consistent with the brain having greater capacity for cerebrospinal fluid (CSF) in ventricles “deflated” due to excessive cerebrospinal fluid (CSF) clearance. Resting intracranial pressure (ICP) and resistance to cerebrospinal fluid (CSF) outflow (R_{CSF}) were unchanged (FIGS. 5H and 5I).

### Example 7.: Enhanced Choroid Plexus (ChP) NKCC1 Function Mitigates Ventriculomegaly in a Model of Obstructive Hydrocephalus

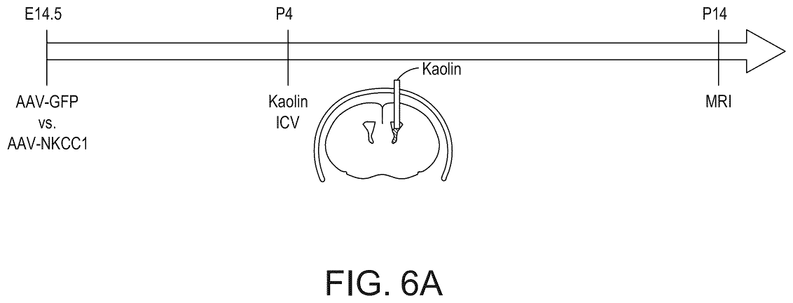

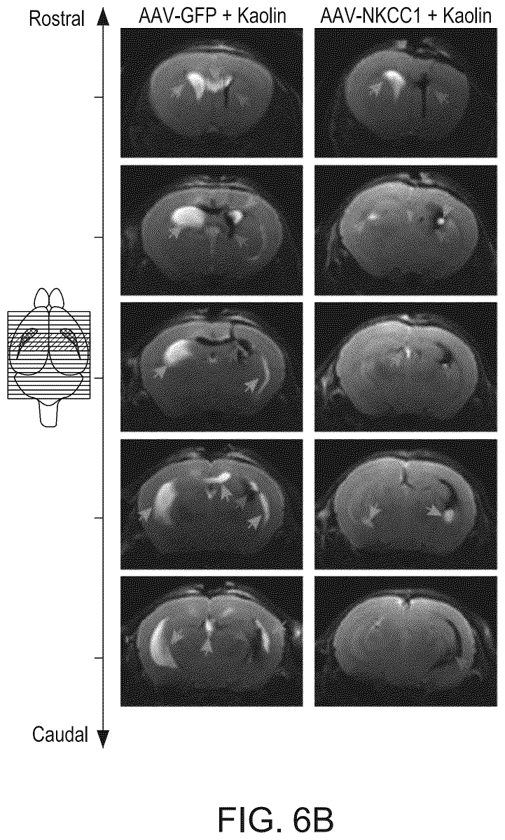

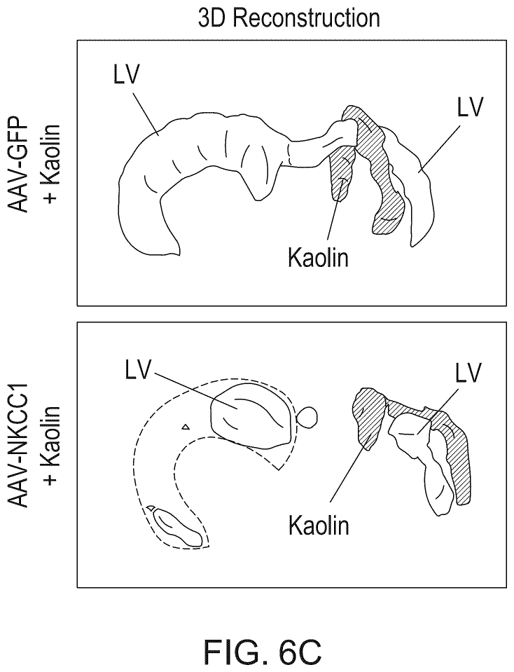

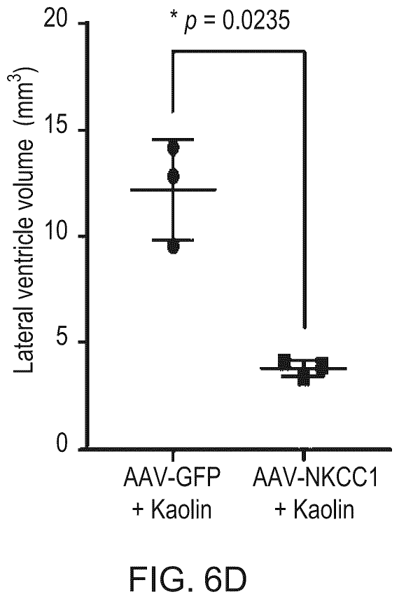

Our findings of enhanced cerebrospinal fluid (CSF) clearance after choroid plexus (ChP) NKCC1 overexpression (OE) indicate that choroid plexus (ChP) NKCC1 can remove excess cerebrospinal fluid (CSF). Therefore, it was hypothesized that choroid plexus (ChP) NKCC1 overexpression (OE) expression could mitigate ventriculomegaly in a model of postnatal obstructive hydrocephalus. choroid plexus (ChP) NKCC1 was first overexpressed at E14.5 by in utero AAV2/5 ICV (intracerebroventricular injection), then obstructive hydrocephalus was induced by a single unilateral injecting of kaolin into the lateral ventricle at P4⁵⁵, and finally the lateral ventricle volumes were evaluated by live T2 MRI at P14 (FIG. 6A). While both NKCC1 overexpression (OE) and control mice had enlarged ventricles at P14, NKCC1 overexpression (OE) mice had reduced ventriculomegaly compared to controls, with the average ventricle volume being less than 1/3 of the controls (FIGS. 6B-6D; ventricles and kaolin deposits are marked by arrows). Taken together, the findings demonstrate that early, choroid plexus (ChP) targeted NKCC1 overexpression (OE) had a sustained and broad impact on specific volumetric and biophysical parameters of the intracranial space with potential therapeutic applications to hydrocephalus.

### Discussion of Examples 1-7

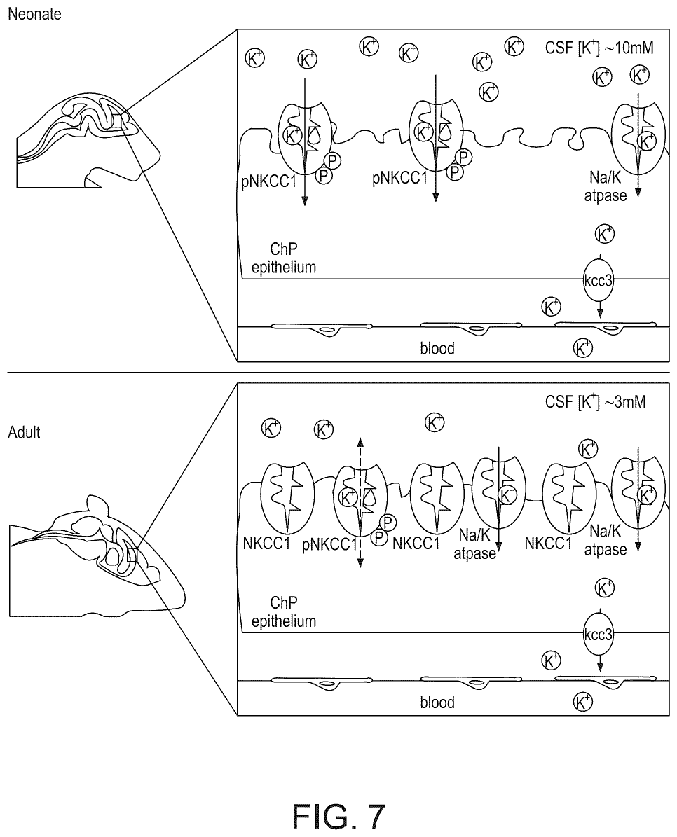

Investigations were completed to understand how cerebrospinal fluid (CSF) is cleared from the brain before the development of suggested cerebrospinal fluid (CSF) outflow routes (e.g. arachnoid granulations, arachnoid villi, perineural and paravascular pathways, and meningeal lymphatics)^{1,8,11-14,56} The intervening time period is a critical, transient phase in brain development when failure of cerebrospinal fluid (CSF) clearance has debilitating consequences⁷. The results suggest that this period is defined by a rapid decrease in cerebrospinal fluid (CSF) K⁺. The choroid plexus (ChP) mediates the cerebrospinal fluid (CSF) K⁺ clearance during this transition period, and thus forms a cerebrospinal fluid (CSF) outflow route through ion and water co-transport by NKCC1 (FIG. 7). This cerebrospinal fluid (CSF) clearance by the choroid plexus (ChP) during normal development contrasts the prevailing view that the choroid plexus (ChP) in a healthy brain always has net secretion of cerebrospinal fluid (CSF). Rather, early cerebrospinal fluid (CSF) clearance emphasizes bi-directional transport at the choroid plexus (ChP), which has been long known to be theoretically possible under physiological conditions and to occur in response to pathological conditions^{15,19-22}, but not received as much attention as the secretory activities. While the mechanism by which choroid plexus (ChP) epithelial cells clear the excess cerebrospinal fluid (CSF) K⁺ from their own cell bodies remains to be identified, a precisely timed function of the developing choroid plexus (ChP) was discovered that clears cerebrospinal fluid (CSF) prior to the formation of other better-studied routes and provides targets for fluid management intervention during a critical transition phase of brain development.

NKCC1 is a bidirectional transporter, recently discovered to be an important co-transporter of water in the adult choroid plexus (ChP)⁴⁰. Although clearly established as a key molecular mechanism of cerebrospinal fluid (CSF) regulation, choroid plexus (ChP) NKCC1 transport direction and its determinants in vivo have been actively debated due to the technical challenges of 1) specifically manipulating choroid plexus (ChP) NKCC1 without affecting NKCC1 in other cerebrospinal fluid (CSF)-contacting cells (ICV application of NKCC1 inhibitors such as bumetanide suffer from this limitation); and 2) accurately determining intracellular ion levels of choroid plexus (ChP) epithelial cells, and therefore ion gradients, under physiological conditions, as summarized in Table 2 and reviewed by Delpire and Gagnon²⁴. The in vivo “gain-of-function” approach effectively bypasses the abovementioned technical limitations. By overexpressing NKCC1 specifically in the choroid plexus (ChP) from a specific developmental stage onward through AAV transduction to amplify its physiological functional impact, the resulting cerebrospinal fluid (CSF) K⁺ and fluid volume changes could be subsequently observed to reveal the transporter's net directionality during a given period. Using this approach, it was found that, in contrast to the common notion that the choroid plexus (ChP) constantly produces cerebrospinal fluid (CSF) under physiological conditions, NKCC1 in the choroid plexus (ChP) mediated cerebrospinal fluid (CSF) clearance when nominal cerebrospinal fluid (CSF) [K⁺] is above adult values, especially during the first postnatal week in mice. Future studies could shed light on how this critical period corresponds to the human developmental timeframe, and whether shared mechanisms underlie the high vulnerability during development to disorders involving cerebrospinal fluid (CSF).

TABLE 2
Summary of publications reporting various values of choroid plexus (ChP) epithelium
intracellular Na⁺, K⁺, Cl⁻ concentrations. (N.D., not determined).

| Publication | Species | Age | [Na+]i | [K+]i | [Cl−]i |
|---|---|---|---|---|---|
| Gregoriades, J. M. C., Madaris, A., | Mouse | ChP | 9.2 ± | ND | 60.7 ± |
| Alvarez, F. J. & Alvarez- | | epithelial | 2.5 mM | | 12.3 mM |
| Leefmans, F. J. Genetic and | | culture | | | |
| pharmacological inactivation of | | collected | | | |
| apical Na(+)- K(+)-2Cl(−) | | from P10- | | | |
| cotransporter 1 in choroid plexus | | 21 mice | | | |
| epithelial cells reveals the | | | | | |
| physiological function of the | | | | | |
| cotransporter. American journal of | | | | | |
| physiology. Cell physiology | | | | | |
| 316, (2019). | | | | | |
| Steffensen, A. B. et al. | Mouse | 8-12 | 31 ± | 141 ± | 35 ± |
| Cotransporter-mediated water | | weeks | 5 mM | 12 mM | 9 mM |
| transport underlying cerebrospinal | | | | | |
| fluid formation. Nat Commun 9, | | | | | |
| (2018). | | | | | |
| Keep, R. F., Xiang, J. & Betz, A. | Necturus | Mature | 30.0 mM | 119 mM | 50 mM |
| L. Potassium cotransport at the rat | maculosus | | | | |
| choroid plexus. The American | | | | | |
| journal of physiology 267, (1994). | | | | | |
| Zeuthen, T. The effects of chloride | | | | | |
| ions on electrodiffusion in the | | | | | |
| membrane of a leaky epithelium. | | | | | |
| Studies of intact tissue by | | | | | |
| microelectrodes. Pflugers Archiv: | | | | | |
| European journal of physiology | | | | | |
| 408(3), (1987). | | | | | |
| Johanson, C. E. & Murphy, V. | Rat | Adult | 48 ± | 95 ± | 62 ± |
| A. Acetazolamide and insulin alter | | | 0.7 mmol/kg | 1.2 mmol/kg | 0.3 mmol/kg |
| choroid plexus epithelial cell | | | | | |
| [Na+], pH, and volume. The | | | | | |
| American journal of physiology | | | | | |
| 258, (1990). | | | | | |
| Saito, Y. & Wright, E. M. | Bullfrog | Mature | 10.5 mM | ND | 24 mM |
| Regulation of intracellular chloride | | | | | |
| in bullfrog choroid plexus. Brain | | | | | |
| Res 417, (1987). | | | | | |

It was next demonstrated that the choroid plexus (ChP) clearance of cerebrospinal fluid (CSF) can be targeted to temper abnormal cerebrospinal fluid (CSF) accumulation. The choroid plexus (ChP) has been targeted for therapeutic manipulation in rodent models of neurologic diseases ranging from Huntington's disease and lysosomal storage disorders, to Alzheimer's disease, where transduction of exogenous gene products into ependymal or choroid plexus (ChP) epithelial cells has improved cardinal symptoms of disease^{57,58}. Enhancing choroid plexus (ChP) epithelial cell NKCC1 transport capacity lessened the severity of ventriculomegaly in a model of obstructive hydrocephalus. The data demonstrate the possibility of treating congenital hydrocephalus by augmenting endogenous choroid plexus (ChP) NKCC1 activity to increase cerebrospinal fluid (CSF) absorption rates during early development when cerebrospinal fluid (CSF) [K⁺] is high. In adult animals, cerebrospinal fluid (CSF) K⁺ is rapidly removed when the concentration exceeds homeostatic levels^{28,29}, and one can speculate that choroid plexus (ChP) NKCC1 may be the route of such timely K⁺ clearance in adults as well. Therefore, the findings emphasize the choroid plexus (ChP) as a targetable, K⁺-sensitive and on-demand cerebrospinal fluid (CSF) drainage route in neurological disorders where cerebrospinal fluid (CSF) homeostasis is disrupted.

Further, in light of recent findings reporting hydrocephalus and ventriculomegaly in children with de novo loss-of-function CHD4 mutations⁴⁶, it was found that the CHD4/NuRD complex is required for the developmental regulation of NKCC1 expression. This connection suggests a possible pathophysiological mechanism whereby lack of CHD4 activity might reduce NKCC1 levels during early development (equivalent to P0-P7 in mice), and lead to insufficient cerebrospinal fluid (CSF) clearance resulting in hydrocephalus. In the 1oxP-cre approach, most CHD4 protein knockdown and resulting stagnation of NKCC1 expression occurred by P14, which is beyond the critical window of NKCC1 activity at P0-P7. As such, developmental ventriculomegaly was not modeled with this approach. Improved genetic tools for early CHD4 knockout and new animal models harboring the de novo patient mutations would be required to fully unravel the regulatory connection between CHD4/NuRD complex and NKCC1.

If the choroid plexus (ChP) NKCC1 is acting as a net outflow pathway during this transitional developmental phase, and mediates net cerebrospinal fluid (CSF) clearance it is possible that other sources may be contributing to influx of early cerebrospinal fluid (CSF) water content and ions. Besides possible choroid plexus (ChP) secretion that is independent of NKCC1, one mechanism that could be acting at this stage is cerebrospinal fluid (CSF) secretion by the developing brain tissue (e.g. progenitor cells that have a cell body at the ventricular zone but extend their basal processes to the developing pia) which have been reported to secrete cerebrospinal fluid (CSF) immediately after neural tube closure⁵¹. Future studies should elucidate whether this progenitor-mediated secretion mechanism extends into this transitional phase. Consistent with progenitor involvement in cerebrospinal fluid (CSF) dynamics, recently identified genes driving pediatric hydrocephalus are expressed predominantly by cortical progenitor cells lining the brain's ventricles⁶⁰, and not the choroid plexus (ChP), suggesting a non-choroidal source of abnormal cerebrospinal fluid (CSF) production.

In addition to fluid regulation, the newly identified choroid plexus (ChP) clearance route provides a key mechanism to regulate extracellular K⁺ during the first postnatal week. Ionic homeostasis of the cerebrospinal fluid (CSF) plays critical roles in brain development and function. The subunits of the major system for moving K⁺ against its individual concentration gradient, the Na⁺/K⁺-ATPase (i.e. Atp1a1 and Atp1b1), were not yet at their full expression levels during this period. The choroid plexus (ChP) NKCC1-mediated K⁺ clearance mechanism might assist in establishing the K⁺ gradient in a timely manner, which is crucial for cellular physiology⁶¹. Notably, the period of rapidly decreasing cerebrospinal fluid (CSF) [K⁺] overlaps with the developmental phase when the excitatory-to-inhibitory “GABA switch” occurs. In early cortical progenitor cells that reside in the ventricular zone and are bathed by cerebrospinal fluid (CSF), the classic inhibitory neurotransmitter GABA leads to excitatory potentials and suppression of DNA synthesis⁶². As newborn cortical neurons differentiate and migrate away from the ventricular zone, GABA begins to play its classic role as an inhibitory neurotransmitter in the context of lower intracellular Cl⁻ ⁶³ which is achieved through coordinated activities of neuronal K⁺/Cl⁻ co-transporters KCC2 and NKCC1^{64,65} Because ions, including K⁺, can traffic from cerebrospinal fluid (CSF) into interstitial fluid^{66,67} any interference with the developmental timeline of choroid plexus (ChP) NKCC1 that resulted in delayed cerebrospinal fluid (CSF) K⁺ clearance could potentially increase extracellular/interstitial fluid [K⁺] and affect neural physiology⁶¹. Specifically, such a change in extracellular/interstitial fluid [K⁺] could fundamentally impact neuronal NKCC1 and KCC2 transport equilibrium, potentially contributing to a delayed GABA switch. Furthermore, extracellular [K⁺] and certain K⁺ channels also regulate activities of microglia⁷³, which are critical in synaptic pruning during postnatal neurodevelopment in mice⁷⁴. Thus, the choroid plexus (ChP) is poised to play important roles in proper CNS formation by creating and maintaining optimal extracellular ionic homeostasis at different developmental stages, with subsequent effects on neuronal maturation, circuit formation, and neuroinflammatory homeostasis. Similarly, since numerous neurobiology research approaches utilize aCSF to mimic CNS extracellular fluid environment, it is important to note that cerebrospinal fluid (CSF) ionic compositions change drastically over development, and that correct aCSF recipes representing the native environment may be crucial for accurate functional evaluations. In the present example, ionically adjusted aCSF recipes were provided with Na⁺, K⁺, Ca²⁺, Mg²⁺, and Cl⁻ values for multiple developmental stages, ranging from embryonic, neonatal, postnatal, to adult.

Beyond key findings and implications during this critical transitional developmental stage, the experiments introduced a murine intracranial pressure (ICP) measurement device combined with constant cerebrospinal fluid (CSF) infusion. This approach provides a much-needed advance in fluid research technology that can be broadly applied to study essentially all cerebrospinal fluid (CSF) dynamic systems across the mouse lifespan. The tool was adapted from clinical practice to provide a range of options for measuring global cerebral fluid states that reflect the interaction between cerebrospinal fluid (CSF) and cranial tissues. In later life, cerebrospinal fluid (CSF) homeostasis is maintained by collaborative efforts from multiple putative players in the brain, including the choroid plexus (ChP), the dural lymphatics¹⁴, leptomeningeal vasculature 75, and the ependyma⁷⁶. While this approach measures overall cranial fluid dynamics as one single unit, future applications could apply mathematical models that have been proposed to isolate the contribution of distinct cerebrospinal fluid (CSF) outflow routes using data acquired from human patients⁷⁷. Such adaptability broadens the relevance of the tool and inspires optimism for further improved resolution in studying brain fluid dynamics.

Availability of this new tool also allows future researchers to obtain measurements in support of the growing comprehensive “systems” view of regulatory mechanisms of cerebrospinal fluid (CSF)-brain interactions. In summary, the findings of Examples 1-7 implicate a critical transient phase when the choroid plexus (ChP) acts as a route for cerebrospinal fluid (CSF) clearance prior to the maturation of other described clearance pathways. choroid plexus (ChP) NKCC1 mediates cerebrospinal fluid (CSF) clearance in a K⁺-dependent manner. The findings reveal that cerebrospinal fluid (CSF) fluid and ion management by the choroid plexus (ChP) exists at an early stage, which may have developmental significance. Targeting this absorption route holds promise for improving fluid management for congenital hydrocephalus and other cerebrospinal fluid (CSF) disorders.

### Example 8.: Improved Models of Intraventricular Hemorrhage (IVH) Using Age-Matched Blood in Mice

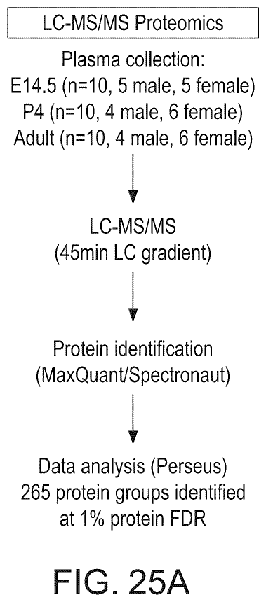

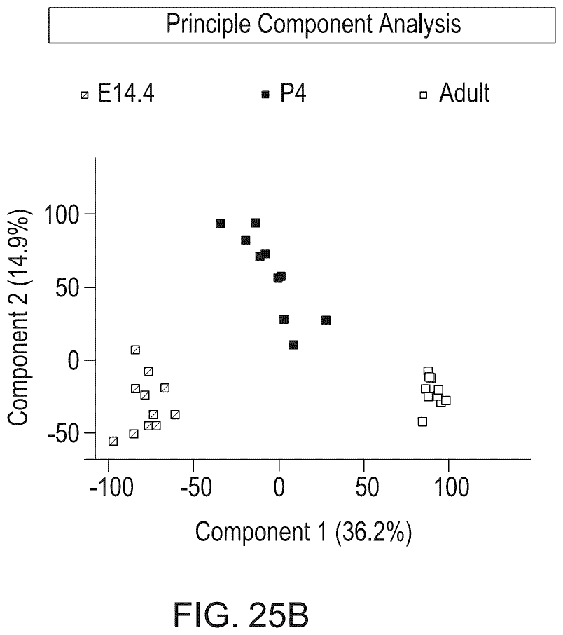

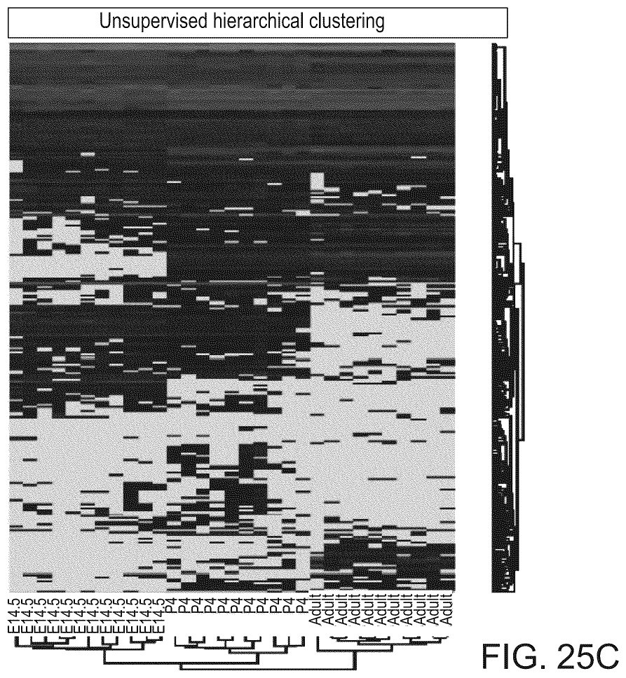

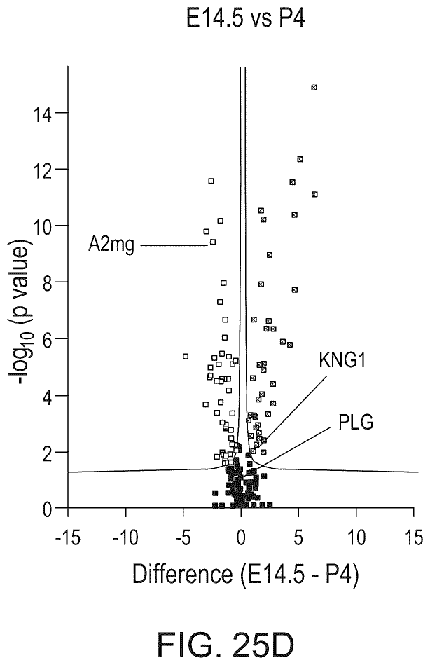

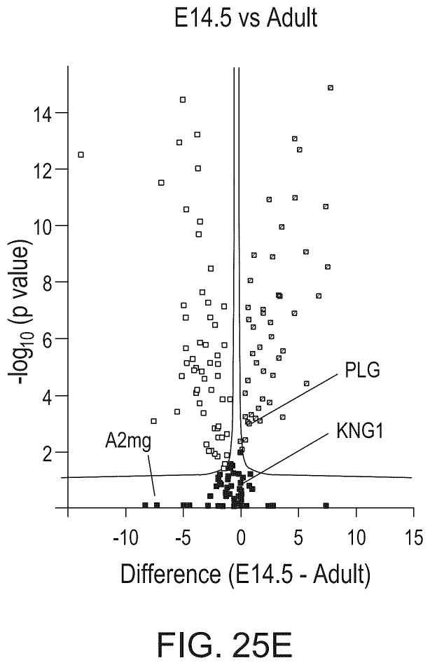

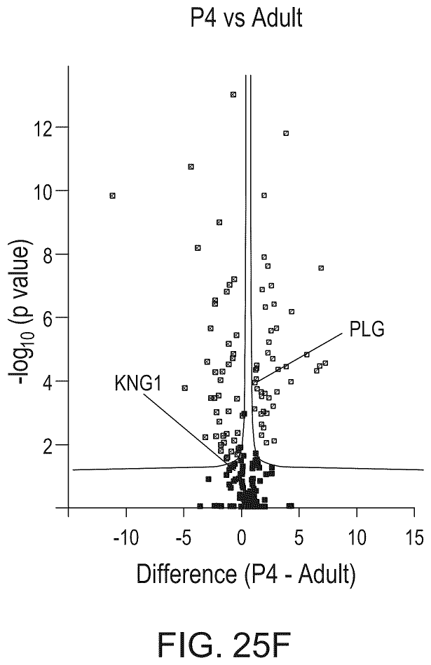

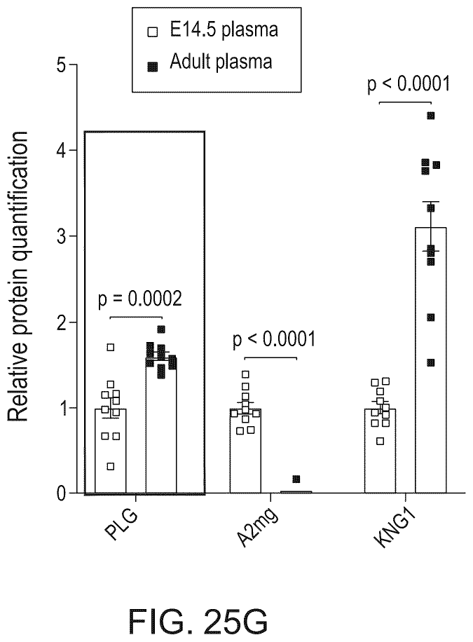

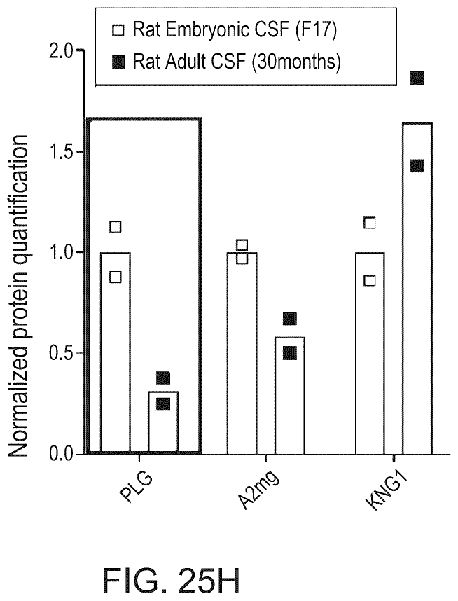

In adult rodent models of intraventricular hemorrhage (IVH) and post-hemorrhagic hydrocephalus (PHH), autologous, fresh, and unmodified blood is commonly used to model intraventricular bleeding and subsequent ventriculomegaly (27). However, in embryonic and neonatal intraventricular hemorrhage (IVH) studies, maternal blood has been more frequently used for experimental ease, despite the mismatch in age, blood composition, and other variables. The proteomic analysis of mouse plasma obtained at different developmental stages revealed striking changes in the blood proteome including key coagulation factors and inflammatory molecules (FIGS. 25A-25H). Therefore, to study the physiologic responses to ventricular blood, pediatric intraventricular hemorrhage (IVH) was mimicked using intracerebroventricular injections of age-matched blood. In addition, a parallel donor/recipient surgical model was employed to rapidly acquire unmodified allogeneic fetal blood from the same mouse strain (CD1), obviating the need for additives such as anti-coagulants which could trigger inflammation or other non-specific effects (28-31).

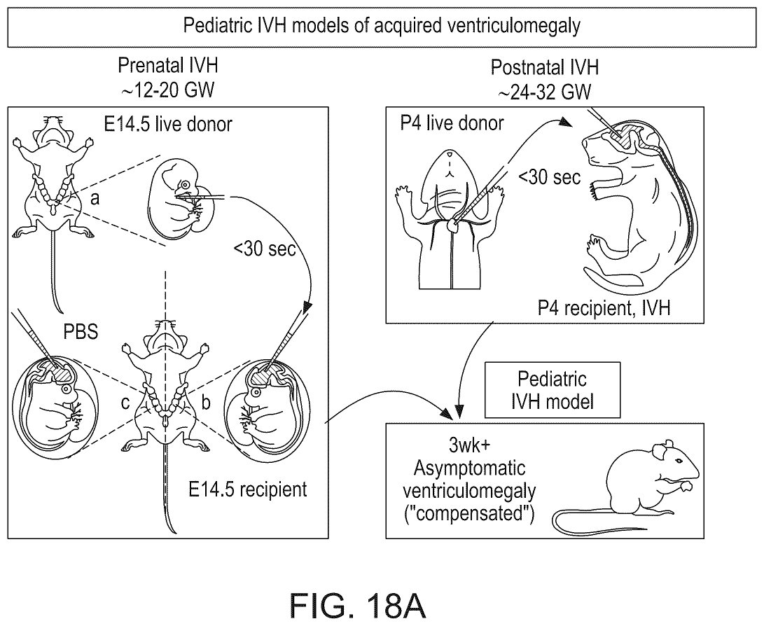

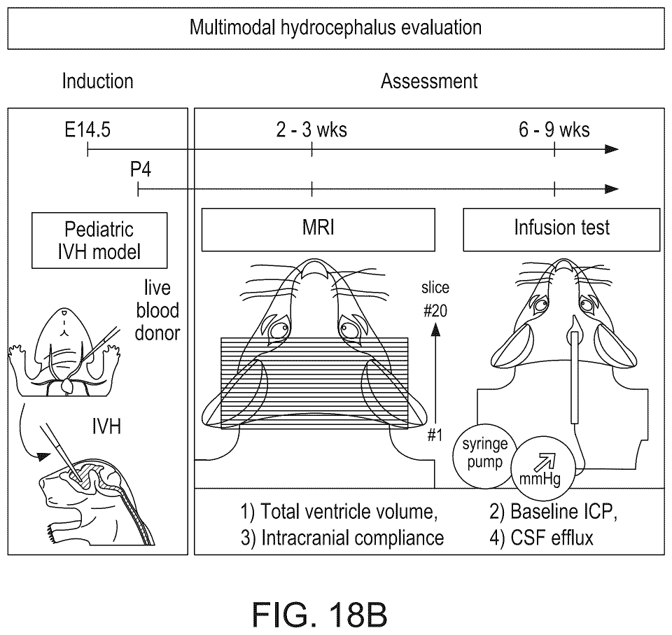

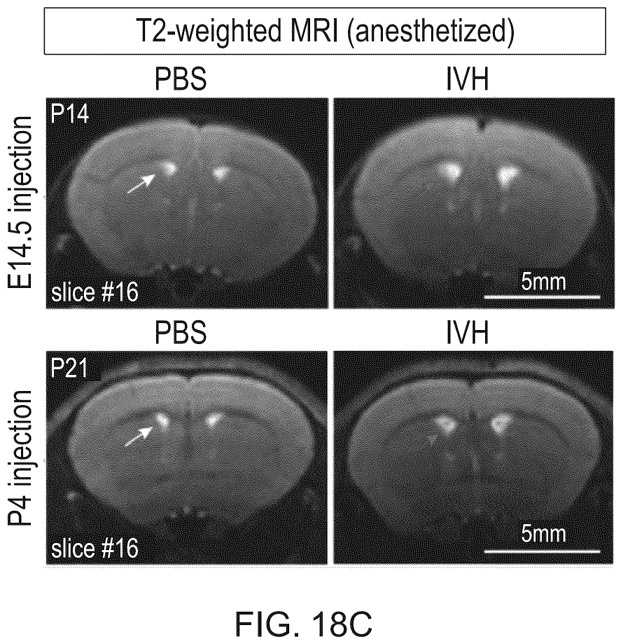

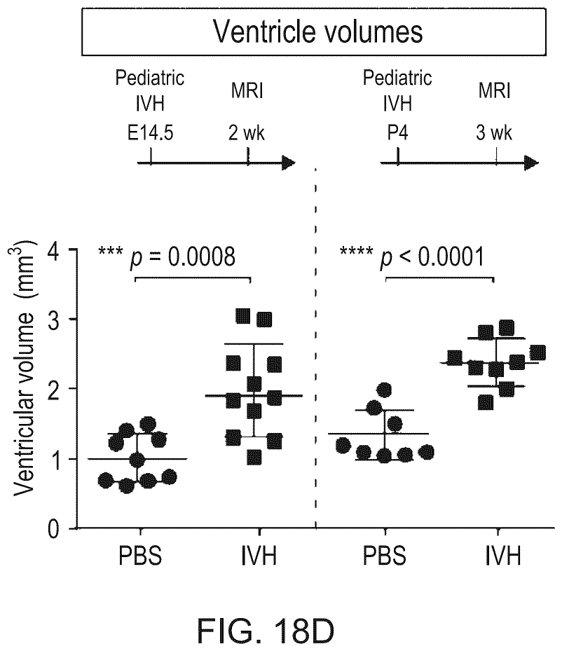

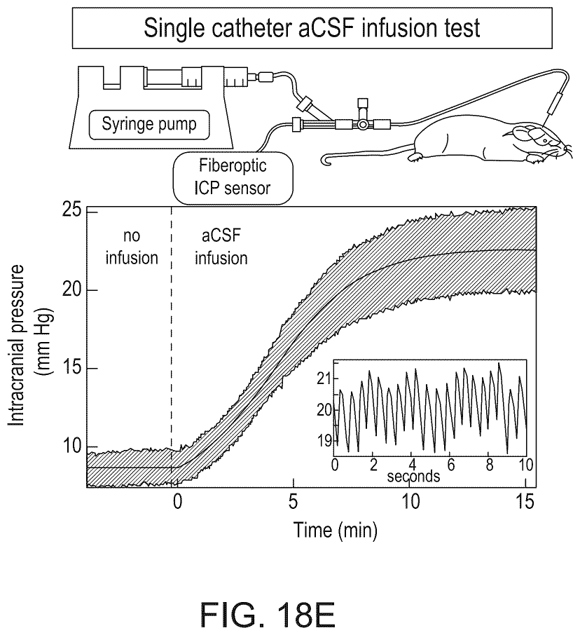

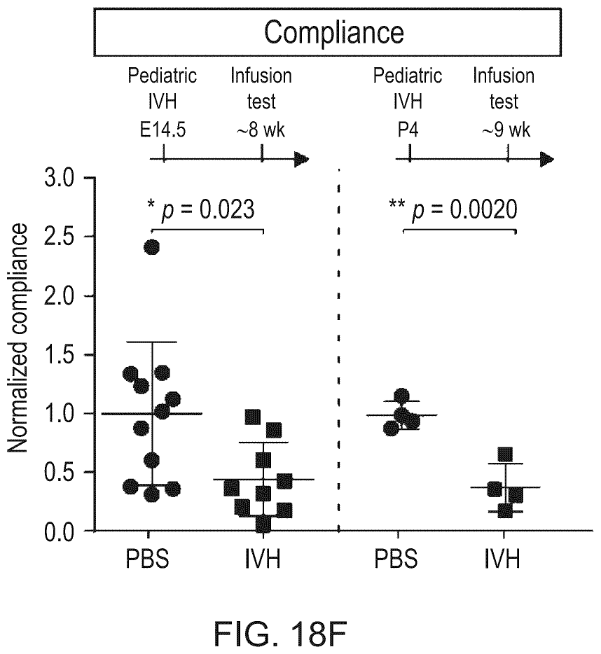

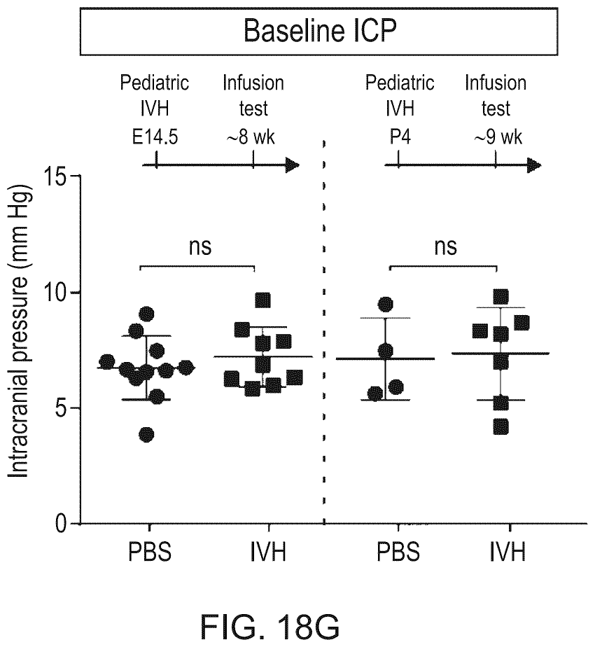

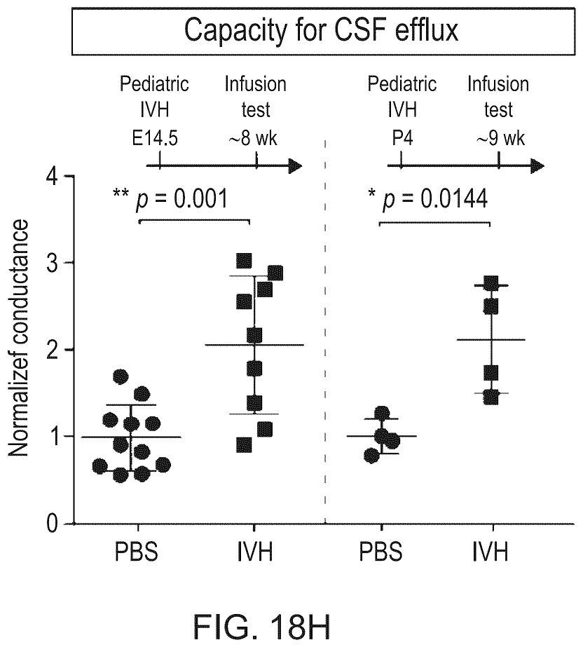

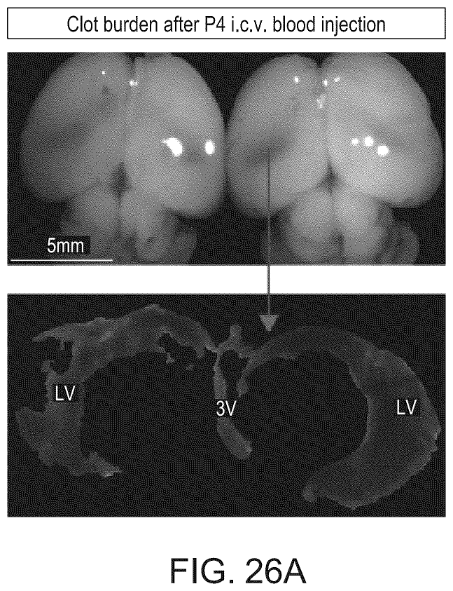

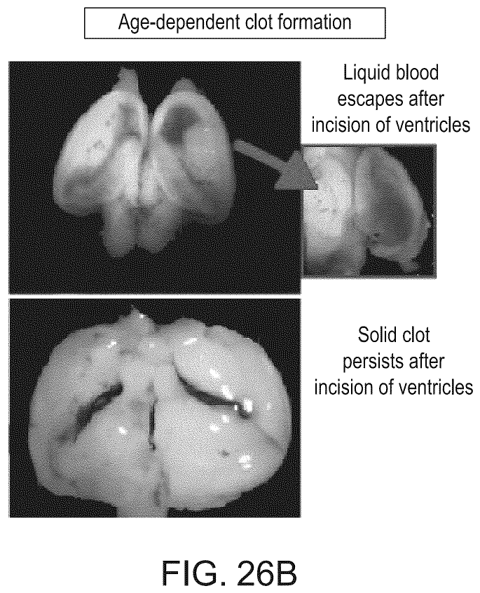

It was decided to study mice at two ages, embryonic day (E)14.5 and postnatal day (P)4, which generally correspond to human early (second trimester) and late (early third trimester) developmental stages, respectively (FIG. 18A). P4 corresponds to the time when preterm infants are most vulnerable to hemorrhagic strokes in the germinal matrix, and also coincides with the typical postnatal timing of intraventricular hemorrhage (IVH), within 72 hrs. after preterm birth. The E14.5 model represents hemorrhage that occurs early in development, which accounts for an estimated 10-20% of idiopathic hydrocephalus cases (32, 33). In order to model Papile grade 3 intraventricular hemorrhage (IVH) (34), volumes of blood corresponding to approximately 50% of total ventricle volume were injected at each age (2 μL blood at E14.5; 3-5 μL blood at P4). Ventricular blood formed solid clots inside P4 brains but remained in liquid form inside E14.5 brains (FIGS. 26A and 26B). It is not known to what extent clot formation, volume, or rate of clearance affects the progression of post-hemorrhagic hydrocephalus (PHH) or clinical outcomes (35), further emphasizing the physiologic importance of using age-matched blood to model intraventricular hemorrhage (IVH). In both models, the mice grew up without gross anatomical changes or obvious changes in behavior (FIG. 18A), consistent with the majority of the clinical cases. This mild post-hemorrhagic hydrocephalus (PHH) phenotype required the development of additional tools for detailed anatomic and hydrodynamic characterization.

### Example 9.: Intraventricular Blood Causes Moderate Ventriculomegaly and Hydrodynamic Changes in Both E14.5 and P4 Mice

Ventricle volume in isoflurane anesthetized mice was assessed approximately 3 weeks following introduction of intraventricular blood at either E14.5 or P4 by live, fast-acquisition, T2-weighted magnetic resonance imaging (MRI) (FIG. 18B). At both pediatric ages, intraventricular blood generated a nearly two-fold increase in the volume of the lateral and third ventricles as compared to control mice receiving phosphate-buffered saline (PBS) injections (FIGS. 18C and 18D); control (PBS injected) mice were not distinguishable from healthy uninjected mice (23). Notably, the doubling of ventricle volume was not apparent by less sensitive methods, including classic histology, post-perfusion brain MRI, and skull size measurements (data not shown), emphasizing the utility of live MRI evaluation of ventricular spaces.

To study cerebrospinal fluid (CSF) hydrodynamics, a clinical double spinal catheter cerebrospinal fluid (CSF) infusion test was adapted to murine research (23, 36-39) (FIGS. 18B and 18E). This approach enables continuous measurement of intracranial pressure (ICP) during ventricular infusion of artificial cerebrospinal fluid (CSF) (aCSF), which provides a quantitative analysis of (1) intracranial compliance (related to increased brain and meningeal displacement due to heightened ventricular forces during cerebrospinal fluid (CSF) infusion, and corresponding to how slowly the brain reaches peak intracranial pressure (ICP)) and (2) cerebrospinal fluid (CSF) clearance capacity (related to the resistance for cerebrospinal fluid (CSF) to flow out of the ventricular system, and corresponding the smallest change in peak intracranial pressure (ICP) following cerebrospinal fluid (CSF) infusion). When evaluated at 6-9 weeks, the baseline intracranial pressure (ICP) values in control saline injected or intraventricular hemorrhage (IVH) model mice remained stable and without significant differences in value; no symptoms of elevated intracranial pressure (ICP) were observed such as weight loss and impaired gait and posture (40). Nonetheless, it was found that mice that had received intraventricular blood had approximately half the intracranial compliance (FIG. 18F) as their controls, suggesting their brains were “stiffer” (reaching peak intracranial pressure (ICP) faster) and could not tolerate increasing cerebrospinal fluid (CSF) volume as well as healthy mice, which is in agreement with the current clinical paradigm of “scarring” of meninges and routes of cerebrospinal fluid (CSF) outflow, leading to reduced ventricular compliance in hydrocephalus (27). Baseline intracranial pressure (ICP) remained unaffected at this time (FIG. 18G). Surprisingly, the capacity for cerebrospinal fluid (CSF) outflow, as evaluated by conductance, was strikingly increased by two-fold in the intraventricular hemorrhage (IVH) model (FIG. 18H). Increased ventricular volume “compensated” by increased cerebrospinal fluid (CSF) clearance is consistent with excessive cerebrospinal fluid (CSF) accumulation, as has been previously described in humans with choroid plexus (ChP) hyperplasia or choroid plexus (ChP) papilloma (41) and in a rat model of post-hemorrhagic hydrocephalus (PHH) (19). Together, the data suggest that intraventricular blood in the pediatric intraventricular hemorrhage (IVH) mouse models causes a partially compensated form of ventriculomegaly that closely mimics the expected clinical sequelae for a majority of post-IVH patients.

### Example 10.: Lateral Ventricle Choroid Plexus (ChP) is an Immediate Early Responder to Intraventricular Blood

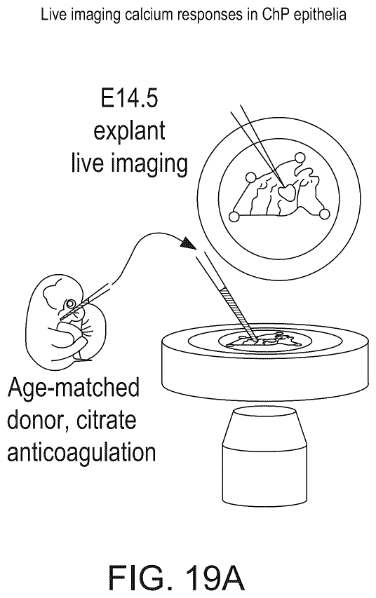

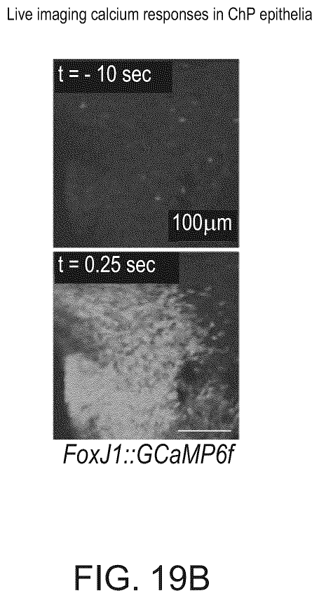

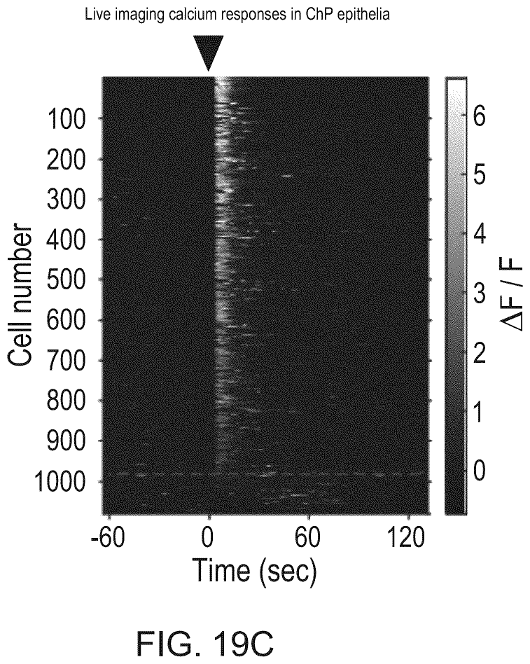

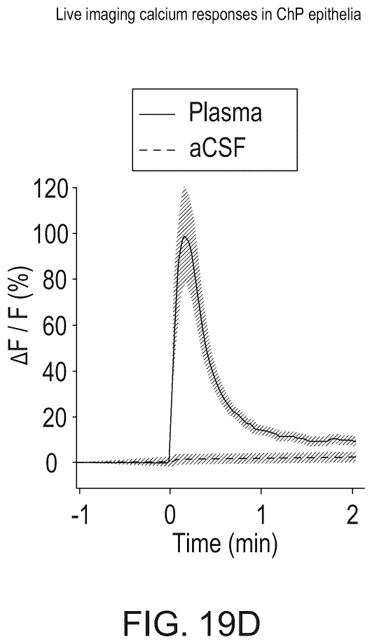

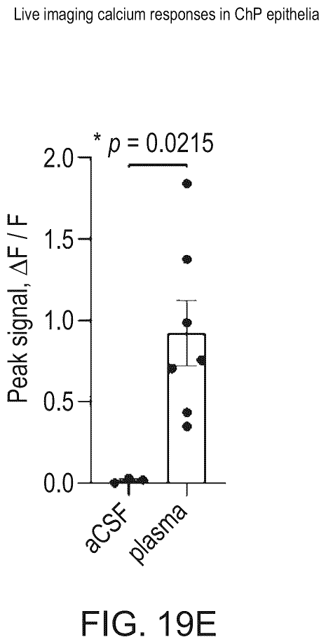

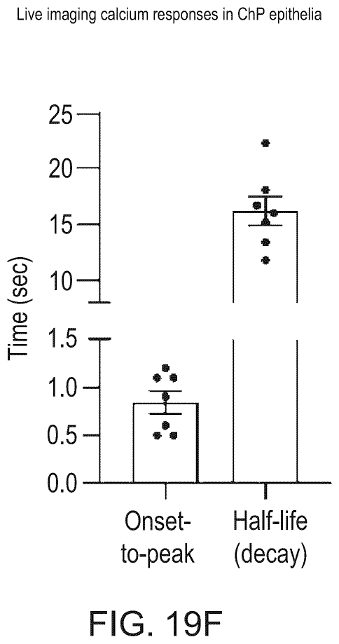

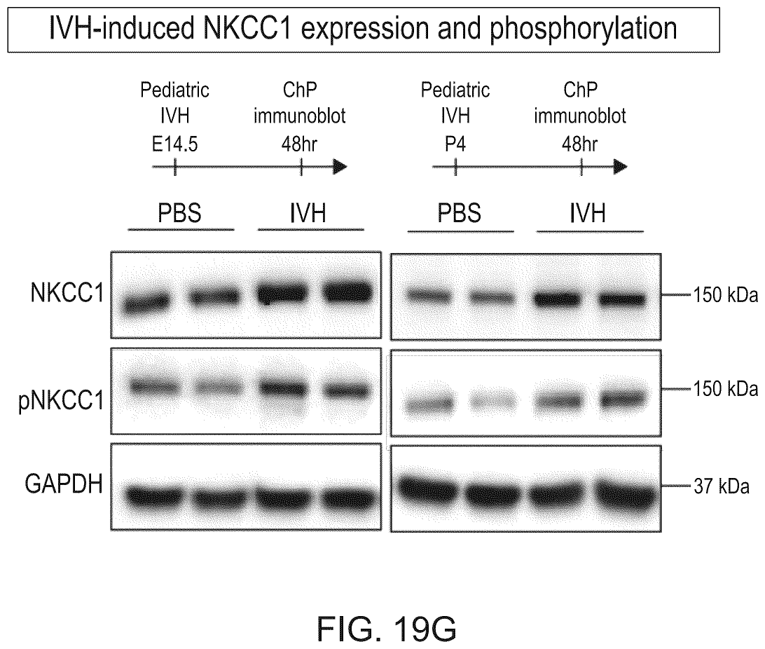

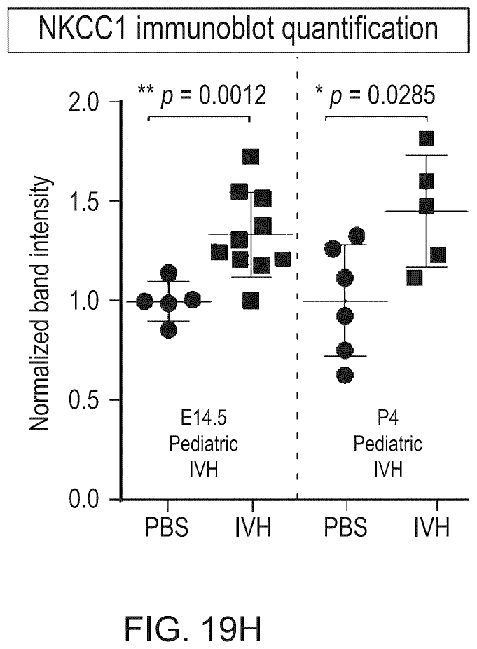

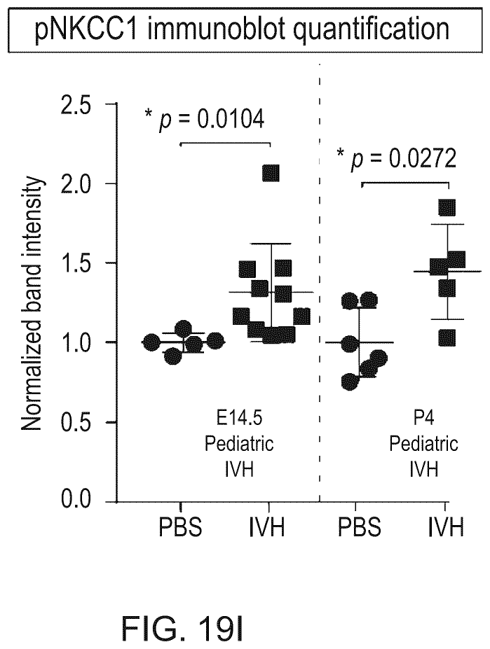

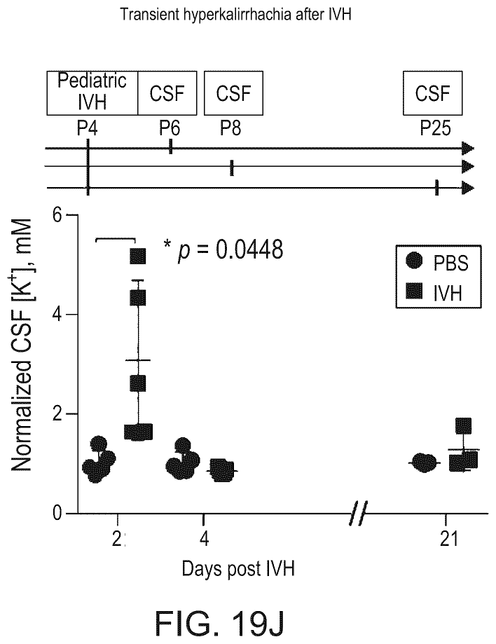

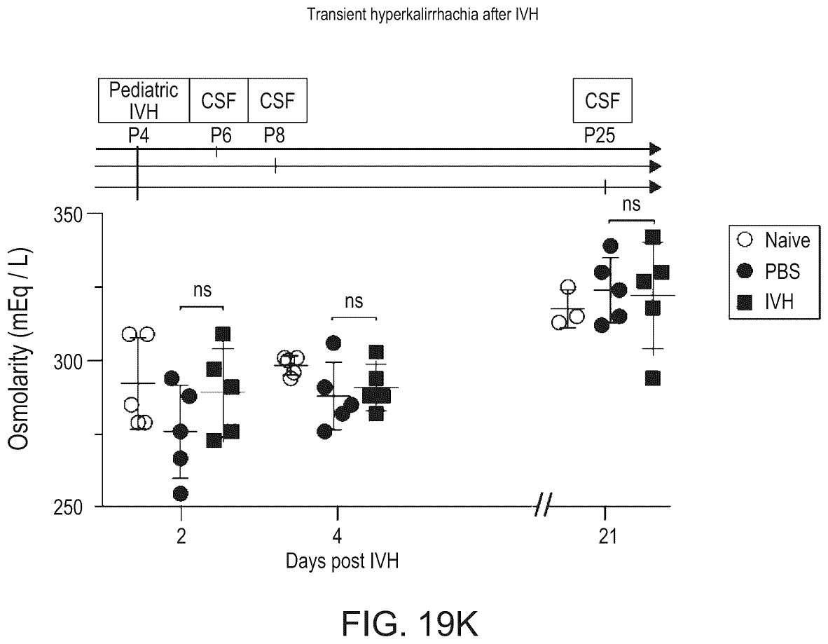

Further experiments revealed a diverse array of choroid plexus (ChP) responses to intraventricular blood over time, ranging from cytosolic calcium spikes within seconds to changes in protein phosphorylation in days, consistent with reported functions of the choroid plexus (ChP) for the surveillance of cerebrospinal fluid (CSF) composition. As is the case for many cell types, cytosolic calcium spikes are an early sign of activation of choroid plexus (ChP) epithelial cells. This activity is readily seen in response to neurotransmitters in the cerebrospinal fluid (CSF)(12, 42). Therefore, choroid plexus (ChP) calcium responses to blood plasma was evaluated. E14.5 choroid plexus (ChP) explants derived from FoxJ1::GCaMP6f transgenic mice were affixed to glass coverslips and incubated in aCSF to allow live cytosolic calcium imaging of FoxJ1-expressing choroid plexus (ChP) epithelial cells during focal blood application (FIG. 19A). choroid plexus (ChP) epithelia increased cytosolic calcium rapidly in response to a single application of cell-free blood plasma (FIG. 19B similar data was obtained with cell-free blood serum, data not shown). After the initial wave of calcium, the cells were transiently more active with increased frequency of spontaneous cytosolic calcium spikes compared to baseline, for ˜ 1 min (FIG. 19C). Cell segmentation analysis by morphology confirmed that >90% of total epithelial cells were activated in response to plasma, which allowed the use of the bulk signal analysis as a surrogate for individual cellular activities and evaluate overall choroid plexus (ChP) tissue responses to blood (FIGS. 19D-19F).

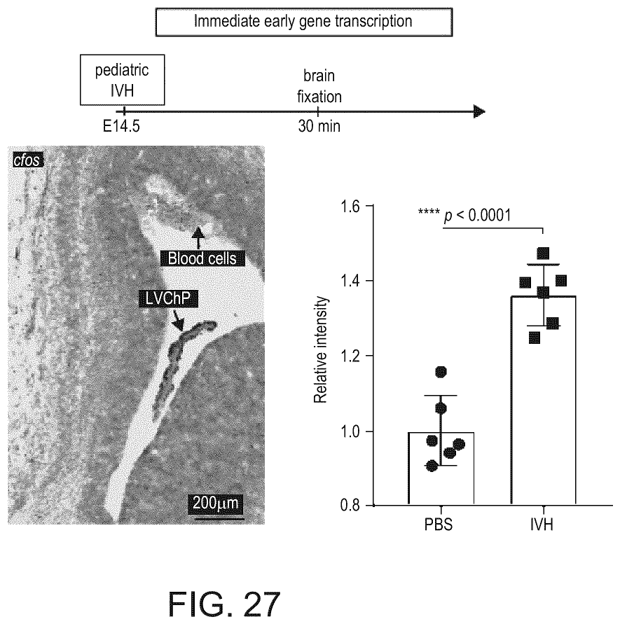

The rapid calcium responses of choroid plexus (ChP) epithelial cells were followed by changes in gene transcription and post-translational modifications when evaluated in vivo. Increased transcription of the immediate early gene c-fos selectively within the choroid plexus (ChP) (and not within the ependymal lining of the ventricle) 30 min. following in vivo intracerebroventricular injection of age-matched whole blood in E14.5 embryos was identified (FIG. 27). Consistent with previous reports in adult intraventricular hemorrhage (IVH) models(18, 19), robust phosphorylation, and therefore activation, of NKCC1 was observed in the E16.5 and P4 choroid plexus (ChP) 48 hrs. following intraventricular hemorrhage (IVH), as well as increased total NKCC1 expression compared to their PBS controls (FIGS. 19G-19I).

Elevated activity of the water-ion co-transporter NKCC1 in the choroid plexus (ChP) could increase or decrease ventricular cerebrospinal fluid (CSF) volume depending upon the direction of transport, which is determined by the combined gradients of Na⁺, K⁺, and Cl⁻, with extracellular K⁺ having a strong influence due to its naturally very low level. These ions were measured in the cerebrospinal fluid (CSF) 48 hrs following intraventricular hemorrhage (IVH) using the P4 age group, and found significant, transient high cerebrospinal fluid (CSF) [K⁺](hereby termed “hyperkalirrhachia”) (FIG. 19J), while the total osmolarity at each time point remain unchanged between intraventricular hemorrhage (IVH) mice and controls (FIG. 19K). The timing of post-hemorrhagic hyperkalirrhachia coincided with transiently increased choroid plexus (ChP) NKCC1 expression and phosphorylation at the same timepoint. This finding provides evidence for the hypothesis that NKCC1 is functioning to normalize the cerebrospinal fluid (CSF) [K⁺], by increasing active transport of [K⁺] and water from ventricular cerebrospinal fluid (CSF) into the choroid plexus (ChP) epithelia. These effects were consistent with the observations of increased cerebrospinal fluid (CSF) clearance in the model of intraventricular hemorrhage (IVH) (FIG. 18H) despite the increase in ventricle size (FIG. 18C-18D).

### Example 11.: Disruption of Choroid Plexus (ChP) Epithelial Calcium Signaling Worsens Post-Hemorrhagic Hydrocephalus

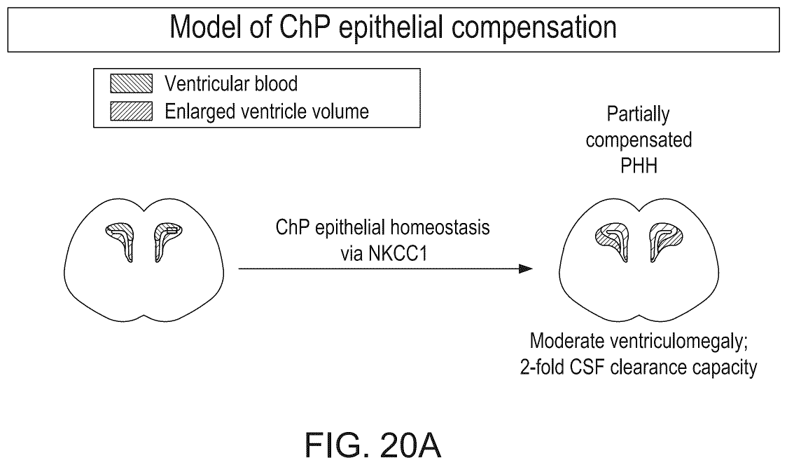

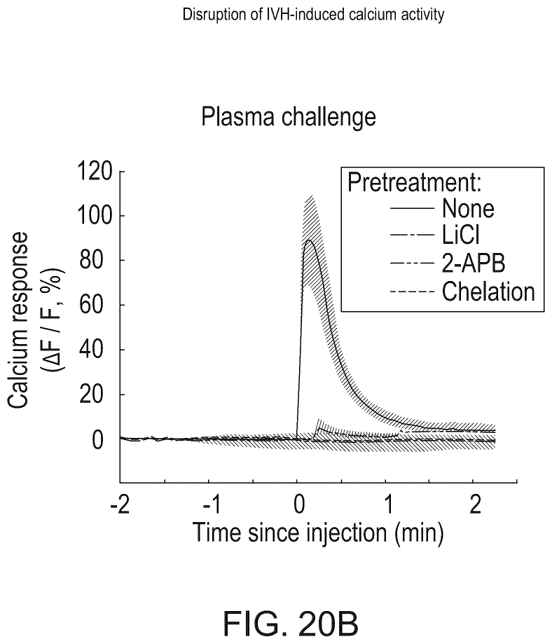

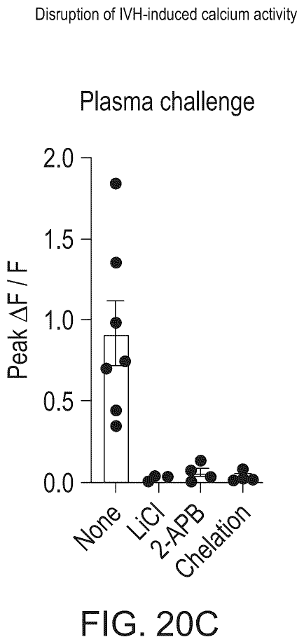

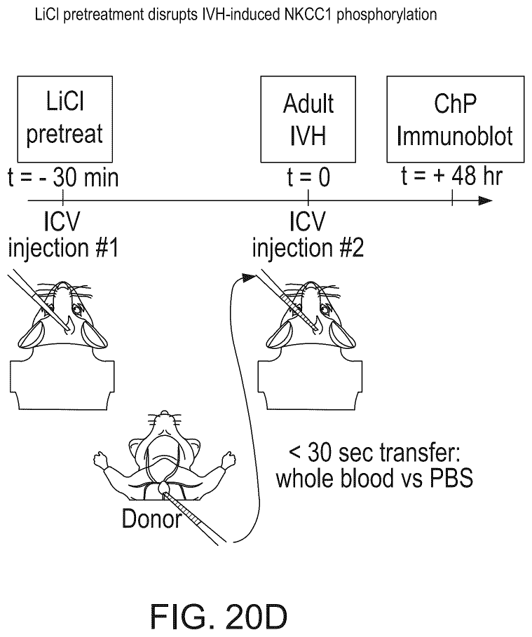

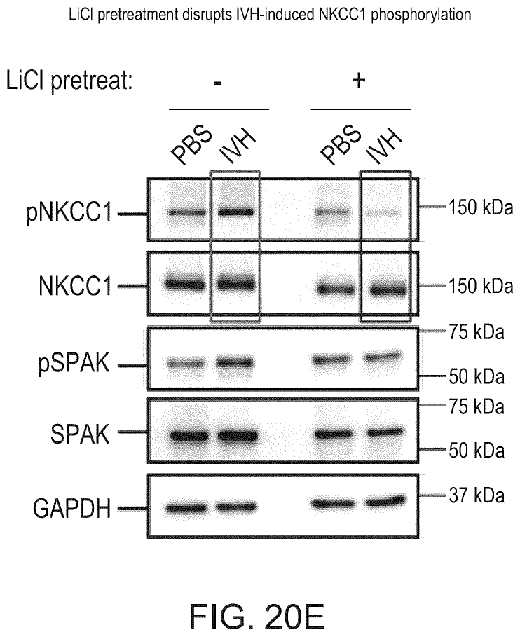

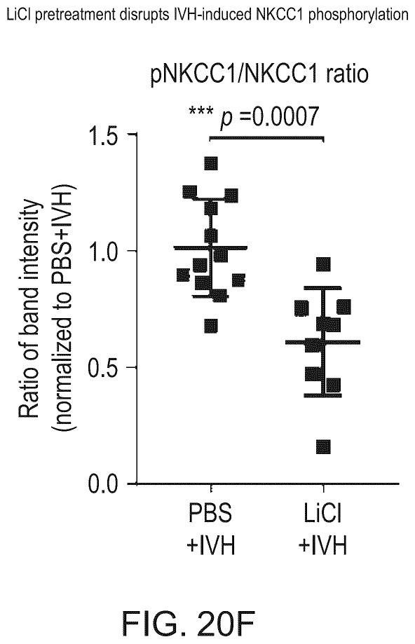

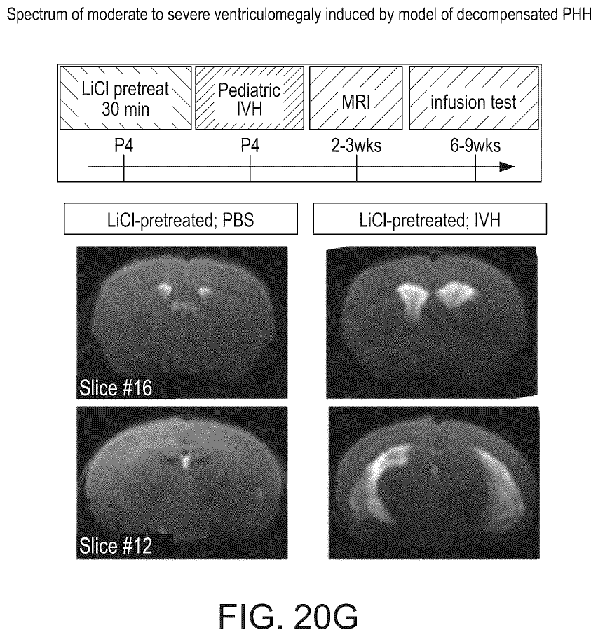

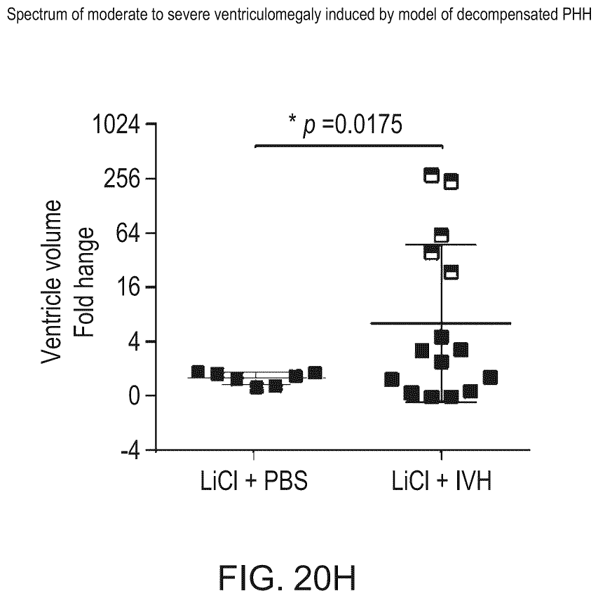

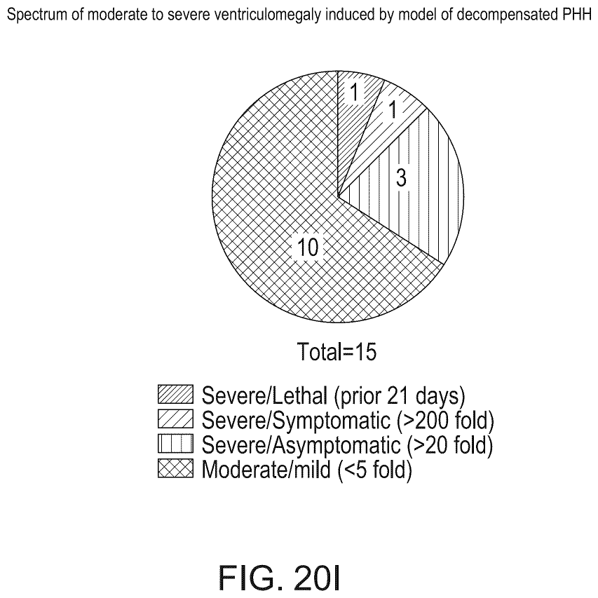

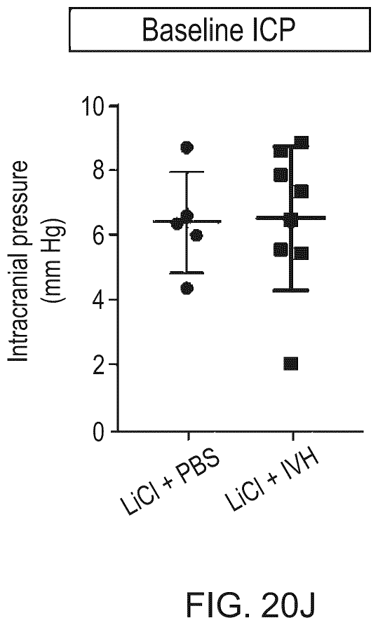

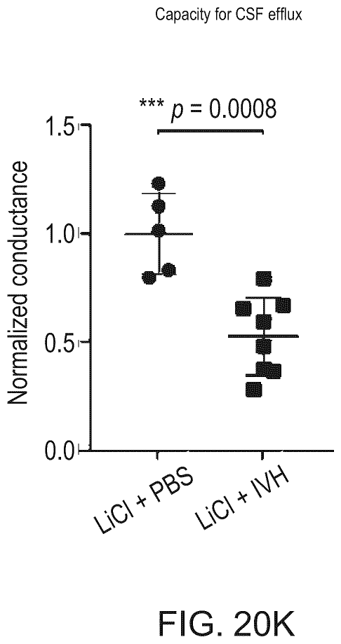

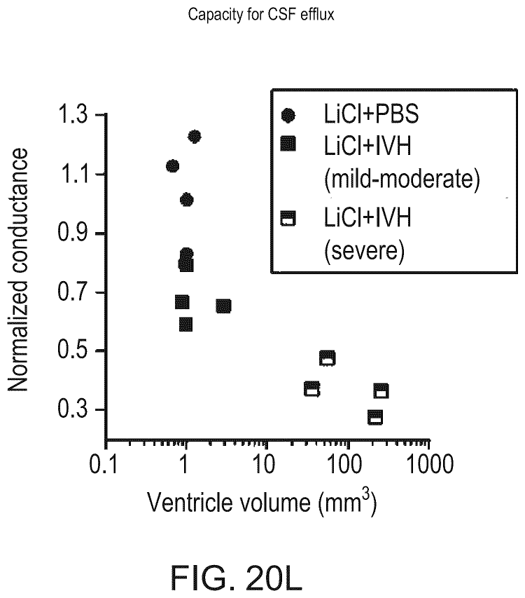

The instantaneous intracellular calcium spikes, subsequent upregulation of phosphorylated and total NKCC1, and concurrent increase in cerebrospinal fluid (CSF) [K⁺] are all consistent with choroid plexus (ChP) as an immediate responder to ventricular blood and mediating the observed increased cerebrospinal fluid (CSF) clearance through NKCC1 (FIG. 20A). If this were the case, disruption of this compensatory response would be expected to worsen intraventricular hemorrhage (IVH) outcomes. To test this hypothesis in choroid plexus (ChP) explants exposed to focal applications of age-matched plasma (FIG. 20B-20C), established methods to disrupt calcium signaling in epithelial cells (43-45) were adapted to successfully abolish choroid plexus (ChP) calcium responses and subsequent NKCC1 phosphorylation. These methods included (1) blocking release of intracellular calcium stores by disrupting 1,4,5-trisphosphate (IP3) signaling with lithium chloride (LiCl) or directly inhibiting the IP3 receptor with 2-aminoethoxydiphenyl borate (2-APB), and (2) abolishing extracellular calcium influx by chelation with ethylenediaminetetraacetic acid (EDTA) or ethylene glycol-bis(β-aminoethyl ether)-N,N,N′,N′-tetraacetic acid (EGTA). The in vivo effect of calcium disruption on choroid plexus (ChP) activity was tested by pre-treating adult mice with intracerebroventricular LiCl (50 mM, 5 μL) 30 min. prior to intraventricular blood exposure and evaluating the level of pNKCC1, which is a sensitive and established readout for choroid plexus (ChP) responses to intraventricular blood after 48 hrs (FIG. 20D). LiCl pre-treatment decreased NKCC1 phosphorylation rate under these conditions (FIGS. 20E-20F), possibly due to a decrease in the phosphorylation of SPAK (FIG. 20E), which is an upstream kinase that is known to phosphorylate NKCC1 (19).

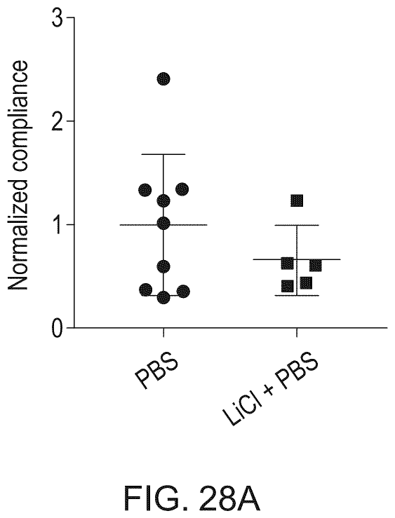

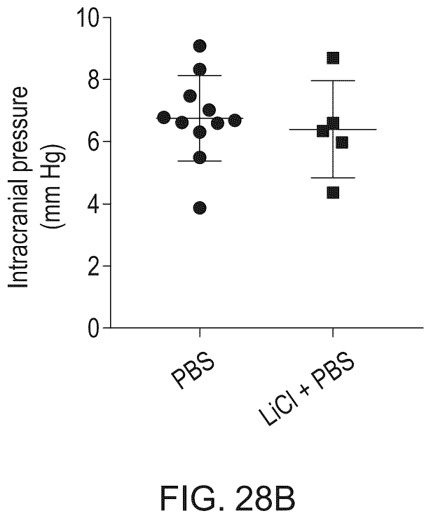

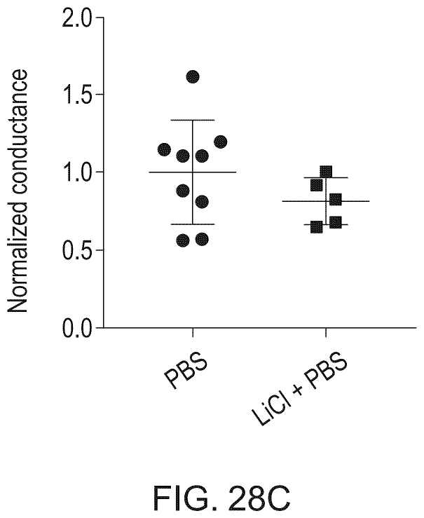

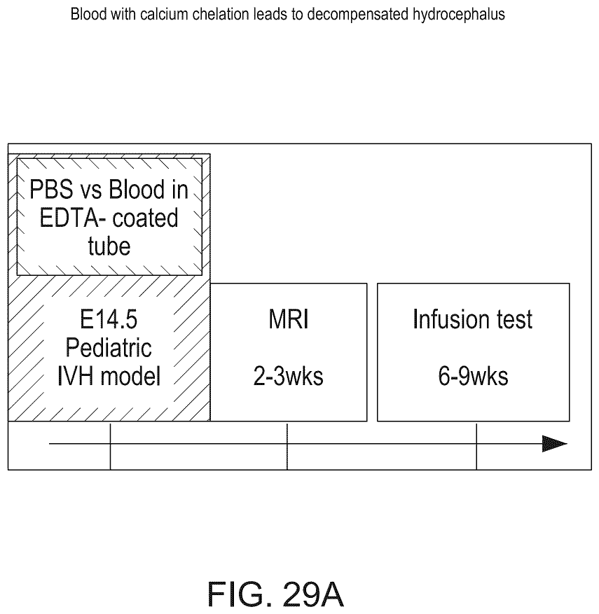

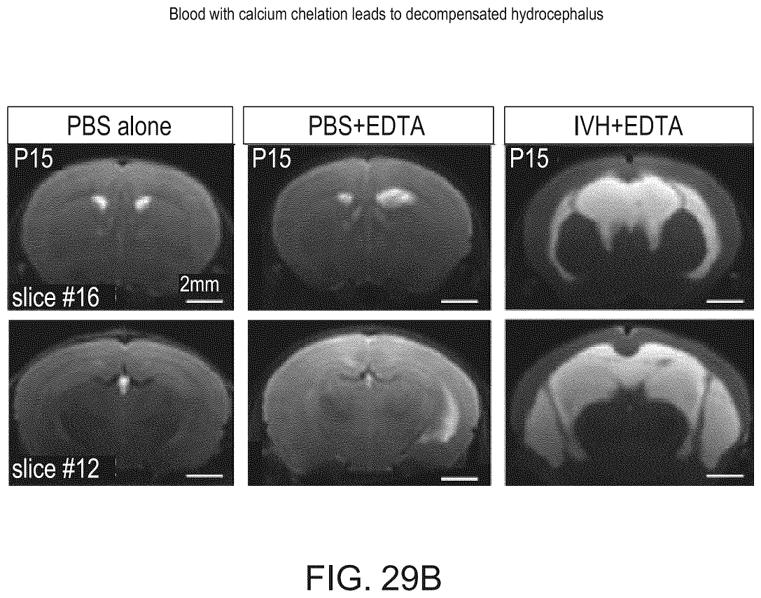

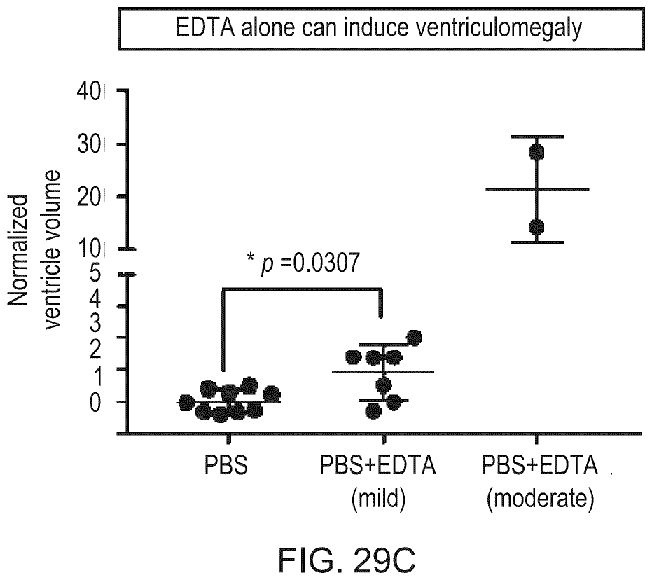

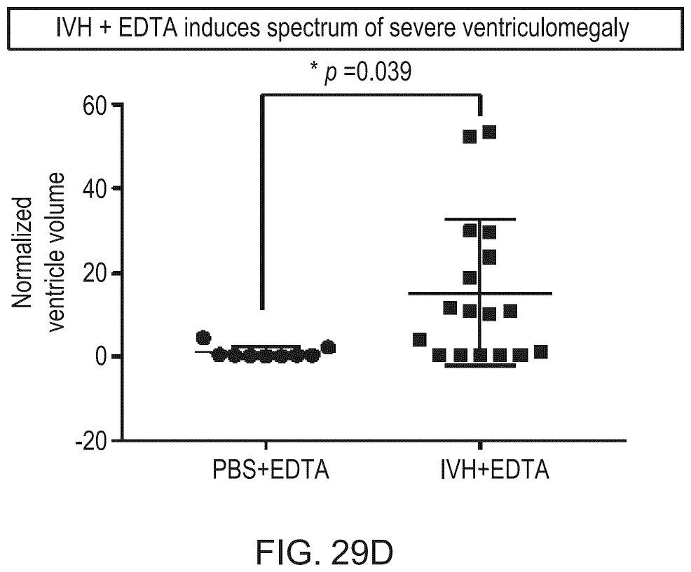

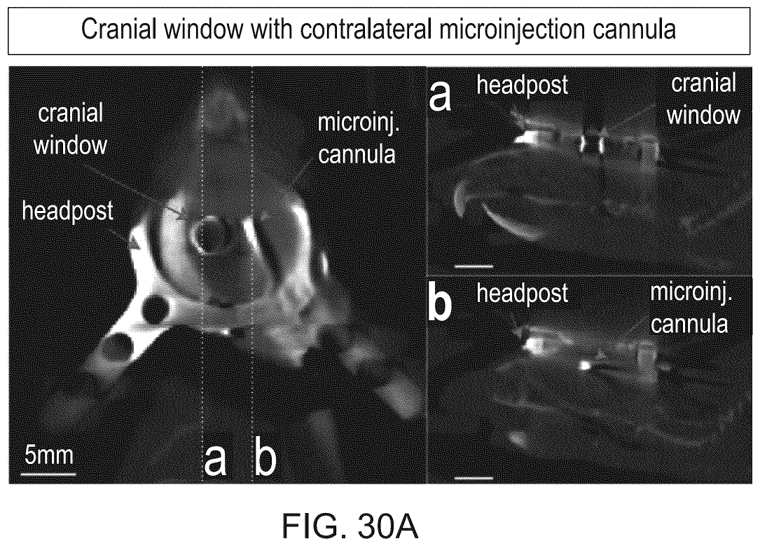

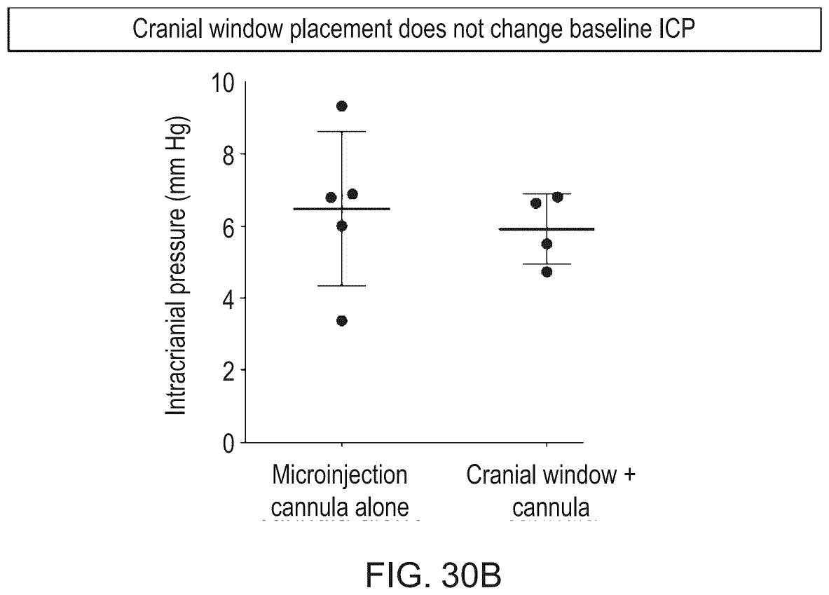

It was next evaluated whether disruption of choroid plexus (ChP) epithelial responses would impair compensatory cerebrospinal fluid (CSF) clearance in the intraventricular hemorrhage (IVH) model. E14.5 embryos were pretreated with intracerebroventricular LiCl 30 min. prior to intraventricular blood versus PBS injection, and then evaluated postnatal mice for hydrocephalic phenotypes by MRI and cerebrospinal fluid (CSF) infusion test as described above (FIG. 20G). Mice receiving intraventricular blood that were pre-treated with LiCl had more pronounced ventriculomegaly as compared to controls, including two extreme cases with over 200-fold ventricular enlargement (FIGS. 20G-20I). cerebrospinal fluid (CSF) infusion testing revealed a decreased, rather than increased, cerebrospinal fluid (CSF) clearance capacity in intracerebroventricular LiCl-Blood mice compared to intracerebroventricular LiCl-PBS mice, with unaltered intracranial pressure (ICP) (FIGS. 20J-20K). Notably, intracranial pressure (ICP) in cases of severe ventriculomegaly is technically difficult to ascertain, given the expected release of a small amount of cerebrospinal fluid (CSF) during dural opening and cannula placement, and despite acquisition of long baseline recordings to identify the true baseline intracranial pressure (ICP). The extent of this decrease in cerebrospinal fluid (CSF) clearance capacity correlated with the severity of ventriculomegaly (FIG. 20L) and LiCl treatment alone did not affect ventricle size (FIGS. 28A-28C). In a complementary experiment, intracerebroventricular injection of blood containing EDTA (providing irreversible calcium chelation) also produced severe ventriculomegaly in E14.5 embryos 3 weeks after the injection (FIGS. 29A-29D). These data support the importance of choroid plexus (ChP) epithelial calcium signaling for the maintenance of a reduced ventricle size in the setting of intraventricular hemorrhage (IVH).

### Example 12.: Choroid Plexus (ChP) Targeted NKCC1 Overexpression Prevents Ventriculomegaly in Pediatric Intraventricular Hemorrhage (IVH) Models

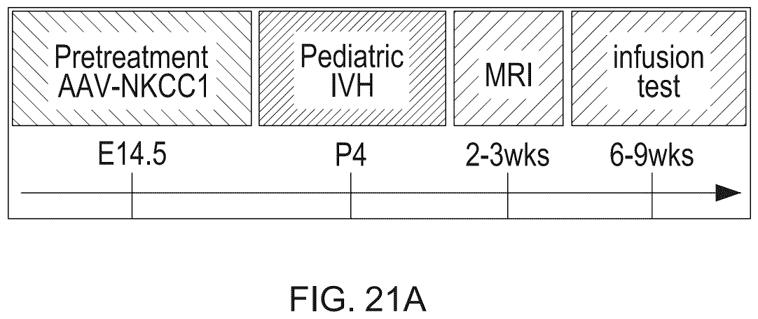

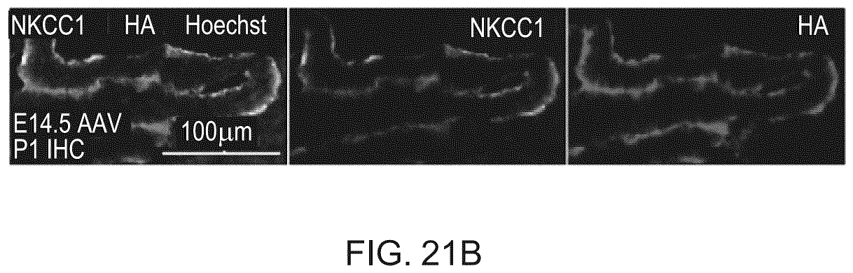

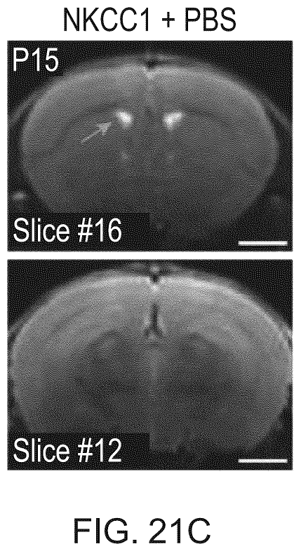

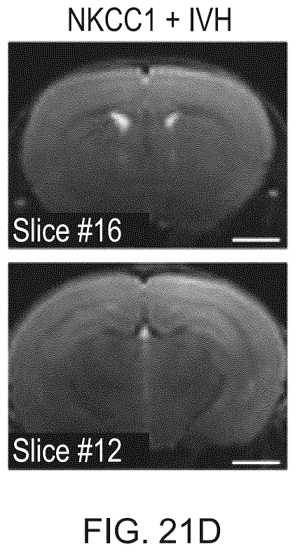

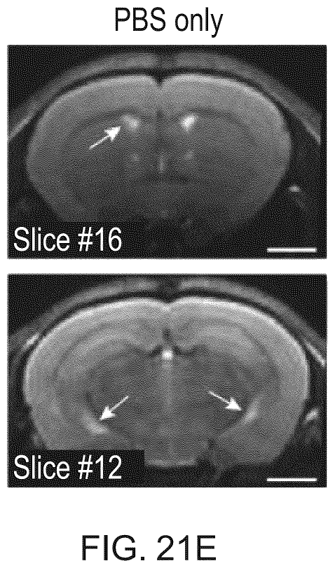

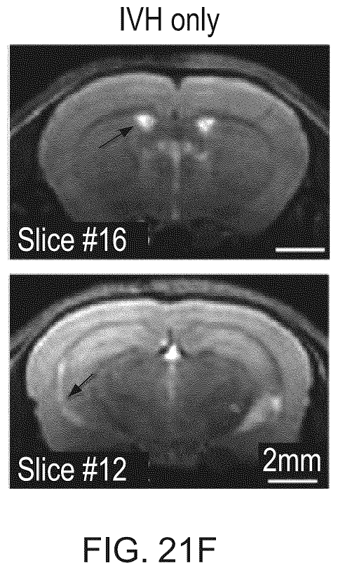

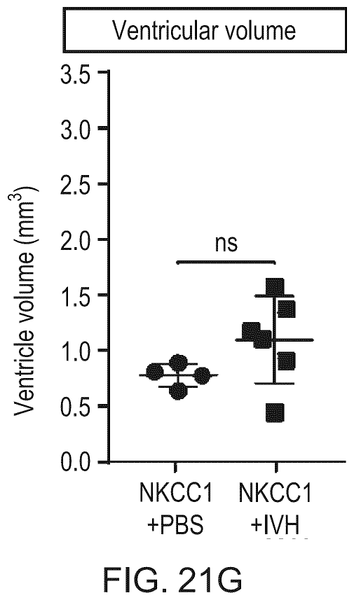

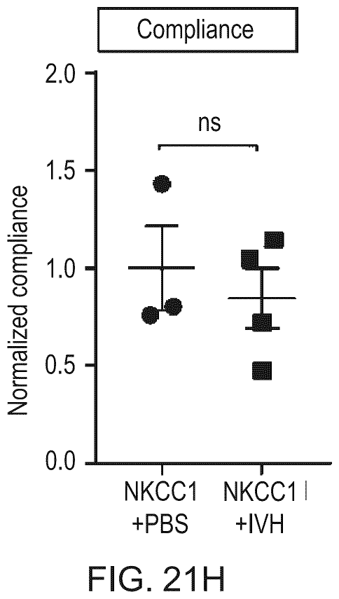

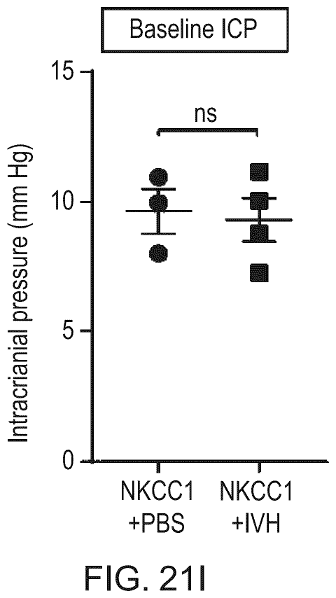

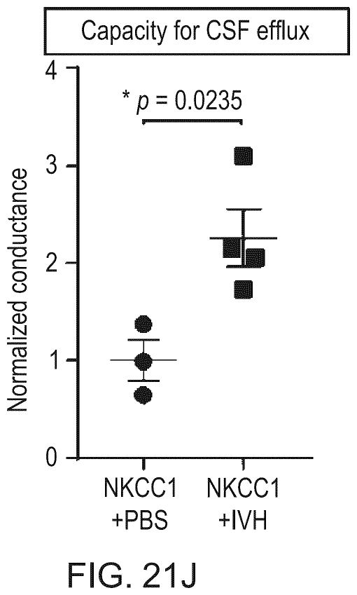

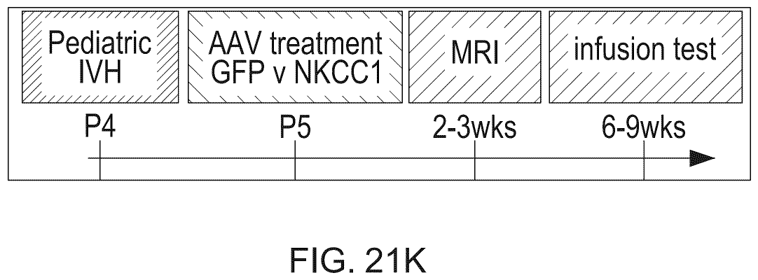

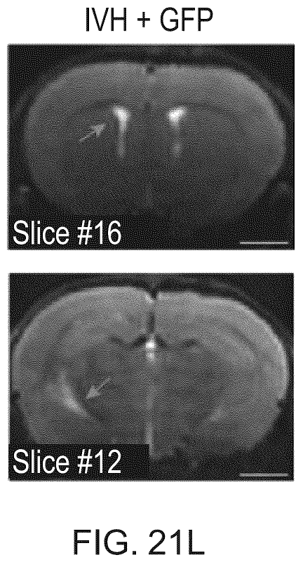

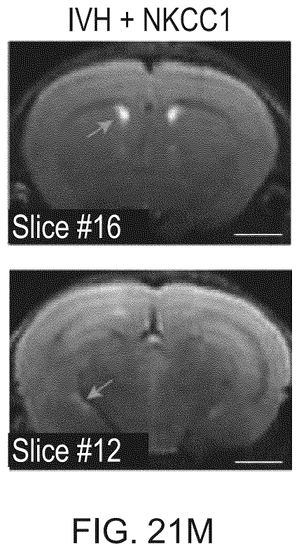

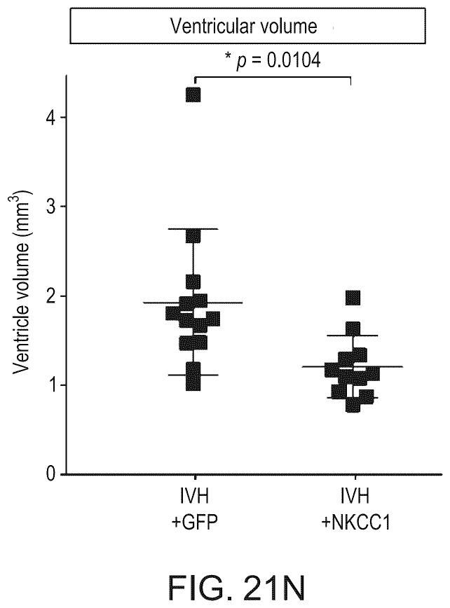

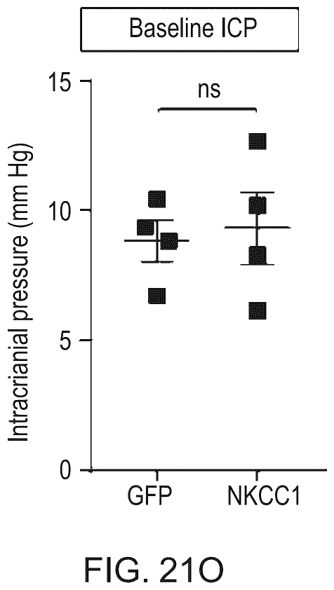

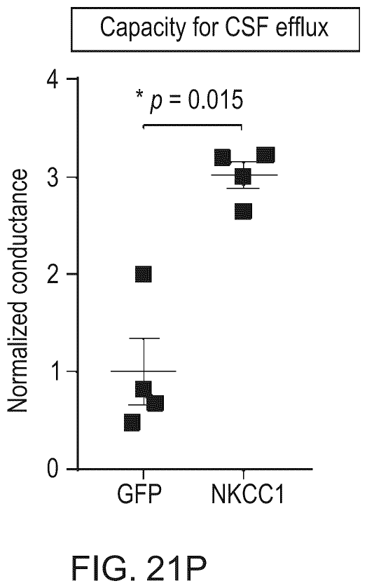

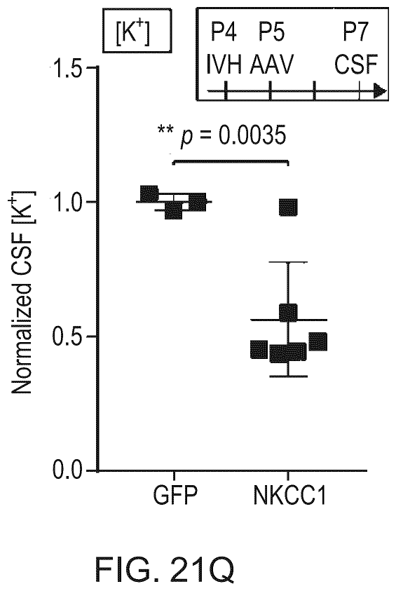

The concept of choroid plexus (ChP) NKCC1 mediating a reduction in ventricle size, in the setting of intraventricular hemorrhage (IVH) and hyperkalirrhachia, provides several testable hypotheses for leveraging its therapeutic potential in humans. It was first asked whether an increase in baseline choroid plexus (ChP) NKCC1 availability would enable a more robust and favorable ventricular response to intraventricular hemorrhage (IVH), rather than worsening ventriculomegaly. Human NKCC1 was overexpressed specifically in E14.5 mice choroid plexus (ChP) epithelial cells using AAV2/5 (FIGS. 21A-21B) (23) and then evaluated ventricle sizes (at 2-3 weeks) and cerebrospinal fluid (CSF) outflow (at 6-9 weeks). choroid plexus (ChP) NKCC1 overexpression (OE) mice with intraventricular hemorrhage (IVH) showed no obvious ventriculomegaly compared to controls (NKCC1 overexpression (OE) mice receiving PBS) (FIGS. 21C-21G). They were also protected from blood-induced decreases in intracranial compliance (FIG. 21H). choroid plexus (ChP) NKCC1 overexpression (OE) mice also had a consistently increased capacity for cerebrospinal fluid (CSF) clearance without changes in intracranial pressure (ICP) following intraventricular blood exposure (FIGS. 21I-21J). These results establish the therapeutic potential of human NKCC1 overexpression to prevent ventriculomegaly in the pediatric intraventricular hemorrhage (IVH) model.

The timing of NKCC1 overexpression (OE) was next explored and it was hypothesized that choroid plexus (ChP) NKCC1 overexpression (OE) introduced after intraventricular hemorrhage (IVH) could still improve the choroid plexus (ChP) response to intraventricular hemorrhage (IVH), as would a therapeutic intervention. Intraventricular blood was introduced in pups at P4 for 24 hrs prior to injecting AAV encoding NKCC1 or GFP into the same lateral ventricle. These mice were evaluated by MRI (at 2-3 weeks) and hydrodynamic testing (6-9 weeks) (FIG. 21K). It was found that choroid plexus (ChP) NKCC1 overexpression (OE) mice had smaller ventricles compared to their AAV-GFP injected littermates following intraventricular blood exposure (FIGS. 21L-21N). Ventricle volumes of choroid plexus (ChP) NKCC1-OE intraventricular hemorrhage (IVH) mice were similar to that of healthy naive mice or sham controls receiving only PBS, indicating that overexpression of NKCC1 fully rescued the blood-induced ventriculomegaly normally seen in intraventricular hemorrhage (IVH) animals. NKCC1 overexpression (OE) mice did not show any change in intracranial pressure (ICP). Notably, NKCC1 overexpression (OE) was accompanied by even higher cerebrospinal fluid (CSF) clearance capacity compared to GFP control mice at two months following intraventricular hemorrhage (IVH) (FIGS. 21O-21P), suggesting persistent improvements in the capacity for intracranial fluid management capacity.

Two days following intraventricular blood injection, NKCC1 overexpression (OE) mice rapidly normalized their cerebrospinal fluid (CSF) [K⁺] to baseline levels compared to controls (FIG. 21Q; AAV-GFP: 8.11±0.35 mM vs. AAV-NKCC1: 4.00±0.53 mM vs. age-matched, naive P7 animals: 4.36±0.92 mM (23)), consistent with the expected function of NKCC1 to mediate cerebrospinal fluid (CSF) ion and water clearance. Collectively, these data support the concept that transiently increased cerebrospinal fluid (CSF) [K⁺] after hemorrhage can drive choroid plexus (ChP)-NKCC1 transport of ions and water away out of the ventricles and into the choroid plexus (ChP). Therefore, AAV-mediated choroid plexus (ChP) NKCC1 augmentation soon after intraventricular hemorrhage (IVH) may provide a valid therapeutic approach for post-hemorrhagic hydrocephalus (PHH).

### Example 13.: Adult Intraventricular Hemorrhage (IVH) Sequelae can be Rescued by Choroid Plexus (ChP) NKCC1 OE

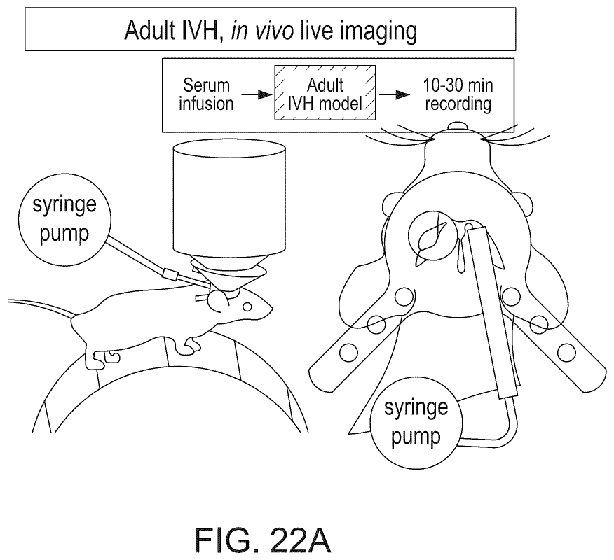

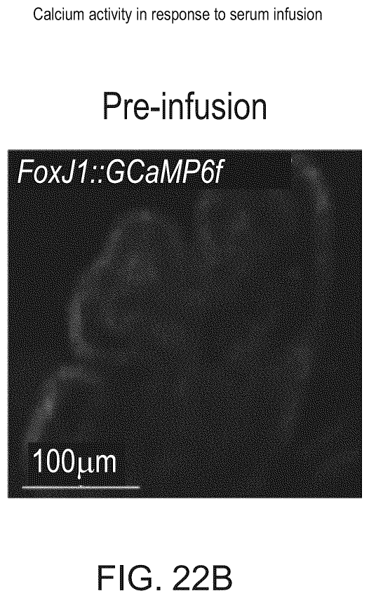

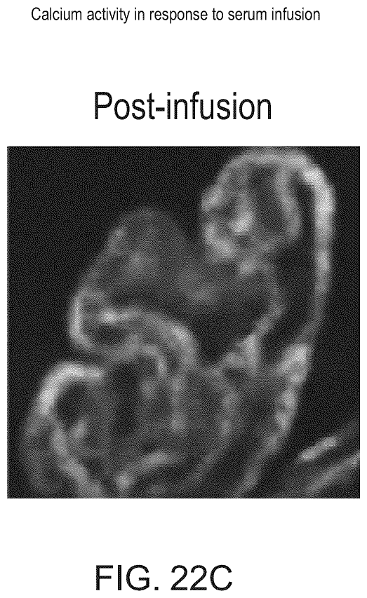

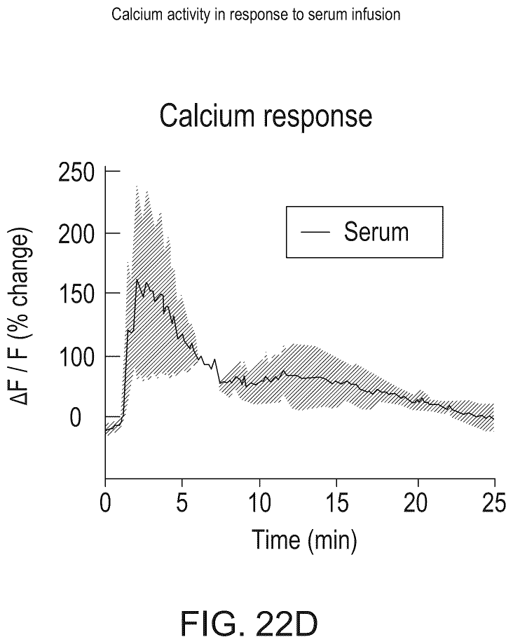

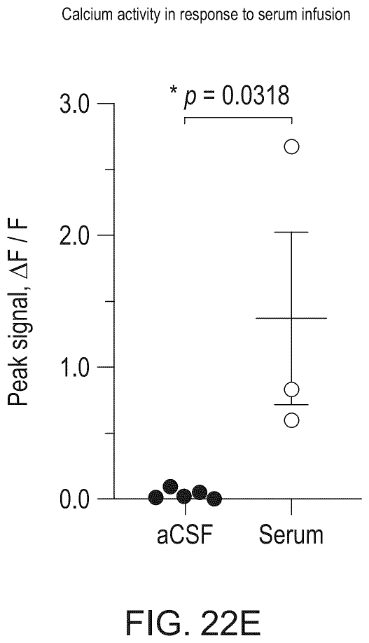

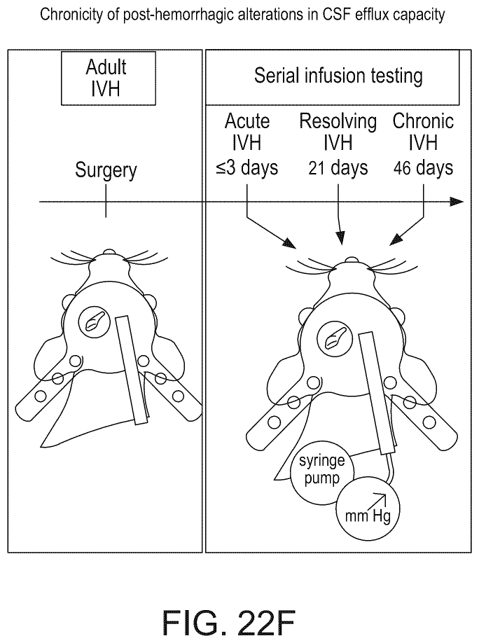

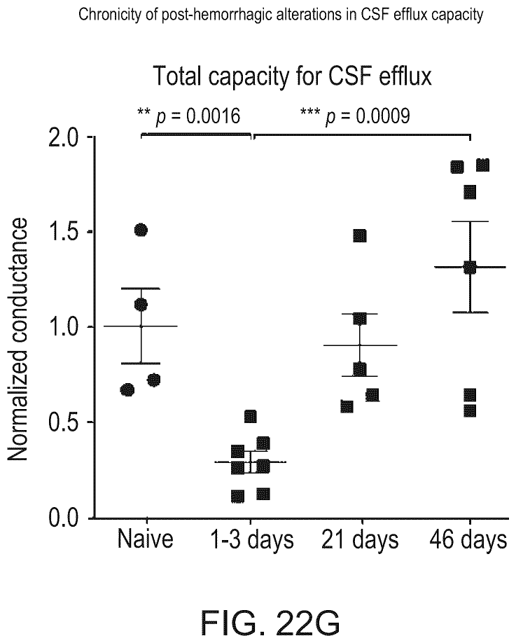

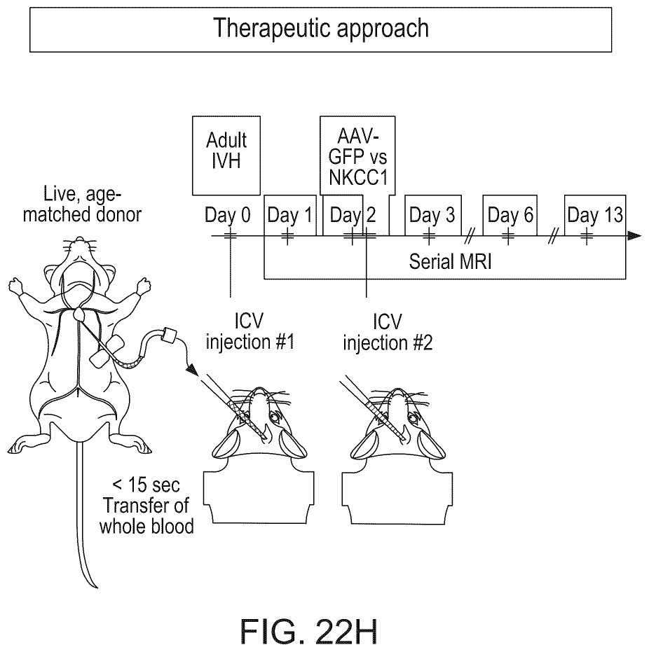

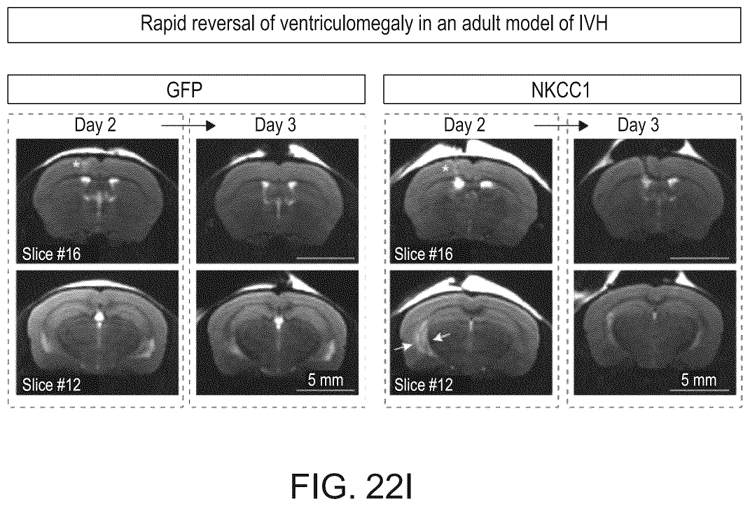

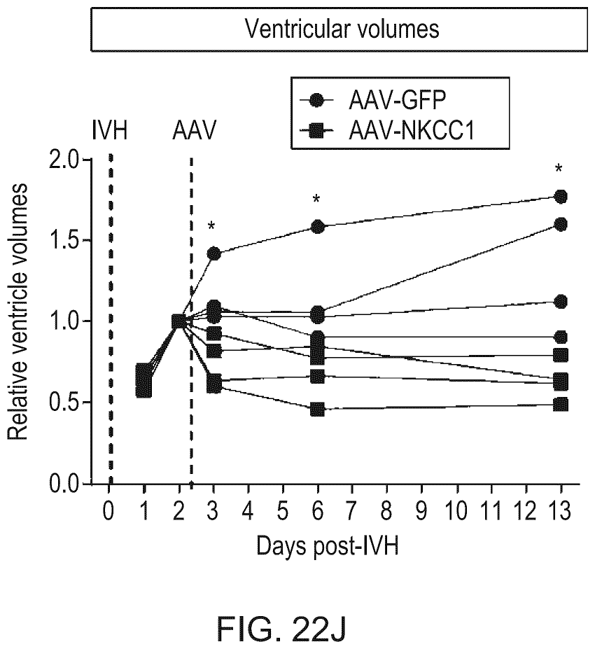

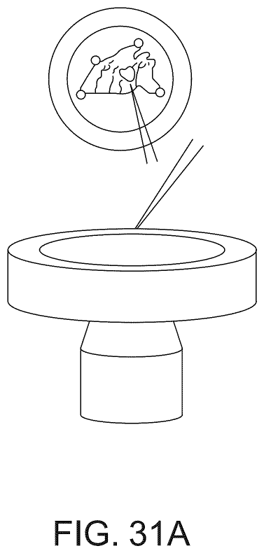

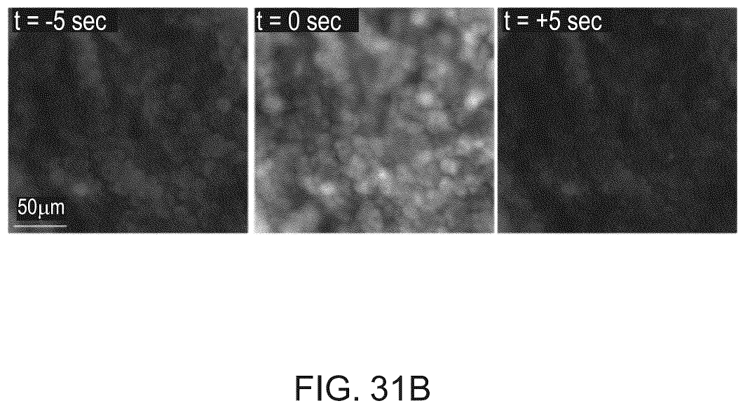

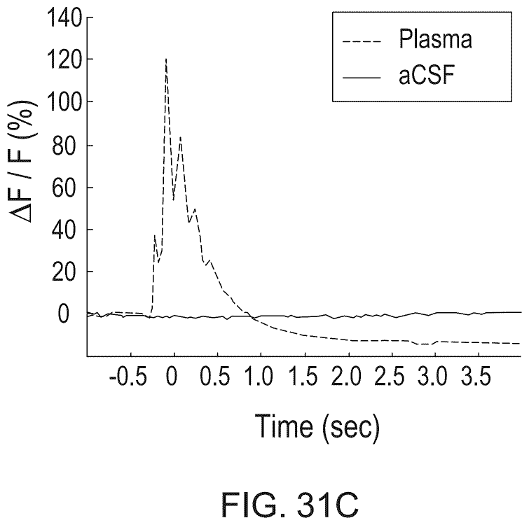

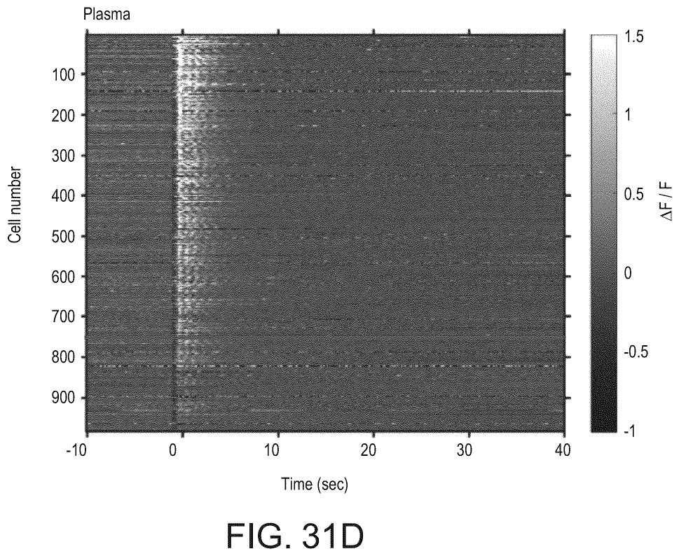

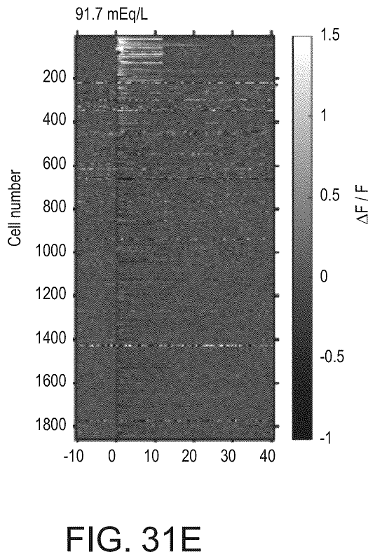

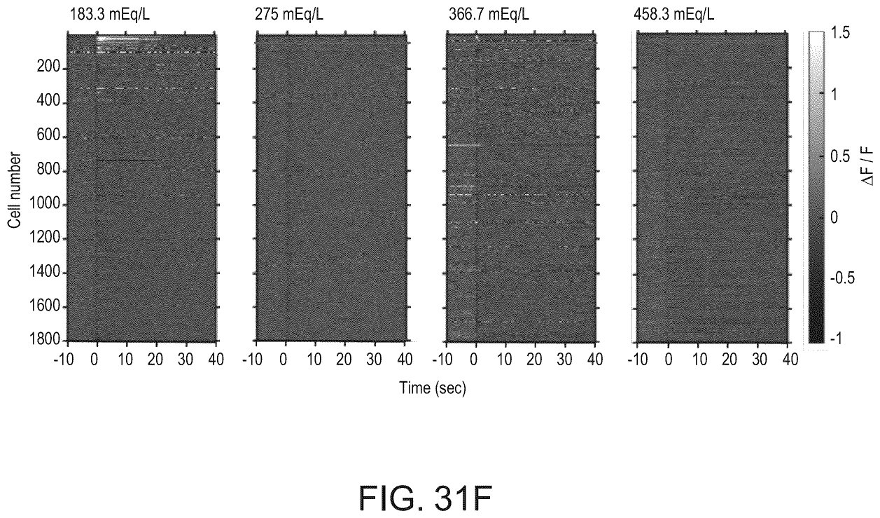

Age may factor into whether NKCC1 overexpression (OE) confers a beneficial response to intraventricular hemorrhage (IVH), as NKCC1 expression and phosphorylation is tightly regulated during late embryonic and early postnatal development(23). Recent work hypothesized that choroid plexus (ChP) NKCC1 activity is detrimental in an adult rat model of intraventricular hemorrhage (IVH) (19). Therefore, the model was tested in adult mice (8-10 weeks). A recently developed in vivo choroid plexus (ChP) imaging technology (12) was utilized to record choroid plexus (ChP) calcium activity through a transcortical cranial window in awake, head-fixed adult mice, and incorporated an injection cannula into the contralateral ventricle for direct intracerebroventricular delivery of reagents with on-going live imaging of the ipsilateral choroid plexus (ChP) (FIGS. 22A 30A, and 30B). Robust cytosolic calcium activity was observed in adult choroid plexus (ChP) rapidly following intraventricular infusion of serum (FIGS. 22B-22E similar data with plasma infusion not shown), consistent with the observations in E14.5 explants (FIGS. 2A-2K). In control experiments infusing aCSF in vivo and in vitro, it was further confirmed that the calcium events were not attributable to mechanical fluid movement or potential osmolarity changes introduced by serum or plasma (FIGS. 31A-31F). Repeated cerebrospinal fluid (CSF) infusion tests were also conducted through the injection cannula (FIG. 22F), which provided information on brain changes immediately after intraventricular blood exposure, with additional temporal resolution that could not be acquired in the pediatric models due to brain size, anesthesia limitations, and technical challenges associated with neonatal surgery. An acute decrease in cerebrospinal fluid (CSF) clearance was first observed 1-3 days following intraventricular blood exposure (as clinically expected), followed by a steady increase thereafter (FIG. 22G), suggesting the overall homeostatic increase in cerebrospinal fluid (CSF) absorption is gradual, and may result from the onset of secondary compensatory mechanisms.

Given the similarities between adult and pediatric post-hemorrhagic hydrocephalus (PHH) models with regard to calcium responses and changes in cerebrospinal fluid (CSF) clearance capacity, it was asked whether choroid plexus (ChP)-NKCC1 overexpression (OE) by AAV can improve ventricle size in the adult post-hemorrhagic hydrocephalus (PHH) model.

Serial live MRI scans were conducted to track ventricle size at 1, 2, 3, 6, and 13 days following delivery of age-matched intraventricular blood. AAV-NKCC1 or control AAV-GFP was introduced 2 days after intraventricular blood (FIG. 22H) and immediately following the second MRI session so that the severity of ventriculomegaly between the two groups was tightly controlled. Continuous enlargement of the lateral ventricles from Day 1 to Day 2 was observed in both groups, consistent with previous studies in adult rodent models (18, 19). However, on Day 3, 24 hours following AAV injection, the mice receiving AAV-NKCC1 demonstrated a reduction in lateral ventricle volumes (FIGS. 22I-22J). Importantly, the AAV-NKCC1 mice maintained a reduction in ventricle volumes over the 2-week duration following intraventricular blood exposure, as compared to control animals (FIG. 22J), suggesting long-lasting prevention of ventriculomegaly attributable to the overexpression of NKCC1. Collectively, the data in this adult model of intraventricular hemorrhage (IVH) validate the utility of AAV-NKCC1 to target choroid plexus (ChP) epithelial cells as a possible gene therapy for intraventricular hemorrhage (IVH)-induced hydrocephalus and broadens its potential applicability across pediatric and adult ages.

### Example 14.: Disproportionately High Cerebrospinal Fluid (CSF) [K⁺] Correlates with Persistent Post-Hemorrhagic Hydrocephalus (PHH) in Humans

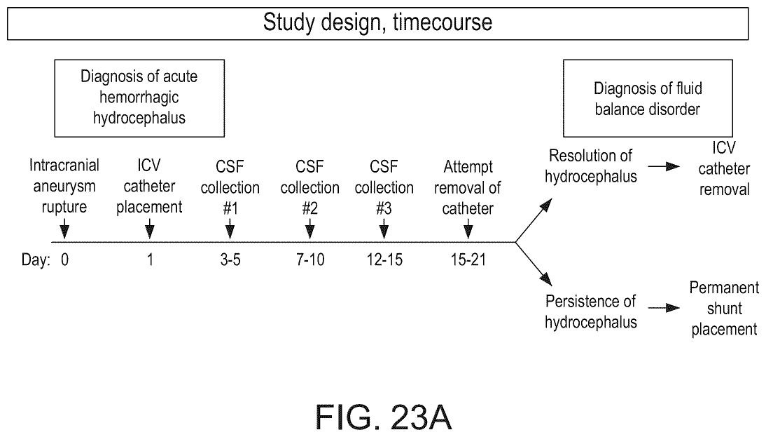

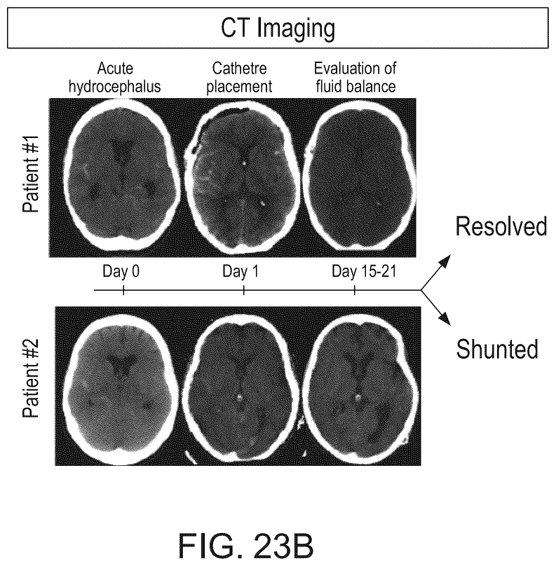

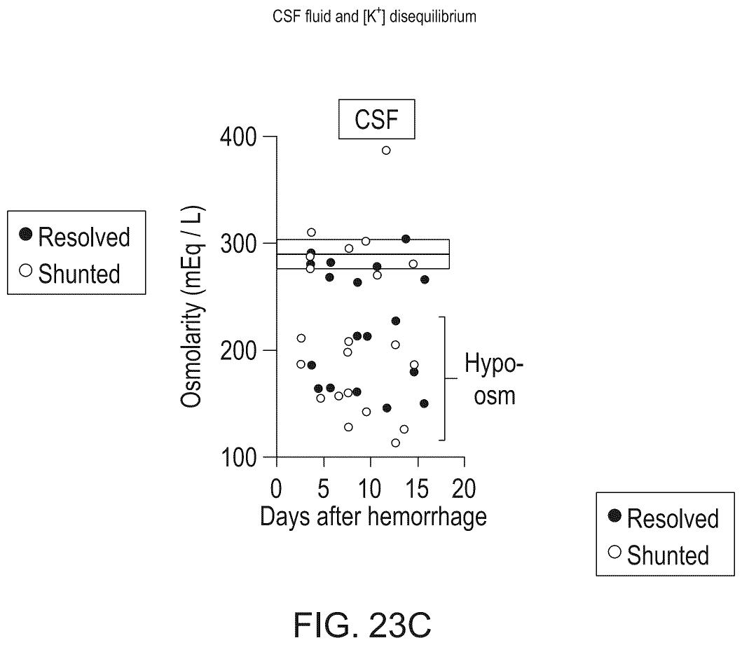

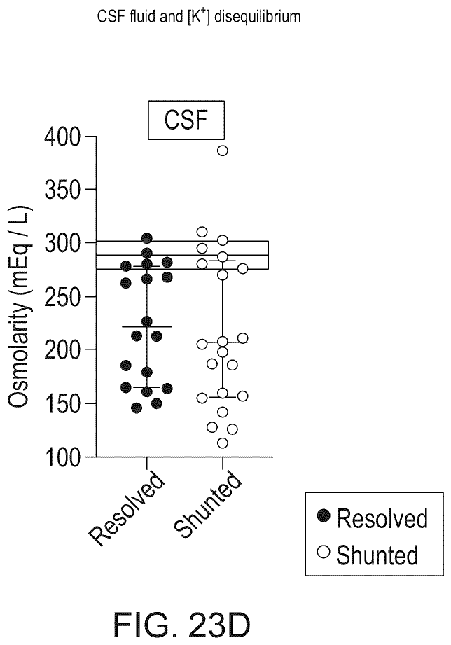

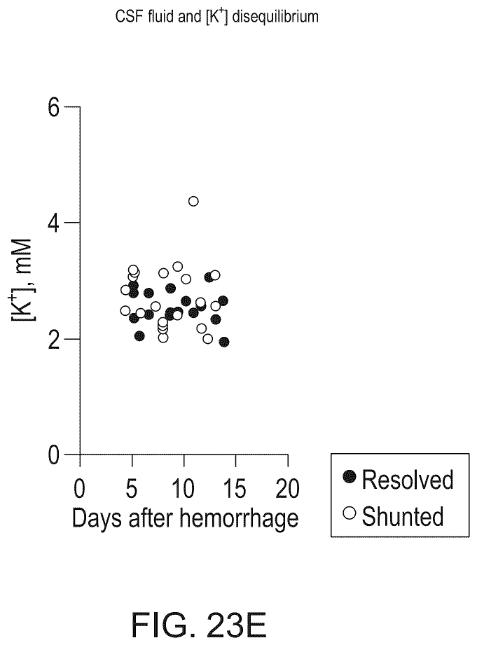

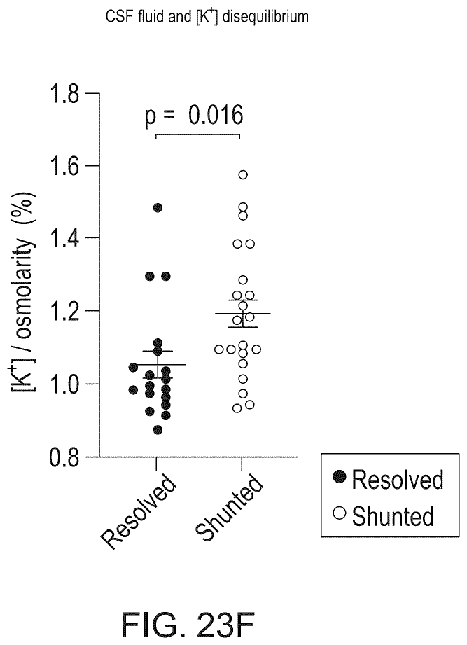

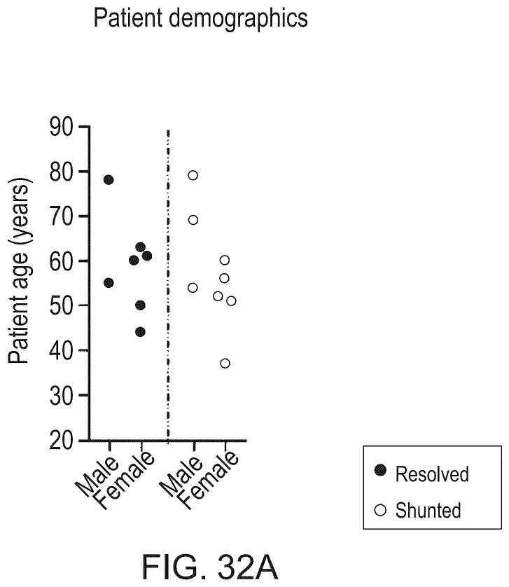

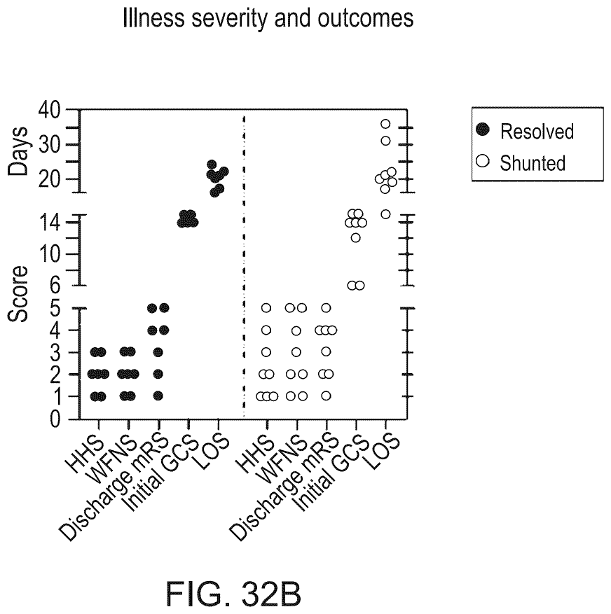

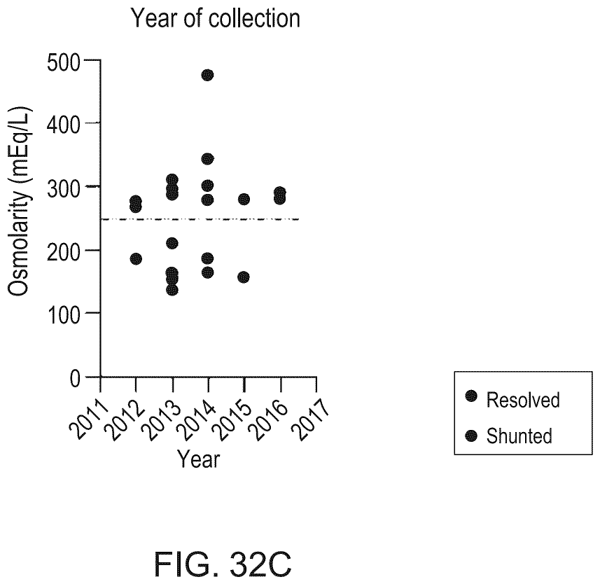

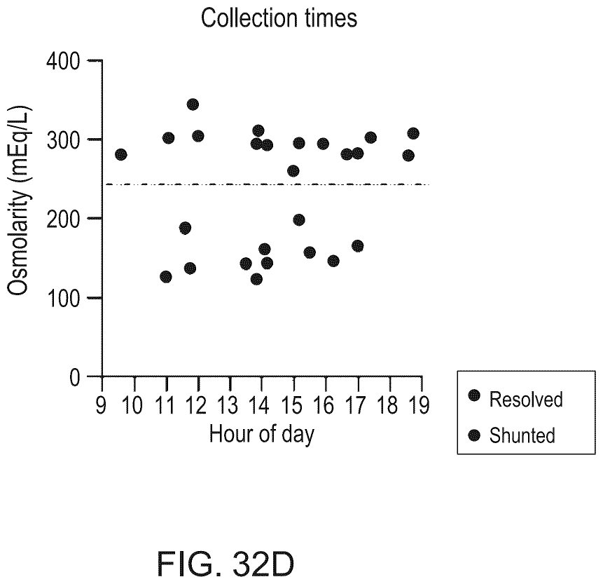

The data are consistent with a scenario whereby extravasated blood in the cerebral ventricles activates choroid plexus (ChP) epithelial cells to maintain cerebrospinal fluid (CSF) homeostasis, via intracellular Ca²⁺ events, changes in gene expression, and phosphorylation, that collectively ultimately increase NKCC1 activity. This activity moves K⁺ and water out of the cerebrospinal fluid (CSF), normalizing both cerebrospinal fluid (CSF) [K⁺] and volume. Thus, higher cerebrospinal fluid (CSF) [K⁺] and persistent ventriculomegaly would reflect deficiencies in choroid plexus (ChP)-mediated homeostasis and might additionally predict a worse clinical outcome. Indeed, it was found that in symptomatic post-hemorrhagic hydrocephalus (PHH) patients requiring acute treatment, intraventricular hemorrhage (IVH) and hemorrhage in contiguous subarachnoid spaces correlated with hyperkalirrhachia, as identified in the mouse model (FIG. 19J). cerebrospinal fluid (CSF) samples from adult patients with aneurysmal subarachnoid hemorrhage (aSAH) were acquired from a published prospective single-center cohort study (46). These patients were afflicted by hemorrhagic stroke due to the acute rupture of an intracranial aneurysm, with subsequent intraventricular hemorrhage (IVH) and acute post-hemorrhagic hydrocephalus (PHH) that was treated by placement of an intracerebroventricular catheter (clinically referred to as an external ventricular drain). cerebrospinal fluid (CSF) was collected at 3-6 day intervals between serial samplings for up to 16 days, totaling 54 unique cerebrospinal fluid (CSF) samples from 20 patients. The outcomes were recorded as either resolution of hydrocephalus (ICV catheter removal) or persistence of hydrocephalus (permanent shunt placement) (FIG. 23A-23B). All other parameters of patients, such as demographics, illness severity, and time of collection, were similar between the two groups (FIGS. 32A-32D). Measurements included osmolarity and [K⁺] using the same assays as used in the above-mentioned mouse studies.

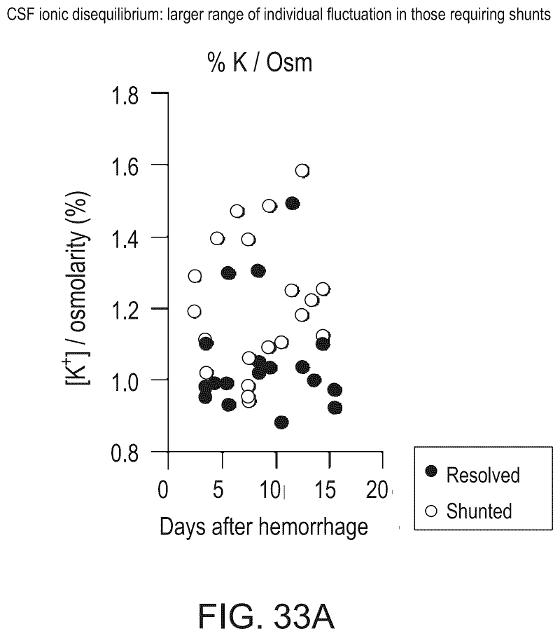

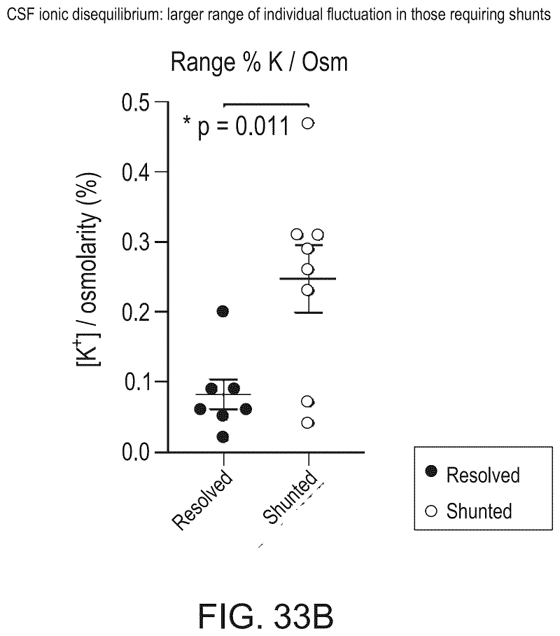

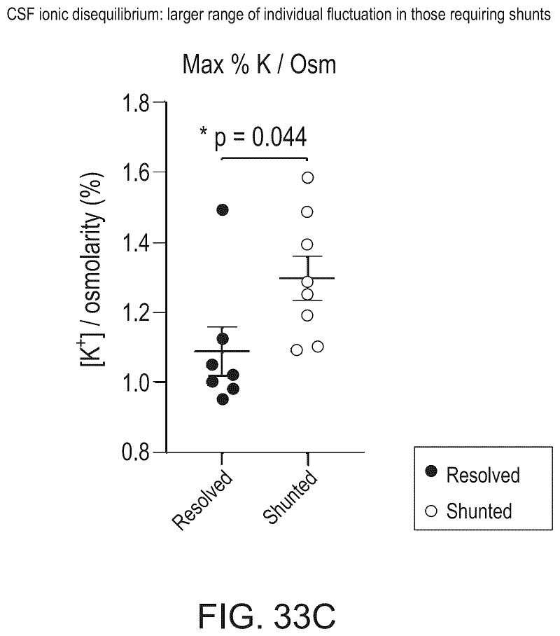

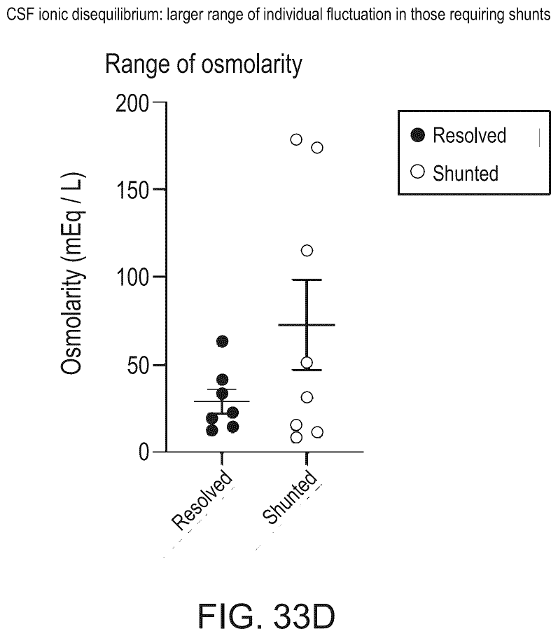

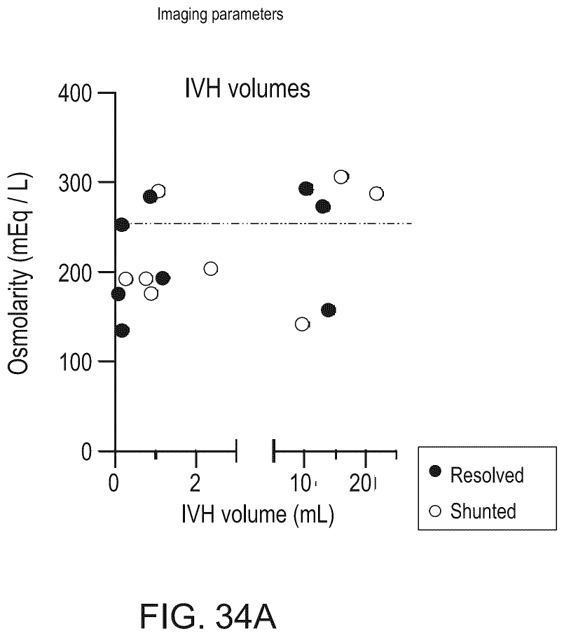

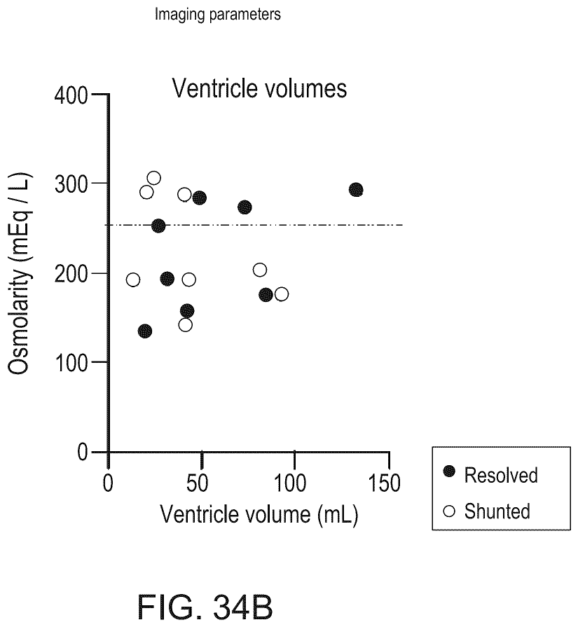

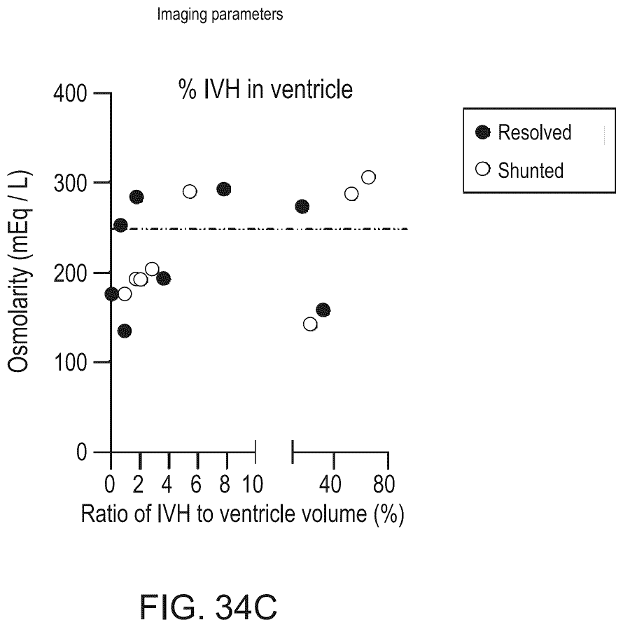

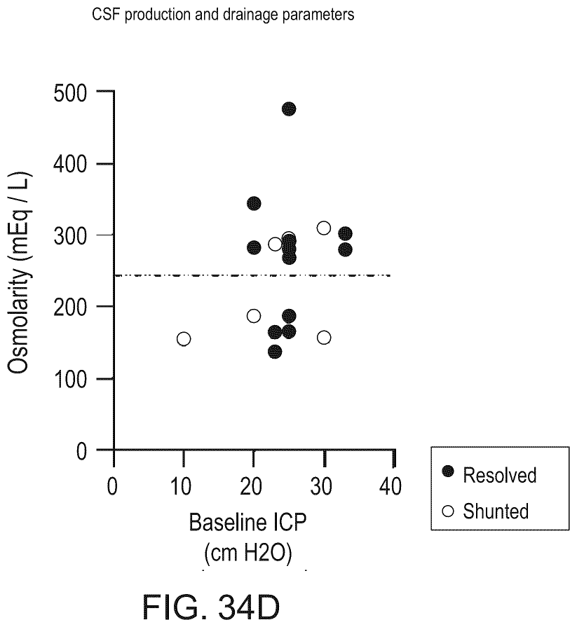

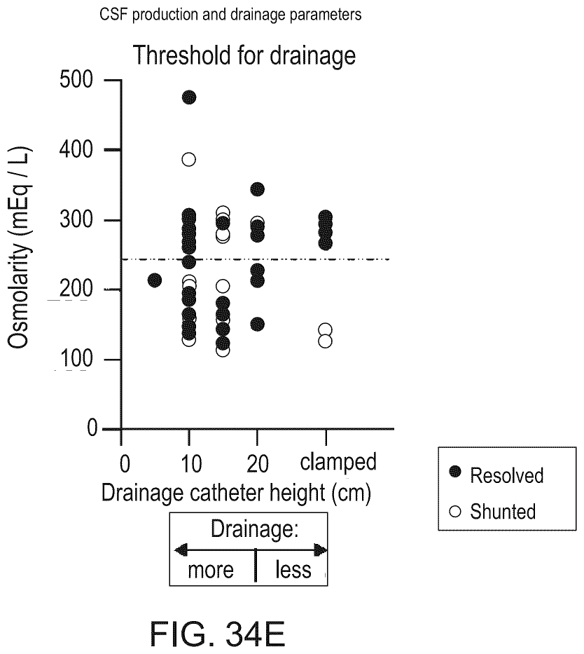

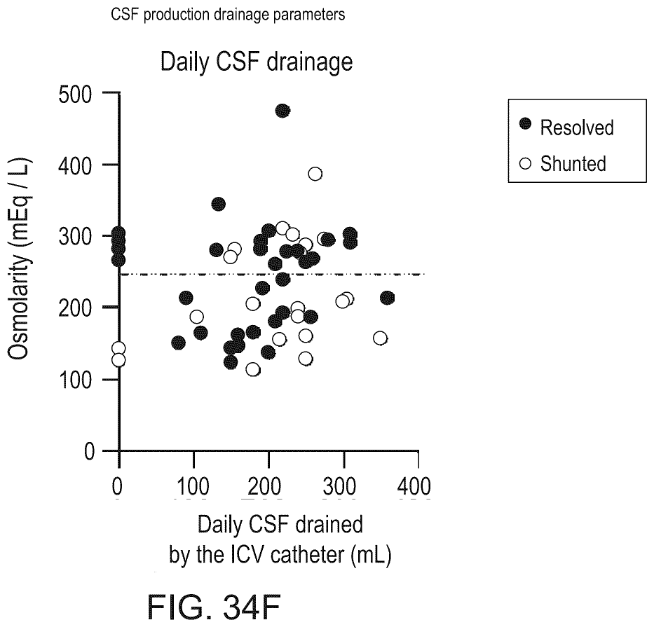

CSF hypo-osmolarity was identified in 60% of patients (mean±SEM.; 201±16 mEq /L) when compared to serum osmolarity measurements (within normal range, acquired in the same patients) (FIGS. 23C-23D), a phenomenon not reported previously and possibly a result from disequilibrium in cerebrospinal fluid (CSF) homeostasis. To take such changes in total osmolarity into consideration, relative cerebrospinal fluid (CSF) [K⁺] was defined to be the potassium concentration (mEq/L) as a percentage of total osmolarity (mEq/L). It was found that, although absolute cerebrospinal fluid (CSF) [K⁺] remained within normal limits for all patients (FIG. 23E), relative cerebrospinal fluid (CSF) [K⁺] was significantly higher in patients requiring shunt surgery (1.20 vs 1.06%, p<0.05) (FIG. 23F), consistent with the presence of relative hyperkalirrhachia observed in the mouse intraventricular hemorrhage (IVH) models (FIGS. 19J-19K). Patients requiring shunting also appeared to have a wider range of relative cerebrospinal fluid (CSF) [K⁺] fluctuation, possibly reflecting weaker modulatory capacity (FIGS. 33A-33D). Therefore, it was hypothesized that cerebrospinal fluid (CSF) [K⁺] and osmolarity changes provide a metric for cerebrospinal fluid (CSF) ionic disequilibrium and an indirect assessment of the choroid plexus (ChP) capacity for maintaining cerebrospinal fluid (CSF) homeostasis. Several anatomic, clinical, and radiographic factors are also associated with the need for permanent shunt surgery following aSAH (47), but none of these factors correlated with outcomes in the cohort of patients (FIGS. 34A-34F). Collectively, the data indicate that the cerebrospinal fluid (CSF) [K⁺] ionic disequilibrium (hyperkalirrhachia) that is observed in mouse models has direct translational implications, and that the “buffering capacity” of the choroid plexus (ChP) in managing cerebrospinal fluid (CSF) [K⁺] homeostasis strongly correlates with surgical outcomes in the clinical management of acute hydrocephalus.

### Discussion of Examples 8-14

There is a dire need for new and durable treatments for the various etiologies of pediatric and adult post-hemorrhagic hydrocephalus (PHH). Herein is reported a candidate gene therapy targeting the choroid plexus (ChP) that decreased brain ventricular volumes and prevented some of the most salient clinical features of post-hemorrhagic hydrocephalus (PHH) observable in mice. This therapeutic approach builds upon the stepwise testing of competing hypotheses regarding choroid plexus (ChP)-based cerebrospinal fluid (CSF) homeostasis following intraventricular hemorrhage (IVH) at multiple ages (embryonic, neonatal, and adult) and leverages newly adapted methods for testing cerebrospinal fluid (CSF) hydrodynamics and performing live imaging of choroid plexus (ChP) responses to intraventricular hemorrhage (IVH).

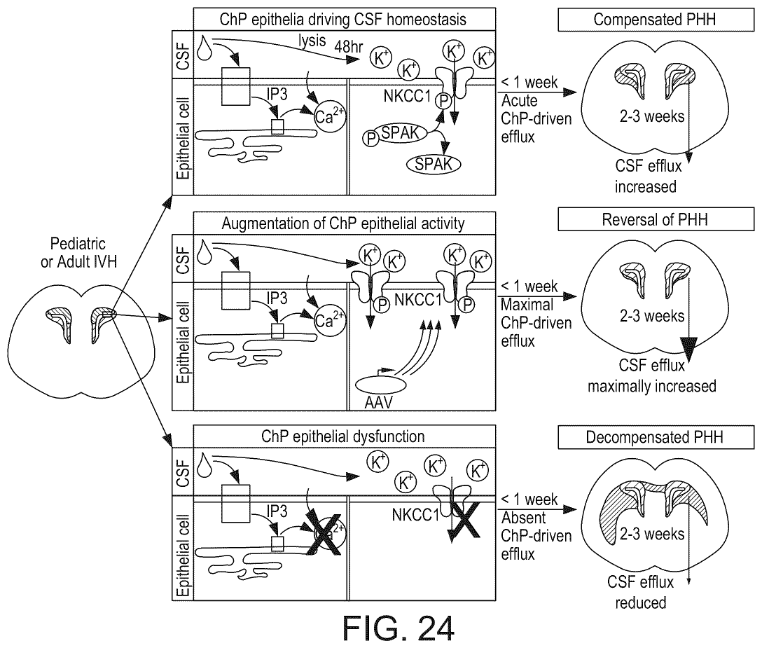

The data provide refinements to the prevailing clinical hypothesis about cerebrospinal fluid (CSF) hydrodynamics following intraventricular hemorrhage (IVH), demonstrating that the choroid plexus (ChP) epithelium supports homeostasis and responds to hemorrhage in a manner that favors cerebrospinal fluid (CSF) clearance and ventricle volume reduction. The choroid plexus (ChP) epithelium reacts to ventricular blood at several time scales ranging from seconds (e.g., intracellular calcium activity), hours (e.g., immediate early gene transcription), to days (e.g., NKCC1 phosphorylation), with potential secondary effects, including increased cerebrospinal fluid (CSF) clearance capacity, lasting weeks. Amplification of these favorable responses to intraventricular hemorrhage (IVH) can be achieved by targeted NKCC1 overexpression in the choroid plexus (ChP), leading to rapid improvements in cerebrospinal fluid (CSF) [K⁺] homeostasis and the resolution of ventriculomegaly. In contrast, disruption of this endogenous mechanism of choroid plexus (ChP) homeostasis leads to a reduction of NKCC1 phosphorylation and worsens intraventricular hemorrhage (IVH) hydrocephalus (summarized in FIG. 24). Together, these findings support a model of a beneficial, but often insufficient, endogenous response of the choroid plexus (ChP) to intraventricular hemorrhage (IVH), which can be augmented for maximal therapeutic gain at early timepoints.

Mouse models of intraventricular hemorrhage (IVH) were created using age-matched, rapidly extracted whole blood without anti-coagulants to best reproduce the full spectrum of endogenous blood-ChP interactions after intraventricular hemorrhage (IVH). Rather than target specific components of blood, the aim was to model the full and aggregate physiologic responses using whole blood when able, and only used serum or plasma when the cellular components would impede imaging of the choroid plexus (ChP). With these expectations, the model consistently generated moderate ventriculomegaly phenotypes in otherwise healthy mice, together with changes in cerebrospinal fluid (CSF) hydrodynamic that suggest compensatory changes in the brain to ensure long-term (>2 months) stability of cerebrospinal fluid (CSF) homeostasis despite the presence of ventriculomegaly. Existing clinical data strongly support this concept of a “borderline-compensated” state of cerebrospinal fluid (CSF) hydrodynamics following hemorrhagic strokes with intraventricular hemorrhage (IVH). For example, when examining the outcomes of intraventricular hemorrhage (IVH) (grades 2, 3, and 4, in the original Papile classification of preterm germinal matrix hemorrhage and intraventricular hemorrhage (IVH)), ˜80% of affected children ultimately regain normal cerebrospinal fluid (CSF) homeostasis (34). These data are confirmed by other groups (3-5, 48) and suggest that only ˜20% of children spiral into a decompensated state (e.g., mismatched cerebrospinal fluid (CSF) production and reabsorption resulting in high intracranial pressure (ICP) and neurologic decline), necessitating neurosurgical treatment. These clinical data highlight a common misunderstanding that intraventricular hemorrhage (IVH) inevitably leads to obstruction of cerebrospinal fluid (CSF) clearance pathways and scarring (27), and suggest that other homeostatic mechanisms are able to prevent higher rates of post-hemorrhagic hydrocephalus (PHH) despite the presence of persistent ventriculomegaly.

To bring these clinical and experimental discrepancies into agreement with the observed low rate of post-hemorrhagic hydrocephalus (PHH), it was proposed that the choroid plexus (ChP) acutely compensates for mismatched cerebrospinal fluid (CSF) production and clearance in the majority of cases. The smaller population of cases developing post-hemorrhagic hydrocephalus (PHH) may have had an insufficient homeostatic response by the choroid plexus (ChP) (either in magnitude or duration of cerebrospinal fluid (CSF) clearance). Motivated by clinical metrics of post-hemorrhagic hydrocephalus (PHH), clinical tools such as in vivo brain MRI and the constant rate infusion test were adapted to the improved mouse models of intraventricular hemorrhage (IVH) in order to investigate changes in cerebrospinal fluid (CSF) hydrodynamics following intraventricular blood exposure. These data provided a more nuanced explanation for the model of borderline-compensated cerebrospinal fluid (CSF) hydrodynamics, and offered insights that could have been masked using histologic methods or isolated measurements of intracranial pressure. With repeated cerebrospinal fluid (CSF) infusion tests on the same group of post-IVH mice over a time course of several days to weeks, it was confirmed that there was a reduced rate cerebrospinal fluid (CSF) clearance within days after intraventricular hemorrhage (IVH), consistent with a transiently higher relative rate of extra-choroidal cerebrospinal fluid (CSF) formation compared to clearance as reported (19), and implies a critical window of potential intervention. The clinically adapted methods revealed these time-dependent changes in cerebrospinal fluid (CSF) clearance and generated several hypotheses regarding ionic homeostasis to understand the spectrum of phenotypes following intraventricular hemorrhage (IVH).

While commonly described as the major source of cerebrospinal fluid (CSF), the choroid plexus (ChP) is best described as a sensitive modulator of cerebrospinal fluid (CSF) composition with the notable ability to bidirectionally transport solutes and water. The therapeutic target, NKCC1, is a water and ion cotransporter that follows the combined gradient of Na⁺, K⁺, and Cl⁻ between cerebrospinal fluid (CSF) and choroid plexus (ChP) epithelial cytosol. Among these ions, cerebrospinal fluid (CSF) K⁺ plays a critical role in determining NKCC1 transport directionality, as the concentration in the cerebrospinal fluid (CSF) is naturally at very low (˜3.5 mM in adult brains) and therefore sensitive to fluctuations. A non-canonical route of cerebrospinal fluid (CSF) clearance via choroid plexus (ChP) NKCC1 in early postnatal mice, when cerebrospinal fluid (CSF) [K⁺] is 2-3 fold higher than that of the adults was recently revealed (23). A similar scenario is apparent following intraventricular hemorrhage (IVH), whereby cerebrospinal fluid (CSF) [K⁺] is acutely increased several fold 48 hrs after intraventricular hemorrhage (IVH), likely a consequence of red blood cell lysis (demonstrated in the spinal cerebrospinal fluid (CSF) of neonates with intraventricular hemorrhage (IVH) (49)). This condition of increased cerebrospinal fluid (CSF) [K⁺] after hemorrhage is the driving force for choroid plexus (ChP) NKCC1-mediated water (hence, cerebrospinal fluid (CSF)) clearance and occurs concurrently with choroid plexus (ChP) NKCC1 phosphorylation. As shown in FIGS. 19A-19K, 20A-20L, and 21A-21Q, the choroid plexus (ChP) responds to alterations in cerebrospinal fluid (CSF) [K⁺] and drives cerebrospinal fluid (CSF) homeostasis by transporting the dominant cerebrospinal fluid (CSF) ions and water out of the ventricles.

Not wishing to be bound by theory, the findings presented herein suggest that the brain initially lags in its cerebrospinal fluid (CSF) clearance capacity following intraventricular hemorrhage (IVH) (FIG. 22G) (providing a definable therapeutic window) but that this cerebrospinal fluid (CSF) clearance capacity gradually increases over time (>6 weeks after initial intraventricular hemorrhage (IVH) in the pediatric model, or >3 weeks in the adult model). The timing of choroid plexus (ChP) NKCC1 phosphorylation and K⁺-driven cerebrospinal fluid (CSF) clearance from the ventricle through NKCC1 aligns well with the acute phase (48 hrs.) of intraventricular hemorrhage (IVH) when cerebrospinal fluid (CSF) clearance is at a minimum and potentially insufficient. This expected deficit in cerebrospinal fluid (CSF) clearance supports the timing of targeted therapy. Overexpression of NKCC1 in the choroid plexus (ChP) by AAV during this early timeframe enhanced cerebrospinal fluid (CSF) clearance after intraventricular hemorrhage (IVH), leading to reduced ventricle sizes and more readily stabilized cerebrospinal fluid (CSF) [K⁺]. With these predictions about cerebrospinal fluid (CSF) clearance capacity, the long-term benefits of choroid plexus (ChP) NKCC1-mediated homeostasis may be self-limited as the blood clears and the cerebrospinal fluid (CSF) [K⁺] returns to normal levels, dissipating the driving K⁺ gradients. However, given the unexpectedly increased capacity for cerebrospinal fluid (CSF) clearance that develops after 3-6 weeks when blood is already cleared from the ventricular system, this may indicate the presence of additional mediators of cerebrospinal fluid (CSF) clearance either within the choroid plexus (ChP) or from other regions of the ventricular system. Strikingly, NKCC1 overexpression in the choroid plexus (ChP) further enhanced this long-term increase in cerebrospinal fluid (CSF) clearance capacity, but the exact cascade of events leading to this outcome will require further investigation. While these long-term mechanisms of cerebrospinal fluid (CSF) clearance remain to be explored, the time course of increasing cerebrospinal fluid (CSF) clearance in the intraventricular hemorrhage (IVH) model is clinically supported by recent evidence in favor of early invasive/surgical interventions for post-hemorrhagic hydrocephalus (PHH) in children to “buy” time for cerebrospinal fluid (CSF) homeostasis and equilibration before permanent interventions are prematurely performed (50).

In addition to the choroid plexus (ChP), multiple other brain and spinal tissue types contribute to cerebrospinal fluid (CSF) clearance, including various lymphatic (dural/meningeal and along cranial nerves, spinal nerve roots, or major blood vessels traversing the skull base) and trans-venous (arachnoid granulation) pathways (14). Recent findings specifically implicate lymphatic drainage to clear erythrocytes and iron in the context of intraventricular hemorrhage (IVH) (51), suggesting that this pathway might complement the role of the choroid plexus (ChP) for the purposes of homeostasis after intraventricular hemorrhage (IVH). The constant rate infusion test incorporates all routes of cerebrospinal fluid (CSF) clearance and therefore leaves open the possibility that longer-term changes in cerebrospinal fluid (CSF) clearance might also be mediated by the lymphatic pathways. While gene therapy enhancing NKCC1 in the choroid plexus (ChP) successfully rescued hyperkalirrhachia and ventriculomegaly in mice exposed to intraventricular blood, it was predicted that future therapies would optimally target both choroid plexus (ChP) and lymphatic pathways of cerebrospinal fluid (CSF) clearance. In pediatric cases, however, the choroid plexus (ChP) may be a more important target, as studies in animal models have shown a lack of mature meningeal lymphatics until later stages in development (e.g. P12 in mice and P7 in rats).

Finally, explored human cerebrospinal fluid (CSF) ionic composition following acute hemorrhagic hydrocephalus was explored to assess the applicability of the model of choroid plexus (ChP)-mediated ionic homeostasis to human disease. The following were identified: 1) evidence for prolonged cerebrospinal fluid (CSF) disequilibrium in humans, suggesting a longer therapeutic window of opportunity; 2) perturbations in cerebrospinal fluid (CSF) osmolarity and relative hyperkalirrhachia that are consistent with some aspects of the model of K⁺-driven ionic homeostasis; and 3) possible prognostic features based on cerebrospinal fluid (CSF) composition. First, using serial sampling of cerebrospinal fluid (CSF) in the same patients, prolonged duration of cerebrospinal fluid (CSF) hypo-osmolarity and relative hyperkalirrhachia spanning up to 2 weeks after the onset of hemorrhage was confirmed. One of the ways in which humans differ from the mouse model involves the duration of persistent intraventricular hemorrhage (IVH); while humans take 1-2 weeks to clear intraventricular hemorrhage (IVH), mice are able to accomplish this in 2-4 days and with variable degrees of clot formation based on the age. This extended duration of intraventricular hemorrhage (IVH) in humans hypothetically might prolong the duration of requisite choroid plexus (ChP) NKCC1-mediated cerebrospinal fluid (CSF) clearance. Extent of intraventricular hemorrhage (IVH) is negative prognostic indicator (52) and surgical intraventricular hemorrhage (IVH) clearance is beneficial (53-55). Next, higher relative cerebrospinal fluid (CSF) [K⁺] and a larger fluctuation of cerebrospinal fluid (CSF) [K⁺] over time was observed in patients that eventually required permanent surgical intervention for post-hemorrhagic hydrocephalus (PHH) (e.g., ventriculo-peritoneal shunt placement) compared to those who recovered. Combined with persistent hyperkalirrhachia, these data suggest that an individual's fluctuations in cerebrospinal fluid (CSF) ionic composition might reflect either a high intraventricular hemorrhage (IVH) burden or the insufficiency of the choroid plexus (ChP)-driven homeostasis mechanisms. Both scenarios support the model of K⁺-driven homeostasis and reinforce the concept of a borderline-compensated state, whereby ˜20% of all cases or ˜50% of severe cases lead to failure of cerebrospinal fluid (CSF) homeostasis.

In summary, the findings of Examples 8-14 support the concept that the choroid plexus (ChP) acts as an immediate responder to intraventricular blood contact and acts as an emergency reservoir to temporarily boost cerebrospinal fluid (CSF) clearance capacity in the acute phase of intraventricular hemorrhage (IVH), when cerebrospinal fluid (CSF) clearance is at its nadir. The choroid plexus (ChP) provides this cerebrospinal fluid (CSF) clearance via NKCC1 when cerebrospinal fluid (CSF) [K⁺] is much higher than normal, and this NKCC1-dependent mechanism can be targeted by AAV for at least short-term benefits in the pediatric and adult models. AAV-based therapeutic approaches have proven efficacy for a growing number of neurological conditions (56). Therefore, enhancing cerebrospinal fluid (CSF) clearance by AAV-mediated choroid plexus (ChP) NKCC1 overexpression may prove useful for ventriculomegaly and management of fluid dynamics in a broader set of diseases involving hemorrhagic events and perhaps other neurologic diseases that involve alterations in cerebrospinal fluid (CSF) composition. This approach, applied alone or paired with existing short-duration treatment options (e.g., lumbar punctures for removal of excess cerebrospinal fluid (CSF)), may ultimately reduce the need for permanent shunting, improve disease outcome and quality of life, and reduce the burden of post-hemorrhagic hydrocephalus (PHH) on the medical system.

### Methods of the Examples

The following methods were employed in Examples 1-7.

Mice

Timed pregnant CD1 dams were obtained from Charles River Laboratories. Mice with germline 1oxP-CHD4-1oxP were imported from MGH and bred in-house. Both male and female mice were equally included in the experiments and were analyzed at postnatal day 0, 7, 14, 21, 28, 5-7 weeks, and 2+ months. Animals were housed in a temperature and humidity controlled room on a 12-hr light/12-hr dark cycle and had free access to food and water. All mice younger than postnatal day 10 were allocated into gestational/postnatal age groups without respect to sex (both males and females were included). Both males and females were included in studies involving mice older than 10 days.

CSF Collection and Metal Detection

CSF was collected by inserting a glass capillary into cisterna magna, and collected cerebrospinal fluid (CSF) was centrifuged at 1000×g for 10 min at 4° C. to remove any tissue debris. Metal quantification was performed by Galbraith Laboratories, Inc (Knoxville, TN, USA). Inductively coupled plasma—optical emission spectrometry (ICP-OES) was used for K and Na quantification, and ion chromatography (IC) was used for the Cl⁻ quantification. All tests were performed using 5-10 μL of cerebrospinal fluid (CSF). The quality control (QC) recovered at 94.4% for K⁺, 97.7% for Na⁺, 100.1% for Cl⁻, 475 95.1-118% for Mg²⁺, and 107.5% for Ca²⁺.

Trap

A total of 9 mice aged 8 weeks and 9 litters of mouse embryos aged E16.5 from the Foxj1:Cre x EGFP-L10a Bacterial Artificial Chromosome (BAC) transgenic lines (N=3 samples of Lateral Ventricle choroid plexus (LVChP) pooled from 3 8-week mice or 3 litters of E16.5 embryos) were used. Brain tissue was immediately dissected and used for TRAP RNA purifications³⁸. choroid plexus (ChP) tissues were dissected in ice-cold dissection buffer (1×HBSS; 2.5 mM HEPES-KOH; 35 mM glucose; 4 mM NaHCO₃; 100 μg/mL CHX) and homogenized in lysis buffer (20 mM HEPES KOH; 5 mM MgCl₂; 150 mM KCl; 0.5 mM DTT; 1×protease inhibitors; 40 U/mL Rnasin; 20 U/mL Superasin) at 900 rpm for 12 strokes on ice with Teflon pestles in glass tubes (Kontes). Post-nuclear supernatant was prepared by centrifugation at 4° C. for 10 min at 2,000×g. Post-mitochondrial supernatant was prepared after incubation with additional 1% NP-40 and 30 mM DHPC, followed by centrifugation at 4° C. for 10 min at 20,000×g. Immunoprecipitation was performed with magnetic streptavidin beads (MyOne T1 Dynabeads; Thermo Fisher #65601) conjugated to biontinylated anti-GFP antibodies (clones 19C8 and 19F7) for 16 hours at 4° C. with gentle end-over-end rotation. Beads were collected on a magnet on ice, washed 4 times with 1000 μL 0.35 M KCl wash buffer (20 mM HEPES-KOH; 5 mM MgCl₂; 350 mM KCl; 1% NP-40; 0.5 mM DTT; 100 μg/mL CHX). RNA was eluted in Stratagene Absolutely RNA Nanoprep Kit (Agilent #400753) lysis buffer (with β-ME) and purified according to kit instructions. RNA quality was assessed using Bioanalyzer Pico Chips (Agilent, 5067-1513) and quantified using Quant-iT RiboGreen RNA assay kit (Thermo Fisher Scientific R11490). Libraries were prepared using Clonetech SMARTer Pico with ribodepletion and Illumina HiSeq to 50NT single end reads. Sequencing was performed at the MIT BioMicroCenter.

Sequencing Data Analysis

The raw fastq data of 50-bp single-end sequencing reads were aligned to the mouse mm10 reference genome using STAR RNA-Seq aligner (v2.4.0j)⁷⁸. The mapped reads were processed by htseq-count of HTSeq software (v 0.6.0)⁷⁹ with mm10 gene annotation to count the number of reads mapped to each gene. The Cuffquant module of the Cufflinks software (v 2.2.1) 80 was used to calculate gene FPKM (Fragments Per Kilobase of transcript per Million mapped reads) values. Gene differential expression test between different animal groups was performed using DESeq2 package (v. 1.26.0)⁸¹ with the assumption of negative binomial distribution for RNA-Seq data. Genes with adjusted p-value<0.05 are chosen as differentially expressed genes. All analyses were performed using genes with FPKM>1, which was considered as the threshold of expression.

Sequencing Pathway and Motif Analysis

Functional annotation clustering was performed using DAVID v6.7⁸². Gene ontology (GO) analysis was performed using AdvaitaBio iPathway guide v.v1702. Enrichment vs. perturbation analysis was performed by AdvaitaBio iPathway guide v.v1702 and allows comparison of pathway output perturbation and cumulative gene set expression changes. In brief, the enrichment analysis is a straightforward gene-set enrichment over-representation analysis (ORA) considering the number of differentially expressed genes (DEGs) that are assigned to a given pathway. The enrichment value is expressed as a proportion of enriched members to total genes in a defined pathway and a p-value (Fisher) is calculated for this score, however false positives have been reported at up to 10% with this method 83. Perturbation, on the other hand, uses pathway data that applies relationships between gene products rather than only using a list. Perturbation assigns an impact score based on a mathematical model that captures the entire topology of the pathway and uses it to calculate how changes in the expression of each gene in the pathway would perturb the absolute output of the pathway 83. Then, these gene perturbations are combined into a total perturbation for the entire pathway and a p-value is calculated by comparing the observed value with what is expected by chance. Motif analyses were performed using SignalP (v5.0;⁸⁴) and TMHMM (v2.0;⁸⁵).

Transmission Electron Microscopy

All tissue processing, sectioning, and imaging was carried out at the Conventional Electron Microscopy Facility at Harvard Medical School. Forebrain tissues were fixed in 2.5% glutaraldehyde/2% paraformaldehyde in 0.1 M sodium cacodylate buffer (pH 7.4). They were then washed in 0.1M cacodylate buffer and postfixed with 1% osmiumtetroxide (OsO4)/1.5% potassium ferrocyanide (KFeCN₆) for one hour, washed in water three times and incubated in 1% aqueous uranyl acetate for one hour. This was followed by two washes in water and subsequent dehydration in grades of alcohol (10 minutes each; 50%, 70%, 90%, 2×10 min 100%). Samples were then incubated in propyleneoxide for one hour and infiltrated overnight in a 1:1 mixture of propyleneoxide and TAAB Epon (Marivac Canada Inc. St. Laurent, Canada). The following day, the samples were embedded in TAAB Epon and polymerized at 60 degrees C. for 48 hours. Ultrathin sections (about 80 nm) were cut on a Reichert Ultracut-S microtome, and picked up onto copper grids stained with lead citrate. Sections were examined in a JEOL 1200EX Transmission electron microscope or a TecnaiG² Spirit BioTWIN. Images were recorded with an AMT 2k CCD camera.

Glycogen and Mitochondrial Quantification

Glycogen and mitochondrial quantification was performed by hand using the ImageJ plugin FIJI^{86,87}. Percentages were calculated by dividing the area of interest by the total area of choroid plexus (ChP) epithelial cell within the field of view. No other cell types were included in the analysis. For each condition, analyses were performed across multiple individual animals (N=3 for each age). From each animal, 10-20 fields of view were imaged at 3,000× for glycogen analysis and 5-10 fields of view were imaged at 3,000× for mitochondrial analysis. Each different field of view represented a unique cell or cells, and fields of view were chosen such that both the apical and basal surfaces of the cell were visible. For mitochondrial distribution, a custom MatLab (v.2018) code was written to extract the centroid from mitochondria data traced in ImageJ ROIs. Then a distance transformation was performed from each mitochondrion centroid to the hand-traced apical or basal surfaces. The shortest distance was extracted to calculate the apical: basal proximity ratio, such that 1=on the apical surface and 0=on the basal surface. The analyses included a total of 1747 adult mitochondria, 2241 P7 mitochondria, 2257 P0 mitochondria, and 1123 embryonic mitochondria. The same groups of embryonic and adult animals were used for the above analysis.

Seahorse Metabolic Analysis ChP explants were dissected in HBSS (Fisher, SH30031FS) and maintained on wet ice until plated. Only the posterior leaflet of the P0, P7, and adult choroid plexus (ChP) was retained for analysis due to empirically determined limitations of the oxygen availability in the XFe96 Agilent Seahorse system. Tissue explants were plated on Seahorse XFe96 spheroid microplates (Agilent, 102905-100) coated with Cell TAK (Corning), in Seahorse XF Base Medium (Agilent, 102353-100) supplemented with 0.18% glucose, 1 mM L-glutamine, and 1 mM pyruvate at pH7.4 and incubated for 1 hour at 37° C. in a non-CO₂ incubator. Extracellular acidification rates (ECAR) and oxygen consumption rates (OCR) were measured via the Cell Mito Stress Test (Agilent, 103015-100) with a Seahorse XFe96Analyzer (Agilent) following the manufacturer's protocols. Data were processed using Wave software (Agilent). ATP production was calculated as the difference in OCR measurements before and after oligomycin injection, as described by the manufacturer's protocol (Agilent, 103015-100). The Cell Mito Stress test was performed 2-5 independent times (5 for adult; 2 for P7; 2 for P0; 3 for E16). The individual analyses were performed by averaging the readings from both the right and left hemisphere lateral ventricle choroid plexus (ChP) for each individual. Data were normalized by Calcein-AM (2 μM in PBS, Life Technologies L-3224) fluorescence measured at the end of the assay. Each datum is the normalized average of the 2 Lateral Ventricle (LV) choroid plexus (ChP) from each individual normalized to the average of the adult levels run on the same plate for each assay to account for any experimental variability.

High K+ Challenge

Fresh Lateral Ventricle choroid plexuses (LVChP) were dissected from P4 pups and adult mice in room temperature HBSS and glued down onto imaging dishes with coverslip bottom. The tissues were incubated at 37° C. with Calcein-AM (Invitrogen L3224; 1:200) for 10 min and then rinsed with 37° C. artificial cerebrospinal fluid (CSF) (aCSF: 119 mM NaCl, 2.5 mM KCl, 26 mM NaHCO₃, 1 mM NaH₂P0₄, 11 mM glucose, with fresh 1.0 mM magnesium chloride and 2.8 mM calcium chloride; for P4 experiments, 5 mM KCl was used to match with cerebrospinal fluid (CSF) [K⁺]). Protein was not added to aCSF because, while in rat, the protein concentration decreases 10× between P2 and adult⁸⁸; this change is only 2× in mouse⁴⁵ and unlikely to contribute substantially to the driving force. The tissues were soaked in 1.8 ml aCSF at the beginning of each imaging session and allowed to stabilize for 10 min. One Z-stack was acquired to reflect the baseline cell volume. Then a 10× KCl solution in aCSF was spiked into the bath to bring the final bath K⁺ concentration to 50 mM immediately before imaging continued. A total of five 3D Z-stacks were acquired throughout a 10-min imaging session to capture changes in cellular volume over time. Each stack acquisition took less than 30 s to minimize changes in cell volume from the beginning to the end of each stack. All imaging studies were carried out at 37° C. Image stacks were imported into Imaris (v7.7.1; Bitplane) software. Individual epithelial cells were identified by shape. Cells with discrete borders that were present at all timepoints and had dark pixels both above and below them in Z for the whole timecourse were selected apriori and then traced by hand using the “Surpass” functionality to create a 3D surface volume through all Z stacks based on Calcein-AM uptake signal. Due to known z-step distance and interpolation between the planes, Imaris calculated the number of voxels for each cell. This analysis was then repeated for the same cell throughout the timecourse. It was verified manually that the same cell was analyzed throughout the time course based on the topology of the surrounding cells, allowing for adjustment for any x-y drifting that occurred. The relative volume was calculated as dV/V₀ for each timepoint (t) where V₀ is the initial volume of 602 the cell, t is each subsequent timepoint after addition of challenge, and dV=Vt-V₀. In Lateral Ventricle choroid plexus (LVChP) from each animal, 5 distinct cells were analyzed and their values at each time point were averaged to represent the average value for each animal. A total of 4 animals (N=4) were analyzed from each age group.

Tissue Processing

Samples were fixed in 4% paraformaldehyde (PFA). For cryosectioning, samples were incubated in the following series of solutions: 10% sucrose, 20% sucrose, 30% sucrose, 1:1 mixture of 30% sucrose and OCT (overnight), and OCT (1 hour). Samples were frozen in OCT.

Immunostaining

Cryosections were blocked and permeabilized (0.3% Triton-X-100 in PBS; 5% serum), incubated in primary antibodies overnight and secondary antibodies for 2 hours. Sections were counterstained with Hoechst 33342 (Invitrogen H3570, 1:10000) and mounted using Fluoromount-G (SouthernBiotech). The following primary antibodies were used: chicken anti-GFP (Abcam ab13970; 1:1000), mouse anti-Aqp1 (Santa Cruz sc-32737; 1:100), rabbit anti-CHD4 (Abcam ab72418, 1:200), rabbit anti-NKCC1 (Abcam ab59791; 1:500), rat anti-HA (Roche 11867423001; 1:1000). Secondary antibodies were selected from the Alexa series (Invitrogen, 1:500). Images were acquired using Zeiss LSM880 confocal microscope with 20× objective and 63× oil objective. ZEN Black software was used for image acquisition and ZEN Blue used for Airy processing.

IP

Tissues were homogenized in NET buffer (150 mM NaCl, 10 mM Tris 8.0, 5 mM EDTA, 10% glycerol and 2% Triton-100) supplemented with protease inhibitors. Protein concentration was determined by BCA assay (Thermo Scientific 23227). Lysates with same amount of total protein (250-1000 μg based on experiments) were pre-cleared at 4° C. for 2 hr with Protein G agarose and then incubated with desired antibody or control antibody at 4° C. overnight (no beads present during antibody incubation). Protein G agarose beads were added to lysate-antibody mixture after overnight incubation for 2 hr. Beads were washed thoroughly and then eluted by boiling in 2% SDS. choroid plexus (ChP) tissues were pooled across 7 litters of P0 pups and 30 adults to achieve sufficient protein for Co-IP.

Immunoblotting

Tissues were homogenized in RIPA buffer supplemented with protease and phosphatase inhibitors. Protein concentration was determined by BCA assay (Thermo Scientific 23227). Samples were denatured in 2% SDS with 2-mercaptoethanol by heating at 37° C. (for NKCC1) or 95° C. (for CHD4 and other NuRD complex proteins) for 5 minutes. Equal amounts of proteins were loaded and separated by electrophoresis in a 4-15% gradient polyacrylamide gel (BioRad #1653320) or NuPAGE 4-12% Bis-Tris gel (Invitrogen #NP0322), transferred to a nitrocellulose membrane (250 mA, 1.5 hours, on ice), blocked in filtered 5% BSA or milk in TBST, incubated with primary antibodies overnight at 4° C. followed by HRP conjugated secondary antibodies (1:5000) for 1 hour, and visualized with ECL substrate. For phosphorylated protein analysis, the phospho-proteins were probed first, and then blots were stripped (Thermo Scientific 21059) and reprobed for total proteins. For co-IP protein analysis, TrueBlot secondary antibody (eBioscience 18-8816-33) was used to detect only non-denatured IgG and avoid background signal from IP antibody. The following primary antibodies were used: rabbit anti-NKCC1 (Abcam ab59791; 1:1000), rabbit anti-pNKCC1 (EMID Millipore ABS1004; 1:1000), rabbit anti-ATP1a1 (Upstate C464.6/05-369; 1:250), goat-anti-klotho (R&D AF1819-sp; 1:200), rabbit anti-GAPDH (Sigma G9545; 1:10000), mouse anti-HA (Abcam ab130275; 1:1000), rabbit anti-CHD4 (Abcam ab72418; 1:2000), rabbit anti-MBD3 (Abcam ab157464; 1:1000), rabbit anti-HDAC1 (Abcam ab7028; 1:2000), mouse anti-HDAC2 (Abcam 51832; 1:2000). The same protein samples were blotted for multiple targets to allow controlled quantitative comparison (i.e. NKCC1 and GAPDH).

Quantitative RT-PCR

For mRNA expression analyses, the choroid plexus (ChP) were dissected and pooled from 2 pups. RNA was isolated using the MirVana miRNA isolation kit (Invitrogen AM1561) following manufacturer's specifications without miRNA enrichment step. Extracted RNA was quantified spectrophotometrically and 100 ng was reverse-transcribed into cDNA using the High Capacity cDNA Reverse Transcription kit (Applied Biosystems #4368814) following manufacturer's specifications. RT-qPCRs were performed in duplicate using Taqman Gene Expression Assays and Taqman Gene Expression Master Mix (Applied Biosystems) with GAPDH as an internal control. Cycling was executed using the StepOnePlus Real-Time PCR System (Invitrogen) and analysis of relative gene expression was performed using the 2^{-ΔΔCT} method. Technical replicates were averaged for their cycling thresholds and further calculations were performed with those means. Each RNA sample was analyzed for multiple genes.

### In Utero Intracerebroventricular Injection (ICV)

Timed pregnant mice (E14.5) were anesthetized with isoflurane and warmed. Laparotomy was performed and the uterine horns exposed. Embryos were stabilized by hand and lateral ventricles were visually identified. AAV solution was delivered into embryonic lateral ventricles using fine glass capillary pipettes with beveled tips. The AAV solution contained (0.01%) Fast Green FCF (Sigma F7252-5G) to aid with visualization. Uterine horns were returned into the abdominal space, and the incision was sutured. Meloxicam analgesia was longitudinally delivered according to IACUC protocol.

### Intraventricular Kaolin Injection in Postnatal Pups

Postnatal day 4 pups (P4) were anesthetized. A small incision was made in the scalp, and 1 μl of sterile kaolin solution (25% in PBS) was injected into the left lateral ventricle using glass capillary pipettes. The injection site with corresponding lateral ventricle localization was determined as between bregma and lambda, and 1 mm from mid-line. The skin incision was sutured and reinforced with Vetbond (3M™ ID 70200742529). The pups were warmed and returned to the dam.

### AAV Production

The original AAV-NKCC1 plasmid was purchased from Addgene (pcDNA3.1 HA CFP hNKCC1 WT (NT15-H) was a gift from Biff Forbush: Addgene plasmid #49077; http://n2t.net/addgene:49077; RRID:Addgene_49077). The plasmid carries an 3×HA tag at the N-terminal of NKCC1 to allow detection and separation from endogenous NKCC1. The CFP tag was removed by BsaI digestion to reduce insert size for AAV production. Virus production and purification were performed by the Penn Vector Core. The very large size of the plasmid resulted in variable transduction efficiency. All mice receiving AAV-NKCC1 were analyzed for HA expression after every experiment to confirm transduction efficiency. AAV-GFP and AAV-Cre were purchased from the Boston Children's Hospital Viral Core.

### Magnetic Resonance Imaging (MRI)

Mice were imaged using Bruker BioSpec small animal MRI (7T) at 2 wk and P50 while under anesthesia by isoflurane. A warm pad was used to maintain body temperature. Breathing rate and heart rate were monitored to reflect the depth of anesthesia. All axial T2 images were acquired using the following criteria: TE/TR=60/4000; Ave=8; RARE=4; slice thickness=0.6 mm. Ventricle volumes were calculated by manual segmentation using FIJI/ImageJ. Brain sizes were calculated by averaging total cross-sectional areas from the 4 highlighted slices (red) with visible Lateral Ventricle (LV) and 3V in control mice (corresponding slices were used in AAV-NKCC1 regardless of visibility of ventricles). Ventricle regions were not excluded from the brain size measurements. In studies with unilateral kaolin injection, 3D reconstruction of the ventricles was performed by 699manual segmentation in ITK-SNAP⁸⁹ and exported through ParaView.

### Constant Rate Cerebrospinal Fluid (CSF) Infusion Test (ICP and Compliance Measurement)

An apparatus was developed to perform a constant infusion test in mice through a single catheter for both infusion of cerebrospinal fluid (CSF) and monitoring of intracranial pressure (ICP). A 20 cc syringe was filled with aCSF and placed in an automated infusion pump (GenieTouch, Kent Scientific Co., Denver) set to deliver a constant rate infusion of 1-4 uL/minute. The syringe was connected via pressure tubing to hemostasis valve Y connector (Qosina, NY). A fiberoptic intracranial pressure (ICP) sensor (Fiso Technologies Inc, Quebec, Canada) was inserted through the other port of the rotating hemostat and then into 0.55 mm diameter catheter until the sensor was flush with the catheter's distal tip. The entire apparatus and tubing was carefully screened to ensure the absence of air bubbles. Adult mice were then deeply anesthetized, placed on a warm pad, and head-fixed with ear bars. The distal end of the infusion device (catheter with fiberoptic sensor) was placed inside the lateral ventricle through a hole made by a hand-held twist drill with a #74 wire gauge bit (−0.4 mm (anterior-posterior) and 1.2 mm (medial-lateral) with respect to Bregma, and a depth of 2 mm from the outer edge of the skull); the catheter was then sealed with Vetbond (3M, Minnesota). Intraventricular access and water-tight seal was confirmed by observation of arterial and respiratory waveforms in the intracranial pressure (ICP) signal and a transient rise in intracranial pressure (ICP) upon gentle compression of the abdomen and thorax. Two minutes of baseline intracranial pressure (ICP) were recorded before initiating the infusion of aCSF. As the infusion proceeded, careful observation was made of the mouse's respiratory rate. After the intracranial pressure (ICP) level reached a new plateau, the infusion was discontinued. Parameters of the Marmarou model of cerebrospinal fluid (CSF) dynamics for constant rate infusions were estimated by a non-linear least squares fit of the model to the intracranial pressure (ICP) data⁵⁴

$$ICP ( t ) = \frac{[ {i}_{infusion} + \frac{{ICP}_{baseline} - {p}_{0}}{{R}_{CSF}} ] \cdot [ {ICP}_{baseline} - {p}_{0} ]}{\frac{{ICP}_{baseline} - {p}_{0}}{{R}_{CSF}} + {i}_{infusion} \cdot [ {e}^{- \frac{[ {i}_{infusion} + \frac{{ICP}_{baseline} - {p}_{0}}{{R}_{CSF}} ] \cdot }{{C}_{i}} t} ]} \qquad ( 1 )$$

where infusion is the rate of infusion, intracranial pressure (ICP)_{baseline} is the intracranial pressure (ICP) level before infusion, p₀ is a pressure in the storage arm of the model, resistance to cerebrospinal fluid (CSF) outflow (R_{CSF}) is the resistance to cerebrospinal fluid (CSF) outflow, and Cᵢ is the compliance coefficient.

### Statistics and Reproducibility

Biological replicates (N) were defined as samples from distinct and biologically independent individual animals, analyzed either in the same experiment or within multiple experiments, with the exception when individual animal could not provide sufficient sample (i.e. cerebrospinal fluid (CSF)), in which case multiple animals were pooled into one distinct biological replicate and most details are stated in the corresponding figure descriptions provided herein. Due to limited space, these metrics for FIG. 18 are listed here: FIGS. 18A and 18B contain data from 3 independent experiments for N-6 biological replicates. FIG. 18C. Data from 2 independent experiments with a total of 3 biologically independent samples. FIGS. 18D-18F. N=3 distinct animals from 2 independent experiments (same images analyzed in FIGS. 18D, 18E, and 18F), 5-10 field of view (FOV) per animal, distinct cells were captured in each FOV. FIGS. 18H-18I N=26 E16.5 embryos (4 litters); N 736=19 P0 pups (2 litters); N=12 P7 pups (2 litters); N=16 adults. Data are from 5 independent experiments for adult; 2 independent experiments for P7 and P0; 3 independent experiments for E16.5. Each datapoint is an average of the 2 Lateral Ventricle choroid plexus (LVChP) from an individual. j-k N=3 animals per age; 433-833 mitochondria per individual.

The following measurements were taken using the same set of samples/animals: mitochondria/glycogen quantification, immunoblotting, qPCR, and different parameters from cerebrospinal fluid (CSF) infusion tests (ICP, compliance, resistance to cerebrospinal fluid (CSF) outflow (R_{CSF})). In these cases the same image, protein or RNA samples, and mice were analyzed for multiple readouts and targets, to allow controlled quantitative comparison.

Statistical analyses were performed using Prism 7 or R. Outliers were excluded using ROUT method (Q=1%). Appropriate statistical tests were selected based on the distribution of data, homogeneity of variances, and sample size. The majority of the analyses were done using one-way ANOVA with multiple comparison correction (Sidak) or Welch's two-tailed unpaired t-test, except for FIGS. 18D-18F, and FIGS. 18H and 18I where the analysis was done by Welch's ANOVA with Dunnett's T3 multiple comparison test, and FIGS. 18K and 18L where the analysis was done using Kolmogorov-Smirnov test. F tests or Bartlett's tests were used to assess homogeneity of variances between data sets. Parametric tests (t-test, ANOVA) were used only if data were normally distributed and variances were approximately equal. Otherwise, nonparametric alternatives were chosen. Data are presented as means±standard deviation (SD). If multiple measurements were taken from a single individual, data are presented as means±standard errors of the mean (SEMs). p values<0.05 were considered significant (* p<0.05, ** p<0.01, ***p<0.001, ****p<0.0001).

The following methods were employed in Examples 8-14.

### Experimental Design

The overall objective was to investigate the roles of the choroid plexus (ChP) in post-hemorrhagic hydrocephalus, and to test the utility of AAV gene therapy targeting choroid plexus (ChP) epithelial cells to improve the outcomes. The mouse post-hemorrhagic hydrocephalus (PHH) models were developed by directly injecting unprocessed whole blood from age-match donor mice into recipient mice to mimic intraventricular hemorrhage. AAV was delivered through intraventricular injection to genetically modify the choroid plexus (ChP) epithelial cells. MRI and cerebrospinal fluid (CSF) infusion tests were used to evaluate hydrocephalic phenotypes. Sample sizes were informed by estimated mean values from preliminary data and previous studies using the same approaches. Timeline of data collection was determined based on technological restrictions (i.e. mouse age and body weight) and biological significance. All data were collected from at least 2 independent experiments on different dates, with consistent protocols. At least 4 animals from different litters/cohorts were used for each study. No outliners were removed from data. Animal sex, housing, and disease severity were randomized to minimize bias. The researchers performing the experiments were not blinded from the different conditions, but data analyses were performed in a blinded manner whenever possible.

### Statistics

Biological replicates (N) were defined as samples from distinct individuals, analyzed either in the same experiment or within multiple experiments. Some samples were pooled across multiple individuals to obtain sufficient starting material for analyses, in which case each pooled sample was considered as one biological replicate. For the blood proteome study, the list of proteins was processed in Perseus 1.6.0, log 2-transformed, and proteins with less than two peptides (razor) were filtered out as described (57). Principal component analysis was performed on the samples by an unsupervised hierarchical clustering-based heat map. A pairwise comparison was realized on the different age groups and the results were shown using a Volcano plot and a permutation-based FDR testing correction was applied for the significant proteins. For the rest of the data, statistical analyses were performed using Prism 7. Appropriate statistical tests were selected based on the distribution of data, homogeneity of variances, and sample size. The majority of the analyses were performed using One-way ANOVA or Welch's unpaired t-test. F tests or Bartlett's tests were used to assess homogeneity of variances between data sets. Parametric tests (t-test, ANOVA) were used only if data were normally distributed and variances were approximately equal. Otherwise, nonparametric alternatives were chosen. Data are presented as mean±standard deviation (SD). p values<0.05 were considered significant (*p<0.05, ** p<0.01, ***p<0.001, ****p<0.0001).

### Mice

All animal care and experimental procedures were approved by the Institutional Animal Care and Use Committees of Boston Children's Hospital. Animals were housed in a temperature-controlled room on a 12-hr light/12-hr dark cycle and had free access to food and water. Mouse lines used include FoxJ1-Cre (58), Ai95D (Jax #024105 (59)), and CD-1 (Charles River Laboratories).

### CSF Collection

CSF was collected by inserting a glass capillary into cisterna magna, and processed as described (60). Briefly, the cerebrospinal fluid (CSF) was visually examined for purity, and samples were centrifuged at 10,000 g for 10 min. at 4° C. prior to immediate analysis or storage.

### Plasma Collection for Proteomics Using Data-Dependent Acquisition (DDA)

E14.5 embryos were isolated and fresh blood was obtained from the jugular vein using an intact glass microcapillary tube (Drummond #5-000-2020, at its widest diameter). P4 pups were anesthetized on ice. Thoracotomy was performed and blood was obtained via a small incision in the right atrium using an intact glass microcapillary tube. Adults (8 weeks) were anesthetized using ketamine/xylazine. Blood was obtained using the same approach as with P4 pups. No needles or sharp-edged tools were used in order to prevent red blood cell lysis (as indicated by excessively pink-colored plasma). All blood samples were collected into standard EDTA coated tubes (BD #365974) to prevent coagulation. Plasma was extracted from the supernatant after centrifugation for 10 min. at 5,000 g/rcf at 4° C. Centrifugation was performed twice to ensure complete absence of a residual cell pellet. Only samples with yellow to salmon color were included (samples with pink/red color were discarded). Plasma samples were stored at −80° C. until processing in LC/MS.

### Mass Spectrometry

Plasma samples were processed using an MStern blotting protocol developed as described (63). In brief, 1 μL of plasma (˜50 μg of proteins) was mixed in 100 μL of 8 M urea buffer. Cysteine residues were reduced using a final concentration of 10 mM of dithiothreitol and then alkylated with a final concentration of 50 mM of iodoacetamide. Thus, 15 μg of proteins were loaded on to a 96-well plate with a polyvinylidene fluoride membrane at the bottom (Millipore-Sigma), previously activated with 70% ethanol and primed with 8M urea buffer. Trypsin digestion was performed onto the membrane by incubation with the protease for 2 h at 37° C. Resulting tryptic peptides were eluted off the membrane with 40% acetonitrile/0.1% formic acid. A C18 clean-up of the samples was realized using a 96-well MACROSPIN C18 plate (TARGA, NestGroup) and stored at −20° C. before LC/MS analysis. Samples were analyzed using a nanoLC system (Eksigent; Dublin, CA) coupled online to a Q Exactive mass spectrometer (Thermo Scientific; Bremen, Germany). From each sample, 1 μg peptide material was separated using a linear gradient from 98% solvent A (0.1% formic acid in water), 2% solvent B (0.1% formic acid in acetonitrile) to 40% solvent B over 45 minutes. The mass spectrometer was operated in DDA mode, selecting up to 12 of the most intense precursor ions for further MS/MS fragmentation. Label-free protein quantitation analysis employed MaxQuant 1.5.3.30 (57, 64). Raw data were downloaded and used to build a matching library and searched against the UniProt Mouse reviewed protein database. Standard search settings were employed with the following modifications: Max missed cleavage 3; fixed modification Carbamidomethylation (C); variable modification Oxidation (M)44. A revert decoy search strategy was employed to filter all proteins and peptides to <1% FDR.

### Headpost, Cranial Window, and Intracerebroventricular Canula Placement

Mice used for in vivo two-photon imaging (8-20 weeks) were outfitted with a headpost and a 3 mm cranial window, as previously described (12). In addition to the cranial window, a trans-occipital approach to cannula insertion was developed in order to provide intracerebroventricular injections simultaneous with two photon imaging. This trans-occipital injection cannula was inserted through the superior- and lateral-most border of the suboccipital bone and fixed to the headpost together with the cranial window. A dummy cannula was inserted and help in place with a screwcap to prevent infection and tissue growth into the cannula.

### Calcium Imaging and Analysis

Two-photon microscopy (Olympus FVMPE-RS two-photon microscope; 30.0 frames/s; 512×512 pixels/frame) was used to record calcium activity in choroid plexus (ChP) explants or in vivo from mice expressing GCaMP6f in epithelial cells (in FoxJ1-Cre::Ai95D mice). Imaging and analyses were performed as described (12). Plasma (using citrate as anti-coagulant instead of EDTA, same protocol as described above) was delivered to explants by topical application from a glass capillary needle and serum was delivered to the lateral ventricle of live mice during imaging through the injection cannula. Serum was prepared by collecting blood using the same approach as described above, incubating on ice for 30 min. and then centrifuging for 10 min. at 5,000 g twice to separate the supernatant.

### In Utero Intracerebroventricular (ICV) Injection

Timed pregnant mice (E14.5) were anesthetized with isoflurane and kept warm. Following laparotomy, AAV solution or age-matched whole blood was delivered into the lateral ventricle of each embryo (located visually) using glass capillary pipettes. The uterine horns were returned to the abdomen and the incisions sutured. When age-matched whole blood was used, the donor mouse was anesthetized with ketamine/xylazine and warmed. Fresh blood was obtained from the jugular veins with a glass capillary pipette and immediately delivered to the recipient.

### Postnatal Intracerebroventricular (ICV) Injection

P4: pups were anesthetized by hypothermia. An incision was made along the midline of the scalp to expose the skull. A 26 G needle was used to pierce the skull directly above the lateral ventricle. AAV solution or age-matched whole blood was delivered into the lateral ventricle through the piercing using glass capillary pipettes. The skin incision was closed with suture and Vetbond (3M, Minnesota). The pup was warmed and returned to cage after recovery. Whole blood was collected transcardially from littermates anesthetized by hypothermia. Adult: adult intracerebroventricular (ICV) injection followed similar procedures as P4 with the following exceptions: (1) adult mice were anesthetized using isoflurane; (2) a hand drill was used directly above the lateral ventricle; (3) blood donor was anesthetized using ketamine/xylazine.

### Potassium Quantification

Potassium quantification was performed by inductively coupled plasma-optical emission spectrometry (ICP-OES) by Galbraith Laboratories, Inc (Knoxville, TN, USA) using 5-7 μL of mouse cerebrospinal fluid (CSF) and 10-20 μL of human cerebrospinal fluid (CSF).

Osmolarity Measurements CSF samples were measured using Wescor VAPRO vapor pressure osmometer. In mouse studies, 5 μL of cerebrospinal fluid (CSF) was first diluted with 10 μL ddH₂O. 10 μL of the diluted sample was loaded to the osmometer, ddH₂O was used to determine blank value, and the osmolarity of cerebrospinal fluid (CSF) was calculated by (sample value—ddH₂O value) in triplicate. 10 μL human cerebrospinal fluid (CSF) was used without dilution.

### Tissue Processing for Cryosectioning

All postnatal animals were perfused with phosphate-buffered saline (PBS) followed by 4% paraformaldehyde (PFA). The brains were quickly dissected and post-fixed with 4% PFA overnight. Samples were cryoprotected and prepared for embedding at 4° C. with 10% sucrose, 20% sucrose, 30% sucrose, 1:1 mixture of 30% sucrose and OCT (overnight), and OCT (1 hour on ice). Samples were frozen in OCT.

### Immunostaining

Cryosections were blocked and permeabilized (0.3% Triton-X-100 in PBS; 5% goat serum), incubated in primary antibodies at 4° C. overnight and secondary antibodies for 2 hours at room temperature. Hoechst 33342 (Invitrogen H3570, 1:10000, 5 min. at room temperature) was used to visualize nuclei. Sections were mounted using Fluoromount-G (SouthernBiotech). The following primary antibodies were used: rabbit anti-NKCC1 (Abcam ab59791; 1:500*lot-to-lot variation in this antibody was noted), rat anti-HA (Roche 11867423001; 1:1000), and rabbit anti-ZO-1 (Thermo-Fisher, cat #61-7300; 1:100). Secondary antibodies were selected from the Alexa series (Invitrogen, 1:500).

### RNAscope

Sections with Lateral Ventricle (LV) choroid plexus (ChP) were washed twice for 2 min. with PBS to remove OCT, baked in an incubator at 60° C. for 30 min. and gradually rehydrated using 100%, 70%, 30% ethanol. The sections were then fixed with 4% PFA for 45 min. at room temperature and washed 2×3 minutes in ddH₂O before beginning the manufacturer's provided protocol for RNAscope Fluorescent Multiplex (ACD, Advanced Cell Diagnostics). The sections were incubated with Target Retrieval Reagent (ACD 322000) in a vegetable steamer for 12 min. Subsequently, sections were washed twice for 2 min. in ddH₂O prior to incubation with Protease III Reagent (ACD 322340) for 8 min. at 40° C., followed by another cycle of two, 3 min. washes in ddH₂O. Target Probes (ACD Bio RNAscope® Probe-Mm-Fos, 316921) were then hybridized and amplified according to the manufacturer's specifications. Sections with RNAScope labeling were imaged using a bright-field microscope. Each Lateral Ventricle (LV) choroid plexus (ChP) was divided into three equal regions. Overall c-fos expression for each section was measured by dividing the sum of all three choroid plexus (ChP) regions by the sum of equally sized medial cortex in the left and right hemispheres. RNAscope values were obtained from two litters and standardized.

### Immunofluorescence Imaging

Samples were blocked with 5% goat serum in PBS and 0.3% Triton X-100, and incubated with primary antibodies overnight at 4° C. Secondary antibodies were applied by incubating at room temperature for 2 hours. Sections were counterstained with Hoechst 33342 (Invitrogen H3570, 1:10,000) and mounted using Fluoromount-G (SouthernBiotech). The following primary antibodies were used: rabbit anti-NKCC1 (Abcam ab59791; 1:500), rat anti-HA (Roche 11867423001; 1:1000). Secondary antibodies were selected from the Alexa series (Invitrogen, 1:500). Images were acquired using Zeiss LSM880 confocal microscope with 20× objective. ZEN Black software was used for image acquisition and ZEN Blue used for Airy processing.

### Immunoblotting

Tissues were quickly dissected in cold HBSS and homogenized in RIPA buffer supplemented with protease and phosphatase inhibitors. The homogenate was centrifuged at maximum speed for 10 min. to remove cellular debris. Protein concentration was determined by BCA assay (Thermo Scientific 23227). Samples were denatured in 2% SDS supplemented with 2-Mercaptoethanol by heating at 37° C. for 5 min. Equal amounts of proteins were loaded and separated by electrophoresis in a 4-15% gradient polyacrylamide gel (BioRad #1653320) or NuPAGE 4-12% Bis-Tris gel (Invitrogen #NP0322), transferred to a nitrocellulose membrane (250 mA, 1.5 hours, on ice), blocked in filtered 5% milk in TBST, incubated with primary antibodies overnight at 4° C. followed by HRP conjugated secondary antibodies (1:5000) for 1 hour, and visualized with ECL substrate. For phosphorylated protein analysis, the phospho-proteins were probed first, and then blots were stripped (Thermo Scientific 21059) and reprobed for total proteins. The following primary antibodies were used: rabbit anti-NKCC1 (Abcam ab59791; 1:1000), rabbit anti-pNKCC1 (EMD Millipore ABS1004; 1:1000), rabbit anti-GAPDH (Sigma G9545; 1:10000), rabbit anti-pSPAK (EMD Millipore 07-2273; 1:500), and mouse anti-SPAK (Cell Signaling 2281; 1:1000). Band intensity was quantified by FIJI.

### Quantitative RT-PCR

For mRNA expression analyses, the RNA was isolated using the MirVana miRNA isolation kit (Invitrogen AM1561) following manufacturer's specifications without miRNA enrichment step. Extracted RNA was quantified by Nanodrop, and 100 ng was reverse-transcribed into cDNA using the High Capacity cDNA Reverse Transcription kit (Applied Biosystems #4368814) according to the manufacturer's specifications. RT-qPCRs were performed in duplicate using Taqman Gene Expression Assays and Taqman Gene Expression Master Mix (Applied Biosystems) with GAPDH as an internal control. Cycling was executed using the StepOnePlus Real-Time PCR System (Invitrogen) and analysis of relative gene expression was performed using the 2^{-ΔΔCT} method (61). Technical replicates were averaged for their cycling thresholds and further calculations were performed with those means.

### MRI

Mice were imaged using Bruker BioSpec small animal MRI (7T) at P14 or P25 while under anesthesia by isoflurane. A warm pad was used to maintain body temperature. Breathing rate and heart rate were monitored to reflect the depth of anesthesia. All T2 images were acquired using the following criteria: TE/TR=60/4000; Ave=4; RARE=4; slice thickness=0.6 mm.

### CT Imaging

Mice were imaged using the Bruker Albira microCT scanner (45 kV, 0.4 mA settings) while under anesthesia (isoflurane). Body temperature was maintained by a warm pad. Breathing rate and heart rate were monitored. Image analysis performed using AMIDE and Slicer software.

Constant aCSF Infusion Test

Constant aCSF infusion testing was performed as previously described (23). In summary, a single catheter was implanted into the lateral ventricle of a mouse under anesthesia. The catheter infused aCSF at a constant rate and monitored intracranial pressure (ICP). The Marmarou model of cerebrospinal fluid (CSF) dynamics for constant rate infusions were used to calculate parameters describing cerebrospinal fluid (CSF) dynamics by a non-linear least squares fit of the model to the intracranial pressure (ICP) data (62).

### AAV Production

Viral production and purification were performed by University of Pennsylvania (Penn) Vector Core (for AAV-NKCC1 (23)) or BCH viral core (for AAV-GFP).

### Human Sample Collection and Analysis

Human cerebrospinal fluid (CSF) samples were collected by physicians at Massachusetts General Hospital and de-identified for research purposes. The samples were analyzed by Galbraith Laboratory for potassium quantification and analyzed by Wescor VAPRO vapor pressure osmometer following the same protocol as described above.

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

The following references are cited in Examples 1-7 as well as in the methods and discussion relating thereto.
1. Fame, R. M. & Lehtinen, M. K. Emergence and Developmental Roles of the Cerebrospinal Fluid System. Dev Cell 52, 261-275, doi:https://doi.org/10.1016/j.devcel.2020.01.027 (2020).
2. Kahle, K. T., Kulkarni, A. V., Limbrick, D. D., Jr. & Warf, B. C. Hydrocephalus in children. Lancet 387, 788-799, doi:10.1016/50140-6736(15)60694-8 (2016).
3. Vinchon, M., Rekate, H. & Kulkarni, A. V. Pediatric hydrocephalus outcomes: a review. Fluids and barriers of the CNS 9, 18, doi:10.1186/2045-8118-9-18 (2012).
4. Steen, R. G., Mull, C., McClure, R., Hamer, R. M. & Lieberman, J. A. Brain volume in first-episode schizophrenia: systematic review and meta-analysis of magnetic resonance imaging studies. The British journal of psychiatry: the journal of mental science 188, 510-518, doi:10.1192/bjp.188.6.510 (2006).
5. Gilmore, J. H. et al. Prenatal and neonatal brain structure and white matter maturation in children at high risk for schizophrenia. Am J Psychiatry 167, 1083-1091, doi:10.1176/appi.ajp.2010.09101492 (2010).
6. Shen, M. D. et al. Increased Extra-axial Cerebrospinal Fluid in High-Risk Infants Who Later Develop Autism. Biological psychiatry 82, 186-193, doi:10.1016/j.biopsych.2017.02.1095 (2017).
7. Volpe, J. J. Neurology of the newborn. 5th edn, (Saunders/Elsevier, 2008).
8. Louveau, A. et al. Understanding the functions and relationships of the glymphatic system and meningeal lymphatics. The Journal of clinical investigation 127, 3210-3219, doi:10.1172/JC190603 (2017).
9. Ahn, J. H. et al. Meningeal lymphatic vessels at the skull base drain cerebrospinal fluid. Nature 572, 62-66, doi:10.1038/s41586-019-1419-5 (2019).
10. Ma, Q., Ineichen, B. V., Detmar, M. & Proulx, S. T. Outflow of cerebrospinal fluid is 799 predominantly through lymphatic vessels and is reduced in aged mice. Nat Commun 8, 1434, doi:10.1038/s41467-017-01484-6 (2017).
11. Koh, L. et al. Development of cerebrospinal fluid absorption sites in the pig and rat: connections between the subarachnoid space and lymphatic vessels in the olfactory turbinates. Anatomy and embryology 211, 335-344, doi:10.1007/s00429-006-0085-1 (2006).
12. Johnston, M., Nagra, G., Koh, L., Zakharov, A. & Armstrong, D. Cerebrospinal fluid transport across the cribriform plate into extracranial lymphatics in rats: development and quantification. Cerebrospinal fluid research 3, S9 (2006).
13. Papaiconomou, C., Bozanovic-Sosic R Fau-Zakharov, A., Zakharov A Fau-Johnston, M. & Johnston, M. Does neonatal cerebrospinal fluid absorption occur via arachnoid projections or extracranial lymphatics? American journal of physiology. Regulatory, integrative and comparative physiology 28, R869-867, doi:10.1152/ajpregu.00173.2002. (2002).
14. Antila, S. et al. Development and plasticity of meningeal lymphatic vessels. The Journal of experimental medicine 214, 3645-3667, doi:10.1084/jem.20170391 (2017).
15. Damkier, H. H., Brown, P. D. & Praetorius, J. Cerebrospinal fluid secretion by the choroid plexus. Physiological reviews 93, 1847-1892, doi:10.1152/physrev.00004.2013 (2013).
16. Bass, N. H. & Lundborg, P. Postnatal development of bulk flow in the cerebrospinal fluid system of the albino rat: clearance of carboxyl-(14 C)inulin after intrathecal infusion. Brain Res 30, 323-332, doi: 10.1016/0006-8993(73)90668-9. (1973).
17. Amtorp, O. & Sørensen, S. C. The ontogenetic development of concentration differences for protein and ions between plasma and cerebrospinal fluid in rabbits and rats. The Journal of physiology 243, 387-400, doi:10.1113/jphysiol.1974.sp010759 (1974).
18. Johanson, C. & Woodbury, D. in Drugs and the Developing Brain (eds A. Vernadakis & N. Weiner) 281-287 (Plenum Press, 1974).
19. Milhorat, T. H., Mosher, M. B., Hammock, M. K. & Murphy, C. F. Evidence for choroid-plexus absorption in hydrocephalus. The New England journal of medicine 283, 286-289, doi:10.1056/NEJM197008062830604 (1970).
20. Oreskovic, D., Rados, M. & Klarica, M. Role of choroid plexus in cerebrospinal fluid hydrodynamics. Neuroscience 354, 69-87, doi:10.1016/j.neuroscience.2017.04.025 (2017).
21. Crum, J. M., Alvarez, F. J. & Alvarez-Leefmans, F. J. in Presented at the Experimental Biology Meeting. (The FASEB Journal).
22. Keep, R. F., Xiang, J. & Betz, A. L. Potassium cotransport at the rat choroid plexus. The American journal of physiology 267, C1616-1622, doi:10.1152/ajpcell.1994.267.6.C1616 (1994).
23. Liddelow, S. A. et al. Mechanisms that determine the internal environment of the developing brain: a transcriptomic, functional and ultrastructural approach. PLoS One 8, e65629, doi:10.1371/journal.pone.0065629 (2013).
24. Delpire, E. & Gagnon, K. B. Elusive role of the Na—K-2C1 cotransporter in the choroid plexus. American journal of physiology. Cell physiology 316, C522-C524, doi:10.1152/ajpcell.00490.2018 (2019).
25. Davson, H. Physiology of the cerebrospinal fluid. (Churchill, 1967).
26. Ferguson, R. F. & Woodbury, D. M. Penetration of 14C-inulin and 14C-sucrose into brain, cerebrospinal fluid, and skeletal muscle of developing rats. Exp Brain Res 7, 181-194, doi:10.1007/BF00239028 (1969).
27. Bradbury, M. W. & Stulcovi, B. Efflux mechanism contributing to the stability of the potassium concentration in cerebrospinal fluid. The Journal of physiology 208, 415-430, doi:10.1113/jphysiol.1970.sp009128 (1970).
28. Bradbury, M. W. & Kleeman, C. R. Stability of the potassium content of cerebrospinal fluid and brain. American Journal of Physiology-Legacy Content 213, 519-528, doi:10.1152/ajplegacy.1967.213.2.519 (1967).
29. Bradbury, M. W. & Davson, H. The transport of potassium between blood, cerebrospinal fluid and brain. The Journal of physiology 181, 151-174, doi:10.1113/jphysiol.1965.sp007752 (1965).
30. Zeuthen, T. & Macaulay, N. Cotransport of water by Na(+)—K(+)-2C1(−) cotransporters expressed in Xenopus oocytes: NKCC1 versus NKCC2. The Journal of physiology 590, 1139-1154, doi:10.1113/jphysiol.2011.226316 (2012).
31. Zeuthen, T. Cotransport of K⁺, Cl⁻ and H₂O by membrane proteins from choroid plexus epithelium of Necturus maculosus. The Journal of physiology 478 (Pt 2), 203-219, doi:10.1113/jphysiol.1994.sp020243 (1994).
32. Hamann, S., Herrera-Perez, J. J., Zeuthen, T. & Alvarez-Leefmans, F. J. Cotransport of water by the Na+-K+-2Cl(−) cotransporter NKCC1 in mammalian epithelial cells. The Journal of physiology 588, 4089-4101, doi:10.1113/jphysiol.2010.194738 (2010).
33. Keep, R. F. & Jones, H. C. A morphometric study on the development of the lateral ventricle choroid plexus, choroid plexus capillaries and ventricular ependyma in the rat. Brain research. Developmental brain research 56, 47-53, doi:10.1016/0165-3806(90)90163-s (1990).
34. Bhargava, P. & Schnellmann, R. G. Mitochondrial energetics in the kidney. Nature reviews. Nephrology 13, 629-646, doi:10.1038/nrneph.2017.107 (2017).
35. Netsky, M. G. & Shuangshoti, S. The choroid plexus in health and disease. (University Press of Virginia, 1975).
36. Schuler, M. H. et al. Mirol-mediated mitochondrial positioning shapes intracellular energy gradients required for cell migration. Molecular biology of the cell 28, 2159-2169, doi:10.1091/mbc.E16-10-0741 (2017).
37. Smith, G. M. & Gallo, G. The role of mitochondria in axon development and regeneration. Dev Neurobiol 78, 221-237, doi:10.1002/dneu.22546 (2018).
38. Heiman, M. et al. A translational profiling approach for the molecular characterization of CNS cell types. Cell 135, 738-748, doi:10.1016/j.cell.2008.10.028 (2008).
39. Zhang, Y. et al. A transgenic FOXJ1-Cre system for gene inactivation in ciliated epithelial cells. American journal of respiratory cell and molecular biology 36, 515-519, doi:10.1165/rcmb.2006-0475RC (2007).
40. Steffensen, A. B. et al. Cotransporter-mediated water transport underlying cerebrospinal fluid formation. Nat Commun 9, 2167, doi:10.1038/s41467-018-04677-9 (2018).
41. Darman, R. B. & Forbush, B. A regulatory locus of phosphorylation in the N terminus of the Na—K—Cl cotransporter, NKCC1. J Biol Chem 277, 37542-37550, doi:10.1074/jbc.M206293200 (2002).
42. Sopjani, M. et al. Regulation of the Na+/K+ ATPase by Klotho. FEBS Lett 585, 1759-1764, doi:10.1016/j.febslet.2011.05.021 (2011).
43. Razzaque, M. S. Klotho and Na⁺,K⁺-ATPase activity: solving the calcium metabolism dilemma? Nephrology, dialysis, transplantation: official publication of the European Dialysis and Transplant Association—European Renal Association 23, 459-461, doi:10.1093/ndt/gfm702 (2008).
44. Goodman, J. V. & Bonni, A. Regulation of neuronal connectivity in the mammalian brain by chromatin remodeling. Current opinion in neurobiology 59, 59-68, doi:10.1016/j.conb.2019.04.010 (2019).
45. Lun, M. P. et al. Spatially heterogeneous choroid plexus transcriptomes encode positional identity and contribute to regional CSF production. J Neurosci 35, 4903-4916, doi:10.1523/JNEUROSCI.3081-14.2015 (2015).
46. Weiss, K. et al. The CHD4-related syndrome: a comprehensive investigation of the clinical spectrum, genotype-phenotype correlations, and molecular basis. Genet Med 22, 389-397, doi:10.1038/s41436-019-0612-0 (2020).
47. Williams, C. J. et al. The chromatin remodeler Mi-2beta is required for CD4 expression and T cell development. Immunity 20, 719-733, doi:10.1016/j.immuni.2004.05.005 (2004).
48. Haddad, M. R., Donsante, A., Zerfas, P. & Kaler, S. G. Fetal Brain-directed AAV Gene Therapy Results in Rapid, Robust, and Persistent Transduction of Mouse Choroid Plexus Epithelia. Mol Ther Nucleic Acids 2, e101, doi:10.1038/mtna.2013.27 (2013).
49. Somasekharan, S., Monette, M. Y. & Forbush, B. Functional expression of human NKCC1 from a synthetic cassette-based cDNA: introduction of extracellular epitope tags and removal of cysteines. PLoS One 8, e82060, doi:10.1371/journal.pone.0082060 (2013).
50. Aquilina, K., Pople, I. K., Sacree, J., Carter, M. R. & Edwards, R. J. The constant flow ventricular infusion test: a simple and useful study in the diagnosis of third ventriculostomy failure. Journal of neurosurgery 116, 445-452, doi:10.3171/2011.10.JNS1140 (2012).
51. Eide, P. K. The pathophysiology of chronic noncommunicating hydrocephalus: lessons from continuous intracranial pressure monitoring and ventricular infusion testing. Journal of neurosurgery 129, 220-233, doi:10.3171/2017.1.JNS162813 (2018).
52. Lalou, A. D. et al. Cerebral autoregulation, cerebrospinal fluid outflow resistance, and outcome following cerebrospinal fluid diversion in normal pressure hydrocephalus. Journal of neurosurgery 130, 154-162, doi:10.3171/2017.7.JNS17216 (2018).
53. Deane, R. & Jones, H. C. Cerebrospinal fluid outflow resistance in the developing rat. Zeitschrift für Kinderchirurgie 38 (1983).
54. Czosnyka, M., Czosnyka, Z., Agarwal-Harding, K. J. & Pickard, J. D. Modeling of CSF dynamics: legacy of Professor Anthony Marmarou. Acta neurochirurgica. Supplement 113, 9-14, doi:10.1007/978-3-7091-0923-6_2 (2012).
55. Shaolin, Z. et al. Hydrocephalus induced via intraventricular kaolin injection in adult rats. Folia neuropathologica 53, 60-68, doi:10.5114/fn.2015.49975 (2015).
56. Munk, A. S. et al. PDGF-B Is Required for Development of the Glymphatic System. Cell Rep 26, 2955-2969 e2953, doi:10.1016/j.celrep.2019.02.050 (2019).
57. Hudry, E. et al. Gene transfer of human Apoe isoforms results in differential modulation of amyloid deposition and neurotoxicity in mouse brain. Sci Transl Med 5, 212ra161, doi:10.1126/scitranslmed.3007000 (2013).
58. Kaler, S. G. Menkes disease. Advances in pediatrics 41, 263-304 (1994).
59. Gato, A. et al. Embryonic cerebrospinal fluid in brain development: neural progenitor control. Croatian medical journal 55, 299-305, doi:10.3325/cmj.2014.55.299 (2014).
60. Furey, C. G. et al. De Novo Mutation in Genes Regulating Neural Stem Cell Fate in Human Congenital Hydrocephalus. Neuron 99, 302-314 e304, doi:10.1016/j.neuron.2018.06.019 (2018).
61. Rasmussen, R., O'Donnell, J., Ding, F. & Nedergaard, M. Interstitial ions: A key regulator of state-dependent neural activity? Progress in neurobiology 193, 101802, doi:10.1016/j.pneurobio.2020.101802 (2020).
62. LoTurco, J. J., Owens, D. F., Heath, M. J., Davis, M. B. & Kriegstein, A. R. GABA and glutamate depolarize cortical progenitor cells and inhibit DNA synthesis. Neuron 15, 1287-1298, doi:10.1016/0896-6273(95)90008-x (1995).
63. Owens, D. F., Liu, X. & Kriegstein, A. R. Changing properties of GABA(A) receptor-mediated signaling during early neocortical development. J Neurophysiol 82, 570-583, doi:10.1152/jn.1999.82.2.570 (1999).
64. Pisella, L. I. et al. Impaired regulation of KCC2 phosphorylation leads to neuronal network dysfunction and neurodevelopmental pathology. Science Signaling 12, eaay0300, doi:10.1126/scisignal.aay0300 (2019).
65. Watanabe, M. et al. Developmentally regulated KCC2 phosphorylation is essential for dynamic GABA-mediated inhibition and survival. Science Signaling 12, eaaw9315, doi:10.1126/scisignal.aaw9315 (2019).
66. Fencl, V., Miller, T. B. & Pappenheimer, J. R. Studies on the respiratory response to disturbances of acid-base balance, with deductions concerning the ionic composition of cerebral interstitial fluid. The American journal of physiology 210, 459-472, doi:10.1152/ajplegacy.1966.210.3.459 (1966).
67. Cserr, H. Potassium exchange between cerebrospinal fluid, plasma, and brain. The American journal of physiology 209, 1219-1226, doi:10.1152/ajplegacy.1965.209.6.1219 (1965).
68. Amin, H., Marinaro, F., De Pietri Tonelli, D. & Berdondini, L. Developmental excitatory-to-inhibitory GABA-polarity switch is disrupted in 22q11.2 deletion syndrome: a potential target for clinical therapeutics. Scientific reports 7, 15752, doi:10.1038/s41598-017-15793-9 (2017).
69. Banerjee, A. et al. Jointly reduced inhibition and excitation underlies circuit-wide changes in cortical processing in Rett syndrome. Proc Natl Acad Sci USA 113, E7287-E7296, doi:10.1073/pnas.1615330113 (2016).
70. He, Q., Nomura, T., Xu, J. & Contractor, A. The developmental switch in GABA polarity is delayed in fragile X mice. J Neurosci 34, 446-450, doi:10.1523/JNEUROSCI.4447-13.2014 (2014).
71. Hyde, T. M. et al. Expression of GABA signaling molecules KCC2, NKCC1, and GAD1 in cortical development and schizophrenia. J Neurosci 31, 11088-11095, doi:10.1523/JNEUROSCI.1234-11.2011 (2011).
72. Deidda, G. et al. Reversing excitatory GABAAR signaling restores synaptic plasticity and memory in a mouse model of Down syndrome. Nature medicine 21, 318-326, doi:10.1038/nm.3827 (2015).
73. Madry, C. et al. Microglial Ramification, Surveillance, and Interleukin-1beta Release Are Regulated by the Two-Pore Domain K(+) Channel THIK-1. Neuron 97, 299-312 e296, doi:10.1016/j.neuron.2017.12.002 (2018).
74. Schafer, D. P. et al. Microglia sculpt postnatal neural circuits in an activity and complement-dependent manner. Neuron 74, 691-705, doi:10.1016/j.neuron.2012.03.026 (2012).
75. Li, Q. et al. Aquaporin 1 and the Na(+)/K(+)/2C1(−) cotransporter 1 are present in the leptomeningeal vasculature of the adult rodent central nervous system. Fluids and barriers of the CNS 17, 15, doi:10.1186/s12987-020-0176-z (2020).
76. Spassky, N. et al. Adult ependymal cells are postmitotic and are derived from radial glial cells during embryogenesis. J Neurosci 25, 10-18, doi:10.1523/JNEUROSCI.1108-04.2005 (2005).
77. Vinje, V., Eklund, A., Mardal, K. A., Rognes, M. E. & Stoverud, K. H. Intracranial pressure elevation alters CSF clearance pathways. Fluids and barriers of the CNS 17, 29, doi:10.1186/s12987-020-00189-1 (2020).
78. Dobin, A. et al. STAR: ultrafast universal RNA-seq aligner. Bioinformatics 29, 15-21, doi:10.1093/bioinformatics/bts635 (2013).
79. Anders, S., Pyl, P. T. & Huber, W. HTSeq—a Python framework to work with high-throughput sequencing data. Bioinformatics 31, 166-169, doi:10.1093/bioinformatics/btu638 (2015).
80. Trapnell, C. et al. Transcript assembly and quantification by RNA-Seq reveals unannotated transcripts and isoform switching during cell differentiation. Nat Biotechnol 28, 511-515, doi:10.1038/nbt.1621 (2010).
81. Love, M. I., Huber, W. & Anders, S. Moderated estimation of fold change and dispersion for RNA-seq data with DESeq2. Genome biology 15, 550, doi:10.1186/s13059-014-0550-8 (2014).
82. Huang da, W., Sherman, B. T. & Lempicki, R. A. Systematic and integrative analysis of large gene lists using DAVID bioinformatics resources. Nature protocols 4, 44-57, doi:10.1038/nprot.2008.211 (2009).
83. Draghici, S. et al. A systems biology approach for pathway level analysis. Genome research 17, 1537-1545, doi:10.1101/gr.6202607 (2007).
84. Almagro Armenteros, J. J. et al. SignalP 5.0 improves signal peptide predictions using deep neural networks. Nat Biotechnol 37, 420-423, doi:10.1038/s41587-019-0036-z (2019).
85. Sonnhammer, E. L. L., Heijne, G. v. & Krogh, A. in In Proc. of Sixth Int. Conf on Intelligent Systems for Molecular Biology (eds J. Glasgow et al.) 175-182 (AAAI Press, 1998).
86. Schneider, C. A., Rasband, W. S. & Eliceiri, K. W. NIH Image to ImageJ: 25 years of image analysis. Nat Methods 9, 671-675 (2012).
87. Schindelin, J. et al. Fiji: an open-source platform for biological-image analysis. Nat Methods 9, 676-682, doi:10.1038/nmeth.2019 (2012).
88. Dziegielewska, K. M. et al. Proteins in cerebrospinal fluid and plasma of fetal rats during development. Dev Biol 83, 193-200, doi:https://doi.org/10.1016/S0012-1606(81)80024-3 (1981).
89. Madan, C. R. Creating 3D visualizations of MRI data: A brief guide. F1000Research 4, 466, doi:10.12688/f1000research.6838.1 (2015).

The following references are cited in Examples 8-14 as well as in the methods and discussion relating thereto.
1. A. V. Kulkarni, J. Riva-Cambrin, J. Butler, S. R. Browd, J. M. Drake, R. Holubkov, J. R. W.

Kestle, D. D. Limbrick, T. D. Simon, M. S. Tamber, J. C. Wellons, W. E. Whitehead, Outcomes of CSF shunting in children: Comparison of Hydrocephalus Clinical Research Network cohort with historical controls, J. Neurosurg. Pediatr. 12, 334-338 (2013).
2. K. T. Kahle, A. V Kulkarni, D. D. Limbrick Jr., B. C. Warf, Hydrocephalus in children, Lancet 387, 788-799 (2016).
3. S. Robinson, Neonatal posthemorrhagic hydrocephalus from prematurity: pathophysiology and current treatment concepts, J Neurosurg Pediatr 9, 242-258 (2012).
4. M. N. Cizmeci, N. Khalili, N. H. P. Claessens, F. Groenendaal, K. D. Liem, A. Heep, I. Benavente-Fernindez, H. L. M. van Straaten, G. van Wezel-Meijler, S. J. Steggerda, J. Dudink, I. Isgum, A. Whitelaw, M. J. N. L. Benders, L. S. de Vries, K. Han, P. Woerdeman, H. J. ter Horst, K. P. Dijkman, D. Ley, V. Fellman, T. R. de Haan, A. J. Brouwer, E. van't Verlaat, P. Govaert, B. J. Smit, T. Agut Quijano, U. Barcik, A. Mathur, A. M. Graca, Assessment of Brain Injury and Brain Volumes after Posthemorrhagic Ventricular Dilatation: A Nested Substudy of the Randomized Controlled ELVIS Trial, J. Pediatr. 208, 191-197.e2 (2019).
5. L. S. de Vries, F. Groenendaal, K. D. Liem, A. Heep, A. J. Brouwer, E. van't Verlaat, I. Benavente-Fernandez, H. L. van Straaten, G. van Wezel-Meijler, B. J. Smit, P. Govaert, P. A. Woerdeman, A. Whitelaw, E. study group, Treatment thresholds for intervention in posthaemorrhagic ventricular dilation: a randomised controlled trial, Arch Dis Child Fetal Neonatal Ed 104, F70-F75 (2019).
6. T. D. Simon, J. Riva-Cambrin, R. Srivastava, S. L. Bratton, J. M. Dean, J. R. Kestle, N. Hydrocephalus Clinical Research, Hospital care for children with hydrocephalus in the United States: utilization, charges, comorbidities, and deaths, J Neurosurg Pediatr 1, 131-137 (2008).
7. N. L. Maitre, D. D. Marshall, W. A. Price, J. C. Slaughter, T. M. O'Shea, C. Maxfield, R. F. Goldstein, Neurodevelopmental outcome of infants with unilateral or bilateral periventricular hemorrhagic infarction, Pediatrics 124, e1153-60 (2009).
8. S. S. Rao, D. Y. Chung, Z. Wolcott, F. Sheriff, A. M. Khawaja, H. Lee, M. M. Guanci, T. M. Leslie-Mazwi, W. T. Kimberly, A. B. Patel, G. A. Rordorf, Intermittent CSF drainage and rapid EVD weaning approach after subarachnoid hemorrhage: association with fewer VP shunts and shorter length of stay, J Neurosurg, 1-6 (2019).
9. R. Spector, R. F. Keep, S. Robert Snodgrass, Q. R. Smith, C. E. Johanson, A balanced view of choroid plexus structure and function: Focus on adult humans, Exp Neurol 267, 78-86 (2015).
10. W. M. Pardridge, CSF, blood-brain barrier, and brain drug delivery, Expert Opin Drug Deliv 13, 963-975 (2016).
11. J. Praetorius, H. H. Damkier, Transport across the choroid plexus epithelium, Am J Physiol Cell Physiol 312, C673-C686 (2017).
12. F. B. Shipley, N. Dani, H. Xu, C. Deister, J. Cui, J. P. Head, C. Sadegh, R. M. Fame, M. L. Shannon, V. I. Flores, T. Kishkovich, E. Jang, E. M. Klein, G. J. Goldey, K. He, Y. Zhang, M. J. Holtzman, T. Kirchhausen, C. Wyart, C. I. Moore, M. L. Andermann, M. K. Lehtinen, Tracking Calcium Dynamics and Immune Surveillance at the Choroid Plexus Blood-Cerebrospinal Fluid Interface, Neuron 108, 623-639.e10 (2020).
13. H. H. Damkier, P. D. Brown, J. Praetorius, Cerebrospinal fluid secretion by the choroid plexus, Physiol Rev 93, 1847-1892 (2013).
14. R. M. Fame, M. K. Lehtinen, Emergence and Developmental Roles of the Cerebrospinal Fluid System Dev. Cell 52, 261-275 (2020).
15. M. R. Haddad, A. Donsante, P. Zerfas, S. G. Kaler, Fetal brain-directed AAV gene therapy results in rapid, robust, and persistent transduction of mouse choroid plexus epithelia, Mol. Ther.—Nucleic Acids 2, e101 (2013).
16. J. Cui, F. B. Shipley, M. L. Shannon, O. Alturkistani, N. Dani, M. D. Webb, A. U. Sugden, M. L. Andermann, M. K. Lehtinen, Inflammation of the Embryonic Choroid Plexus Barrier following Maternal Immune Activation, Dev. Cell 55, 617-628 (2020).
17. E. Hudry, J. Dashkoff, A. D. Roe, S. Takeda, R. M. Koffie, T. Hashimoto, M. Scheel, T. Spires-Jones, M. Arbel-Ornath, R. Betensky, B. L. Davidson, B. T. Hyman, Gene transfer of human Apoe isoforms results in differential modulation of amyloid deposition and neurotoxicity in mouse brain, Sci. Transl. Med. 5 (2013), doi:10.1126/scitranslmed.3007000.
18. P. F. Simard, C. Tosun, L. Melnichenko, S. Ivanova, V. Gerzanich, J. M. Simard, Inflammation of the Choroid Plexus and Ependymal Layer of the Ventricle Following Intraventricular Hemorrhage, Transl. Stroke Res. 2, 227-231 (2011).
19. J. K. Karimy, J. Zhang, D. B. Kurland, B. C. Theriault, D. Duran, J. A. Stokum, C. G. Furey, X. Zhou, M. S. Mansuri, J. Montejo, A. Vera, M. L. DiLuna, E. Delpire, S. L. Alper, M. Gunel, V. Gerzanich, R. Medzhitov, J. M. Simard, K. T. Kahle, Inflammation-dependent cerebrospinal fluid hypersecretion by the choroid plexus epithelium in posthemorrhagic hydrocephalus, Nat Med 23, 997-1003 (2017).
20. A. B. Steffensen, E. K. Oernbo, A. Stoica, N. J. Gerkau, D. Barbuskaite, K. Tritsaris, C. R. Rose, N. MacAulay, Cotransporter-mediated water transport underlying cerebrospinal fluid formation, Nat Commun 9, 2167 (2018).
21. Q. Li, N. N. Aalling, B. Forstera, A. Erturk, M. Nedergaard, K. Mollgard, A. L. R. Xavier, Aquaporin 1 and the Na(+)/K(+)/2C1(−) cotransporter 1 are present in the leptomeningeal vasculature of the adult rodent central nervous system, Fluids Barriers CNS 17, 15 (2020).
22. E. Delpire, K. B. Gagnon, Elusive role of the Na—K-2C1 cotransporter in the choroid plexus, Am. J. Physiol.—Cell Physiol. 316, C522-C524 (2019).
23. H. Xu, R. Fame, C. Sadegh, J. Sutin, C. Naranjo, D. Syau, J. Cui, F. Shipley, A. Vernon, F. Gao, Y. Zhang, M. Holtzman, B. Heiman, B. C. Warf, P. Lin, M. Lehtinen, Choroid plexus NKCC1 mediates cerebrospinal fluid clearance during mouse early postnatal development, Nat. Commun. (in Press. (2021), doi:Preprint available at BioRxiv “Non-canonical, potassium-driven cerebrospinal fluid clearance”: https://www.biorxiv.org/content/10.1101/2020.08.03.234260v1.
24. J. M. C. Gregoriades, A. Madaris, F. J. Alvarez, F. J. Alvarez-Leefmans, Genetic and pharmacological inactivation of apical Na⁺—K⁺-2C1-cotransporter 1 in choroid plexus epithelial cells reveals the physiological function of the cotransporter, Am. J. Physiol.—Cell Physiol. 316, C525-C544 (2019).
25. T. H. Milhorat, M. B. Mosher, M. K. Hammock, C. F. Murphy, Evidence for Choroid-Plexus Arsorption in Hydrocephalus, N. Engl. J. Med. 283, 286-289 (1970).
26. D. Oreskovic, M. Rados, M. Klarica, Role of choroid plexus in cerebrospinal fluid hydrodynamics, Neuroscience 354, 69-87 (2017).
27. J. Strahle, H. J. L. Garton, C. O. Maher, K. M. Muraszko, R. F. Keep, G. Xi, Mechanisms of Hydrocephalus After Neonatal and Adult Intraventricular Hemorrhage Transl. Stroke Res. 3, 25-38 (2012).
28. Y. C. Yung, T. Mutoh, M. E. Lin, K. Noguchi, R. R. Rivera, J. W. Choi, M. A. Kingsbury, J. Chun, Lysophosphatidic acid signaling may initiate fetal hydrocephalus, Sci. Transl. Med. 3 (2011), doi:10.1126/scitranslmed.3002095.
29. R. Park, U. Y. Moon, J. Y. Park, L. J. Hughes, R. L. Johnson, S. H. Cho, S. Kim, Yap is required for ependymal integrity and is suppressed in LPA-induced hydrocephalus, Nat. Commun. 7 (2016), doi:10.1038/ncomms10329.
30. N. C. Lummis, P. Sanchez-Pavon, G. Kennedy, A. J. Frantz, Y. Kihara, V. A. Blaho, J. Chun, LPA1/3 overactivation induces neonatal posthemorrhagic hydrocephalus through ependymal loss and ciliary dysfunction, Sci Adv 5, eaax2011 (2019).
31. S. Robinson, F. S. Conteh, A. Y. Oppong, T. R. Yellowhair, J. C. Newville, N. El Demerdash, C. L. Shrock, J. R. Maxwell, S. Jett, F. J. Northington, L. L. Jantzie, Extended Combined Neonatal Treatment With Erythropoietin Plus Melatonin Prevents Posthemorrhagic Hydrocephalus of Prematurity in Rats, Front. Cell. Neurosci. 12 (2018), doi:10.3389/fncel.2018.00322.
32. H. M. Tully, W. B. Dobyns, Infantile hydrocephalus: a review of epidemiology, classification and causes, Eur J Med Genet 57, 359-368 (2014).
33. H. M. Tully, G. E. Ishak, T. C. Rue, J. C. Dempsey, S. R. Browd, K. J. Millen, D. Doherty, W. B. Dobyns, Two Hundred Thirty-Six Children With Developmental Hydrocephalus: Causes and Clinical Consequences, J Child Neurol 31, 309-320 (2016).
34. L. A. Papile, J. Burstein, R. Burstein, H. Koffler, Incidence and evolution of subependymal and intraventricular hemorrhage: A study of infants with birth weights less than 1,500 gm, J. Pediatr. 92, 529-534 (1978).
35. S. S. Roeder, M. I. Sprugel, J. A. Sembill, A. Giede-Jeppe, K. Macha, D. MadZar, H. Lucking, P. Hoelter, S. T. Gerner, J. B. Kuramatsu, H. B. Huttner, Influence of the Extent of Intraventricular Hemorrhage on Functional Outcome and Mortality in Intracerebral Hemorrhage, Cerebrovasc. Dis. 47, 245-252 (2019).
36. S. F. Dias, A. D. Lalou, R. Spang, K. Haas-Lude, M. Garnett, H. Fernandez, M. Czosnyka, M. U. Schuhmann, Z. Czosnyka, Value of computerized shunt infusion study in assessment of pediatric hydrocephalus shunt function—a two center cross-sectional study, Child's Nerv. Syst. 36, 59-71 (2020).
37. P. K. Eide, A. Brean, Cerebrospinal fluid pulse pressure amplitude during lumbar infusion in idiopathic normal pressure hydrocephalus can predict response to shunting, Cerebrospinal Fluid Res. 7 (2010), doi:10.1186/1743-8454-7-5.
38. K. Aquilina, I. K. Pople, J. Sacree, M. R. Carter, R. J. Edwards, The constant flow ventricular infusion test: A simple and useful study in the diagnosis of third ventriculostomy failure—Clinical article, J. Neurosurg. 116, 445-452 (2012).
39. P. K. Eide, The pathophysiology of chronic noncommunicating hydrocephalus: Lessons from continuous intracranial pressure monitoring and ventricular infusion testing, J. Neurosurg. 129, 220-233 (2018).
40. L. da Silva Lopes, I. Slobodian, M. R. Del Bigio, Characterization of juvenile and young adult mice following induction of hydrocephalus with kaolin, Exp. Neurol. 219, 187-196 (2009).
41. J. K. Karimy, D. Duran, J. K. J. K. Hu, C. Gavankar, J. R. Gaillard, Y. Bayri, H. Rice, M. L. DiLuna, V. Gerzanich, J. M. Simard, K. T. Kahle, Cerebrospinal fluid hypersecretion in pediatric hydrocephalus, Neurosurg. Focus 41 (2016), doi:10.3171/2016.8.FOCUS16278.
42. C. H. Mazucanti, Q. R. Liu, D. Lang, N. Huang, J. F. O'Connell, S. Camandola, J. M. Egan, Release of insulin produced by the choroid plexis is regulated by serotonergic signaling, JCI Insight 4 (2019), doi:10.1172/jci.insight.131682.
43. M. J. Berridge, C. P. Downes, M. R. Hanley, Neural and developmental actions of lithium: A unifying hypothesis Cell 59, 411-419 (1989).
44. C. Pisanu, C. Melis, A. Squassina, Lithium Pharmacogenetics: Where Do We Stand?, Drug Dev Res 77, 368-373 (2016).
45. T. Maruyama, T. Kanaji, S. Nakade, T. Kanno, K. Mikoshiba, 2APB, 2-aminoethoxydiphenyl borate, a membrane-penetrable modulator of Ins(1,4,5)P3-induced Ca2+ release, J. Biochem. 122, 498-505 (1997).
46. C. J. Stapleton, A. Acharjee, H. J. Irvine, Z. C. Wolcott, A. B. Patel, W. T. Kimberly, High-throughput metabolite profiling: identification of plasma taurine as a potential biomarker of functional outcome after aneurysmal subarachnoid hemorrhage, J Neurosurg, 1-8 (2019).
47. C. D. Wilson, S. Safavi-Abbasi, H. Sun, M. Y. Kalani, Y. D. Zhao, M. R. Levitt, R. A. Hanel, E. Sauvageau, T. B. Mapstone, F. C. Albuquerque, C. G. McDougall, P. Nakaji, R. F. Spetzler, Meta-analysis and systematic review of risk factors for shunt dependency after aneurysmal subarachnoid hemorrhage, J Neurosurg 126, 586-595 (2017).
48. B. P. Murphy, T. E. Inder, V. Rooks, G. A. Taylor, N. J. Anderson, N. Mogridge, L. J. Horwood, J. J. Volpe, Posthaemorrhagic ventricular dilatation in the premature infant: natural history and predictors of outcome, Arch Dis Child Fetal Neonatal Ed 87, F37-41 (2002).
49. P. R. Stutchfield, R. W. I. Cooke, Electrolytes and glucose in cerebrospinal fluid of premature infants with intraventricular haemorrhage: Role of potassium in cerebral infarction, Arch. Dis. Child. 64, 470-475 (1989).
50. M. N. Cizmeci, F. Groenendaal, K. D. Liem, I. C. van Haastert, I. Benavente-Fernindez, H. L. M. van Straaten, S. Steggerda, B. J. Smit, A. Whitelaw, P. Woerdeman, A. Heep, L. S. de Vries, K. S. Han, H. J. ter Horst, K. P. Dijkman, D. Ley, V. Fellman, T. R. de Haan, A. J. Brouwer, M. J. N. L. Benders, J. Dudink, E. van t. Verlaat, P. Govaert, R. M. C. Swarte, M. Rijken, G. van Wezel-Meijler, T. A. Quijano, U. Barcik, A. M. Mathur, A. M. Graca, Randomized Controlled Early versus Late Ventricular Intervention Study in Posthemorrhagic Ventricular Dilatation: Outcome at 2 Years, J. Pediatr. 226, 28-35.e2 (2020).
51. J. Chen, L. Wang, H. Xu, L. Xing, Z. Zhuang, Y. Zheng, X. Li, C. Wang, S. Chen, Z. Guo, Q. Liang, Y. Wang, Meningeal lymphatics clear erythrocytes that arise from subarachnoid hemorrhage, Nat. Commun. 11 (2020), doi:10.1038/s41467-020-16851-z.
52. G. Trifan, B. Arshi, F. D. Testai, Intraventricular Hemorrhage Severity as a Predictor of Outcome in Intracerebral Hemorrhage, Front. Neurol. 10 (2019), doi:10.3389/fneur.2019.00217.
53. M. Schulz, C. Buhrer, A. Pohl-Schickinger, H. Haberl, U. W. Thomale, Neuroendoscopic lavage for the treatment of intraventricular hemorrhage and hydrocephalus in neonates: Clinical article, J. Neurosurg. Pediatr. 13, 626-635 (2014).
54. K. Luyt, S. L. Jary, C. L. Lea, G. J. Young, D. E. Odd, H. E. Miller, G. Kmita, C. Williams, P. S. Blair, W. Hollingworth, M. Morgan, A. P. Smith-Collins, S. Walker-Cox, K. Aquilina, I. Pople, A. G. Whitelaw, Drainage, irrigation and fibrinolytic therapy (DRIFT) for posthaemorrhagic ventricular dilatation: 10-year follow-up of a randomised controlled trial, Arch. Dis. Child. Fetal Neonatal Ed. 105, 466-473 (2020).
55. D. F. Bauer, L. C. Baird, P. Klimo, C. A. Mazzola, D. C. Nikas, M. S. Tamber, A. M. Flannery, Congress of Neurological Surgeons Systematic Review and Evidence-Based Guidelines on the Treatment of Pediatric Hydrocephalus: Update of the 2014 Guidelines, Neurosurgery 87, 1071-1075 (2020).
56. E. Hudry, L. H. Vandenberghe, Therapeutic AAV Gene Transfer to the Nervous System: A Clinical Reality Neuron 101, 839-862 (2019).
57. S. Tyanova, T. Temu, P. Sinitcyn, A. Carlson, M. Y. Hein, T. Geiger, M. Mann, J. Cox, The Perseus computational platform for comprehensive analysis of (prote)omics data Nat. Methods 13, 731-740 (2016).
58. Y. Zhang, G. Huang, L. P. Shornick, W. T. Roswit, J. M. Shipley, S. L. Brody, M. J. Holtzman, A transgenic FOXJ1-Cre system for gene inactivation in ciliated epithelial cells, Am. J. Respir. Cell Mol. Biol. 36, 515-519 (2007).
59. L. Madisen, A. R. Garner, D. Shimaoka, A. S. Chuong, N. C. Klapoetke, L. Li, A. van der Bourg, Y. Niino, L. Egolf, C. Monetti, H. Gu, M. Mills, A. Cheng, B. Tasic, T. N. Nguyen, S. M. Sunkin, A. Benucci, A. Nagy, A. Miyawaki, F. Helmchen, R. M. Empson, T. Knöpfel, E. S. Boyden, R. C. Reid, M. Carandini, H. Zeng, Transgenic mice for intersectional targeting of neural sensors and effectors with high specificity and performance, Neuron 85, 942-958 (2015).
60. M. W. Zappaterra, A. S. LaMantia, C. A. Walsh, M. K. Lehtinen, Isolation of cerebrospinal fluid from rodent embryos for use with dissected cerebral cortical explants., J. Vis. Exp. (2013), doi:10.3791/50333.
61. K. J. Livak, T. D. Schmittgen, Analysis of relative gene expression data using real-time quantitative PCR and the 2-ΔΔCT method, Methods 25, 402-408 (2001).
62. M. Czosnyka, Z. Czosnyka, K. J. Agarwal-Harding, J. D. Pickard, Modeling of CSF dynamics: Legacy of professor Anthony Marmarou, Acta Neurochir. Suppl. 113, 9-14 (2012).
63. T. B. Bennike, M. D. Bellin, Y. Xuan, A. Stensballe, F. T. Møller, G. J. Beilman, O. Levy, Z. Cruz-Monserrate, V. Andersen, J. Steen, D. L. Conwell, H. Steen, A Cost-Effective High-Throughput Plasma and Serum Proteomics Workflow Enables Mapping of the Molecular Impact of Total Pancreatectomy with Islet Autotransplantation, J. Proteome Res. 17, 1983-1992 (2018).
64. J. Cox, M. Mann, MaxQuant enables high peptide identification rates, individualized p.p.b.-range mass accuracies and proteome-wide protein quantification, Nat. Biotechnol. 26, 1367-1372 (2008).
65. T. Akaishi, T. Takahashi, I. Nakashima, M. Abe, M. Aoki, T. Ishii, Osmotic pressure of serum and cerebrospinal fluid in patients with suspected neurological conditions, Neural Regen. Res. 15, 944-947 (2020).

What is claimed is:

1. A method for restoring cerebrospinal fluid ionic homeostasis in a subject, the method comprising administering to a subject in need thereof an agent that increases NKCC1 activity, expression, or level in a cell, thereby restoring cerebrospinal fluid ionic homeostasis in the subject.

2. A method for mitigating an intracranial fluid imbalance associated with a hemorrhage in a subject, the method comprising administering to a subject in need thereof an agent that increases NKCC1 activity, expression, or level in a cell, thereby mitigating the intracranial fluid imbalance.

3. A method for treating a cerebrospinal fluid disorder in a subject, the method comprising administering to a subject in need thereof an agent that increases NKCC1 activity, expression, or level in a cell, thereby treating the cerebrospinal fluid disorder.

4. The method of claim 1, wherein the cell is a choroid plexus cell or an epithelial cell.

5. The method of claim 1, wherein the agent comprises a polypeptide, polynucleotide, or small molecule chemical compound.

6. The method of claim 5, wherein the polynucleotide is a polynucleotide encoding an NKCC1 polypeptide or wherein the polynucleotide is an AAV2/5 vector comprising an NKCC1 polynucleotide.

7. The method of claim 3, wherein the cerebrospinal fluid disorder is associated with a congenital disorder, a trauma, or an ischemic event.

8. The method of claim 7, wherein the cerebrospinal fluid disorder is hydrocephalus, hyperkalirrhachia, or ventriculomegaly.

9. The method of claim 1, wherein the subject has or has propensity to develop an intraventricular hemorrhage or wherein the subject has a loss-of-function CHD4 mutation.

10. The method of claim 1, wherein the subject is a human prenatal, neonatal, pediatric, or adult subject.

11. The method of claim 1, wherein the agent is administered by intracerebroventricular injection.

12. The method of claim 1, wherein administration of the agent is associated with a decrease in cerebrospinal fluid levels, an increase in cerebrospinal fluid clearance, a decrease in intracranial pressure, or a decrease in ventricle size.

13. A cell comprising the vector of claim 5.

14. A vector comprising a polynucleotide encoding NKCC1.

15. A method for treating a subject having or having a propensity to develop an intraventricular hemorrhage, the method comprising administering to the subject an adeno-associated virus (AAV) vector comprising a polynucleotide encoding an NKCC1 polypeptide, thereby increasing NKCC1 polypeptide expression levels in choroid plexus epithelial cells in the subject and reducing an intracranial fluid imbalance associated with the intraventricular hemorrhage.

16. The method of claim 15, wherein the AAV vector is an AAV2/5 vector.

17. The method of claim 15, wherein the increase in NKCC1 polypeptide expression levels is associated with a reduction in incidence or severity of hydrocephalus and/or ventriculomegaly.

18. The method of claim 15, wherein the increase in NKCC1 polypeptide expression levels is associated with an increase in cerebrospinal fluid K⁺ clearance, increased cerebrospinal compliance, and reduced circulating cerebrospinal fluid in the brain.

19. The method of claim 15, wherein intracranial pressure is reduced or remains unchanged following the increase in NKCC1 polypeptide expression levels.

20. The method of claim 15, wherein the agent is administered by intracerebroventricular injection.

21. A kit for use in the method of claim 1, wherein the kit comprises the agent that increases NKCC1 activity, expression, or level in a cell.


SEQUENCE LISTING (2 sequences)

SEQ ID NO: 1 - protein, 1196 aa, Homo sapiens
MEPRPTAPSSGAPGLAGVGETPSAAALAAARVELPGTAVPSVPEDAAPASRDGGGVRDEGPAAAGDGLGRPLGPTPSQSRFQVDLVSENAGRAAAAAAAAAAAAAAAGAGAGAKQTPADGEASGESEPAKGSEEAKGRFRVNFVDPAASSSAEDSLSDAAGVGVDGPNVSFQNGGDTVLSEGSSLHSGGGGGSGHHQHYYYDTHTNTYYLRTFGHNTMDAVPRIDHYRHTAAQLGEKLLRPSLAELHDELEKEPFEDGFANGEESTPTRDAVVTYTAESKGVVKFGWIKGVLVRCMLNIWGVMLFIRLSWIVGQAGIGLSVLVIMMATVVTTITGLSTSAIATNGFVRGGGAYYLISRSLGPEFGGAIGLIFAFANAVAVAMYVVGFAETVVELLKEHSILMIDEINDIRIIGAITVVILLGISVAGMEWEAKAQIVLLVILLLAIGDFVIGTFIPLESKKPKGFFGYKSEIFNENFGPDFREEETFFSVFAIFFPAATGILAGANISGDLADPQSAIPKGTLLAILITTLVYVGIAVSVGSCVVRDATGNVNDTIVTELTNCTSAACKLNFDFSSCESSPCSYGLMNNFQVMSMVSGFTPLISAGIFSATLSSALASLVSAPKIFQALCKDNIYPAFQMFAKGYGKNNEPLRGYILTFLIALGFILIAELNVIAPIISNFFLASYALINFSVFHASLAKSPGWRPAFKYYNMWISLLGAILCCIVMFVINWWAALLTYVIVLGLYIYVTYKKPDVNWGSSTQALTYLNALQHSIRLSGVEDHVKNFRPQCLVMTGAPNSRPALLHLVHDFTKNVGLMICGHVHMGPRRQAMKEMSIDQAKYQRWLIKNKMKAFYAPVHADDLREGAQYLMQAAGLGRMKPNTLVLGFKKDWLQADMRDVDMYINLFHDAFDIQYGVVVIRLKEGLDISHLQGQEELLSSQEKSPGTKDVVVSVEYSKKSDLDTSKPLSEKPITHKESKGPIVPLNVADQKLLEASTQFQKKQGKNTIDVWWLFDDGGLTLLIPYLLTTKKKWKDCKIRVFIGGKINRIDHDRRAMATLLSKFRIDFSDIMVLGDINTKPKKENIIAFEEIIEPYRLHEDDKEQDIADKMKEDEPWRITDNELELYKTKTYRQIRLNELLKEHSSTANIIVMSLPVARKGAVSSALYMAWLEALSKDLPPILLVRGNHQSVLTFYS

SEQ ID NO: 2 - DNA, 6826 bp, Homo sapiens
  source 1..6826: mol_type = other DNA
acactcgcgcgctcgctcggctgccggtggcctctgtggccgtccaggctagcggcggcccgcaggcggcggggagaaagactctctcacctggtcttgcggctgtggccaccgccggccaggggtgtggagggcgtgctgccggagacgtccgccgggctctgcagttccgccgggggtcgggcagctatggagccgcggcccacggcgccctcctccggcgccccgggactggccggggtcggggagacgccgtcagccgctgcgctggccgcagccagggtggaactgcccggcacggctgtgccctcggtgccggaggatgctgcgcccgcgagccgggacggcggcggggtccgcgatgagggccccgcggcggccggggacgggctgggcagacccttggggcccaccccgagccagagccgtttccaggtggacctggtttccgagaacgccgggcgggccgctgctgcggcggcggcggcggcggcggcagcggcggcggctggtgctggggcgggggccaagcagacccccgcggacggggaagccagcggcgagagcgagccggctaaaggcagcgaggaagccaagggccgcttccgcgtgaacttcgtggacccagctgcctcctcgtcggctgaagacagcctgtcagatgctgccggggtcggagtcgacgggcccaacgtgagcttccagaacggcggggacacggtgctgagcgagggcagcagcctgcactccggcggcggcggcggcagtgggcaccaccagcactactattatgatacccacaccaacacctactacctgcgcaccttcggccacaacaccatggacgctgtgcccaggatcgatcactaccggcacacagccgcgcagctgggcgagaagctgctccggcctagcctggcggagctccacgacgagctggaaaaggaaccttttgaggatggctttgcaaatggggaagaaagtactccaaccagagatgctgtggtcacgtatactgcagaaagtaaaggagtcgtgaagtttggctggatcaagggtgtattagtacgttgtatgttaaacatttggggtgtgatgcttttcattagattgtcatggattgtgggtcaagctggaataggtctatcagtccttgtaataatgatggccactgttgtgacaactatcacaggattgtctacttcagcaatagcaactaatggatttgtaagaggaggaggagcatattatttaatatctagaagtctagggccagaatttggtggtgcaattggtctaatcttcgcctttgccaacgctgttgcagttgctatgtatgtggttggatttgcagaaaccgtggtggagttgcttaaggaacattccatacttatgatagatgaaatcaatgatatccgaattattggagccattacagtcgtgattcttttaggtatctcagtagctggaatggagtgggaagcaaaagctcagattgttcttttggtgatcctacttcttgctattggtgatttcgtcataggaacatttatcccactggagagcaagaagccaaaagggttttttggttataaatctgaaatatttaatgagaactttgggcccgattttcgagaggaagagactttcttttctgtatttgccatcttttttcctgctgcaactggtattctggctggagcaaatatctcaggtgatcttgcagatcctcagtcagccatacccaaaggaacactcctagccattttaattactacattggtttacgtaggaattgcagtatctgtaggttcttgtgttgttcgagatgccactggaaacgttaatgacactatcgtaacagagctaacaaactgtacttctgcagcctgcaaattaaactttgatttttcatcttgtgaaagcagtccttgttcctatggcctaatgaacaacttccaggtaatgagtatggtgtcaggatttacaccactaatttctgcaggtatattttcagccactctttcttcagcattagcatccctagtgagtgctcccaaaatatttcaggctctatgtaaggacaacatctacccagctttccagatgtttgctaaaggttatgggaaaaataatgaacctcttcgtggctacatcttaacattcttaattgcacttggattcatcttaattgctgaactgaatgttattgcaccaattatctcaaacttcttccttgcatcatatgcattgatcaatttttcagtattccatgcatcacttgcaaaatctccaggatggcgtcctgcattcaaatactacaacatgtggatatcacttcttggagcaattctttgttgcatagtaatgttcgtcattaactggtgggctgcattgctaacatatgtgatagtccttgggctgtatatttatgttacctacaaaaaaccagatgtgaattggggatcctctacacaagccctgacttacctgaatgcactgcagcattcaattcgtctttctggagtggaagaccacgtgaaaaactttaggccacagtgtcttgttatgacaggtgctccaaactcacgtccagctttacttcatcttgttcatgatttcacaaaaaatgttggtttgatgatctgtggccatgtacatatgggtcctcgaagacaagccatgaaagagatgtccatcgatcaagccaaatatcagcgatggcttattaagaacaaaatgaaggcattttatgctccagtacatgcagatgacttgagagaaggtgcacagtatttgatgcaggctgctggtcttggtcgtatgaagccaaacacacttgtccttggatttaagaaagattggttgcaagcagatatgagggatgtggatatgtatataaacttatttcatgatgcttttgacatacaatatggagtagtggttattcgcctaaaagaaggtctggatatatctcatcttcaaggacaagaagaattattgtcatcacaagagaaatctcctggcaccaaggatgtggtagtaagtgtggaatatagtaaaaagtccgatttagatacttccaaaccactcagtgaaaaaccaattacacacaaagaatccaaaggccctattgtgcctttaaatgtagctgaccaaaagcttcttgaagctagtacacagtttcagaaaaaacaaggaaagaatactattgatgtctggtggctttttgatgatggaggtttgaccttattgataccttaccttctgacgaccaagaaaaaatggaaagactgtaagatcagagtattcattggtggaaagataaacagaatagaccatgaccggagagcgatggctactttgcttagcaagttccggatagacttttctgatatcatggttctaggagatatcaataccaaaccaaagaaagaaaatattatagcttttgaggaaatcattgagccatacagacttcatgaagatgataaagagcaagatattgcagataaaatgaaagaagatgaaccatggcgaataacagataatgagcttgaactttataagaccaagacataccggcagatcaggttaaatgagttattaaaggaacattcaagcacagctaatattattgtcatgagtctcccagttgcacgaaaaggtgctgtgtctagtgctctctacatggcatggttagaagctctatctaaggacctaccaccaatcctcctagttcgtgggaatcatcagagtgtccttaccttctattcataaatgttctatacagtggacagccctccagaatggtacttcagtgcctagtgtagtaactgaaatcttcaatgacacattaacatcacaatggcgaatggtgacttttctttcacgatttcattaatttgaaagcacacaggaaagttgctccattgataacgtgtatggagacttcggttttagtcaattccatatctcaatcttaatggtgattcttctctgttgaactgaagtttgtgagagtagttttcctttgctacttgaatagcaataaaagcgtgttaactttttgattgatgaaagaagtacaaaaagcctttagccttgaggtgccttctgaaattaaccaaatttcatccatatatcctcttttataaacttatagaatgtcaaactttgccttcaactgtttttatttctagtctcttccactttaaaacaaaatgaacactgcttgtcttcttccattgaccatttagtgttgagtactgtatgtgttttgttaattctataaaggtatctgttagatattaaaggtgagaattagggcaggttaatcaaaaatggggaaggggaaatggtaaccaaaaagtaaccccatggtaaggtttatatgagtatatgtgaatatagagctaggaaaaaaagcccccccaaatacctttttaacccctctgattggctattattactatatttattattatttattgaaaccttagggaagattgaagattcatcccatacttctatataccatgcttaaaaatcacgtcattctttaaacaaaaatactcaagatcatttatatttatttggagagaaaactgtcctaatttagaatttccctcaaatctgagggacttttaagaaatgctaacagatttttctggaggaaatttagacaaaacaatgtcatttagtagaatatttcagtatttaagtggaatttcagtatactgtactatcctttataagtcattaaaataatgtttcatcaaatggttaaatggaccactggtttcttagagaaatgtttttaggcttaattcattcaattgtcaagtacacttagtcttaatacactcaggtttgaacagattattctgaatattaaaatttaatccattcttaatattttaaaacttttgttaagaaaaactgccagtttgtgcttttgaaatgtctgttttgacatcatagtctagtaaaattttgacagtgcatatgtactgttactaaaagctttatatgaaattattaatgtgaagtttttcatttataattcaaggaaggatttcctgaaaacatttcaagggatttatgtctacatatttgtgtgtgtgtgtgtatatatatgtaatatgcatacacagatgcatatgtgtatatataatgaaatttatgttgctggtattttgcattttaaagtgatcaagattcattaggcaaactttggtttaagtaaacatatgttcaaaatcagattaacagatacaggtttcatagagaacaaaggtgatcatttgaagggcatgctgtaatttcacacaattttccagttcaaaaatggagaatacttcgcctaaaatactgttaagtgggttaattgatacaagtttctgtggtggaaaatttatgcaggttttcacgaatccttttttttttttttttttttttttgagacggagtcttgctctgttgccacgctggaatgcagtaacgtgatcttggctcactgcgacctccacctccccagttcaagcgattctcctgcctcagcctccctagtagctgggactacgggtgcacgccaccatgcccagctaatttttgtattttgagtagagacagggtttcaccgtgttggctaggatggtgtctatctcttgaccttgtgatccacccgcctcagcctcccagagtgctgggattacaggtgcgagccactgcgcctggctggttttcatgaatcttgatagacatctataacgttattattttcagtggtgtgcagcatttttgcttcatgagtatgacctaggtatagagatctgataacttgaattcagaatattaagaaaatgaagtaactgattttctaaaaaaaaaaaaaaaaaaaatttctacattataactcacagcattgttccattgcaggttttgcaatgtttgggggtaaagacagtagaaatattattcagtaaacaataatgtgtgaacttttaagatggataatagggcatggactgagtgctgctatcttgaaatgtgcacaggtacacttaccttttttttttttttttttaagtttttcccattcaggaaaacaacattgtgatctgtactacaggaaccaaatgtcatgcgtcatacatgtgggtataaagtacataaaatatatctaactattcataatgtggggtgggtaatactgtctgtgaaataatgtaagaagcttttcacttaaaaaaaatgcattactttcacttaacactagacaccaggtcgaaaattttcaaggttatagtacttatttcaacaattcttagagatgctagctagtgttgaagctaaaaatagctttatttatgctgaattgtgatttttttatgccaaattttttttagttctaatcattgatgatagcttggaaataaataattatgccatggcatttgacagttcattattcctataagaattaaattgagtttagagagaatggtggtgttgagctgattattaacagttactgaaatcaaatatttatttgttacattattccatttgtattttaggtttccttttacattctttttatatgcattctgacattacatattttttaagactatggaaataatttaaagatttaagctctggtggatgattatctgctaagtaagtctgaaaatgtaatattttgataatactgtaatatacctgtcacacaaatgcttttctaatgttttaaccttgagtattgcagttgctgctttgtacagaggttactgcaataaaggaagtggattcattaaaccta